(19) United States
(12) Patent Application Publication
(10) Pub. No.: US 2022/0130493 A1
(43) Pub. Date: Apr. 28, 2022

(54) METHOD AND SYSTEMS FOR PHYTOMEDICINE ANALYTICS FOR RESEARCH OPTIMIZATION AT SCALE

(71) Applicant: GBS Global BioPharma, Inc., Ottawa (CA)

(72) Inventors: Helen C. Turner, Honolulu, HI (US); Alexander J. Stokes, Honolulu, HI (US); Andrea L. Small-Howard, Norwalk, CA (US)

(21) Appl. No.: 17/501,498

(22) Filed: Oct. 14, 2021

Related U.S. Application Data
(60) Provisional application No. 63/091,816, filed on Oct. 14, 2020.
(60) Provisional application No. 63/221,334, filed on Jul. 13, 2021.
(60) Provisional application No. 63/221,358, filed on Jul. 13, 2021.
(60) Provisional application No. 63/221,364, filed on Jul. 13, 2021.
(60) Provisional application No. 63/221,366, filed on Jul. 13, 2021.
(60) Provisional application No. 63/221,367, filed on Jul. 13, 2021.
(60) Provisional application No. 63/221,371, filed on Jul. 13, 2021.

(51) Int. Cl. G16C 20/40; G16C 20/70

(52) U.S. Cl. CPC G16C 20/40; G16C 20/70

(57) ABSTRACT

Disclosed herein are phytomedicine analytics for research optimization at scale (PhAROS) methods for discovering and/or optimizing polypharmaceutical medicines, the PhAROS method comprising: analyzing, in a single computational space, data from a plurality of traditional medicine systems (TMS), wherein the analysis uses transcultural dictionaries to allow searches within distinct TMS data sets embodying different epistemologies and terminologies, Wherein the analysis uses data returned by a query to identify new polypharmaceutical and/or optimized polypharmaceutical compositions.

### 1. CROSS-REFERENCE TO RELATED APPLICATIONS

This application claims the benefit of and priority to U.S. Provisional Application Nos. 63/091,816, filed Oct. 14, 2020; 63/221,334, filed Jul. 13, 2021; 63/221,358, filed Jul. 13, 2021; 63/221,364, filed Jul. 13, 2021; 63/221,366, filed Jul. 13, 2021; 63/221,367, filed Jul. 13, 2021; and 63/221,371, filed Jul. 13, 2021, the disclosures of which are hereby incorporated by reference in their entireties.

### 2. BACKGROUND

The metabolomes of plants, fungi and other prokaryotic and eukaryotic organisms contain bioactive molecules that can affect physiological and pathophysiological processes if introduced into living human and animal biological systems. Contemporary pharmacological discovery practices analyze these compounds by screening large repositories of thousands of individual compounds to observe putative biological effects, and outcomes in cell lines and model organisms and diseases. The screening and characterization of individual compounds is laborious and costly. Current biopharmaceutical research and development programs are highly inefficient at yielding newly approved drugs for government-regulated, prescription-based markets. Therefore, methods for increasing the efficiency of both drug discovery and the prediction of clinical efficacy of new disease-specific therapies from within contemporary natural product metabolomes are needed.

The bioactive molecules in the metabolomes of plants, fungi, and other prokaryotic and eukaryotic organisms have implicitly been used as the basis for traditional medicines (TM) that incorporate ethno medical beliefs and traditions specific to individual cultures, as well as traditional medical systems practiced in multiple locales. The World Health Organization (WHO) defines traditional medicine as “the sum total of the knowledge, skills, and practices based on the theories, beliefs, and experiences indigenous to different cultures, whether explicable or not, used in the maintenance of health as well as in the prevention, diagnosis, improvement or treatment of physical and mental illness” (World Health Organization, 2013). Each culture has its set of ethno medical beliefs and practices associated with health and illness, which shape diagnosis, treatment, and expected outcome.

Pathways for potentially efficacious medicines to move from contemporary and historical TM systems to government-regulated, prescription-based, medical systems are currently inadequate, relying on either (a) painstaking, high cost, compound-by-compound testing of TM pharmacopeias in pharmaceutical company-sponsored preclinical and clinical efficacy paradigms, or (b) on ‘rediscovery’ of components during high-throughput screening in academic or pharmaceutical industry research settings. Current pathways for medicines to move from TM systems to Western medicine are inefficient and unsatisfactory due to: (1) Over-simplification—the diminution of complex efficacious polypharmaceutical mixtures to a single component results in loss of synergies and interactions between components, and/or (2) Epistemology—TM formulations contain both efficacious bioactive components and chemicals for which the inclusion rationale is anachronistic or pseudoscientific, and these need to be differentiated. There is a need to identify the ‘Goldilocks’ formulation for a particular indication, where the minimal essential complexity that reflects the polypharmacutical nature of the TM is preserved and excess or irrelevant components are omitted.

Moreover, since contemporary and historical TM systems are inherently polypharmaceutical while government-regulated, prescription-based medical system approaches are typically ‘single drug-single target’, simple preclinical or clinical screening will miss compounds that only work when contextualized by other components.

Contemporary and historical TM pharmacopeias are also highly siloed along cultural dividing lines, tending to be examined in isolation by scientists from the originating country. This misses opportunities to identify consonant approaches that are duplicated across pharmacopeias, which could help pre-validate drug-target-indication relationships. In addition, it misses a major opportunity to combine efficacious components across cultural lines to design optimal new polypharmaceutical medicines.

Other challenges exist in modernizing, unlocking, and deconvolving the inherent knowledge in contemporary and historical traditional medical systems. Side-by-side comparisons of databases, for example, performed for Traditional Chinese Medicines (TCM), highlight issues with completeness, redundancy, and inconsistency, especially in the dating (and therefore rapid aging) of the source data on plant-chemical composition linkages. Currency and real-time updating are major data management issues in this field. Unification and integration of databases within TCM have been called for, recognizing the current fractured state of resources. These same issues persist in databases of other TM; therefore, there is a need for unification and integration of databases across multiple TM. There is also currently a missed opportunity for integration with data layers that reflect the wealth of biomedical data available in the era of the ‘omics revolution.

Other salient weaknesses of extant TM databases include a lack of ability to weigh for content (i.e., researchers focus on the chemical composition rather than on the proportion of each compound in a formulation) which limits moves to assign priorities to compounds when assembling novel formulations informed by the traditional medical system. The lack of consideration of the contributions of microorganisms that form stable, associated microbiomes of medicinal plants/fungi is also a weakness of current network pharmacology. These associated microorganisms have their own secondary metabolomes that may contribute to formulations in currently unrecognized ways. They may also pro-biotically, anti-biotically, or pre-biotically, be interacting with the patient's gut-microbiome axis and therefore influencing ADME and pharmacodynamics.

There is a need for an AI/ML-enabled drug discovery platform that would increase the efficiency and accuracy of the discovery of novel multi-component therapies from natural products and that would predict the potential efficacy of these novel multi-component therapies using analyses within an integrated and layered TM dataset with the appropriate applications of machine learning and deep learning modules.

### 3. SUMMARY

The present invention addresses the following needs in the art: a) to increase efficiency and accuracy of the identification of novel, multi-component therapeutics based on compounds derived from the metabolomes of plants, fungi, and other prokaryotic and eukaryotic organisms; b) to further increase the efficiency and accuracy of the identification of novel, multi-components therapeutics based on the manner that active compounds derived from the metabolomes of plants, fungi, and other prokaryotic and eukaryotic organisms are used in and substantially informed by the epistemology of contemporary and historical TM systems; c) to predict the efficacy of novel multi-component therapeutics based on convergence analysis of drug-target-indication relationships in these multi-component mixtures across multiple contemporary and historical TM systems; d) to unify and integrate the databases from as many contemporary and historical TM systems as possible; e) to layer additional epistemological, translational, ecological, and relative content (% API) information onto the contemporary and historical TM systems; and f) to provide flexibility in the system for queries originating with disease, target, compound, organism, and others that would result in the identification and prediction of efficacy for a novel, multi-compound therapeutic.

Embodiments of the present disclosure may include a method of effectively and rapidly transferring and importing very large traditional medicine datasets, efficiently reducing the size of the data (without losing the integrity of the data), translating, comparing, normalizing, analyzing, and assessing the data, correlating with intradata variables, and metadata, as well as other external datasets, displaying, sorting, ranking and visualizing the data for viewing by the user, using specialized methods and systems designed to manage the large extent of the data. Through multiple interfaces, the system allows the user to interact with the data, tabulate in various ways, and use graphical representations, zoom in or out, re-plot on different axes, re-scale, pick specific data of interest, refine and redefine data queries based on user data interaction with tabular, menu and graphical selections and groupings, as well as graphical gating, to initiate further, and subsequent processing depending on the user's questions, hypotheses and use case.

User choices, algorithmic processing, and machine learning algorithms can be initiated, and utilized to identify; specific patterns of interest, targets for subsequent processing, metadata groupings that correlate with indications across traditional medicines, identification of missing plants, components or compounds from specific plants or across the whole dataset, identification of unknown indications for traditional medicines, identification of toxic and non-toxic components and compounds, identification of plant, component and compound mixtures with ranked therapeutic potential, identification of plant, component and compound combination that would not be obvious, and/or would have greater therapeutic potential, than existing mixtures in isolated traditional medicines.

Additionally, the method may include, in silico processing to simulate and thus predict therapeutic phenotypic results, disease treatment outcomes, that have yet to be assessed in real-world analysis, testing, clinical trials, or laboratory-based experiments. This saves the resources needed to perform real-world assessment and renders tractable pharmaceutical problems that have previously been impossible to address using extant technologies.

Aspects of the present disclosure include a phytomedicine analytics for research optimization at scale (PhAROS) method for discovering and/or optimizing polypharmaceutical medicines. The PhAROS method comprises: analyzing, in a single computational space, data from a plurality of traditional medicine systems (TMS), wherein the analysis uses transcultural dictionaries to allow searches within distinct TMS data sets embodying different epistemologies and terminologies, wherein the analysis uses data returned by a query to identify new polypharmaceutical and/or optimized polypharmaceutical compositions.

In some embodiments, the data from the plurality of TMS comprise at least one of: medical formulations; organisms; medical compound data sets; therapeutic indications; processed and normalized formalized pharmacopeias from one or more geographic regions associated with TMS; therapeutic indication dictionaries related to traditional medical systems that reflect modern and historical terminology; Western and non-Western epistemologies; temporal and geographical data indicating historical and contemporary geographical, cultural and epistemology origins; raw and optionally pre-processed data from a plurality of traditional medicine data sets, plant data sets, and literature-based text documents (corpus).

In some embodiments, the one or more geographic regions is selected from: Japan, China, India, Korea, South East Asia, Middle East, North America, South America, Russia, India, Africa, Europe, and Australia.

In some embodiments, the one or more processed and normalized formalized pharmacopeias comprises at least one of processed data, translated normalized data, individual published datasets, or case reports in the scientific literature that document relationships between medicinal plants and disease indications.

In some embodiments, the one or more processed and normalized formalized pharmacopeias comprises at least one of processed data, curated ethical partnerships, indigenous phytomedical formulations, and cultural (African, Oceanic) phytomedical formulations.

In some embodiments, the one or more processed and normalized formalized pharmacopeias comprises processed contemporary and historical herbologies that document relationships between medicinal plants and disease indications, wherein the herbologies are optionally selected from Hildegard of Bingen, Causae et Curae, and Physica.

In some embodiments, the one or more processed and normalized formalized pharmacopeias comprises processed translations from original languages, wherein the process uses methods selected from one or more of: machine literal translation, natural language processing, multilingual concept extraction or conventional translation, Optical character recognition (OCR) of historical materials, and artificial intelligence (AI)-driven intent translation.

In some embodiments, the medical compound data sets comprise chemical and biological data of medical compounds.

In some embodiments, the chemical and biological data of medical compounds comprise one or more of: chemical structure, physicochemical properties, known and/or algorithmically calculated or predicted PD/PK properties, putative biological effects, data with respect to receptor binding, docking, regulation of signaling pathways, metabolism, drug-target relationships, mechanism of action, CYP interactions, or published studies and clinical trials of the medical compounds.

In some embodiments, the raw and optionally pre-processed data normalized from a plurality of traditional medicine data sets comprises one or more of: meta-pharmacopeia associated temporally, geographical, botanical, climatological, environmental, genomic, metagenomic, and metabolomic data on originating plants, components or other organisms; meta-pharmacopeias with de novo metabolomic data for plants and organisms that are not currently in medicinal use, supplemental metabolomic data secured for known medicinal plants and/or associated organisms; and toxicological and side-effect profile data of medical compound data sets, de novo experimentally-derived data of medical compound data sets, and/or in silico predicted toxicological and side-effect data of medical compound data sets.

In some embodiments, analyzing comprises, first, receiving a user query from a user.

In some embodiments, analyzing comprises, second, using the user query to search the data in the plurality of TMS for data that are associated with the first user query input.

In some embodiments, analyzing comprises, third, processing the searched data to create processed data.

In some embodiments, analyzing comprises, fourth, outputting the processed data for review by the user.

In some embodiments, analyzing comprises, fifth, optionally further processing the processed data if further requested by the user.

In some embodiments, analyzing comprises outputting the processed data returned by the query to the user for review by the user or for further analysis initiated by a second user query to identify the new polypharmaceutical and/or optimized polypharmaceutical compositions.

In some embodiments, processing the searched data comprises performing an in silico convergence analysis to search drug-target-indication relationships associated with the user query input. In some embodiments, processing the searched data comprises performing an in silico convergence analysis comprising identifying commonalities between two or more of: a disease, a therapeutic indication, one or more compounds derived from one or more organisms, and therapeutic approaches from biogeographically and culturally separated locales, coincidence or convergence of one or more compounds across a plurality of TMS, and coincidence or convergence of one or more organisms across a plurality of TMS.

In some embodiments, the in silico convergence analysis further comprises using processed data returned by the query to rank new polypharmaceutical compositions for subsequent preclinical and clinical testing for a given therapeutic indication.

In some embodiments, processing the searched data from the plurality of TMS using the in silico convergence analysis predicts efficacy of the new and/or optimized polypharmaceutical compositions.

In some embodiments, processing the searched data from the plurality of TMS using the in silico convergence analysis identifies minimal essential compounds required for efficacy of the new and/or optimized polypharmaceutical compositions.

In some embodiments, processing the searched data comprises performing an in silico divergence analysis to search drug-target-indication relationships associated with the user query input.

In some embodiments, processing the searched data comprises performing an in silico divergence analysis comprising identifying alternative compounds derived from one or more organisms, and therapeutic approaches from biogeographically and culturally separated locales across the plurality of TMS.

In some embodiments, the in silico divergence analysis further comprises using processed data returned by the query to rank new polypharmaceutical compositions for subsequent preclinical and clinical testing for a given therapeutic indication.

In some embodiments, processing the searched data from the plurality of TMS using the in silico divergence analysis predicts efficacy of the new and/or optimized polypharmaceutical compositions.

In some embodiments, a first user input query comprises one or more user selected clinical indications. In some embodiments, the one or more user selected clinical indications is selected from cancer, cancer pain, and cancer and cancer pain.

In some embodiments, outputting the processed data returned by the query comprises outputting: a list of compounds associated with the user selected clinical indication, a list of prescription formulae for a given TMS, a list of organisms associated with the user selected clinical indication, or a combination thereof.

In some embodiments, the outputting comprises outputting a list of compounds that is associated with a first user selected clinical indication, wherein the list of compounds that is associated with the first user selected clinical indication does not overlap with a list of compounds that is associated with a second user selected indication.

In some embodiments, the new polypharmaceutical and/or optimized polypharmaceutical compositions comprise one or more compounds derived from metabolomes of prokaryotic, Archaea, or eukaryotic organisms.

In some embodiments, the new polypharmaceutical and/or optimized polypharmaceutical compositions comprise one or more compounds derived from metabolomes of plants or fungi.

In some embodiments, the optimized polypharmaceutical compositions comprise one or more substitution compounds of an existing transcultural medicinal formulation.

In some embodiments, the optimized polypharmaceutical composition comprises a reduced number of compounds within the optimized polypharmaceutical composition as compared to an existing transcultural medicinal formulation, wherein the optimized polypharmaceutical composition comprises a minimal number of essential compounds to achieve a therapeutic outcome.

In some embodiments, further analysis includes, after outputting one or more selected from: developing training data sets for one or more machine learning models to optimize the transcultural dictionaries; populating the transcultural dictionaries with additional data developed by a machine learning algorithm; and creating, updating, annotating, processing, downloading, analyzing, or manipulating the data from the plurality of TMS.

In some embodiments, the method further includes iteratively training the one or more machine learning models with the one or more training data sets. In some embodiments, method further includes applying a machine learning model to identify the new polypharmaceutical and/or optimized polypharmaceutical compositions. In some embodiments, the machine learning model is iteratively trained with one or more training data sets. In some embodiments, the machine learned model comprises a set of rules, wherein the set of rules are configured to: identify specific patterns of interest, therapeutic targets for subsequent processing, metadata groupings that correlate with indications across traditional medicines, identify missing plants, components or compounds, identify unknown indications for traditional medicines, identify toxic and non-toxic components and compounds, identify plant, component and compound mixtures with ranked therapeutic potential, identify plant, component and compound combination that would not be obvious or have greater therapeutic potential, than existing mixtures in isolated traditional medicines. In some embodiments, the method includes applying the machine-learned model to identify the new polypharmaceutical and/or optimized polypharmaceutical compositions.

In some embodiments, at least one transcultural dictionary of the transcultural dictionaries comprises a search dictionary that collates Western and non-Western epistemological understanding of migraine and migraine-like patient presentations.

In some embodiments, populating the transcultural dictionaries with additional data developed by the machine learning algorithm comprises generating a therapeutic indication dictionary.

In some embodiments, the first user input query comprises one or more user selected clinical indications.

In some embodiments, the one or more user selected clinical indications is migraine.

In some embodiments, the outputting the processed data returned by the query comprises outputting: a list of compounds associated with the user selected clinical indication, a list of prescription formulae for a given TMS associated with the user selected clinical indication, or a combination thereof.

In some embodiments, the list of compounds is ranked by efficacy with statistical significance.

In some embodiments, the outputting further comprises outputting molecular targets for the list of compounds that are clinically indicated for migraine across one or more TMS.

In some embodiments, the molecular targets comprise: Prelamin-A/C; Lysine-specific demethylase 4D-like; Microtubule-associated protein tau; Microtubule-associated protein tau; Endonuclease 4; Peripheral myelin protein 22; Nonstructural protein 1; Bloom syndrome protein; Bloom syndrome protein; Neuropeptide S receptor; Geminin; Histone-lysine N-methyltransferase, H3 lysine-9 specific 3; Geminin; Thioredoxin reductase 1, cytoplasmic; Acetylcholinesterase; Cholinesterase; Solute carrier organic anion transporter family member 1B1; Solute carrier organic anion transporter family member 1B3 Nuclear factor NF-kappa-B p65 subunit; p53-binding protein Mdm-2; Huntingtin; Ras-related protein Rab-9A; Survival motor neuron protein; Tyrosyl-DNA phosphodiesterase 1; Microtubule-associated protein tau; Microtubule-associated protein tau; Microtubule-associated protein tau; Nuclear receptor ROR-gamma; Aldehyde dehydrogenase 1A1; Thioredoxin glutathione reductase; 4′-phosphopantetheinyl transferase ffp; 4′-phosphopantetheinyl transferase ffp; Nonstructural protein 1; Microtubule-associated protein tau; Microtubule-associated protein tau; Type-1 angiotensin II receptor; Niemann-Pick C1 protein; MAP kinase ERK2; Nuclear receptor ROR-gamma; Alpha-galactosidase A; DNA polymerase beta; Beta-glucocerebrosidase; Nuclear factor erythroid 2-related factor 2; X-box-binding protein 1; Histone acetyltransferase GCN5; G-protein coupled receptor 55; Histone-lysine N-methyltransferase, H3 lysine-9 specific 3; DNA damage-inducible transcript 3 protein; ATPase family AAA domain-containing protein 5; Vitamin D receptor; Vitamin D receptor; Chromobox protein homolog 1; Thioredoxin reductase 1, cytoplasmic; DNA polymerase iota; DNA polymerase eta; Regulator of G-protein signaling 4; Beta-galactosidase; Regulator of G-protein signaling 4; Mothers against decapentaplegic homolog 3; Geminin; Alpha trans-inducing protein (VP16); ATPase family AAA domain-containing protein 5; ATPase family AAA domain-containing protein 5; ATPase family AAA domain-containing protein 5; DNA dC->dU-editing enzyme APOBEC-3G; Photoreceptor-specific nuclear receptor; Geminin; Ataxin-2; Glucagon-like peptide 1 receptor; ATPase family AAA domain-containing protein 5; ATPase family AAA domain-containing protein 5; ATPase family AAA domain-containing protein 5; ATPase family AAA domain-containing protein 5; Tyrosyl-DNA phosphodiesterase 1; Isocitrate dehydrogenase [NADP] cytoplasmic; Tyrosyl-DNA phosphodiesterase 1; Transcriptional activator Myb; Transcriptional activator Myb; Ubiquitin carboxyl-terminal hydrolase 1; Parathyroid hormone receptor; ATPase family AAA domain-containing protein 5; ATPase family AAA domain-containing protein 5; Telomerase reverse transcriptase; Telomerase reverse transcriptase Survival motor neuron protein; Thyroid hormone receptor beta-1; Arachidonate 15-lipoxygenase; Chromobox protein homolog 1; Geminin; Guanine nucleotide-binding protein G(s), subunit alpha; Pregnane X receptor; Nuclear receptor subfamily 1 group I member 2; Nuclear receptor subfamily 1 group I member 3; Pregnane X receptor; Pregnane X receptor; Pregnane X receptor; Pregnane X receptor; Nuclear receptor subfamily 1 group I member 2; Nuclear receptor subfamily 1 group I member 2; Pregnane X receptor; Pregnane X receptor; Nuclear receptor subfamily 1 group I member 2; Nuclear receptor subfamily 1 group I member 2; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; and Nuclear receptor subfamily 1 group I member 3.

In some embodiments, the second user query input comprises the list of compounds.

In some embodiments, further analysis initiated by the second user query input comprising the list of compounds comprises post-hoc screening for toxicity, chemical activity, or toxicity and chemical activity of the list of compounds.

In some embodiments, further analysis comprises using the second user query input to search the data from the plurality of TMS associated with the second user query input.

In some embodiments, further analysis comprises processing the data associated with the second user query input to create a second processed data returned by the second query user input.

In some embodiments, further analysis comprises processing the data associated with the second user query input to create a second processed data returned by the second query user input, and retrieving the second processed data based on the second query input for review by the user.

In some embodiments, the second processed data comprises a ranked list of potential minimal essential compounds required for efficacy of the new and/or optimized polypharmaceutical compositions.

In some embodiments, the list of compounds is categorized by class, identified as migraine dictionary search results, and are convergent between a plurality of TMS.

In some embodiments, the method further comprises further analysis initiated by a third user query input to identify the new polypharmaceutical and/or optimized polypharmaceutical compositions.

In some embodiments, further analysis comprises processing the data associated with the third user query input to create a third processed data returned by the query, and retrieving and outputting the third processed data based on the third user query input for review by the user.

In some embodiments, the third user query input comprises a query of neurotropic fungi associated with migraines in the plurality of TMS. In some embodiments, the third processed data comprises one or more convergent compounds considered as alternative compounds of an existing transcultural compound with convergence between a plurality of TMS.

In some embodiments, the user query input comprises one or more phytomedical compounds or formulations, and optionally a current source (plant or animal) and supply of the compound or formulation.

In some embodiments, the processed data comprises a list of plant sources, known clinical indications associated with the phytomedical compounds or formulations and the TMS in which each compound was referenced. In some embodiments, the processed data further comprises a relative abundance of the one or more compounds or formulations, wherein the relative abundance is the relative amount of the one or more compounds or formulations available. In some embodiments, the processed data further comprises growing locations of the list of plant sources. In some embodiments, the processed data is cross ranked by one or more of frequency, relative abundance, availability, potency, and supply.

In some embodiments, analyzing comprises outputting the processed data returned by the query to the user for review by the user or for further analysis initiated by a second user query input to identify the new polypharmaceutical and/or optimized polypharmaceutical compositions. In some embodiments, the new polypharmaceutical and/or optimized polypharmaceutical compositions comprise one or more compounds derived from metabolomes of an alternative source of plants or fungi that were not previously identified for a specific use or indication. In some embodiments, the optimized polypharmaceutical compositions comprise one or more substitution compounds of an existing transcultural medicinal formulation, wherein a source origin of the substitution compound is not found in an existing transcultural medicinal formulation.

In some embodiments, populating the transcultural dictionaries with additional data developed by the machine learning algorithm comprises generating a therapeutic indication dictionary. In some embodiments, at least one transcultural dictionary of the transcultural dictionaries comprises a search dictionary that collates Western and non-Western epistemological understanding of pain, pain-like patient symptoms.

In some embodiments, the first user input query comprises a user selected clinical indication. In some embodiments, the user selected clinical indication is pain.

In some embodiments, the processed data returned by the query comprises: a list of compounds associated with pain, a list of prescription formulae associated with pain, a list of organisms associated with pain, a list of chemicals associated with pain, or a combination thereof. In some embodiments, the list of compounds, prescription formulae, organisms, and chemicals are indicated for pain across one or more TMS. In some embodiments, the processed data further comprises: the identity of each TMS identified by an in silico convergent analysis, each TMS linked to one or more of: a number of compounds within the list of compounds associated with pain, a number of prescription formulae within the list of prescription formulae associated with pain, a number of organisms within the list of organisms associated with pain, and a number of chemicals within the list of chemicals associated with pain.

In some embodiments, the list of compounds comprises a list of alkaloids or terpenes. In some embodiments, the list of compounds comprises: a list of opioids and/or alkaloid candidate analgesics, a list of ligands for nociceptive ion channels, a list of compounds with demonstrated neuroactivity, a list of compounds with bioactivity, and a list of compounds with bioactivity associated with pain.

In some embodiments, the second user query input comprises the list of compounds.

In some embodiments, further analysis initiated by the second user query input comprising the list of compounds comprises post-hoc screening for toxicity, chemical activity, or toxicity and chemical activity of the list of compounds. In some embodiments, further analysis comprises using the second user query input to search the data from the plurality of TMS, the data from the plurality of TMS associated with the second user query input. In some embodiments, further analysis comprises processing the data associated with the second user query input to create a second processed data returned by the second query user input, and retrieving the second processed data based on the second query input for review by the user.

In some embodiments, the second processed data comprises a ranked list of potential minimal essential compounds required for efficacy of the new and/or optimized polypharmaceutical compositions for treating pain.

In some embodiments, the second processed data comprises a second list of compounds ranked by one or more of: class, target, pathway, and coincidence or convergence of each of the compounds across specific TMS. In some embodiments, the second processed data comprises a list of convergent compounds within the list of compounds between one or more TMS. In some embodiments, the convergent compounds within the list of convergent compounds is considered as alternative compounds of an existing transcultural compound convergent between or more TMS.

In some embodiments, the list of compounds comprises a list of alkaloids, convergent between two or more TMS and associated with pain. In some embodiments, the list of alkaloids comprises: niacin, berberine, palmatine, trigonelline, jatrorrhizine, d-pseudoephedrine, candicine, protopine, stachydrine, harmane, liriodenine, caffeine, sinoacutine, ephedrine, niacinamide, 3-hydroxytyramine, anonaine, magnoflorine, sanguinarine, cryptopine, piperine, dihydrosanguinarine, papaverine, codeine, narcotoline, higenamine, roemerine, gentianine, xanthine, theophylline, ricinine, morphine, pelletierine, meconine, narceine, xanthaline, harmine, and reserpine.

In some embodiments, the list of compounds comprises a list of terpenes convergent between one or more TMS and associated with pain. In some embodiments, the list of terpenes comprise: alpha-pinene, linalool, terpineol, oleanolic acid, beta-sitosterol, p-cymene, myrcene, beta-bisabolene, beta-humulene, carvacrol, beta-caryophyllene, gamma-terpinene, geraniol, 1,8-cineole, alpha-farnesene, limonene, ursolic acid, beta-selinene, terpilene, spinasterol, beta-eudesmol, citral, sabinene, stigmasterol, limonene, beta-elemenene, d-cadinene, terpinene-4-ol, uralenic acid, borneol, beta-pinene, limonin, camphene, campesterol, citronellal, isocyperol, ruscogenin, crocetin, squalene, brassicasterol, piperitenone, lycopene, toralactone, phytofluene, alpha-carotene, ecdysone, neomenthol, auroxanthin, soyasapogenol-e, cyasterone, neodihydrocarveol, guaiazulene, alpha-pinene, crataegolic acid, violaxanthin, and pathoulene.

In some embodiments, the user input query is pain type. In some embodiments, the processed data returned by the query comprises: a list of pain types across one or more TMS. In some embodiments, the list of pain types comprises: abdominal, cardiac/chest, mouth, muscle, back, inflammation, joint, eye, chronic pain/inflammation, labor/postpartum, skin, throat, limb, bone, breast, ear, pelvic, intestinal, anal, pain sensitivity, rib, neuropathic, bladder, kidney, lung, menstruation, facial, liver, arthritis, fallopian tube, urethra, and vaginal, pain.

In some embodiments, for each pain type, the processed data comprises a list of TMS referenced from the plurality of TMS, associated with the pain type. In some embodiments, the processed data returned by the query comprises a list of compounds associated with each pain type. In some embodiments, the processed data further comprises a list of organisms for which the compounds within the list of compounds is derived. In some embodiments, the processed data comprises the list of pain types and a list of organisms, wherein one or more pain types is associated with one or more organisms.

In some embodiments, the processed data comprises the list of pain types and a list of compounds, wherein one or more pain types is associated with one or more compounds.

In some embodiments, for each pain type, the processed data comprises identity of a plurality of TMS linked to one or more selected from: the pain type, one or more compounds associated with the pain type, and one or more organisms associated with the pain type.

In some embodiments, at least one transcultural dictionary of the transcultural dictionaries comprises a search dictionary that collates Western and non-Western epistemological understanding of Piper species associated with a therapeutic indication.

In some embodiments, populating the transcultural dictionaries with additional data developed by the machine learning algorithm comprises generating a dictionary for Piper species.

In some embodiments, the therapeutic indication is selected from pain, sedation, anxiety, depression, epilepsy, mood, and sleep. In some embodiments, the therapeutic indication is selected from: hydropisy, gout, acne, coma, generalized hypopigmentation of hair, abnormal intrinsic pathway, abnormal female internal genitalia, pterygium, pain, gout, apoplexy, atony, headache, cancer giddiness, ring worm, epilepsy, otalgia, sciatica, hallucinations, alopecia, leucoderma/vitiligo, paralysis/hemiplegia, quartan fever ichthyosis, arthralgia, ptyriasis alba, congenital deafness alopecia furfuracea, hepatic obstruction, psychosis/insanity/mania, diseases of head and neck, bronchial asthma scrofula/cervical lymphadenitis, paroxysmal fever/intermittent fever bellas palsy, cramp/convulsion/spasm, strangury/dribbling of urine flaccidity, dyspnea, tremor, vertigo, tenesmus, poisoning flatulence, jaundice, toothache, hemorrhage, arthritis, lumbago backache, urinary incontinence, colic, weakness of stomach, sexual debility/anaphrodisia, palpitation, delerium, ptyriasis nigra, gastric dyscrasia, piles/ano rectal mass/haemorrhoids, fever with vata predominance, fatigue, insect bite, phlegmetic cough, splenic obstruction, blurring of vision, night blindness, corneal opacity, indigestion, vata-kaphaja, oedema/inflammation, anemia, chronic obstructive jaundice/chlorosis, cough/bronchitis, emaciation/cachexia, seminal disorders, pulmonary cavitation, gaseous/flatulence, disease with kapha predominance, tubercular cough/cough due to weakness or emaciation, pyrexia, diseases of spleen, dyspepsia/loss of appetite sprue/malabsorption syndrome, urinary disorders/polyuria curable disease of severe nature, obesity, cholera, asthma insomnia, sedative, diarrhea, anorexia, dysentery, dyspepsia, gonorrhea, rheumatism, bronchitis, cholagogue, emmenagogue, abdominal lump, angina pectoris, pleurodynia and intercostal neuralgia, stiffness, dryness of mouth, diseases of the mouth, diseases of head, and disease with vata predominance.

In some embodiments, the user input query comprises a list of Piper species of the family Piperaceae. In some embodiments, outputting the processed data returned by the query comprises outputting: a list of Piper species associated with one or more therapeutic indications.

In some embodiments, the one or more therapeutic indications is selected from pain, sedation, anxiety, depression, epilepsy, mood, and sleep. In some embodiments, the therapeutic indication is selected from: hydropisy, gout, acne, coma, generalized hypopigmentation of hair, abnormal intrinsic pathway, abnormal female internal genitalia, pterygium, pain, gout, apoplexy, atony, headache, cancer giddiness, ring worm, epilepsy, otalgia, sciatica, hallucinations, alopecia, leucoderma/vitiligo, paralysis/hemiplegia, quartan fever ichthyosis, arthralgia, ptyriasis alba, congenital deafness alopecia furfuracea, hepatic obstruction, psychosis/insanity/mania, diseases of head and neck, bronchial asthma scrofula/cervical lymphadenitis, paroxysmal fever/intermittent fever bellas palsy, cramp/convulsion/spasm, strangury/dribbling of urine flaccidity, dyspnea, tremor, vertigo, tenesmus, poisoning flatulence, jaundice, toothache, hemorrhage, arthritis, lumbago backache, urinary incontinence, colic, weakness of stomach, sexual debility/anaphrodisia, palpitation, delerium, ptyriasis nigra, gastric dyscrasia, piles/ano rectal mass/haemorrhoids, fever with vata predominance, fatigue, insect bite, phlegmetic cough, splenic obstruction, blurring of vision, night blindness, corneal opacity, indigestion, vata-kaphaja, oedema/inflammation, anemia, chronic obstructive jaundice/chlorosis, cough/bronchitis, emaciation/cachexia, seminal disorders, pulmonary cavitation, gaseous/flatulence, disease with kapha predominance, tubercular cough/cough due to weakness or emaciation, pyrexia, diseases of spleen, dyspepsia/loss of appetite sprue/malabsorption syndrome, urinary disorders/polyuria curable disease of severe nature, obesity, cholera, asthma insomnia, sedative, diarrhea, anorexia, dysentery, dyspepsia, gonorrhea, rheumatism, bronchitis, cholagogue, emmenagogue, abdominal lump, angina pectoris, pleurodynia and intercostal neuralgia, stiffness, dryness of mouth, diseases of the mouth, diseases of head, and disease with vata predominance.

In some embodiments, outputting the processed data returned by the query comprises outputting: the list of Piper species that are convergent across one or more TMS using the in silico convergent analysis. In some embodiments, the list of Piper species comprises Piper attenuatum, Piper betle, Piper boehmeriaefolium, Piper borbonense, Piper capense, Piper chaba, Piper cubeba, Piper cubeba, Piper cubeba, Piper cubeba, Piper futokadsura, Piper futokadzura, Piper guineense, Piper hamiltonii, Piper kadsura, Piper kadsura, Piper laetispicum, Piper longum, Piper longum, Piper longum, Piper longum, Piper mullesua, Piper nigrum, Piper nigrum, Piper nigrum, Piper nigrum, Piper nigrurml., Piper puberulum, Piper pyrifolium, Piper retrofractum, Piper retrofractum, Piper retrofractum, Piper schmidtii, Piper sylvaticum, Piper sylvestre, and Piper umbellatum.

In some embodiments, each Piper species within the list of Piper species is associated with one or more TMS, therapeutic indications within the one or more TMS, sets of chemical components linked to each Piper species and associated with the therapeutic indication, or a combination thereof.

In some embodiments, the list of chemical components for the list of piper species associated with the therapeutic indication, anxiety, comprises piperine, guineensine, piperlonguminine, unk, arecaidine, arecoline, beta-cadinene, beta-carotene, beta-caryophyllene, carvacrol, chavicol, diosgenin, estragole, eucalyptol, eugenol, gamma-terpinene, p-cymene, 1-triacontanol, 4-allyl-1,2-diacetoxybenzene, 4-allylbenzene-1,2-diol, 4-aminobutyric acid, allylpyrocatechol, calcium, dl-alanine-15n, dl-arginine, dl-asparagine, dl-aspartic acid, dl-valine, glutamate, glycine, hentriacontane, hydrogen oxalate, 1-ascorbic acid, 1-leucine, 1-methionine, l-proline, 1-serine, 1-threonine, malic acid, methyleugenol, nicotinate, octadecanoate, orn, phenylalanine, phytosterols, retinol, riboflavin, tyrosine cation radical, vitamin e, 4-allylcatechol, norcepharadione b, piperolactam a, piperolactam c, unk, unk, piperine, piperlongumine, d-fructose, d-glucose, phytosterols, (+)-sesamin, (−)-hinokinin, (−)-yatein, 1,4-cineole, 1,8-cineol, 1,8-cineole, 1-4-cineol, alpha-cubebene, alpha-pinene, alpha-terpinene, alpha-terpineol, beta-bisabolene, beta-caryophyllene, beta-cubebene, beta-pinene, caryophyllene, cineol, d-limonene, delta-cadinene, dipentene, gamma-terpinene, humulene, ledol, limonene, linalol, linalool, myrcene, ocimene, p-cymene, piperine, sabinene, terpineol, (+)-sabinene, (+)-zeylenol, (−)-clusin, (−)-cubebinin, (−)-cubebininolide, 2,4,5-trimethoxybenzaldehyde, allo-aromadendrene, alpha-muurolene, alpha-phellandrene, alpha-thujene, apiole, asarone, aschantin, azulene, beta-elemene, beta-phellandrene, bicyclosesquiphellandrene, cadinene, calamene, calamenene, copaene, cubebin, cubebinolide, cubebol, cubenol, dillapiole, eo, epicubenol, gamma-humulene, heterotropan, muurolene, nerolidol, piperenol a, piperenol b, piperidine, sabinol, safrole, terpinolene, (+)-4-iso-propyl-1-methyl-cyclohex-1-en-4-ol, (+)-car-4-ene, (+)-crotepoxide “(−)-5-o-methoxy-hinokinin” (−)-cadinene, (−)-cubebinone, (−)-di-o-methyl-thujaplicatin methyl ether, (−)-dihydro-clusin, (−)-dihydro-cubebin, (−)-isoyatein, 1-isopropyl-4-methylene-7-methyl-1,2,3,6,7,8,9-heptahydro . . . , 10-(alpha)-cadinol, “3(r)-3-4-dimethoxy-benzyl-2(r)-3-4-methylenedioxy-benzyl-butyrolactone”, alpha-o-ethyl-cubebin, beta-o-ethyl-cubebin, cadina-1-9(15)-diene, cesarone, cubebic acid, d-delta-4-carene, gum, hemi-ariensin, 1-cadinol, manosalin, resinoids, resins, trans-terpinene, (e)-citral, (z)-citral, citral, dihydroanhydropodorhizol, dihydrocubebin “(8r,8r)-4-hydroxycubebinone”, “(8r,8r,9s)-5-methoxyclusin”, 1-(2,4,5-trimethoxyphenyl)-1,2-propanedione, cubeben camphor, cubebin, ethoxyclusin, heterotropan, magnosalin, (+)-cubenene, (+)-delta-cadinene, 1,4-cineole, arachidic acid, beta-cadinene, dihydrocubebin, docosanoic acid, eucalyptol, hinokinin, oleic acid, palmitic acid, yatein, (+)-piperenol b, (+)-sabinene, (+)-zeylenol, (−)-clusin, (−)-cubebinin, (−)-cubebininolide, (−)-dihydroclusin “(8r,8r)-4-hydroxycubebinone”, “(8r,8r,9s)-5-methoxyclusin” 1-epi-bicyclosesquiphellandrene, 2,4,5-trimethoxybenzaldehyde, alpha-muurolene, calamenene, chemb1501119, chemb1501260, crotepoxide, cubebin, cubebinone, cubebol, cyclohexane, epizonarene, ethoxyclusin, hexadecenoic acid, isohinokinin, isoyatein, 1-asarinin, lignans machilin f, octadeca-9,12-dienoic acid, octadecanoate, picrotoxinum, piperidine, thujaplicatin, unii-5vg84p9unh, zonarene, (+)-deoxy, (+)-piperenol a, acetic acid-((r)-6,7-methylenedioxy-3-piperonyl-1,2-dihydro-2naphthylmethyl ester), cubebinol, hibalactone, isocubebinic ether, podorhizon, unk, unk, unk, unk, kadsurin a, isodihydrofutoquinol b, denudatin b, kadsurenone, elemicin, futoquinol, kadsurin a, sitosterol, î′-sitosterol, stigmasterol, (+)-acuminatin, (e,7s,11r)-3,7,11,15-tetramethylhexadec-2-en-1-ol, phytol, (â±)-galgravin, 4-(2r,3r,4s,5s)-5-(1,3-benzodioxol-5-yl)-3,4-dimethyl-2-tetrahydrofuranyl-2-methoxyphenol, machilin f, asaronaldehyde, asarylaldehyde, chicanine, crotepoxide, futoxide, futoamide, futoenone, futokadsurin a, futokadsurin b, futokadsurin c, galbacin, galbelgin, kadsurenin b, kadsurenin c, kadsurenin k, kadsurenin l, kadsurenin m, machilusin, n-isobutyldeca-trans-2-trans-4-dienamide, piperlactam s, veraguensin, zuonin a, unk, artecanin, unk, piperine, piperitenone, piplartine, pisatin, sesamin, undulatone, 1,2,15,16-tetrahydrotanshiquinone, 1-undecylenyl-3,4-methylenedioxybenzene, guineensine, hexadecane, laurotetanine, lawsone, piperidine, piperlonguminine, sesamol, beta-caryophyllene, p-cymene, piperine, piperlongumine, 2-phenylethanol “4-methoxyacetophenone”, 6,7-dibromo-4-hydroxy-1h,2h,3h,4h-pyrrolol,2-apyrazin-1-one, alpha thujene, aristololactam, diaeudesmin, dihydrocarveol, eicosane, ent-zingiberene, fargesin, guineensine, heneicosane, heptadecane, hexadecane, 1-asarinin, lignans machilin f, methyl 3,4,5-trimethoxycinnamate, nonadecane, octadecane, phytosterols, piperlonguminine, pipernonaline, piperundecalidine, pluviatilol, terpinolene, triacontane, (2e,4e)-n-isobutyl-2,4-decadienamide, isobutyl amide, unk, yangonin, 10-methoxyyangonin, 11-methoxyyangonin, 11-hydroxyyangonin, desmethoxyyangonin, 11-methoxy-12-hydroxydehydrokavain, 7,8-dihydroyangonin, kavain, 5-hydroxykavain, 5,6-dihydroyangonin, 7,8-dihydrokavain, 5,6,7,8-tetrahydroyangonin, 5,6-dehydromethysticin, methysticin, 7,8-dihydromethysticin, (−)-bornyl ferulate, (−)-bornyl-caffeate, (−)-bornyl-p-coumarate, 1-cinnamoylpyrrolidine, 11-hydroxy-12-methoxydihydrokawain, 2,5,8-trimethyl-1-napthol, 3,4-methylene dioxy cinnamic acid, 3a,4a-epoxy-5b-pipermethystine, 5-methyl-1-phenylhexen-3-yn-5-ol, 5,6,7,8-tetrahydroyangonin2, 9-oxononanoic acid, benzoic acid, bornyl cinnamate, caproic acid, cinnamalacetone, cinnamalacetone2, cinnamic acid, desmethoxyyangonin, dihydro-5,6-dehydrokawain, dihydro-5,6-dehydrokawain2, dihydrokavain, dihydrokavain2, dihydromethysticin, flavokawain a, flavokawain b, flavokawain c, glutathione, methysticin2, mosloflavone, octadecadienoic acid methyl ester, p-hydroxy-7,8-dihydrokavain, p-hydroxykavain, phenyl acetic acid, pipermethystine, prenyl caffeate, nectandrin b, neferine, (+)-limonene, 1,8-cineole, alpha-bulnesene, alpha-cubebene, alpha-guaiene, alpha-gurjunene, alpha-humulene, alpha-pinene, alpha-terpinene, alpha-terpineol, alpha-terpineol acetate, alpha-trans-bergamotene, arachidic acid, astragalin, behenic acid, beta-bisabolene, beta-carotene, beta-caryophyllene, beta-cubebene, beta-farnesene, beta-pinene, beta-selinene, beta-sitosterol, borneol, butyric acid, caffeic acid, campesterol, camphene, camphor, carvacrol, caryophyllene, cedrol, cinnamic acid, cis-carveol, citral, d-limonene, delta-cadinene, dl-limonene, eugenol, fat, gamma-terpinene, hexanoic acid, hyperoside, isocaryophyllene, isoquercitrin, kaempferol, l-alpha-phellandrene, 1-limonene, lauric acid, limonene, linalol, linalool, linoleic acid, monoterpenes, myrcene, myristic acid, myristicin, myrtenal, myrtenol, niacin, ocimene, oleic acid, p-coumaric acid, p-cymene, palmitic acid, perillaldehyde, piperine, quercetin, quercitrin, rhamnetin, rutin, sabinene, sesquiterpenes, stearic acid, stigmasterol, trans-carveol, trans-pinocarveol, (−)-cubebin, (z)-ocimenol, 1(7),2-p-menthadien-4-ol, 1(7),2-p-menthadien-6-ol, 1-terpinen-4-ol, 1-terpinen-5-ol, 2,8-p-menthadien-1-ol, 2-methyl-pentanoic acid, 2-undecanone, 3,8(9)-p-menthadien-1-ol, 3-methyl-butyric acid, 4-methyl-triacontane, acetophenone, alpha-bisabolene, alpha-copaene, alpha-linolenic acid, alpha-phellandrene, alpha-santalene, alpha-selinene, alpha-thujene, alpha-tocopherol, alpha-zingiberene, ar-curcumene, ascorbic acid, benzoic acid, beta-bisabolol, beta-caryophyllene alcohol, beta-elemene, beta-phellandrene, beta-pinone, boron, calamene, calamenene, calcium, car-3-ene, carvetonacetone, carvone, caryophyllene alcohol, caryophyllene-oxide, chavicine, chlorine, choline, chromium, cis-nerolidol, cis-ocimene, cis-p-2-menthen-1-ol, citronellal, citronellol, clovene, cobalt, copper, cryptone, cubebine, cuparene, delta-3-carene, delta-elemene, dihydrocarveol, dihydrocarvone, elemol, eo, feruperine, fluoride, gaba, gamma-cadinene, gamma-muurolene, germacrene-b, germacrene-d, globulol, guineensine, heliotropin, hentriacontan-16-ol, hentriacontan-16-one, hentriacontane, hentriacontanol, hentriacontanone, iodine, iron, isochavicine, isopiperine, isopulegol, limonen-4-ol, lipase, magnesium, manganese, methyl-eugenol, n-formylpiperidine, n-hentriacontane, n-heptadecane, n-nonadecane, n-nonane, n-pentadecane, n-tridecane, nerolidol, nickel, oxalic acid, p-cymen-8-ol, p-cymene-8-ol, p-menth-8-en-1-ol, p-menth-8-en-2-ol, p-methyl-acetophenone, pellitorine, phenylacetic acid, phosphorus, phytosterols, piperanine, pipercide, piperettine, pipericine, piperidine, piperitone, piperonal, piperonic acid, piperylin, piperyline, potassium, pyrrolidine, pyrroperine, retrofractamide-a, riboflavin, safrole, sesquisabinene, silica, sodium, spathulenol, starch, sulfur, terpinen-4-ol, terpinolene, thiamin, thujene, tocopherols, trans-nerolidol, trichostachine, ubiquinone, water, zinc, (−)-3,4-dimethoxy-3,4-demethylenedioxy-cubebin, (−)-phellandrene, 1,1,4-trimethylcyclohepta-2,4-dien-6-one, 1,8(9)-p-menthadien-4-ol, 1,8(9)-p-menthadien-5-ol, 1,8-menthadien-2-ol, 1-(2,4-decadienoyl)-pyrrolidine, 1-(2,4-dodecadienoyl)-pyrrolidine, 1-alpha-phellandrene, 1-piperyl-pyrrolidine, 2-trans-4-trans-8-trans-piperamide-c-9-3, 2-trans-6-trans-piperamide-c-7-2, 2-trans-8-trans-piperamide-c-9-2, 2-trans-piperamide-c-5-1, 3,4-dihydroxy-6-(n-ethyl-amino)-benzamide, 4,10,10-trimethyl-7-methylene-bicyclo-(6.2.0)decane-4-car . . . , 4-methyl-tritriacontane, 5,10(15)-cadinen-4-ol, 6-trans-piperamide-c-7-1, 8-trans-piperamide-c-9-1, acetyl-choline, alpha-amorphene, alpha-cis-bergamotene, alpha-cubebine, beta-cubebine, carvone-oxide, caryophylla-2,7(15)-dien-4-beta-ol, caryophylla-2,7(15)-dien-4-ol, caryophylla-3(12),7(15)dien-4-beta-ol, caryophyllene-ketone, cis-2,8-menthadien-2-ol, cis-sabinene-hydrate, cis-trans-piperine, citronellyl-acetate, cumaperine, dihydropipercide, epoxydihydrocaryophyllene, eugenol-methyl-ether, geraniol-acetate, geranyl-acetate, isobutyl-caproate, isobutyl-isovalerate, isochavinic acid, kaempferol-3-o-arabinosyl-7-o-rhamnoside, linalyl-acetate, m-mentha-3(8),6-diene, m-methyl-acetophenone, methyl-caffeic acid-piperidide, methyl-carvacrol, methyl-cinnamate, methyl-cyclohepta-2,4-dien-6-one, methyl-heptanoate, methyl-octanoate, n-(2-methylpropyl)-deca-trans-2-trans-4-dienamide, n-5-(4-hydroxy-3-methoxy-phenyl)-pent-trans-2-dienoyl-piperidine, n-butyophenone, n-heptadecene, n-isobutyl-11-(3,4-methylenedioxy-phenyl)-undeca-trans-2-trans-4-trans-10-trienamide, n-isobutyl-13-(3,4-methylenedioxy-phenyl)-trideca-trans-2-trans-4-trans-12-trienamide, n-isobutyl-eicosa-trans-2-trans-4-cis-8-trienamide, n-isobutyl-eicosa-trans-2-trans-4-dienamide, n-isobutyl-octadeca-trans-2-trans-4-dienamide, n-methyl-pyrroline, n-pentadecene, n-trans-feruloyl-piperidine, nerol-acetate, p-cymene-8-methyl-ether, p-menth-cis-2-en-1-ol, p-menth-trans-2-en-1-ol, phytin-phosphorus, piperolein-a, piperolein-b, piperolein-c, piperoleine-b, polysaccharides, quercetin-3-o-alpha-d-galactoside, rhamnetin-o-triglucoside, terpin-1-en-4-ol, terpinyl-acetate, trans-cis-piperine, trans-sabinene-hydrate, trans-trans-piperine, chavicol, pinocembrin, piperine, piperitenone, piplartine, trans-pinocarveol, 1(7),2-p-menthadien-4-ol, 1(7),2-p-menthadien-6-ol, 1(7),8(10)-p-menthadien-9-ol, 3,8(9)-p-menthadien-1-ol, chavicine, cis-p-2-menthen-1-ol, cryptone, cryptopimaric acid, dihydrocarveol, piperanine, piperettine, piperidine, piperitone, piperitylhonokiol, piperonal, sarmentosine, sesquisabinene, (+)-alpha-phellandrene, (+)-endo-beta-bergamotene, (−)-camphene, (−)-linalool, alpha-humulene, beta-caryophyllene, beta-pinene, capsaicin, d-citronellol, dipentene, eucalyptol, eugenol, gamma-terpinene, myrcene, p-cymene, piperine, testosterone, (+)-sabinene, (z)-.beta.-ocimenol, 1,8-menthadien-4-ol, 16-hentriacontanone, 2,6-di-tert-butyl-4-methylphenol, 3-carene, 7-epi-.alpha.-eudesmol, aclnahmy, acetic acid, alpha thujene, amide 4, beta-alanine, bicyclogermacrene, butylhydroxyanisole, carotene, caryophyllone oxide, cepharadione a, chebi:70093, cholesterol formate, cis-.alpha.-bergamotene, crypton, cubebin, Curcuma longa, dehydropipernonaline, dextromethorphan, dl-arginine, guineensine, hedycaryol, hentriacontane, isobutyramide, kakoul, 1-ascorbic acid, 1-serine, 1-threonine, menthadien-5-ol, methylenedioxycinnamic acid, moupinamide, nonane, octane, oxirane, p-anisidine, p-mentha-2,8-dien-1-ol, paroxetine, pellitorine, phytosterols, piperettine, piperidine, piperidine-2-carboxylic acid, pipernonaline, piperolactam d, piperolein a, piperolein b, piperonal, pyrocatechol, retrofractamide a, retrofractamide b, retrofractamide c, sarmentine, sodium nitroprussiate, tannic acid, terpinen-4-ol, trichostachine, wisanine, (2e,4e,8z)-n-isobutyl-eicosa-2,4,8-trienamide, (2e,4z)-5-(4-hydroxy-3-methoxyphenyl)-1-(1-piperidinyl)-2,4-pentadien-1-one, (e,e)-, 1-piperoyl-, n-idobutyl-13-(3,4-methylenedioxyphenyl)-2e,4e,12e-tridecatrienamide, pyrrolidine, unk, asarinin, grandisin, piperine, piperlonguminine, piplartine, sesamin, trans-pinocarveol, î″-fagarine, (+)-bornyl piperate, (1-oxo-3-phenyl-2e-propenyl)pyrrolidine, “(7r,8r)-3,4-methylenedioxy-4,7-epoxy-8,3-neolignan-7e-ene”, “(7s,8r)-4-hydroxy-4,7-epoxy-8,3-neolignan-(7e)-ene”, “(7s,8r)-4-hydroxy-8,9-dinor-4,7-epoxy-8,3-neolignan-7-aldehyde”, (â±)-erythro-1-(1-oxo-4,5-dihydroxy-2e-decaenyl)piperidine, (â±)-threo-1-(1-oxo-4,5-dihydroxy-2e-decaenyl)piperidine, (â±)-threo-n-isobutyl-4,5-dihydroxy-2e-octaenamide, 1(7),2-p-menthadien-4-ol, 1(7),2-p-menthadien-6-ol, 1-(1,6-dioxo-2e,4e-decadienyl)piperidine, 1-(1-oxo-2e,4e-dodedienyl)pyrrolidine, 1-(1-oxo-2e-decaenyl) piperidine, 1-(1-oxo-3-phenyl-2e-propenyl)piperidine, 1-1-oxo-3(3,4-methylenedioxy-5-methoxyphenyl)-2zpropenyl piperidine, 1-1-oxo-3(3,4-methylenedioxyphenyl)-2e-propenylpiperidine, 1-1-oxo-3(3,4-methylenedioxyphenyl)-2e-propenylpyrrolidine, 1-1-oxo-3(3,4-methylenedioxyphenyl)-2z-propenylpiperidine, 1-1-oxo-3(3,4-methylenedioxyphenyl)propylpiperidine, 1-1-oxo-5(3,4-methylenedioxyphenyl)-2e,4e-pentadienylpyrrolidine, 1-1-oxo-5(3,4-methylenedioxyphenyl)-2e,4z-pentadienyl pyrrolidine, 1-1-oxo-5(3,4-methylenedioxyphenyl)-2e,4z-pentadienylpiperidine, 1-1-oxo-5(3,4-methylenedioxyphenyl)-2z,4e-pentadienyl piperidine, 1-1-oxo-5(3,4-methylenedioxyphenyl)-2z,4e-pentadienyl pyrrolidine, 1-1-oxo-7(3,4-methylenedioxyphenyl)-2e,4e,6e-heptatrienylpyrrolidine, 1-1-oxo-9(3,4-methylenedioxyphenyl)-2e,8e-nonadienyl piperidine, pipernonaline, 1-terpinen-5-ol, 3,8(9)-p-menthadien-1-ol “4-desmethylpiplartine”, “5-hydroxy-7,3,4-trimethoxyflavone” cenocladamide, chavicine, cis-p-2,8-menthadien-1-ol, cis-p-2-menthen-1-ol, cryptone, dehydropipernonaline, guineensine, kaplanin, menisperine, methyl piperate, “methyl-(7r,8r)-4-hydroxy-8,9-dinor-4,7-epoxy-8,3-neolignan-7-ate”, n-isobutyl-(2e,4e)-octadecadienamide, n-isobutyl-(2e,4e)-octadienamide, n-isobutyl-(2e,4e,14z)-eicosatrienamide, n-isobutyl-2e,4e,12z-octadecatrienamide, n-isobutyl-2e,4e-dodedienamide, n-isobutyldeca-trans-2-trans-4-dienamide, neopellitorine b, pipataline, piperamide c 7:1(6e), piperamide c 9:1(8e), piperamide c 9:2(2e,8e), piperamide c 9:3(2e,4e,8e), piperamine, piperanine, piperchabamide a, piperchabamide b, piperchabamide c, piperchabamide d, pipercide, retrofractamide b, piperenol a, piperettine, piperitone, piperlonguminine, piperolactam a, piperolein a, piperolein b, piperonal, pipnoohine, pipyahyine, “rel-(7r,8r,7r,8r)-3,4-methylenedioxy-3,4,5,5-tetramethoxy-7,7-epoxylignan”, “rel-(7r,8r,7r,8r)-3,4,3,4-dimethylenedioxy-5,5-dimethoxy-7,7-epoxylignan”, retrofractamide a, retrofractamide b, sarmentine, sarmentosine, sesquisabinene, xanthoxylol, zp-amide a, zp-amide b, zp-amide c, zp-amide d, zp-amide e, n-isobutyl-4,5-dihydroxy-2e-decaenamide, n-isobutyl-4,5-epoxy-2e-decaenamide, pipercycliamide, wallichinine, unk, unk, brachystamide d, friedlein, phytosterols, unk, piperine, piperlongumine, 1-asarinin, phytosterols, piperine, asperphenamate, aurantiamide, phytosterols, piperettine, and sylvatine.

In some embodiments, the list of chemical components for at least one piper species comprises bis-noryangonin, 11-methoxy-nor-yangonin, 5,6-dehydrokawain, dihydromethysticin, and yangonin. In some embodiments, the at least one piper species is Piper methysticum.

In some embodiments, the second user query input for further analysis initiated by the second user query input comprises the list of chemical components: bis-noryangonin, 11-methoxy-nor-yangonin, 5,6-dehydrokawain, dihydromethysticin, and yangonin. In some embodiments, further analysis initiated by the second user query input comprising the list of chemical components comprises using the second user query input to search transcultural dictionaries, the data from the plurality of TMS associated with the second user query input. In some embodiments, further analysis comprises processing the data associated with the second user query input to create a second processed data returned by the second query user input, and retrieving the second processed data based on the second query user input. In some embodiments, the second processed data comprises a list of non-piper species comprising the list of chemical components. In some embodiments, the list of non-piper species comprises Petroselinum crispum, Dioscorea collettii, Dioscorea hypoglauca, Gentiana algida, Rubia cordifolia, and Alpinia speciosa. In some embodiments, processing the data associated with the second query user input comprises screening for non-piper species comprising the list of chemical components.

In some embodiments, further analysis comprises processing the data associated with the second user query input to create a second processed data returned by the second query user input, and retrieving the second processed data based on the second query user input.

In some embodiments, the second user query input comprises a biogeography of P. methysticum and a list of therapeutic indications, wherein the list of therapeutic indications comprises anxiety, mood, and depression.

In some embodiments, the second processed data comprises a list of non-piper species associated with anxiety, mood, depression, or a combination thereof found in non-piper species within the biogeography of P. methysticum.

In some embodiments, the list of non-piper species comprises Glycyrhizza uralensis/radix, Paeonia lactiflora, Scutellaria baicalensis, Panax ginseng, Saposhnikovia divaicata, and Poria cocos.

In some embodiments, populating the transcultural dictionaries with additional data developed by the machine learning algorithm comprises generating a therapeutic indication dictionary.

In some embodiments, at least one transcultural dictionary of the transcultural dictionaries comprises a search dictionary that collates Western and non-Western epistemological understanding of cancer, cancer-like patient presentations, cytotoxic agents within TMS formulations for cancer, and cancer pain.

In some embodiments, at least one transcultural dictionary of the transcultural dictionaries comprises a list of compounds associated with cancer pain, and a list of compounds known for treating pain.

In some embodiments, the first user input query comprises one or more user selected clinical indications. In some embodiments, the one or more user selected clinical indications is selected from cancer, cancer pain, and cancer and cancer pain.

In some embodiments, outputting the processed data returned by the query comprises outputting: a list of compounds associated with the user selected clinical indication, a list of prescription formulae for a given TMS, a list of organisms associated with the user selected clinical indication, or a combination thereof.

In some embodiments, the outputting further comprises outputting cytotoxic agents within the list of compounds that are indicated for pain and cancer across one or more TMS.

In some embodiments, outputting further comprises outputting the list of organisms associated with cancer and pain across one or more TMS.

In some embodiments, the list of compounds is categorized by class, identified as migraine dictionary hits, and are convergent between two or more TMS.

In some embodiments, the outputting further comprises outputting a list of compounds that is associated with a first user selected clinical indication, wherein the list of compounds that is associated with the first user selected clinical indication does not overlap with a list of compounds that is associated with a second user selected indication.

In some embodiments, the first user selected clinical indication is cancer, and the second user selected indication is pain.

Aspects of the present disclosure include a phytomedicine analytics for research optimization at scale (PhAROS) system for analyzing a plurality of traditional medical systems in a single computational space, the PhAROS system comprising: a computer server configured to communicate with one or more user clients (PhAROS_USER), comprising: (a) a database (PhAROS_BASE) comprising a memory configured to store a collection of data, the collection of data comprising: raw and optionally pre-processed data from a plurality of traditional medicine data sets; and optionally one or more of: plant data sets; literature-based text documents (corpus); and machine learning data sets; (b) a computer core processor (PhAROS_CORE), wherein the PhAROS_CORE is configured to receive and process the collection of data from the PhAROS_BASE to generate processed data; (c) one or more searchable repositories having data and optionally pre-processed data, wherein each searchable repository comprises a memory configured to store data entries, wherein the PhAROS_CORE is configured to send the processed data to and receive data from each of the searchable repositories, wherein each of the searchable repositories is configured to receive processed data from the PhAROS_CORE and send data and optionally pre-processed data to the PhAROS_CORE; (d) a computer-readable storage medium storing executable instructions that, when executed by a hardware processor, cause the PhAROS_CORE to communicate with the PhAROS_BASE and one or more of the searchable repositories to analyze data from a plurality of the traditional medicine data sets to produce an output responsive to a user query input into the PhAROS system.

In some embodiments, the PhAROS_CORE is further configured to manage, direct, collect, parse, and filter the collection of data from the PhAROS_BASE to generate processed data. In some embodiments, the PhAROS system further comprises one or more user clients (PhAROS_USER). In some embodiments, at least one PhAROS_USER client has a graphical user interface (GUI). In some embodiments, at least one PhAROS_USER client is configured to allow the user to communicate with the PhAROS_CORE. In some embodiments, at least one PhAROS_USER client is configured to allow the user to communicate with at least one of the searchable repositories. In some embodiments, at least one PhAROS_USER client is configured to allow the user to communicate with the PhAROS_CORE, PhAROS_BASE, and the searchable repositories.

In some embodiments, at least one searchable repository comprises: a first meta-pharmacopeia database (PhAROS_PHARM) comprising (i) data from PhAROS_BASE; and (ii) pre-processed data processed from data in the PhAROS_BASE related to at least one of: medical formulations; organisms; medical compound data sets; therapeutic indications; processed and normalized formalized pharmacopeias from one or more geographic regions associated with traditional medicines.

In some embodiments, the one or more geographic regions is selected from: Japan, China, India, Korea, South East Asia, Middle East, North America, South America, Russia, India, Africa, Europe, and Australia.

In some embodiments, the one or more processed and normalized formalized pharmacopeias comprises processed, translated normalized, individual published datasets or case reports in the scientific literature that document relationships between medicinal plants and disease indications.

In some embodiments, the one or more processed and normalized formalized pharmacopeias comprises processed, appropriate ethical partnerships, indigenous, cultural phytomedical formulations.

In some embodiments, the one or more processed and normalized formalized pharmacopeias comprises processed contemporary and historical herbologies that document relationships between medicinal plants and disease indications (e.g., Hildegard of Bingen, Causae et Curae, Physica).

In some embodiments, the one or more processed and normalized formalized pharmacopeias comprises processed, translation of resources from original languages processed using approaches selected from one or more of: machine literal translation, natural language processing, multilingual concept extraction or conventional translation, Optical character recognition (OCR) of historical materials, and artificial intelligence (AI)-driven intent translation.

In some embodiments, at least one searchable repository (PhAROS_CONVERGE) comprises data and pre-processed data that allow identification of commonalities in therapeutic approaches from biogeographically and culturally traditional medical systems (TMS). In some embodiments, the data and pre-processed data of the PhAROS_CONVERGE is further configured to allow identification of efficacious medical components across traditional medicine systems. In some embodiments, the data and pre-processed data of the PhAROS_CONVERGE is further configured to allow ranking optimization of de novo compound formulations and compound mixtures by utilizing transcultural components for subsequent preclinical and clinical testing for a given therapeutic indication.

In some embodiments, the data and pre-processed data of the PhAROS_CONVERGE comprises at least one of: therapeutic indication dictionaries related to traditional medical systems that reflect modern and historical terminology, and/or Western and non-Western epistemologies; medical formulation compositions related to traditional medical systems; compound data sets for a given therapeutic indication; and a proprietary digital composition index (n-dimensional vector and/or fingerprint).

In some embodiments, the computer-readable storage medium storing executable instructions, when executed by the hardware processor, cause the hardware processor to: develop training data sets for one or more machine learning algorithms to optimize the searchable repositories for a user; populate the one or more searchable repositories with additional data developed by the machine learning algorithm; and create, update, annotate, process, download, analyze, or manipulate the collection of data received by the Pharos_CORE. In some embodiments, the computer-readable storage medium storing executable instructions, when executed by the hardware processor, cause the PhAROS_CORE to: initiate a user to provide the user query input on the PhAROS_USER client, wherein the PhAROS_USER client is configured to communicate with the PhAROS_core and optionally the searchable repositories; search the user query input within the PhAROS_CORE, the searchable repositories, or a combination thereof; retrieve the processed data based on the user's query input for review by the user in PhAROS_USER; optionally initiate further processing of the retrieved processed data, if inquired by the user.

In some embodiments, the PhAROS_USER client further comprises a graphical data processing environment (PhAROS_FLOW) configured to allow the user to process data without or with reduced amount of at least one of: coding, system modeling tools comprising machine learning, or artificial intelligence (AI) tools.

In some embodiments, the machine learning and AI tools are selected from one or more of: support vector machine, artificial neural networks, deep learning, Naïve Bayesian, K-nearest neighbors, random forest, AdaBoost wisdom of crowds and ensemble predictors, and others, validation (such as MonteCarlo cross-validation, Leave-One-Out cross validation, Bootstrap Resampling, and y-randomization).

### 4. BRIEF DESCRIPTION OF THE DRAWINGS

FIG. 1A shows for illustrative purposes only an example of a client and server computer system of one embodiment.

FIG. 1B shows a block diagram of an overview of a remote user process, for access to a PhAROS system of one embodiment.

FIG. 1C shows a block diagram of an overview of a local user process, for access to the PhAROS system of one embodiment.

FIG. 1D shows a block diagram of an overview of an administrative user process, for access to the PhAROS platform server of one embodiment.

FIG. 2A shows for illustrative purposes only an example of a schematic of major subsystems of the PhAROS platform of one embodiment.

FIG. 2B shows for illustrative purposes only an example of a table describing the major systems and subsystems of the PhAROS platform, with icon key of one embodiment.

FIG. 2C shows for illustrative purposes only an example of a schematic of major systems and subsystems of the PhAROS platform, with icon key of one embodiment.

FIG. 2D shows for illustrative purposes only an example of a schematic of major systems and subsystems of the PhAROS platform, with user interaction description of one embodiment.

FIG. 3A shows for illustrative purposes only an example of a schematic of major sub-functions of the PhAROS_BRAIN system, indicating grouped PhAROS_BRAIN functions utilized by the PhAROS platform and PhAROS_USER, to create, update, annotate, process, download, analyze and manipulate data within the PhAROS system of one embodiment.

FIG. 3B shows for illustrative purposes only an example of a schematic of major sub-functions of the PhAROS_BRAIN system, and the PhAROS_FLOW subsystem utilized by the PhAROS system and PhAROS_USER subsystem, to create, update, annotate, process, download, analyze and manipulate data within the PhAROS system, utilizing a graphical no-code/low code worksheet environment, without the need for coding of one embodiment.

FIG. 4 shows for illustrative purposes only an example of a generalized example of a user interaction to the system through PhAROS_USER within the PhAROS systems and PhAROS subsystems of one embodiment.

FIG. 5 shows for illustrative purposes only an example of a generalized example of user interaction with the PhAROS system and PhAROS subsystems of one embodiment.

FIG. 6 shows for illustrative purposes only an example of a schematic of major components of the PhAROS system and subsystems, used in an example of importing data into the PhAROS_BASE system, and creation of a new database to contain this data of one embodiment.

FIG. 7 shows for illustrative purposes only an example of a Schematic of major systems and subsystems of the PhAROS platform, used in an example of processing, mining, and parsing specific data into the PhAROS_PHARM system, from multiple raw data sources in the PhAROS_BASE subsystem of one embodiment.

FIG. 8 provides a demonstration of the flexibility and adaptability of the PhAROS Drug Discovery Platform by outlining the progression from Input to Output through various PhAROS subsystems indicated with an “X”. In some embodiments, Input (1) a ‘Medical Condition’ produces Output(s) through the PhAROS process that include: ‘Ranked Compounds’ & ‘Ranked Minimum Essential Mixtures’. As in Example 1, Input (1) in this figure describes the progression (system and subsystem involvement that are indicated by an “X” in each corresponding PhAROS system/subsystem in that row) from Input to Output of the search for novel pain formulations in the PhAROS_PHARM database as described herein (See Example 1: Proof-of-Concept Demonstration for in silico Convergence Analysis: PAIN). In some embodiments, Input (2) a ‘Medical Condition with a Desired Sub-type’ produces Output(s) through the PhAROS process that include: ‘Ranked Minimum Essential Mixtures by Clinical Sub-type’. Input (2) describes the progression from Input to Output of Example 2 (i.e., Methods and Compositions for Novel Pain Therapies Targeted to Specific Pain Subtypes Identified using the PhAROS in silico Drug Discovery Platform). In some embodiments, using Input (3) a ‘Medical Condition, with a Desired Organism(s)’ produces Output(s) through the PhAROS process that include: ‘Ranked Compounds’ & ‘Ranked Minimum Essential Mixtures’. Input (3) describes the progression from Input to Output for Example 3 (i.e., “Piper Species Study”) and Example 6 (i.e., “MIGRAINE: Transcultural Formulations, Minimal Essential Formulations”). In some embodiments, Input (4) a ‘Divergence Analysis with Overlapping Conditions’ produces Output(s) through the PhAROS process that include: ‘Ranked Compounds’ & ‘Ranked Minimum Essential Mixtures’. Input (4) describes the progression from Input to Output for Example 4 (i.e., “PhAROS_PHARM Divergence Analysis of Cancer & Pain in Database to find Novel Cytotoxic Agents”). In some embodiments, Input (5) ‘Medical Condition, within a Geographical Region’ produces Output(s) through the PhAROS process that include: ‘Ranked Formulas’ based on the PhAROS_USER's Geographical Location. Input (5) describes the progression from Input to Output for Example 5 (i.e., “World Health Initiatives & Alternative Supply Chain Proof-of Concept”). In some embodiments, Input (6) ‘Desired Compounds’ produces Output(s) through the PhAROS process that include: ‘Ranked Plant Sources’, ‘Relative Compound Abundance’, and ‘Geography’. Input (6) describes the progression from Input to Output of two examples: Example 2 (i.e., “Methods and Compositions for Novel Pain Therapies Targeted to Specific Pain Subtypes Identified using the PhAROS in silico Drug Discovery Platform”) and Example 5 (i.e., “World Health Initiatives & Alternative Supply Chain Proof-of Concept”). In some embodiments, Input (7) is a ‘Current Plant Source with desired Components’ that produces Output(s) through the PhAROS process that include: ‘Alternative Plant Sources’, ‘Relative Compound Abundance’, and ‘Geography’. Input (7) describes the progression from Input to Output of two examples: Example 2 (i.e., “Methods and Compositions for Novel Pain Therapies Targeted to Specific Pain Subtypes Identified using the PhAROS in silico Drug Discovery Platform”) and Example 5 (i.e., “World Health Initiatives & Alternative Supply Chain Proof-of Concept”).

FIGS. 9A-C show for illustrative purposes only some in-process examples of the utility of the PhAROS Platform In Process Designing of Data Analytics for Drug Discovery. FIG. 9A provides an in-process view of using the PhAROS platform to select regions, type of phytochemical, TRP Assoc., components, etc. for use in novel drug discovery activities. FIG. 9B shows in-process views from PhAROS of convergent compounds from multiple TMS within a specific plant, Abrus precatorius. FIG. 9C shows in-process views from the PhAROS platform of interrogations of multiple TMS searching by specific TM formula(s).

FIG. 10 shows for illustrative purposes only an example of extracted database processing of one embodiment.

FIG. 11 shows for illustrative purposes only an example of an example of a PhAROS_USER process with a PhAROS_METAB subsystem of one embodiment.

FIG. 12 shows for illustrative purposes only an example of an example of a user process through PhAROS_USER with a PhAROS_EPIST subsystem of one embodiment.

FIG. 13 shows for illustrative purposes only an example of an example of a user process with a PhAROS_BIOGEN Subsystem of one embodiment.

FIGS. 14A-C shows for illustrative purposes only an example of Metrics of the PhAROS computational space of one embodiment. FIG. 14A summarizes the content and features of the PhAROS_PHARM proprietary data set. FIG. 14B “Inclusion Criteria for Phase I” development of PhAROS, showing a table and a schematic map summarizing the included and excluded features of TMS in the PhAROS_PHARM proprietary data set. FIG. 14C shows a schematic representation in-group and out-group TMS features used to decide inclusion in PhAROS.

FIGS. 15A-C shows for illustrative purposes only characterization of PhAROS computational space of one embodiment. FIG. 15A shows a graphic characterization of PhAROS computational space, including formula count by TMS. FIG. 15B shows characterization of PhAROS computational space, including ingredient organism type by TMS. FIG. 15C shows characterization of PhAROS computational space using a chord diagram representation of shared ingredient plants by occurrence in indicated TMS.

FIG. 16 shows for illustrative purposes only an example of a Schematic architecture of one embodiment. PhAROS_PHARM includes therapeutic indication, composition, organism composition, history, culture and biogeography. PhAROS_PHARM is layered with multiple additional data layers for multidimensional interrogation using multiple axes of query. Additional data layers: PhAROS_CHEMBIO, PhAROS_TOX, PhAROS_METAB, PhAROS_BIOGEO, PhAROS_CLINICAL, PhAROS_POPGEN, and PhAROS_EPIST, among others.

FIG. 17 shows for illustrative purposes only an example of a concept underlying Transcultural Formulations of one embodiment: biogeocultural boundaris for artemisinin. FIG. 17 shows biogeographical distribution of biogeographical distribution of Artemisia annua, and PhAROS outputs that include artemisinin.

FIG. 18 shows for illustrative purposes only an example of an in silico convergence analysis (ICSA), including convergence (e.g., PhAROS_CONVERGE) and divergence (e.g., PhAROS_DIVERGE). This schematic representation illustrates the concept of de-risking translation of phytomedical therapies from TMS to Western pipelines through identifying commonalities in approaches from biogeographically and culturally separated locales. Both groups of Convergent Compounds and groups of Divergent Compounds can be used for specific areas of drug design.

FIG. 19 shows for illustrative purposes only an example of a Minimal Essential Formulations of one embodiment. This schematic representation illustrates the concept of reducing complexity of TMS polypharmaceutical preparations to identify minimal essential efficacious components that are candidates for translation from TMS to Western discovery pipelines. TMS are complex polypharmaceutical mixtures. Sometimes they contain anachronistic and quasi-beneficial ingredients that we sort out of the database. The Minimal Essential Formulations are guided by the principals of Jun, Chen, Zuo, and Shi (Minister, Advisor, Soldier, and Envoy), which translates to therapeutic mixtures that in practice contain a principal and a supporting therapeutic, as well as ingredients to treat associated side effects/symptoms or reduce toxicity and finally, ingredients that help with delivery of the drug mixture.

FIG. 20 shows for illustrative purposes only an example of PhAROS_PHARM machine learning of one embodiment. This PhAROS_PHARM machine learning output is a correlation analysis reflecting co-occurrence/association of major chemical type with one another across the entire compound space.

FIG. 21 shows for illustrative purposes only an example of indication dictionaries of one embodiment. This schematic explains that the dictionaries used to interrogate PhAROS reflect modern and historical terminology, Western and non-Western epistemologies embedded in TMS. The dictionaries are used for database filtering and as features for subsequent AI/ML. Without the clinical indication dictionaries, it would be impossible to interrogate across the cultural boundaries in many instances because different cultures use unique terms to describe clinical symptoms and disorders. Some search terms like PAIN translate fairly easily across cultural boundaries, but terms like MIGRAINE are much more varied in their clinical descriptions across cultures.

FIG. 22A-D shows for illustrative purposes only an example of in silico convergence analysis (ISCA) for transcultural pain therapy. FIGS. 22A-B show the initial in in silico convergence analysis for Pain using PhAROS Platform when the initiating step is assembly of a clinical indication dictionary or “CID” (FIG. 22A) or when the initiating step is identification of formulae using literature mining (FIG. 22B). FIG. 22C shows PhAROS outputs including the numbers of formulations, indications, ingredient organisms and chemical components found in PhAROS across the indicated TMS. FIG. 22D shows PhAROS outputs resulting from in silico convergence analysis for pain. This schematic shows that 121 compounds were indicated for pain in 4 or more TMS.

FIGS. 23A-C shows for illustrative purposes only an example of PhAROS outputs: resulting from an in silico convergence analysis for pain of one embodiment. FIG. 23A shows for illustrative purposes only a schematic of steps in in silico convergence analysis for Pain. FIG. 23B shows PhAROS outputs resulting from an in silico convergence analysis for pain. The table shows the number and type of candidate analgesics identified by PhAROS in ISCA for pain. FIG. 23C. PhAROS outputs: results of in silico convergence analysis for pain. This table is an example of a ranking by PhAROS of the most convergent compounds in a class (alkaloids and opioids, with other classes summarized in the inset), representing the compounds with broadest agreement between TMS for inclusion in pain formulations.

FIG. 24A-C shows for illustrative purposes only an example of PhAROS output results from an in silico convergence analysis. FIGS. 24A-24B shows an in silico analysis and output in the form of a chord diagram (Circos plot) that can be generated (PhAROS_MODVIZ) to represent overlap and lineages between TMS. FIG. 24C shows a frequency ranking by PhAROS of the most convergent compounds in a class separated by level of agreement between TMS (convergence across 5 regions, convergence across 4 regions) (e.g., outputs ranked by coincidence across specific TMS).

FIGS. 25A-25C shows for illustrative purposes only an example of a series of wet laboratory experiments that confirmed the PhAROS predictions in the PhAROS outputs of one embodiment disclosed as Example 1. FIG. 25A shows comparison plots for the relative intensity of the intracellular free calcium mobilization initiated by each terpene with the diameter of each circle representing the peak intensity (middle panel), and as peak intensity summarized in histograms (lower panel). FIG. 25B shows ligand-target modeling. Left panel shows two-dimensional representation of molecular docking of Myrcene at the nociceptive ion channel TRPV1, including ligand interactions of Myrcene at binding site 4 of TRPV1. Left panel also shows similarities in chemical moieties between specific terpenes found in plant sources. Right panel shows a three-dimensional representation of Myrcene docked at binding site 4 of TRPV1. FIG. 25C shows data on the functional effects of terpenes at the nociceptive ion channel TRPV1. Left panel shows Fluo-4 Ca2+ response in wild type BEK or BEK over-expressing TRPV1 treated with vehicle or with 10 μM mixture of terpenes derived from phytomedical plants identified using PhAROS. Right panel: whole cell patch clamp electrophysiology, myrcene was shown to activate TRPV1 conductance. Together, these experiments validated the use of the alkaloid and terpene compounds selected by PhAROS for use in combination in diminishing the perception of pain signals through TRPV1.

FIG. 26 shows for illustrative purposes only an example of an indication (e.g., pain) across TM systems from multiple cultures of one embodiment. FIG. 26 summaries ISCA for two Kampo and two TCM formulations indicated for pain. Formulation component lists (˜800-2000 components) were generated using databases such as BATMAN-TCM and KAMPO-DB and triaged for obviously non-bioactive components (leading to lists of ˜200-400 compounds). These were then re-categorised using literature analysis into opioid/alkaloid candidate analgesics (alkaloids related to known opioid receptor ligands, 4 convergent compounds), potential ligands for nociceptive ion channels (terpenes, 49 convergent compounds), components with other demonstrated neuroactivity (15 convergent compounds), components with bioactivity indirectly related to pain (anti-inflammatory, anti-oxidants, 16 convergent compounds) and compounds with other types of bioactivity but no obvious link to analgesia (56 convergent compounds).

FIG. 27 shows for illustrative purposes only a schematic of a process for opioid alternative pain medication design based on PhAROS outputs.

FIG. 28 shows for illustrative purposes only use of PhAROS_CHEMBIO for Target Identification. FIG. 28 shows an example of PhAROS OUTPUT: all molecular targets associated with chemical components of TMS formulations indicated for pain.

FIG. 29 show for illustrative purposes only use of PhAROS_PHARM to match compounds to subtypes of an indication. FIGS. 29A-C show hypothesis testing for whether TMS differentiate between pain sub-types and able to match chemical components and ingredient organisms to specific pain types and performed PhAROS_PHARM text mining to collapse >1000 pain indications across 5 TMS to 37 major categories. FIG. 29A shows a PhAROS output example: regional convergence and associated number of formulations for 37 major pain subtypes identified using PhAROS.

FIGS. 30A-C show for illustrative purposes only an example use of PhAROS_PHARM to identify putative broad spectrum analgesic candidates. Text mining was performed to collapse >1000 pain indications to 37 major categories, then ranked filtering of outputs to identify putative broad spectrum analgesic candidates. FIG. 30A shows a PhAROS output example: Top 10 Ingredient organisms with broadest pain subtype associations in PhAROS_PHARM. FIG. 30B shows a PhAROS output example: Top 10 Alkaloids with broadest pain subtype associations in PhAROS_PHARM. FIG. 30C shows a PhAROS output example: Top 10 Terpenes with broadest pain subtype associations in PhAROS_PHARM.

FIG. 31 shows for illustrative purposes only an example use of PhAROS_PHARM to identify putative narrow spectrum analgesic candidates suitable for treating specific pain subtypes. Text mining was performed mining to collapse >1000 pain indications to 37 major categories, then ranked filtering of outputs to identify putative narrow spectrum analgesic candidates (based on narrowest pain spectrum). This schematic shows the top-ranking alkaloid chemical components associated with the indicated pain subtypes in PhAROS_PHARM.

FIG. 32 shows for illustrative purposes only an example use of PhAROS_PHARM to identify putative narrow spectrum analgesic candidates suitable for treating specific pain subtypes. Text mining was performed to collapse >1000 pain indications to 37 major categories, then ranked filtering of outputs to identify putative narrow spectrum analgesic candidates (based on narrowest pain spectrum). This schematic shows the top-ranking terpene chemical components associated with the indicated pain subtypes in PhAROS_PHARM.

FIG. 33 shows for illustrative purposes only example use of PhAROS_PHARM to generate searchable network visualizations of ingredient-formula linkages associated with a pain subtype.

FIG. 34 shows for illustrative purposes only an example use of PhAROS_PHARM to identify putative narrow spectrum analgesic candidates suitable for treating joint pan. We performed text mining to collapse >1000 pain indications to 37 major categories, then ranked filtering of outputs to identify putative narrow spectrum analgesic candidates where the indications specified joint pain. This schematic shows the top-ranking chemical components associated with the joint pain subtype in PhAROS_PHARM.

FIG. 35 shows for illustrative purposes only an example use of PhAROS to look for a clinical indication in a specific organism. An example PhAROS_PHARM Output list is shown in the inset that includes a list of Piper spp occurring in 1 or more formulation from 1 or more TMS in PhAROS_PHARM.

FIGS. 36A-B show for illustrative purposes only an example use of PhAROS_PHARM output for Piper spp. studies. FIG. 36A shows an example of a PhAROS_PHARM output, including an example of differential indications for Piper spp between distinct TMS, underscoring the potential for transcultural Piper-based medicines. FIG. 36B shows an example of a PhAROS_PHARM output, including an example of differential indications for Piper spp between distinct TMS, underscoring the potential for transcultural Piper-based medicines.

FIG. 37 shows for illustrative purposes only a representation of Piper spp in formulations derived from the various TMS in PhAROS_PHARM and associated with indications mined using a custom dictionary that included pain, epilepsy, anxiety, depression, mood and sleep.

FIG. 38 shows for illustrative purposes only an example of PhAROS_PHARM Data Integration using comparative biogeography of Piper spp that are indicated for the disorders of interest.

FIGS. 39A-B show for illustrative purpose only an example of PhAROS_PHARM Output. FIG. 39A shows association of P. methysticum active ingredients with formulations in non-Pacific TMS. FIG. 39B shows an example PhAROS_PHARM output: alternative non-Piper spp sources for 1 or more active ingredients of P. methysticum.

FIG. 40 shows for illustrative purposes only an example of PhAROS_PHARM Output: Complete compound set for all Piper ingredient organisms associated with anxiety in PhAROS_PHARM.

FIG. 41 shows for illustrative purposes only an example of PhAROS_PHARM Machine Learning Output: histogram shows specific chemical type features most predictive of anxiety/mood/depression utility of a formulation were Alkaloid, Terpene, Fatty acid-related compounds, Flavonoid, and Phenyl propanoid.

FIG. 42 shows for illustrative purposes only an example of PhAROS_PHARM Machine Learning Output: histogram shows specific ingredient organisms most predictive of anxiety/mood/depression utility of a formulation were: Glycyrhizza uralensis/radix, Paeonia lactiflora, Scutellaria baicalensi, Panax ginseng, Saposhnikovia divaicata, and Poria cocos.

FIG. 43 shows for illustrative purposes only post-hoc evaluation of ML top ranked ingredient organism features for anxiety/mood/depression.

FIG. 44 shows for illustrative purposes only an example of PhAROS to discover novel cancer therapies based on a DIVERGENCE ANALYSIS between PAIN and CANCER in the PhAROS_PHARM database. Cancer and pain medicine component overlap most of the time. A CANCER.PAIN master list of compounds was compiled for subsequent comparison with ALLPAIN compounds.

FIG. 45 shows for illustrative purposes only an example of PhAROS machine learning (ML) predictions showing that >80% of the chemical components of cancer medications in PhAROS are also found in pain medications. A divergent chemical component subset was identified between cancer and pain indications, which can now be mined for cytotoxic components using PhAROS_CHEMBIO and PhAROS_TOX.

FIG. 46 shows for illustrative purposes only an example of PhAROS_ML used to assess the ingredient organisms most likely to contain chemical components that diverge between cancer and pain (i.e., most likely cytotoxic or non-analgesic ingredients).

FIG. 47 shows for illustrative purposes only an example of PhAROS outputs identifying source organisms for 10 medically important phytomedical compounds. A list of phytomedically important compounds for indications ranging from cancer to pain was assembled using PubMed searches. This test set was used to interrogate PhAROS_PHARM to identify plant sources, known indications and TM systems in which the compound was used, and for what indication.

FIGS. 48A-B show for illustrative purposes only an example of PhAROS outputs from FIG. 47. FIG. 48B shows biogeography figures for source organisms, demonstrating use of PhAROS as a supply chain decision support tool (www.gbif.org). Additional species identified as parthenolide, paclitaxel, or tanshinone sources in PhAROS alter the geographical range of the PTL supply chain dramatically when compared to the archetypal source (e.g. Feverfew, Parthenium tanacetum for parthenolide).

FIGS. 49A-B show for illustrative purposes only an example of data integration of PhAROS outputs with NCBI analysis. Source organisms for parthenolide and paclitaxel suggested by PhAROS analysis of TMS data were assessed for their linkages to the compounds using PubMed. FIGS. 49A-49B show total number of publications linking organism and compound, suggesting that at least one of these relationships Tripterygium wilfordii/Parthenolide has not previously been reported in the peer reviewed literature.

FIGS. 50A-C show for illustrative purposes only a PhAROS output of an input query for migraine. FIG. 50A shows an example therapeutic indication dictionary for migraine. FIG. 50B shows a summary of the processed data grouped by region, formulations that contain a migraine indication dictionary hit, and the total formulas. FIG. 50C shows the molecular targets for all compounds identified in Example 6.

FIG. 51 shows for illustrative purposes only a PhAROS_PHARM in silico convergence analysis outputs for de novo transcultural formulation design, identification of minimal essential and prioritization for inclusion of phytomedical components. This table shows lists of compounds by class that are identified as migraine dictionary hits and which are convergent (shared) between either 5 (left column) or 4 (right column) TMS. The lower panel indicates the PhAROS stage (validation) represented by this output and provides a key to color coding of hits: (**) indicates compounds previously identified as TRPA1 or TRPV1 ligands, which are know targets for migraine (see inset publication, PhAROS_CHEMBIO). (*) indicates compounds in current clinical use for migraine. These data both validate the outputs of PhAROS and provide the potential for new design of novel formulations based on combinations of compounds from these PhAROS output lists. This illustrates the decision support capability of PhAROS for de novo medication design.

FIG. 52 shows for illustrative purposes only PhAROS_PHARM in silico convergence analysis of neurotropic TMS components to identify new or alternative migraine medications. Text mining was used to assemble a list of 209 neurotropic fungi. This neurotropic fungi dictionary was then used to interrogate PhAROS_PHARM for use of the neurotropic organisms in formulations that were indicated as hits for the migraine dictionary. The PhAROS outputs show that 2 neurotropic fungi species appeared in any TMS (Claviceps purpurea (TCM) and Amanita muscaria (TIM)) associated with migraine. In silico convergence analysis presented in the schematic show that 2 convergent compounds are candidate alternatives to ergotamine with agreement between 2 TMS (see, e.g., “ISCA potential alternatives to ergotamine”). Several other alternative ergot family compounds are identified as candidates for inclusion in novel formulations (see, e.g., “Inclusion candidates with documented anti-migraine potential”). Non-ergot compounds that appear in 1 or 2 TMS for migraine and which have a plausible rationale for inclusion based on a subsequent validation step (literature review) are also candidates for inclusion in novel formulations (see, e.g., “Other potential alternatives to ergotamine”). This system also cleanly differentiated and excluded non-migraine (in this case anti-poison) components of the initial hit list, demonstrating the utility of PhAROS in compound prioritization and inclusion/exclusion decision support.

### 5. DETAILED DESCRIPTION

In the following description, reference is made to the accompanying drawings, which form a part hereof. It is to be understood that other embodiments may be utilized and structural changes may be made without departing from the scope of the present invention.

### General Overview:

It should be noted that the description that follows, for example of a method and systems for phytomedicine analytics for research optimization at scale (PhAROS), is described for illustrative purposes and the underlying system can apply to any number and multiple types of phytomedicine analyses. In one embodiment of the present invention, the method and systems for phytomedicine analytics for research optimization at scale can be configured using multiple searchable databases. The method and systems for phytomedicine analytics for research optimization at scale can be configured to include algorithmic processing and machine learning algorithms and can be configured to include silico processing in order to simulate and thus predict therapeutic phenotypic results using the present invention.

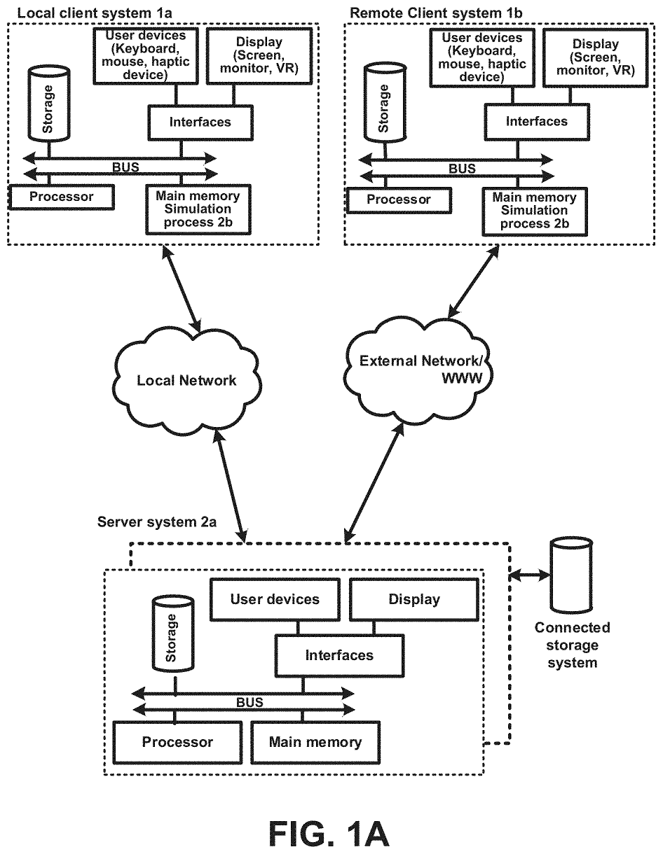

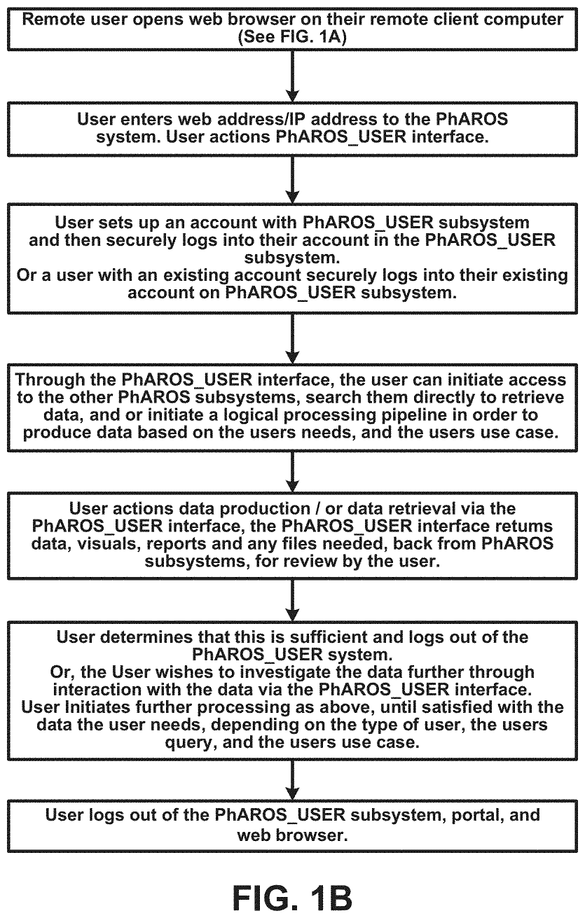

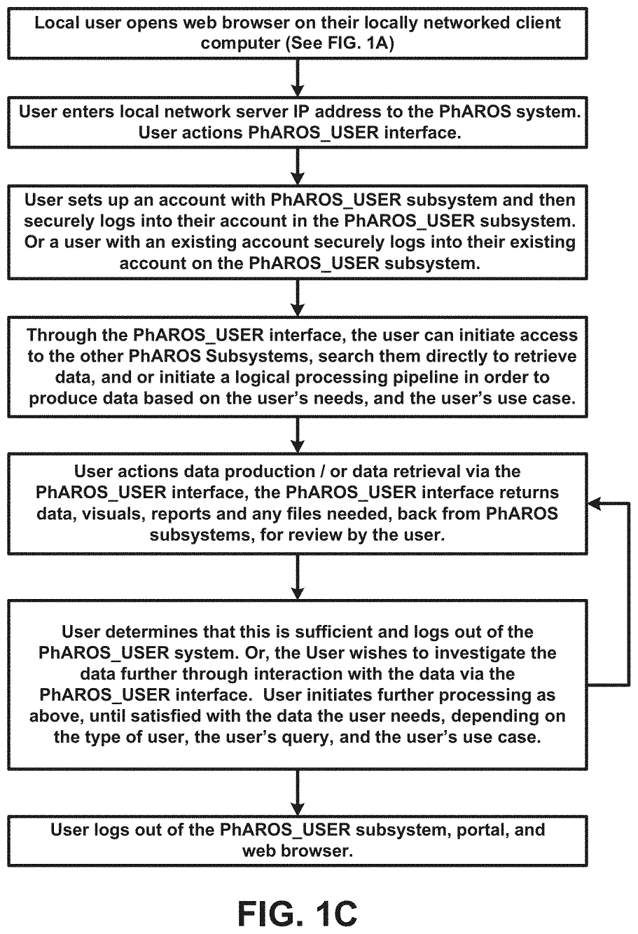

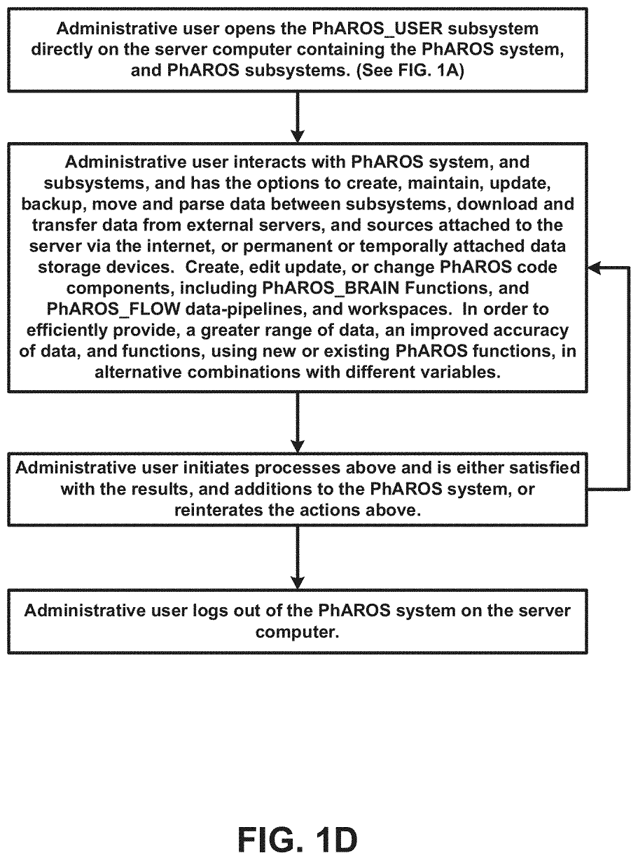

FIG. 1A shows for illustrative purposes only an example of a client and server computer system of one embodiment. FIG. 1A shows a client and server computer system. A local client system 1a is configured with user devices (keyboard, mouse, haptic device). The local client system 1a includes a display (screen, monitor, VR). Interfaces are coupled to a system bus that is coupled to storage devices, processor and a main memory simulation process 2b.

FIG. 1A shows a client and server computer system. A remote client system 1b is configured with user devices (keyboard, mouse, haptic device). The local client system 1a includes a display (screen, monitor, VR). Interfaces are coupled to a system bus that is coupled to storage devices, processor and a main memory simulation process 2b.

The local client system 1a is wirelessly coupled to a local network. The local network is wirelessly coupled to a server system 2a. The remote client system 1b is wirelessly coupled to an external network/WWW. The external network/WWW is wirelessly coupled to the server system 2a. The server system 2a is configured with user devices, a display, interfaces coupled to a system bus that is coupled to storage devices, processor and a main memory of one embodiment

In accordance with some embodiments, the systems and methods described here as the PhAROS discovery platform for computational phyto-pharmacology (PhAROS) consist as a science gateway and virtual research environment for drug discovery user interfaces. As well, data repositories and data processing components not accessible to general users are accessible and maintained by administrator users.

Through a series of servers and computer systems; that downloads, pre-processes, cleans, processes, analyses, normalizes, dynamically normalizes or pre-process normalizes, correlates, translates, and sorts traditional medicine data and other correlative data, users can access the system, processing methods and data, and then rapidly and accurately view and compare processed tabular, graphical and non-text visual interpretations of the data. Users can also choose options that further process and reduce the data, depending on the users final wishes, this will depend on their choice of indication, medicinal plant component and/or compound, biological target, the users own competence and/or users domain of expertise. User options, filters and directions for generating an in silico hypothesis are customized based on the background of the user, including a basic biological researcher, clinical researcher, epidemiologist, pharmaceutical/therapeutic development professional, educator, environmentalist, war fighter resilience researcher, behavioral health researcher, xenobiologist, pharmacological logistics manager, chemical sourcing agent, medical doctor, field doctor, traditional medicine practitioner, NGO professional etc.

In some embodiments, the computing system can be any sort of server computing system (FIG. 1A) that processes, and delivers the data, for access by local user client devices on the same network (FIG. 1A), or via remote user client devices connected to external network, via the world wide web/internet (FIG. 1A), via a display, virtual reality display system, or other interactive visual devise, associated with the client device (e.g., personal computer, tablet computer, smart phone) or can be a stand-alone display that receives the generated/retrieved data and rendered processed data via the server.

In some embodiments generally PhAROS will integrate data sets, tools, and applications as a web-based portal with a graphical user interface PhAROS. PhAROS will connect an academic, industry and public health community of users with a pre-processed data repository, through cyberinfrastructure and computational resources (e.g., HPC). As a science gateway, PhAROS will allow users to query details of their scientific questions without the need for advanced expertise in areas such as supercomputing or data visualization. PhAROS will support user communities by providing advanced software applications (fully containerized workflows, analysis, simulation, prediction and modeling), human-in-the-loop intermediary analysis and cloud-based data repositories linked to cluster-, cloud- and super-computing services.

FIG. 1B shows a block diagram of an overview of a remote user process, for access to a PhAROS system of one embodiment. FIG. 1B shows a remote user process, for access to a PhAROS system. A remote user opens a web browser on their remote client computer. (See FIG. 1A). The user enters a web address/IP address to the PhAROS system. User actions are inputted into a PhAROS_USER interface. The user sets up an account with a PhAROS_USER subsystem. The user securely logs into their account in the PhAROS_USER subsystem.

A user with an existing account securely logs into their existing account on PhAROS_USER subsystem. Through the PhAROS_USER interface, the user can initiate access to the other PhAROS subsystems. The user can search them directly to retrieve data, and/or initiate a logical processing pipeline in order to produce data based on the user's needs and the user's use case.

The other PhAROS subsystems process user actions for data production or data retrieval via the PhAROS_USER interface. The PhAROS user interface returns data; visuals, reports and any files needed back from the PhAROS subsystems, for review by the user. The user determines that this is sufficient and logs out of the PhAROS_USER system. Should the user wish to investigate the data further through interaction with the data via the PhAROS_USER interface the user initiates further processing as above until satisfied with the data the user needs, depending on the type of user, the user's query and the users use case. Upon completion the user logs out of the PhAROS_USER subsystem portal and web browser of one embodiment.

FIG. 1C shows a block diagram of an overview of a local user process, for access to the PhAROS system of one embodiment. FIG. 1C shows a local user process, for access to the PhAROS system. A local user opens a web browser on their locally networked client computer. (See FIG. 1A). The user enters local network server IP address to the PhAROS system. User actions are inputted into the PhAROS_USER interface. The user sets up an account with a PhAROS_USER subsystem. The user securely logs into their account in the PhAROS_USER subsystem.

A user with an existing account securely logs into their existing account on PhAROS_USER subsystem. Through the PhAROS_USER interface, the user can initiate access to the other PhAROS subsystems. The user can search them directly to retrieve data, and/or initiate a logical processing pipeline in order to produce data based on the user's needs and the user's use case.

The other PhAROS subsystems process user actions for data production or data retrieval via the PhAROS_USER interface. The PhAROS_USER interface returns data; visuals, reports and any files needed back from the PhAROS subsystems, for review by the user. The user determines that this is sufficient and logs out of the PhAROS_USER system. Should the user wish to investigate the data further through interaction with the data via the PhAROS_USER interface the user initiates further processing as above until satisfied with the data the user needs, depending on the type of user, the user's query and the users use case. Upon completion the user logs out of the PhAROS_USER subsystem portal and web browser of one embodiment.

FIG. 1D shows a block diagram of an overview of an administrative user process, for access to the PhAROS system server of one embodiment. FIG. 1D shows an administrative user process, for access to the PhAROS system server. An administrative user opens the PhAROS USER subsystem directly on the server computer containing the PhAROS system and PhAROS_subsystems. (See FIG. 1A).

The administrative user interacts with PhAROS system and subsystems and has the options to create, maintain, update, backup, move and parse data between subsystems, download and transfer data from external servers, and sources attached to the server via the internet or permanent or temporally attached data storage devices, create, edit, update or change PhAROS code components including PhAROS_BRAIN Functions and PhAROS_FLOW data-pipelines and workspaces.

In order to efficiently provide, a greater range of data, an improved accuracy of data, and data searching ability, to backup data, to create machine learning modules and functions, using new or existing PhAROS functions, in alternative combinations with different variables. The administrative user initiates processes above and is either satisfied with the results, and additions to the PhAROS system, or reiterates the actions above. The administrative user logs out of the PhAROS system on the server computer of one embodiment.

### Definitions

As used herein, the term “PhAROS_USER” refers to the user interactive system of the PhAROS platform, and includes but is not limited to functional user tools designed to aid in coordinating user defined in silico analysis across multiple sub repositories and tools, in part by coordinating with PhAROS_CORE to utilize processes, connect and retrieve data and present user requested data, in an accessible manner. Basic and administrative levels of access limit possible disruption of data resources and tools.

As used herein, the term “PhAROS_CORE” refers to the core functional system of the PhAROS system, including but not limited to tools designed to collect, parse and maintain sub-systems, raw data repositories, pre-processed repositories, training data, data tools, automated and manual processing and task management.

As used herein, the term “PhAROS_BRAIN” refers to a repository of integrated data and a data processing/assessing tool. PhAROS_BRAIN includes but is not limited to a system that links the PhAROS_USER interactive system to advanced analysis tools. PhAROS_BRAIN functions enable de novo analysis, as well as being able to populate PhAROS subsystems with data.

As used herein, the term “PhAROS_FLOW” refers to a graphical data processing environment that provides users and administrators with the ability to process data using the PhAROS_BRAIN functions without extensive coding. PhAROS_FLOW includes, but is not limited to, at least one of subsystem modeling tools including machine learning and AI tools such as support vector machine, artificial neural networks, deep learning, Naïve Bayesian, K-nearest neighbors, random forest, AdaBoost wisdom of crowds and ensemble predictors, and validation tools such as Monte Carlo cross-validation, Leave-One-Out cross validation, Bootstrap Resampling, and y-randomization.

As used herein, the term “PhAROS_PHARM” refers to a proprietary pre-processed repository and computational space. PhAROS_PHARM comprises, but is not limited to, at least one of:

the first ‘meta-pharmacopeia’, processed and normalized formalized pharmacopeias, formulations, associated plant/organisms, associated available compound sets, and indications, temporal and geographical data, indicating historical, and contemporary geographical, cultural and epistemology origins;

processed and normalized formalized pharmacopeias from Japan, China, India, Korea, South East Asia, Middle East, North/South America, Russia, India, Africa, Europe, Australia; processed, translated normalized, individual relevant published datasets or case reports in the scientific literature that document relationships between medicinal plants and disease indications;

processed, curated ethical partnerships, indigenous, cultural (e.g., African, Oceanic) phytomedical formulations;

processed open source contemporary and historical herbologies that document relationships between medicinal plants and disease indications (e.g., Hildegard of Bingen, Causae et Curae, Physica);

processed, translation of resources from original languages processed using approaches such as machine literal translation, natural language processing, multilingual concept extraction or conventional translation; OCR of historical materials, and AI driven intent translation.

As used herein, the term “PhAROS_CONVERGE” refers to a pre-processed repository that includes, but is not limited to, at least one of an unbiased in silico convergence analysis of formulation composition explicitly between medical systems, predictions of minimal and/or essential compound sets for a given indication, a proprietary digital composition index (n-dimensional vector and/or fingerprint) identifying efficacy across traditional medicine systems, ranked optimized de novo formulations and mixtures utilizing transcultural components for subsequent preclinical and clinical testing in particular indications.

As used herein, the term “PhAROS_CHEMBIO” refers to a pre-processed repository of chemical and biological data, including but not limited to at least one of chemical structure, physicochemical properties, known and/or algorithmically calculated or predicted PD/PK properties, putative biological effects, data informing of receptor binding, docking, regulation of signaling pathways, metabolism, drug-target relationships, and mechanism of action, CYP interactions, as well as published studies and clinical trials.

As used herein, the term “PhAROS_BIOGEO” refers to a pre-processed repository of integrated data, including but not limited to the meta-pharmacopeia, associated temporally, geographical, botanical, climatological, environmental, genomic, metagenomic, and metabolomic data on originating plants, components or other organisms.

As used herein, the term “PhAROS_METAB” refers to a pre-processed repository of integrated data of, including but not limited to, the meta-pharmacopeia with de novo metabolomic data for plants, and/or organisms that are not currently in medicinal use, supplemental metabolomic data secured for known medicinal plants and/or associated organisms.

As used herein, the term “PhAROS_MICRO” refers to a pre-processed repository of integrated data of, including but not limited to, the meta-pharmacopeia with microbiome data on microorganisms associated with plants/organisms/components of interest, and their secondary metabolome compositions.

As used herein, the term “PhAROS_CURE” refers to a pre-processed repository of integrated data, including but not limited to, the meta-pharmacopeia with documented spontaneous regression/remission events associated with botanical medicine or supplement usage, organized by organism, including plant, compound set and clinical manifestation/ICD codes.

As used herein, the term “PhAROS_QUANT” refers to a pre-processed repository of integrated data of, including but not limited to, the meta-pharmacopeia with component weighting data based on either proportional components using standardized measurements and normalizations, for formulations and/or de novo quantitative analysis of formulated components.

As used herein, the term “PhAROS_POPGEN” refers to a pre-processed repository of integrated data of, including but not limited to, the genetic admixtures, SNP characteristics and genetic/ethnic variability in populations in whom the formulations within the meta-pharmacopeia have been tested geographically and temporally.

As used herein, the term “PhAROS_TOX” refers to a pre-processed repository of integrated data of, including but not limited to, the meta-pharmacopeia with toxicological and side-effect profile data, and/or de novo experimentally-derived data, and/or in silico predicted toxicological and side-effect data.

As used herein, the term “PhAROS_BH” refers to a pre-processed repository of integrated data and a data processing/assessing tool, including but not limited to, contextualization data of meta-pharmacopeia datasets within a novel proprietary Bradford-Hill decision support framework, predicting data interpretation and assessing the evidence base for assertions of potential efficacy.

As used herein, the term “PhAROS_EPIST” refers to a pre-processed repository of integrated data and a data processing/assessing tool, including but not limited to, parsed of formulation components data, plant, compound, a proprietary PhAROS correlation tool, that links composition to underlying epistemology for inclusion of a component (e.g., TCM/Kampo concept of JUN-CHEN-ZUO-SHI (‘Monarch, Minister, Assistant and Envoy’).

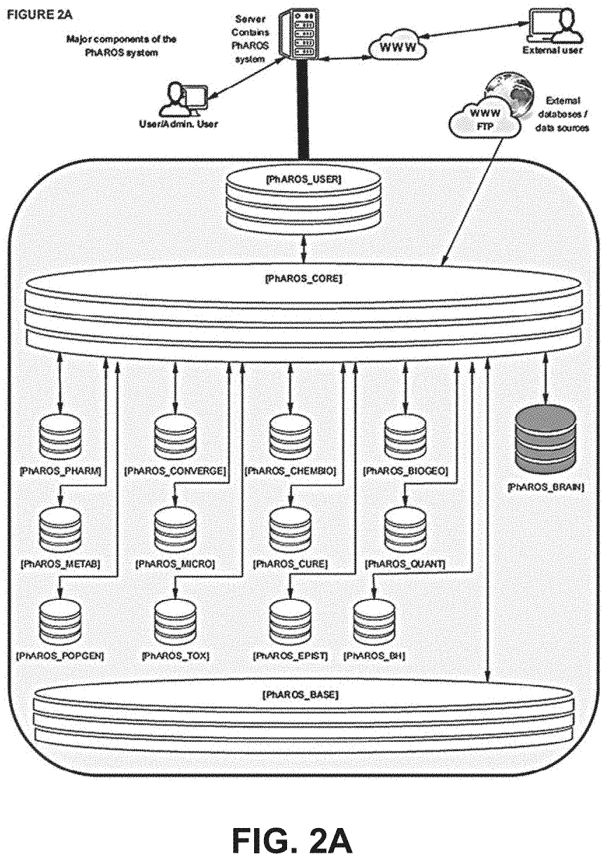

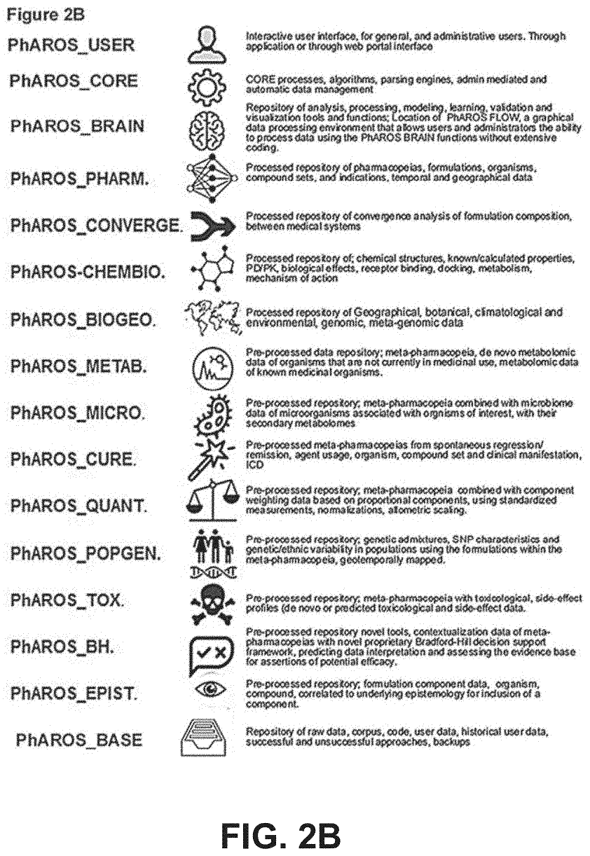

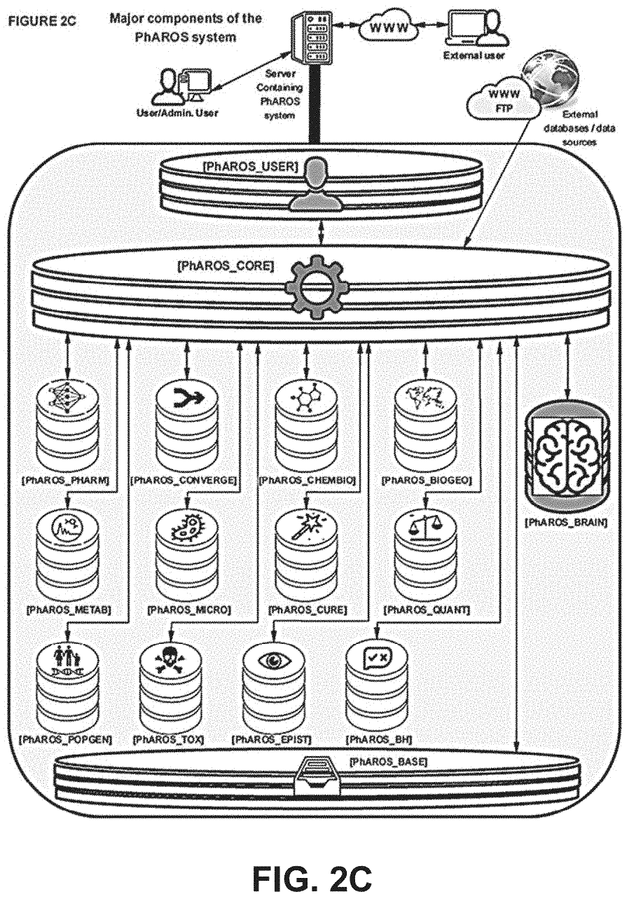

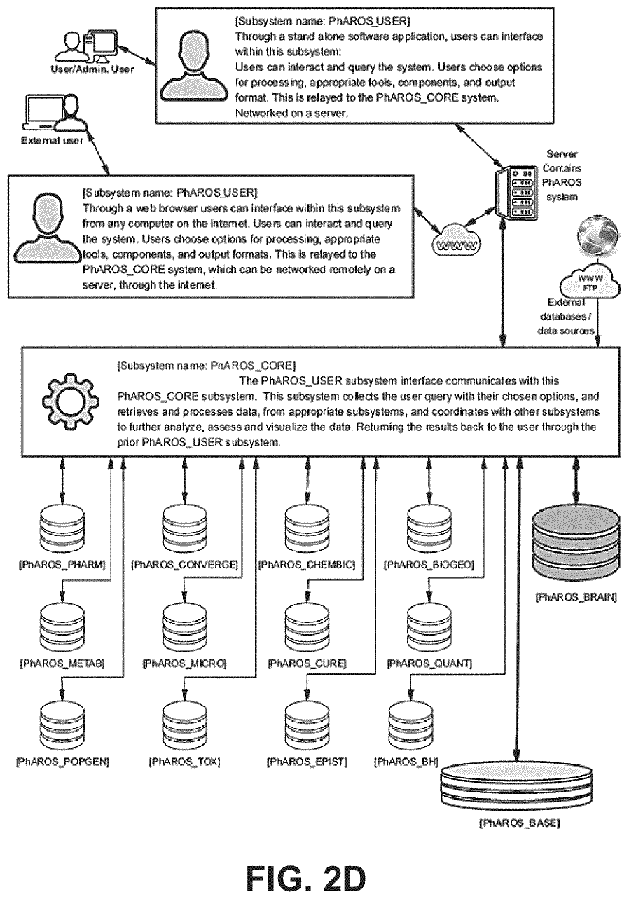

As used herein, the term “PhAROS_BASE” refers to a structured raw and pre-processed data repository of all data used to develop all the integrated data repositories in PhAROS subsystems, full and partially constructed data processing/assessing tools, backups, user data, user process history, machine learning data sets, and PhAROS_CORPUS, a repository of texts utilized and maintained to extract and parse data, and for text mining purposes. FIG. 2B shows for illustrative purposes only an example of a table describing the major components of the PhAROS system, with icon key of one embodiment.

As used herein, the term “PhAROS_DIVERGE” refers to a pre-processed repository including but not limited to, an unbiased in silico divergence analysis comprising identifying alternative compounds derived from one or more organisms, and therapeutic approaches from biogeographically and culturally separated locales across the plurality of TMS.

As used herein, the term “transcultural dictionaries” refers to a search dictionary that collates Western and non-Western epistemological understanding of terms including, but not limited to, medical formulations, organisms, medical compound data sets, and therapeutic indications.

As used herein, the term “therapeutic indications” refers to information on the use of a medicine, where the information can include, but is not limited to, disease and/or condition, severity of disease and/or condition, target population, and aim of the treatment (e.g., diagnostic indication, prevention, or treatment).

### PBS Embodiments

### Methods

Aspects of the present disclosure include a phytomedicine analytics for research optimization at scale (PhAROS) method for discovering and/or optimizing polypharmaceutical medicines. The PhAROS method comprises: analyzing, in a single computational space, data from a plurality of traditional medicine systems (TMS), wherein the analysis uses transcultural dictionaries to allow searches within distinct TMS data sets embodying different epistemologies and terminologies, wherein the analysis uses data returned by a query to identify new polypharmaceutical and/or optimized polypharmaceutical compositions.

For example, in some embodiments, the method includes receiving from a user in a graphical user interface (GUI), a user query input. The method uses the user query input (or user query) to search the data from the plurality of TMS, the data from the plurality of TMS associated with the first user query input. The method then processes the searched data to create processed data returned by the query from the plurality of TMS associated with the user query input. The analysis of the method uses data returned by the query to identify new polypharmaceutical and/or optimized polypharmaceutical compositions. However, the method can also include further processing the processed data, if further inquired by the user.

In some embodiments, analyzing comprises outputting the processed data returned by the query to the user for review by the user or for further analysis initiated by a second user query input.

User query Inputs can include, but are not limited to: (1) a medical condition, (2) a medical condition with a desired sub-type, (3) a medical condition, with a desired organism(s), (4) a divergence analysis with overlapping conditions, (5) a medical condition, with a geographical region, (6) desired compounds, or (7) current plant source with desired components.

For example, the analysis of the method can include outputting, for each of the respective inputs: Ranked Compounds & Ranked Minimum Essential Mixtures, Ranked Minimum Essential Mixtures by Clinical Sub-type, Ranked Compounds & Ranked Minimum Essential Mixtures, Ranked Compounds & Ranked Minimum Essential Mixtures, Ranked Formulas based on User's Geographical Location, Ranked Plant Sources, Relative Compound Abundance, Geography, and/or Alternative Plant Sources, Relative Compound Abundance, Geography.

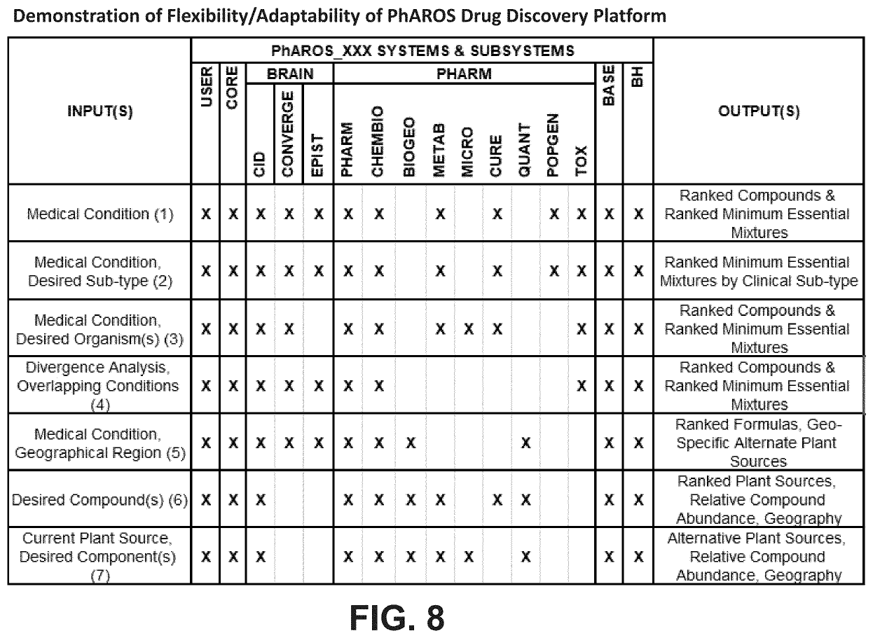

In some embodiments, the analysis of the method can include any combination of input and outputs as described in FIG. 8.

In some embodiments, Input (1) a medical condition includes an Output: Ranked Compounds & Ranked Minimum Essential Mixtures (see, e.g., FIG. 8). Input (1) describes progression from input to output of the original pain search/formulations as described herein (see, e.g., Example 1, Proof-of-Concept Demonstration for in silico Convergence Analysis: PAIN).

In some embodiments, Input (2) a Medical Condition with a Desired Sub-type includes an Output: Ranked Minimum Essential Mixtures by Clinical Sub-type (see, e.g., FIG. 8). Input (2) describes the progression from input to output of Example 2 (i.e., Methods and Compositions for Novel Pain Therapies Targeted to Specific Pain Subtypes Identified using the PhAROS in silico Drug Discovery Platform).

In some embodiments, Input (3) a Medical Condition, with a Desired Organism(s) includes an Output: Ranked Compounds & Ranked Minimum Essential Mixtures (see, e.g., FIG. 8). Input (3) describes the progression from input to output for Example 3 (i.e., “Piper Species Study”) and Example 6 (i.e., “MIGRAINE: Transcultural Formulations, Minimal Essential Formulations”).

In some embodiments, Input (4) a divergence analysis with overlapping conditions includes an Output: Ranked Compounds & Ranked Minimum Essential Mixtures (see, e.g., FIG. 8). Input (4) describes the progression from input to output for Example 4 (i.e., “PhAROS_PHARM Divergence Analysis of Cancer & Pain in Database to find Novel Cytotoxic Agents”).

In some embodiments, Input (5) Medical Condition, with a Geographical Region includes an Output: Ranked Formulas based on User's Geographical Location (see, e.g., FIG. 8). Input (5) describes the progression from input to output for Example 5 (i.e., “World Health Initiatives & Alternative Supply Chain Proof-of Concept”).

In some embodiments, Input (6) Desired Compounds includes an Output: Ranked Plant Sources, Relative Compound Abundance, Geography (see, e.g., FIG. 8). Input (6) describes the progression from input to output of two examples: Example 2 (i.e., “Methods and Compositions for Novel Pain Therapies Targeted to Specific Pain Subtypes Identified using the PhAROS in silico Drug Discovery Platform”) and Example 5 (i.e., “World Health Initiatives & Alternative Supply Chain Proof-of Concept”).

In some embodiments, Input (7) Current Plant Source with desired components include an Output: Alternative Plant Sources, Relative Compound Abundance, Geography (see, e.g., FIG. 8). Input (7) describes the progression from input to output of two examples: Example 2 (i.e., “Methods and Compositions for Novel Pain Therapies Targeted to Specific Pain Subtypes Identified using the PhAROS in silico Drug Discovery Platform”) and Example 5 (i.e., “World Health Initiatives & Alternative Supply Chain Proof-of Concept”).

In some embodiments, outputting the processed data returned by the query to the user for review by the user or for further analysis comprises outputting a list of compounds associated with the user selected clinical indication, a list of prescription formulae for a given TMS, a list of organisms associated with the user selected clinical indication, or a combination thereof. In some embodiments, the processed data returned by the query to the user for review by the user or for further analysis comprises outputting molecular targets for the list of compounds that are clinically indicated for a therapeutic indication across one or more TMS.

In some embodiments, outputting the processed data returned by the query to the user for review by the user or for further analysis comprises outputting: a list of species associated with one or more therapeutic indications.

In some embodiments, the outputting further comprises outputting cytotoxic agents within the list of compounds that are indicated for a therapeutic indication across one or more TMS.

In some embodiments, outputting further comprises outputting the list of organisms associated with a therapeutic indication across more TMS.

In some embodiments, the list of compounds is categorized by class, identified as indication dictionary hits, and are convergent between two or more TMS.

In some embodiments, the outputting further comprises outputting a list of compounds that is associated with a first user selected clinical indication, wherein the list of compounds that is associated with the first user selected clinical indication does not overlap with a list of compounds that is associated with a second user selected indication.

User

As described above, in some embodiments, the method includes, first, receiving from a user in a graphical user interface (GUI), a user query input.

The user of the PhAROS method and system can include users with various access to outputs or data returned by a query. For example, the user can perform a user query input to retrieve data, and/or initiate a logical processing pipeline in order to produce data based on the user's needs and the user's use case.

In some embodiments, each user will be able to perform actions for data production or data retrieval via the PhAROS_USER interface according to their credentials (e.g. type of access the user will have to the PhAROS system). In some embodiments, the PhAROS user interface returns data; visuals, reports and any files needed back from the PhAROS subsystems, for review by the user. The user determines that this is sufficient and logs out of the PhAROS_USER system. Should the user wish to investigate the data further through interaction with the data via the PhAROS_USER interface the user initiates further processing as above until satisfied with the data the user needs, depending on the type of user, the user's query and the users use case. Upon completion the user logs out of the PhAROS_USER subsystem portal and web browser of one embodiment.

Non-limiting examples of the type of users with different access rights to the PhAROS system include, but are not limited to: administrative user having administrative access to the system on behalf of the stakeholder, direct but limited access to the system as a user by the stakeholder, direct unlimited access to the system as a user/administrator; clinician user having direct but limited access to the system for a particular therapeutic use, a user having direct but limited access to the system for a therapeutic use in a particular geographical region, a user having direct but limited access to the system for global health initiatives (e.g., world health organization (WHO) or for non-profit), a user having direct but limited access to the system for searching alternative compounds (e.g., compounds isolated from plant or other organism in a particular geographical region). For example, one user can include a user that lives in a rural geographical location that is interested in developing compounds or compound mixtures from organisms that are grown in that particular geographical location.

For example, the PhAROS methods of the present disclosure are applicable to global health challenges linked to medicine availability and quality in locales classified by the UN as developing economies, economies in transition, heavily indebted poor countries (HIPC), emerging economies and small island developing states (SIDS). Herbal and phytomedicines are major pillars of medical provisioning in national health systems for WHO member nations. The National Essential Medicines List of 34 WHO member nations contain representation of herbal medicines (spanning WHO African, eastern Mediterranean, Americas, European, South-East Asia and Western Pacific regions). Up to 65% of the global population rely wholly or in part on non-Western pharmaceutical approaches to morbidity.

PhAROS Global Health (PhAROS_GH) is an initiative to enable users within developing, emerging economies to access medical optimizations and rationalization data to improve safety and efficacy of TMS as they are currently deployed.

In some embodiments, the user is a PhAROS_GH user group. Non-limiting examples of a PhAROS_GH user includes: global and regional agencies/NGO concerned with healthcare quality and safety in non-developed economies; governmental and private healthcare systems and/or organizations; for-profit entities located in non-developed economies; Non-profit entities located in non-developed economies; and grassroots and community healthcare organizations, systems and providers. In some embodiments, a PhAROS_GH user group has direct but limited access to the system for global health initiatives, such as a user having direct but limited access to the system for: searching alternative compounds (e.g., compounds isolated from plant or other organism in a particular geographical region); supply chain optimization, where the PhAROS_GH user can use PhAROS data on organism-chemical component relationships that expand the potential source organisms for preparation of specific formulations, allowing substitution of ingredients across biogeographical boundaries and decreasing supply chain limitations; medicine rationalization/optimization, where the PhAROS_GH user can the PhAROS method to improve upon current formulations in a given locale by incorporating transcultural elements to build new formulations that leverage information generated across cultures, locations and biogeograhies; medicine rationalization/optimization, where the PhAROS_GH user can use the PhAROS method to reduce complexity of formation by identifying minimal essential component for a given indication (potential decreasing supply chain limitations, increasing safety and consistency, decreasing undesirable side effects, decreasing use of non-essential or anachronistic components); rational design, where the PhAROS_GH user can use the PhAROS method to identify phytomedical solutions that are customized to specific locations, ingredient resources, populations and needs.

In some embodiments, the method comprises second, using the user query input to search the data from the plurality of TMS, the data from the plurality of TMS associated with the first user query input.

In some embodiments, the method includes third, processing the searched data to create processed data returned by the query from the plurality of TMS associated with the user query input.

In some embodiments, the method includes fourth, retrieving processed data based on the user query input for review by the user.

In some embodiments, the method comprises fifth, further processing the processed data, if further inquired by the user.

Data from Traditional Medicine Systems (TMS)

In some embodiments, data from the plurality of TMS comprises at least one of: medical formulations; organisms; medical compound data sets; therapeutic indications, processed and normalized formalized pharmacopeias from one or more geographic regions associated with TMS; therapeutic indication dictionaries related to traditional medical systems that reflect modern and historical terminology; Western and non-Western epistemologies; temporal and geographical data indicating historical, and contemporary geographical, cultural and epistemology origins; raw and optionally pre-processed data from a plurality of traditional medicine data sets, plant data sets, and literature-based text documents (corpus). In some embodiments, the one or more geographic regions (as such region is presently defined) is selected from Japan, China, Taiwan, India, Korea, South East Asia, Middle East, North America, South America, Russia, India, Africa, Europe, Australia, and Oceania.

In certain embodiments, data from the TMS comprises medical formulations. In certain embodiments, data from the TMS comprises organisms. In certain embodiments, data from the TMS comprises medical compound data sets. In certain embodiments, data from the TMS comprises therapeutic indications. In certain embodiments, data from the TMS comprises processed and normalized formalized pharmacopeias from one or more geographic regions associated with TMS. In certain embodiments, data from the TMS comprises therapeutic indication dictionaries related to traditional medical systems that reflect modern and historical terminology. In certain embodiments, data from the TMS comprises Western and non-Western epistemologies. In certain embodiments, data from the TMS comprises temporal and geographical data indicating historical, and contemporary geographical, cultural and epistemology origins.

In certain embodiments, data from the TMS comprises raw and optionally pre-processed data from a plurality of traditional medicine data sets, plant data sets, and/or literature-based text documents (corpus). In some embodiments, data from the TMS comprises plant data sets. In certain embodiments, data from the TMS comprises traditional medicine data sets. In some embodiments, the data from the TMS comprises literature-based text documents.

In some embodiments, the data from the TMS comprises one or more of: compounds, ingredient lists, formulations and their associated therapeutic indications, e.g., associated with formalized publicly-available pharmacopeias from Japan, China, India, Korea, South East Asia, Middle East, North America, South America, Russia, India, Africa, Europe, and Australia.

In some embodiments, data from the TMS comprises datasets from three continents, five contemporary and historical cultural medical systems, spanning over 5000 years of human medical endeavor and the biogeography of >16.9M square miles of medicinal plant growth.

In some embodiments, data from the TMS comprises datasets of gene expression curated profiles maintained by NCBI and included in the Gene Expression Omnibus.

Transcultural Dictionaries

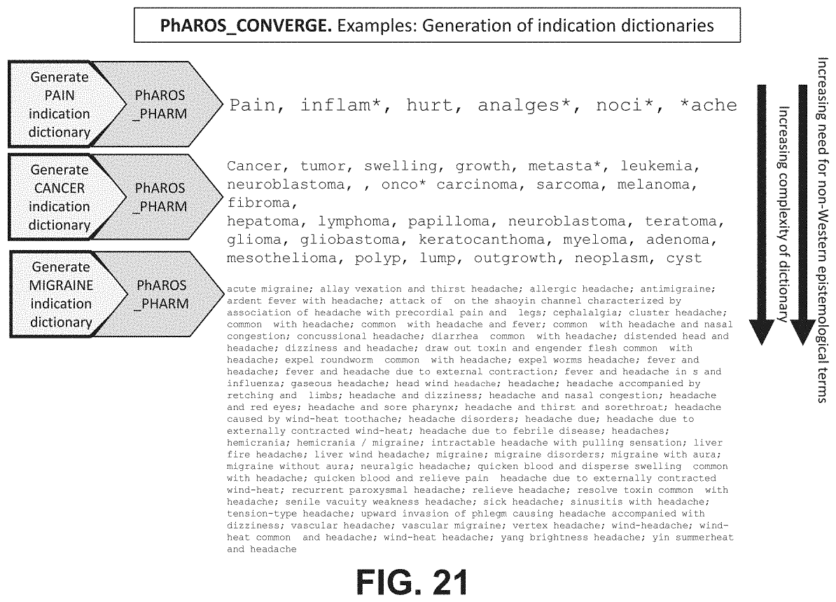

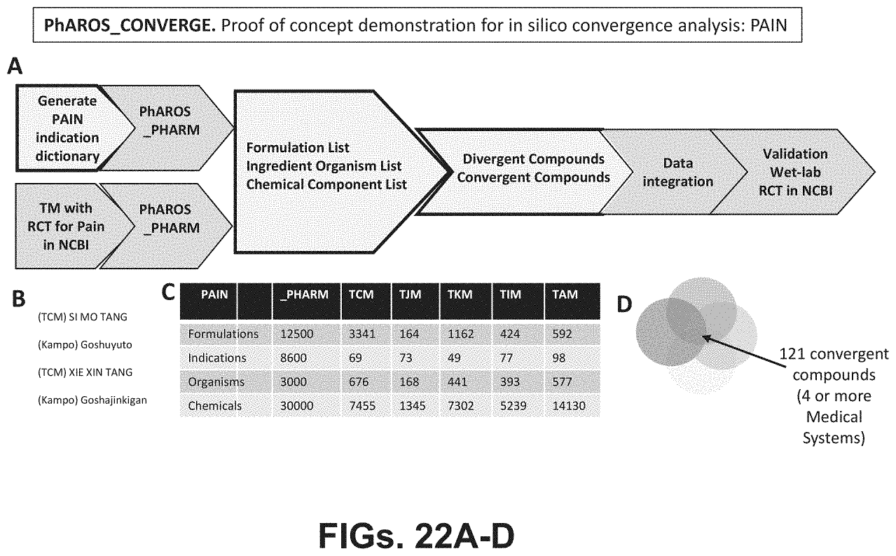

In some embodiments, the transcultural dictionary is a search dictionary that collates Western and non Western epistemological understanding of indication dictionaries (e.g., therapeutic indications), therapeutic indication dictionaries related to traditional medical systems that reflect modern and historical terminology, culture-specific terminology (modern and historical), organism dictionaries, compound lists, compound lists associated with a plant-source and/or therapeutic indication within a geographic location, and the like. In certain embodiments, the transcultural dictionaries comprise therapeutic indication dictionaries related to traditional medical systems that reflect modern and historical terminology. In certain embodiments, the transcultural dictionaries comprise therapeutic indication dictionaries related to organism dictionaries. In certain embodiments, the transcultural dictionaries comprise therapeutic indication dictionaries related to compound lists, and/or compound lists associated with a plant-source and/or therapeutic indication within a geographic location. Non-limiting examples of therapeutic indication dictionaries are provided in FIG. 21 and FIG. 22.

In some embodiments, the transcultural dictionaries comprise a search dictionary that collates Western and non-Western epistemological understanding of migraine and migraine-like patient presentations.

In some embodiments, one transcultural dictionary of the transcultural dictionaries comprises a list of compounds associated with cancer pain, and a list of compounds known for treating pain. In certain embodiments, at least one transcultural dictionary of the transcultural dictionaries comprises a search dictionary that collates Western and non-Western epistemological understanding of pain, pain-like patient symptoms. In certain embodiments, at least one transcultural dictionary of the transcultural dictionaries comprises a search dictionary that collates Western and non-Western epistemological understanding of Piper species associated with a therapeutic indication.

In certain embodiments, at least one transcultural dictionary of the transcultural dictionaries comprises a search dictionary that collates Western and non-Western epistemological understanding of cancer, cancer-like patient presentations, cytotoxic agents within TMS formulations for cancer, and cancer pain.

Processed and Normalized Formalized Pharmacopeias

In some embodiments, one or more processed and normalized formalized pharmacopeias comprises processed, translated normalized, individual published datasets or case reports in the scientific literature that document relationships between medicinal plants and disease indications. In some embodiments, one or more processed and formalized pharmacopeias comprises processed, translated normalized, individual published datasets or case reports in the scientific literature that document relationships between medicinal plants and disease indications.

In some embodiments, one or more processed and normalized formalized pharmacopeias comprises processed, curated ethical partnerships, indigenous, cultural (e.g., African, Oceanic, and the like) phytomedical formulations.

In some embodiments, the one or more processed and normalized formalized pharmacopeias comprises processed contemporary and historical herbologies that document relationships between medicinal plants and disease indications (e.g., Hildegard of Bingen, Causae et Curae, Physica).

In some embodiments, the one or more processed and normalized formalized pharmacopeias comprises processed, translation of resources from original languages processed using approaches selected from one or more of: machine literal translation, natural language processing, multilingual concept extraction or conventional translation, Optical character recognition (OCR) of historical materials, and artificial intelligence (AI)-driven intent translation.

In some embodiments, the one or more processed and normalized formalized pharmacopeias comprises data from one or more databases selected from: chemical compound databases, metabolic pathway databases, gene-disease databases, traditional medicine databases, plant metabolomics database, databases for references and abstracts on life sciences and biomedical topics, and variant-phenotype relation database that may provide data regarding the association among a phenotype and one or more genetic loci or single nucleotide polymorphisms (SNPs). Example external data servers from which the data can be taken from include, but are not limited to: ClinVar, PubMed, DrugBank, STITCH for drugs, drug actions and drug-target interactions, PubChem, ChEMBL, Natural Products Atlas, MoleculeNet, ATC for chemical information databases, KEGG for Metabolic pathways, OMIM for Gene-disease relationships, TCM Data Warehouse, Clinical Trials.gov for clinical trials databases, PlantMetabolomics.org, Metabolights, SetUpX, SWMD, MetaboAnalyst for metabolomes, HPRD, BioGRID, DIP for protein databases, HPRD, BIND, DIP, HAPPI, MINT, STRING, PDZBase for biomolecular interactions, Cytoscape, Pajek, VisANT, GUESS, WIDAS, PATIKA, PATIKAweb, CADLIVE for networking and visualization tools, TOXNET, CTD, DSSToxicology, FDA Poisonous Plants database, National Poison Center for network toxicology and poison databases. Other processed and normalized formalized pharmacopeias include data from databases that store clinical study data, scientific papers, medical records, and suitable university databases.

In some embodiments, the one or more processed and normalized formalized pharmacopeias comprises data from one or more databases selected from: Chinese traditional medicines (ETCM, MESH), Japanese traditional medicines (kampo, Kegg), Korean traditional medicines (KTKP), Indian Traditional Medicines (TKDL, IMPPAT), African Traditional Medicines (SANCDB, ETMDB, and Prelude).

Medical Compound Datasets

In some embodiments, data from the plurality of TMS comprises medical compound data sets.

In some embodiments, the medical compound data sets chemical and/or biological data of medical compounds. In some embodiments, chemical and biological data of medical compounds comprise one or more of: chemical structure, physicochemical properties, known and/or algorithmically calculated or predicted PD/PK properties, putative biological effects, data informing of receptor binding, molecular docking sites on human receptors, regulation of signaling pathways, metabolism, drug-target relationships, mechanism of action, CYP interactions, or published studies and clinical trials of the medical compounds.

In certain embodiments, the medical compound data set comprises phytomedical compounds. In certain embodiments, the medical compound data set comprises one or more of: traditional Chinese medicine compounds, traditional Japanese medicine compounds, traditional Indian medicine compounds, traditional Korean medicine compounds, traditional South East Asian medicine compounds, traditional Middle Eastern medicine compounds, traditional North American compounds, traditional South American compounds, traditional Russian medicine compounds, traditional Indian medicine compounds, traditional African medicine compounds, traditional European medicine compounds, and traditional Australian medicine compounds.

In certain embodiments, the medical compound comprises compounds derived from the metabolomes of plants, fungi, and other prokaryotic and eukaryotic organisms.

Raw and Optionally Processed Data Normalized from a Plurality of Traditional Medicine Data Sets

In some embodiments, the raw and optionally pre-processed data normalized from a plurality of traditional medicine data sets comprises one or more selected from: meta-pharmacopeia, associated temporally, geographical, botanical, climatological, environmental, genomic, metagenomic, and metabolomic data on originating plants, components or other organisms; meta-pharmacopeia with de novo metabolomic data for plants, and organisms that are not currently in medicinal use, supplemental metabolomic data secured for known medicinal plants and/or associated organisms, and toxicological and side-effect profile data of medical compound data sets, de novo experimentally-derived data of medical compound data sets, and in silico predicted toxicological and side-effect data of medical compound data sets.

In certain embodiments, the raw and optionally pre-processed data normalized from a plurality of traditional medicine data sets comprises meta-pharmacopeia, associated temporally, geographical, botanical, climatological, environmental, genomic, metagenomic, and metabolomic data on originating plants, components or other organisms.

In certain embodiments, the raw and optionally pre-processed data normalized from a plurality of traditional medicine data sets comprises toxicological and side-effect profile data of medical compound data sets, de novo experimentally-derived data of medical compound data sets, and/or in silico predicted toxicological and side-effect data of medical compound data sets.

In certain embodiments, the raw and optionally pre-processed data normalized from a plurality of traditional medicine data sets comprises meta-pharmacopeia with de novo metabolomic data for plants, and organisms that are not currently in medicinal use, supplemental metabolomic data secured for known medicinal plants and/or associated organisms.

In certain embodiments, the raw and pre-processed data is stored in a data repository of all data used to develop all the integrated data repositories in PhAROS subsystems, full and partially constructed data processing/assessing tools, backups, user data, user process history, machine learning data sets, and PhAROS_CORPUS, a repository of texts utilized and maintained to extract and parse data, and for text mining purposes. FIG. 2B shows for illustrative purposes only an example of a table describing the major components of the PhAROS system, with icon key of one embodiment.

In some embodiments, the raw data can include raw text data, as well as specific sets of data are predominantly stored in the PhAROS_CORPUS, in the PhAROS_CORE subsystem. In some embodiments, Raw data, as well as specific sets of data are predominantly stored in the PhAROS_CORE subsystem, or processed and added to PhAROS subsystems, for access by various types of user, depending on their use case.

Analysis of Data, from a Plurality of Traditional Medicine Systems (TMS) in in a Single Computational Space

Aspects of the present methods include analyzing data from a plurality of TMS in a single computational space.

As described above in the “user” section, the method includes receiving a user query input, using the user query input to search the data from the plurality of TMS, the data from the plurality of TMS associated with the first user query input, processing the searched data to create processed data returned by the query from the plurality of TMS associated with the user query input, retrieving processed data based on the user query input for review by the user, and further processing the processed data, if further inquired by the user. In some embodiments, analyzing comprises outputting the processed data returned by the query to the user for review by the user or for further analysis.

Further processing the processed data can include a variety of analysis options, for example, performing: an in silico convergence analysis (PhAROS_CONVERGE), an in silico divergence analysis (PhAROS_DIVERGE), PhAROS_BIOGEO analysis, PhAROS_PHARM analysis, PhAROS_CHEMBIO analysis, PhAROS_METAB analysis, PhAROS_MICRO analysis, PhAROS_CURE analysis, PhAROS_QUANT analysis, PhAROS_POPGEN analysis, PhAROS_TOX analysis, PhAROS_BH analysis, PhAROS_BRAIN analysis, and/or PhAROS_EPIST analysis.

In some embodiments, further analysis can include a variety of analysis options, for example, performing: an in silico convergence analysis (PhAROS_CONVERGE), an in silico divergence analysis (PhAROS_DIVERGE), PhAROS_BIOGEO analysis, PhAROS_PHARM analysis, PhAROS_CHEMBIO analysis, PhAROS_METAB analysis, PhAROS_MICRO analysis, PhAROS_CURE analysis, PhAROS_QUANT analysis, PhAROS_POPGEN analysis, PhAROS_TOX analysis, PhAROS_BH analysis, PhAROS_BRAIN analysis, and/or PhAROS_EPIST analysis.

In-Silico Convergence Analysis

In some embodiments, the method includes processing the searched data to create processed data returned by the query from the plurality of TMS associated with the user query input.

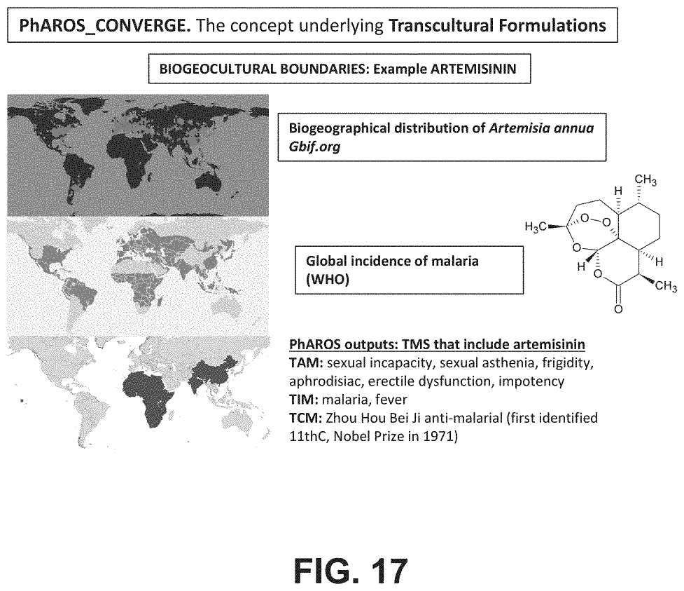

In certain embodiments, processing the searched data comprises performing an in silico convergence analysis to search drug-target-indication relationships associated with the user query input. For example, the method can include a convergence as an analysis mode to search for “derisked compound mixtures”, for example, when searching for the same compounds in different TMS. The in silico convergence analysis reduces the complexity and de-risks translation of phytomedical therapies from TMS to Western pipelines through identifying commonalities in approaches from biogeographically and culturally separated locales. For example, as shown in FIG. 17, the in silico convergence analysis can improve on existing TMS formulations by aggregating knowledge across cultures, biogeographries and time. Commonalities are then de-risked and pre-validated for entry into, for example, a drug development pipeline.

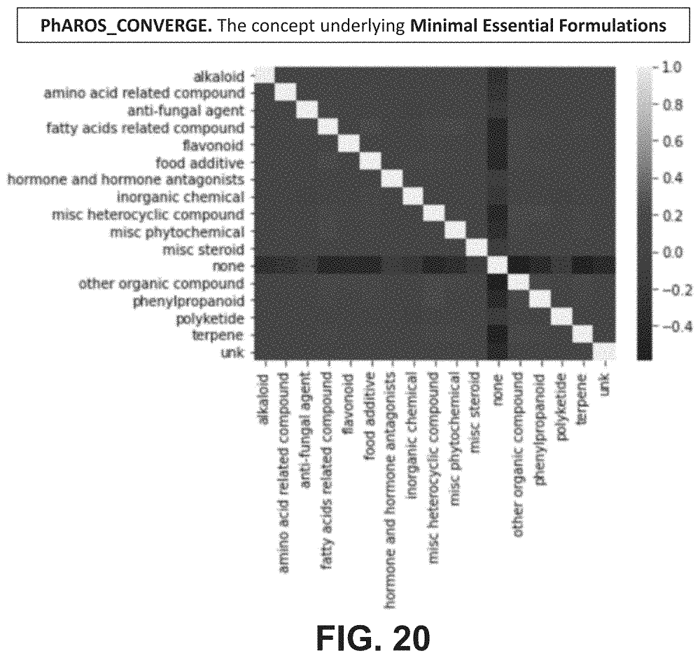

In another embodiment, an in silico convergence analysis can reduce complexity of TMS polypharmaceutical preparations to identify minimal essential efficacious components that are candidates for translation from TMS to Western discovery pipelines (FIG. 20). In yet another embodiment, methods can include performing an in silico convergence analysis to generate indication dictionaries for database filtering and as features of the artificial intelligence and machine learning that reflect the knowledge systems underlying diagnosis (FIG. 21).

In certain embodiments, performing a convergence analysis provides improved and/or optimized polypharmaceutical and/or optimized polypharmaceutical compositions that have higher chances to be efficacious.

In certain embodiments, processing the searched data comprises performing an in silico convergence analysis comprising identifying commonalities between two or more of: a disease, a therapeutic indication, one or more compounds derived from one or more organisms, and therapeutic approaches from biogeographically and culturally separated locales, coincidence or convergence of one or more compounds across a plurality of TMS, and coincidence or convergence of one or more organisms across a plurality of TMS.

In certain embodiments, the in silico convergence analysis further comprises using processed data returned by the query to rank new polypharmaceutical compositions for subsequent preclinical and clinical testing for a given therapeutic indication.

In certain embodiments, processing the searched data from the plurality of TMS using the in silico convergence analysis predicts efficacy of the new and/or optimized polypharmaceutical compositions.

In some embodiments, processing the searched data from the plurality of TMS using the in silico convergence analysis identifies minimal essential compounds required for efficacy of the new and/or optimized polypharmaceutical compositions. Non-limiting examples of performing the convergence analysis of the methods described herein are provided in FIGS. 17-25.

In Silico Divergence Analysis

In some embodiments, processing the searched data comprises performing an in silico divergence analysis. An in silico divergence analysis provides region-specific solutions that can be included in de novo designed formulations that overcome biogeocultural boundaries. For example, performing an in silico divergence analysis provides for searching drug-target-indication relationships associated with the user query input.

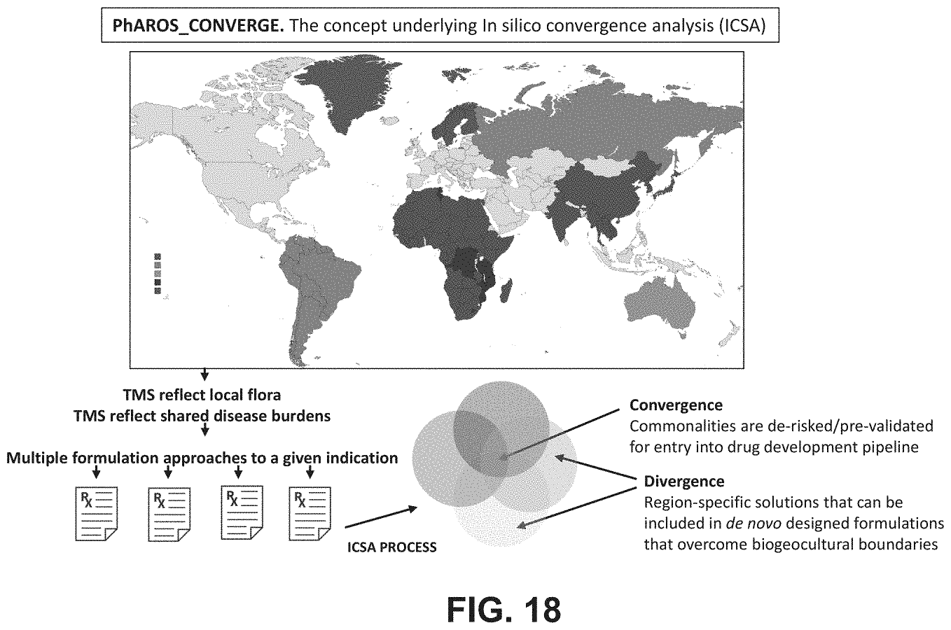

In some embodiments, processing the searched data comprises performing an in silico divergence analysis comprising identifying alternative compounds derived from one or more organisms, and therapeutic approaches from biogeographically and culturally separated locales across a plurality of TMS. An example of a divergence analysis is illustrated in FIG. 18. FIG. 18 shows that for multiple formulation approaches to a given indication, a divergence analysis (non-overlapping formulation approach regions) provides region-specific solutions that can be included in de novo designed formulations that overcome biogeocultural boundaries.

In some embodiments, the in silico divergence analysis further comprises using processed data returned by the query to rank new polypharmaceutical compositions for subsequent preclinical and clinical testing for a given therapeutic indication.

In some embodiments, processing the searched data from the plurality of TMS using the in silico divergence analysis predicts efficacy of the new and/or optimized polypharmaceutical compositions.

In some embodiments, a first user input query comprises one or more user selected clinical indications. In some embodiments, the one or more user selected clinical indications is selected from cancer, cancer pain, and cancer and cancer pain.

In some embodiments, the method includes outputting processed data returned by the query. In certain embodiments, outputting the processed data returned by the query comprises outputting: a list of compounds associated with the user selected clinical indication, a list of prescription formulae for a given TMS, a list of organisms associated with the user selected clinical indication, or a combination thereof.

In some embodiments, outputting comprises outputting a list of compounds that is associated with a first user selected clinical indication, wherein the list of compounds that is associated with the first user selected clinical indication does not overlap with a list of compounds that is associated with a second user selected indication.

New Polypharmaceutical and/or Optimized Polypharmaceutical Compositions

In some embodiments, new polypharmaceutical and/or optimized polypharmaceutical compositions comprise one or more compounds derived from metabolomes of prokaryotic, Archaea, or eukaryotic organisms.

In some embodiments, the new polypharmaceutical and/or optimized polypharmaceutical compositions comprise one or more compounds derived from metabolomes of plants or fungi.

In some embodiments, the optimized polypharmaceutical compositions comprise one or more substitution compounds of an existing transcultural medicinal formulation.

In some embodiments, the optimized polypharmaceutical composition comprises a reduced number of compounds within the optimized polypharmaceutical composition as compared to an existing transcultural medicinal formulation, wherein the optimized polypharmaceutical composition comprises a minimal number of essential compounds to achieve a therapeutic outcome.

PhAROS_Brain

In some embodiments, the methods of the present disclosure comprise outputting the processed data returned by the query to the user for review by the user or for further analysis initiated by a second user query input, e.g., to identify the new polypharmaceutical and/or optimized polypharmaceutical compositions.

In certain embodiments, further analysis comprises, after outputting one or more selected from: developing training data sets for one or more machine learning models to optimize the transcultural dictionaries; populate the transcultural dictionaries with additional data developed by the machine learning algorithm; and creating, updating, annotating, processing, downloading, analyzing, or manipulating the data from the plurality of TMS. In certain embodiments, further analysis comprises developing training data sets for one or more machine learning models to optimize the transcultural dictionaries. In certain embodiments, further analysis comprises populating the transcultural dictionaries with additional data developed by a machine learning algorithm. In some embodiments, further analysis comprises creating, updating, annotating, processing, downloading, analyzing, or manipulating the data from the plurality of TMS.

In certain embodiments, populating the transcultural dictionaries with additional data developed by the machine learning algorithm comprises generating a therapeutic indication dictionary. In certain embodiments, at least one transcultural dictionary of the transcultural dictionaries comprises a search dictionary that collates Western and non-Western epistemological understanding of migraine and migraine-like patient presentations. In certain embodiments, at least one transcultural dictionary of the transcultural dictionaries comprises a search dictionary that collates Western and non-Western epistemological understanding of pain, pain-like patient symptoms. In certain embodiments, at least one transcultural dictionary of the transcultural dictionaries comprises a search dictionary that collates Western and non-Western epistemological understanding of Piper species associated with a therapeutic indication. In certain embodiments, populating the transcultural dictionaries with additional data developed by the machine learning algorithm comprises generating a dictionary for Piper species.

In certain embodiments, at least one transcultural dictionary of the transcultural dictionaries comprises a search dictionary that collates Western and non-Western epistemological understanding of cancer, cancer-like patient presentations, cytotoxic agents within TMS formulations for cancer, and cancer pain.

In certain embodiments, at least one transcultural dictionary of the transcultural dictionaries comprises a list of compounds associated with cancer pain, and a list of compounds known for treating pain.

In some embodiments, the method further comprises iteratively training the one or more machine learning models/algorithms with the one or more training data sets.

In some embodiments, the method further comprises applying a machine learning model to identify the new polypharmaceutical and/or optimized polypharmaceutical compositions. In some embodiments, the machine learning model is iteratively trained with one or more training data sets.

In some embodiments, wherein the machine learned model comprises a set of rules, wherein the set of rules are configured to: identify specific patterns of interest, therapeutic targets for subsequent processing, metadata groupings that correlate with indications across traditional medicines, identify missing plants, components or compounds, identify unknown indications for traditional medicines, identify toxic and non-toxic components and compounds, identify plant, component and compound mixtures with ranked therapeutic potential, identify plant, component and compound combination that would not be obvious or have greater therapeutic potential, than existing mixtures in isolated traditional medicines.

In some embodiments, the method comprises applying the machine-learned model to identify the new polypharmaceutical and/or optimized polypharmaceutical compositions.

In some embodiment, the PhAROS method comprises a computing server. In some embodiments, the computer server may include one or more computing devices that aggregates data in a federated database, analyzes various compilations of data entries, performs convergence analyses or divergence analyses, deconvolves modes and mechanisms associated with data entries, and trains and applies various predictive models such as machine learning models. The computing server may be referred to as data analytics platforms and, in some embodiments, a phytomedicine analytics platform for research optimization at scale. The computing server may receive, from a user device, an input that includes one or more terms, each of which may correspond to a data entry, a formula that include multiple data entries, a target, an indication, or a compound. In response, the computing server may automatically retrieve information and attributes related to the terms by parsing data from various external data sources and performing a query for data in the data store. The computing server may in turn aggregate the data and perform convergence analysis or divergence analysis to reconcile or identify the differences and conflicts in data entries retrieved from different data sources. The computing server may also apply one or more predictive models to predict the attributes of a combination of items that correspond to the data entries selected by the user. The computing server may transmit the results of its analyses directly to the client device via the network to be displayed and visualized in the interface or may transfer the results to data store, which may be accessible by client device.

In some embodiments, the computer server comprises a prediction and machine learning engine. The prediction and machine learning engine may train and apply different machine learning models to predict the attributes of a combination of data entries, such as a formulation based on several components obtained from different traditional medicine sources. The prediction and machine learning engine may predict de novo transcultural formulations reflecting integration of components derived from geographically and culturally separated locales and minimal essential therapeutic component list for a selected indication. The prediction and machine learning engine may also predict the properties of a new formulation and the efficacy of the formulation for a certain treatment or salutogenesis purpose. The prediction and machine learning engine may also be used to identify new therapeutic candidates from an input specified by the user.

In various embodiments, the prediction and machine learning engine may use various machine learning techniques and models. Example machine learning techniques include clustering, regression, classification and dimensionality reduction tailored to a specific data set and problems. Unsupervised machine learning may use data sets that are treated as ‘blind’ samples (without a label) or when classification and categorical labels are unavailable or incomplete. Supervised machine learning models such as SVM (support vector machine), ANN (artificial neural networks), which may include convolutional neural networks (CNN), recurrent neural networks (RNN) and long short-term memory networks (LSTM), DL (deep learning), Bayesian models, KNN (K-nearest neighbors), RF (random forest), ADA (AdaBoost), wisdom of crowds and ensemble predictors, virtual screening and others. The prediction and machine learning engine may also include validation models such as Monte Carlo cross-validation, Leave-One-Out (LOO) cross validation, Bootstrap Resampling, and y-randomization.

The training and use of a machine learning model may include generating a machine learning model, iteratively training the model with one or more sets of training samples, and applying the model. In various embodiments, the training techniques for a machine learning model may be supervised, semi-supervised, or unsupervised. In supervised learning, the machine learning models may be trained with a set of training samples that are labeled. For example, for a machine learning model trained to classify property of a component in a traditional medicine, the training samples may be known components labeled with their properties. In some cases, an unsupervised learning technique may be used. The samples used in training are not labeled. Various unsupervised learning technique such as clustering may be used. In some cases, the training may be semi-supervised with training set having a mix of labeled samples and unlabeled samples. A machine learning model may be associated with an objective function, which generates a metric value that describes the objective goal of the training process. For example, the training may intend to reduce the error rate of the model in generating predictions. In such a case, the objective function may monitor the error rate of the machine learning model. The objective function of the machine learning algorithm may be the training error rate in predicting properties in a training set. Such an objective function may be called a loss function. Other forms of objective functions may also be used, particularly for unsupervised learning models whose error rates are not easily determined due to the lack of labels.

Alternative Supply Chain

The PhAROS method of the present disclosure can be used to identify alternative sources for medically important phytomedical compounds. In order to widely adopt phytomedical components into mainstream medicine, the issue of supply chain availability can be addressed using the methods described herein. For example, the methods of the present disclosure can provide alternative sources of phytomedical components that may be easier to extract leading to production efficiencies.

In some embodiments, the method of the present disclosure includes first, receiving from a user in a graphical user interface (GUI), a user query input.

The user of the PhAROS method and system can include users with various access to outputs or data returned by a query. For example, the user can perform a user query input to retrieve data, and/or initiate a logical processing pipeline in order to produce data based on the user's needs and the user's use case.

In some embodiments, the user input query comprises one or more phytomedical compounds or formulations, and optionally a current source (plant or animal) and supply of the compound or formulation.

In some embodiments, the method includes processing the searched data to create processed data returned by the query from the plurality of TMS associated with the user query input. In certain embodiments, the processed data comprises a list of plant sources, known clinical indications associated with the phytomedical compounds or formulations and the TMS in which each compound was referenced. In certain embodiments, the processed data further comprises a relative abundance of the one or more compounds or formulations, wherein the relative abundance is the relative amount of the one or more compounds or formulations available. In certain embodiments, the processed data further comprises growing locations of the list of plant sources.

In certain embodiments, the processed data is cross ranked by one or more of frequency, relative abundance, availability, potency, and supply.

In some embodiments, the method includes outputting the processed data returned by the query to the user for review by the user or for further analysis initiated by a second user query input to identify the new polypharmaceutical and/or optimized polypharmaceutical compositions.

In some embodiments, the new polypharmaceutical and/or optimized polypharmaceutical compositions comprise one or more compounds derived from metabolomes of an alternative source of plants or fungi that were not previously identified for a specific use or indication. In certain embodiments, the optimized polypharmaceutical compositions comprise one or more substitution compounds of an existing transcultural medicinal formulation, wherein a source origin of the substitution compound is not found in an existing transcultural medicinal formulation.

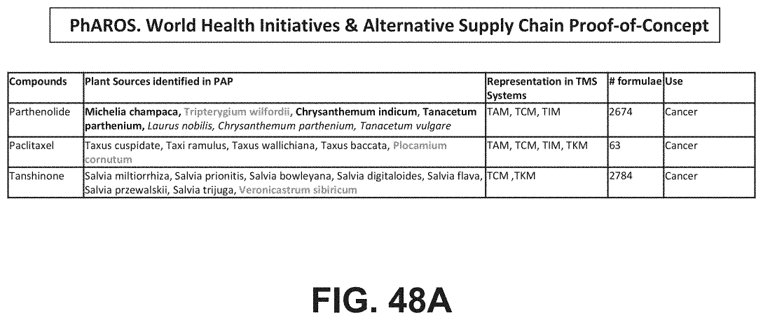

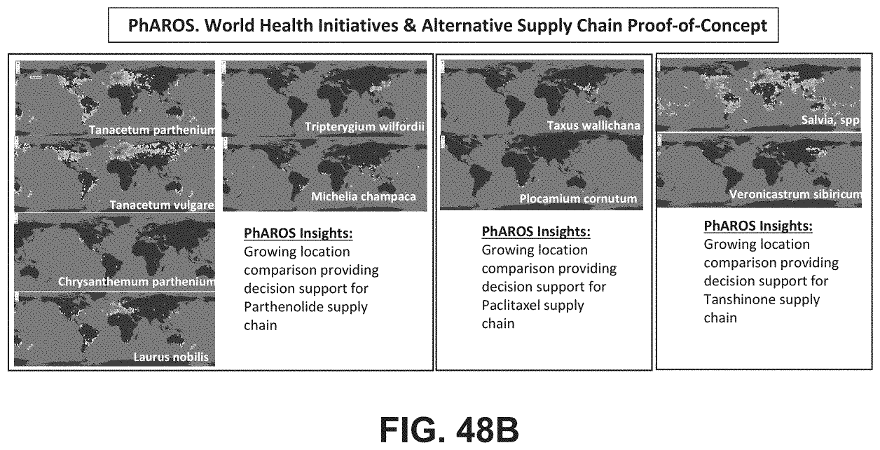

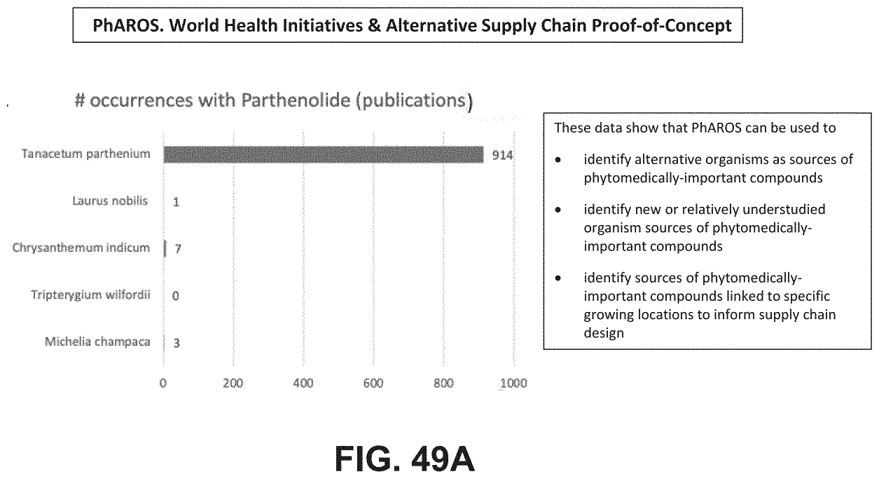

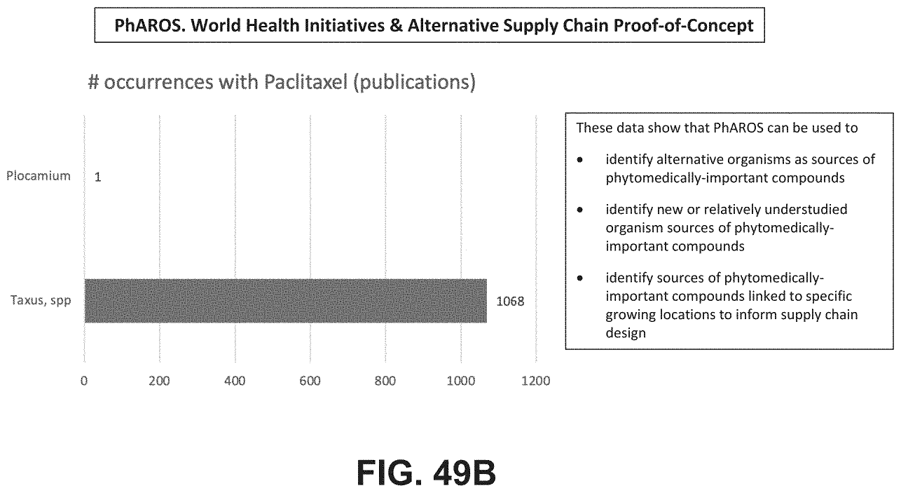

In some embodiments, the method includes outputting a growing location comparison of a phytomedical component providing decision support for the phytomedical component supply chain (see e.g., FIGS. 48A-B and FIGS. 49A-B).

In some embodiments, the method includes outputting one or more of: alternative organisms as sources of phytomedically-important compounds, new or relatively understudied organism sources of phytomedically-important compounds, and sources of phytomedically-important compounds linked to specific growing locations to inform supply chain design.

Additional Descriptions of the Pharos Methods

In some embodiments, the first user input query of the PhAROS method comprises one or more user selected clinical indications.

Migraine

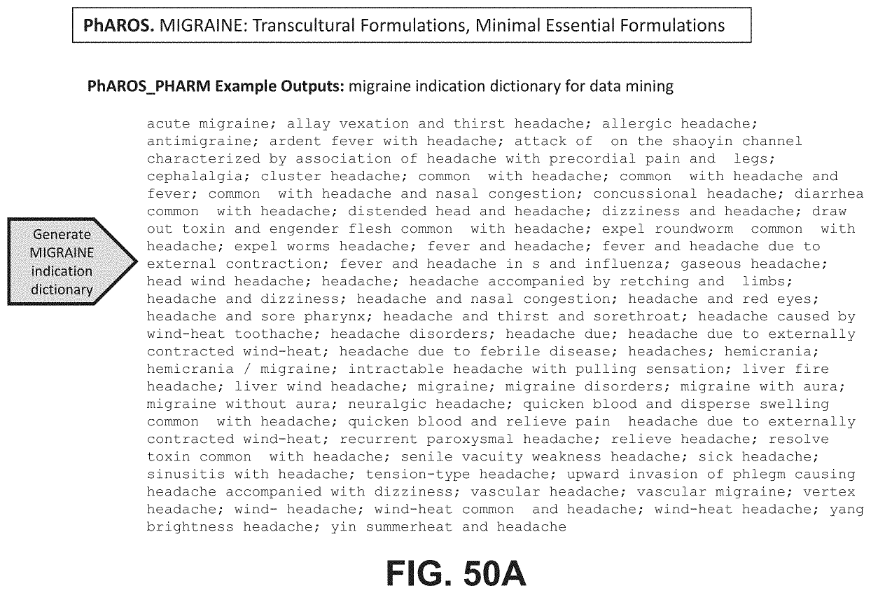

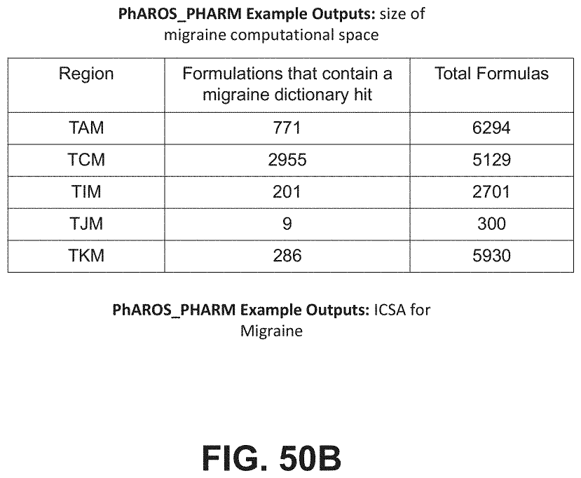

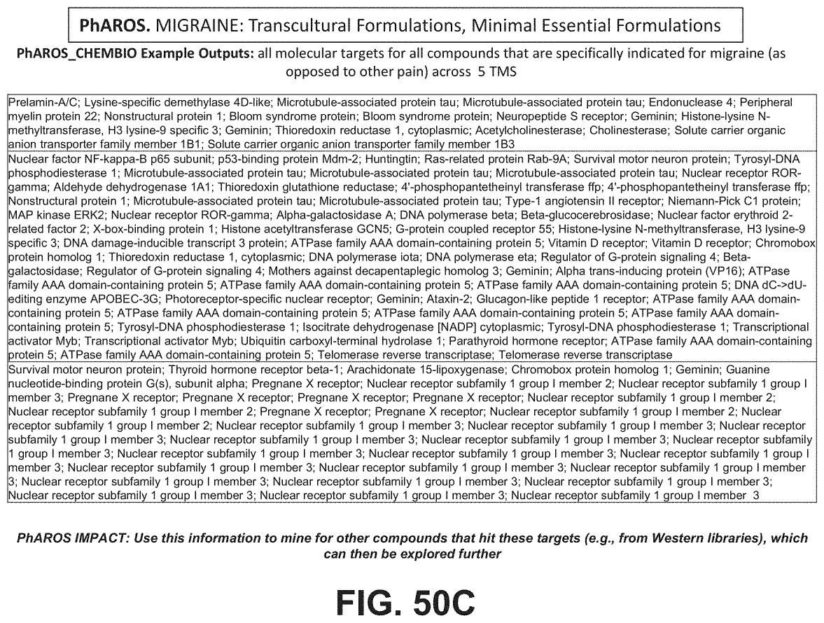

In some embodiments, the one or more user selected clinical indications is migraine. In such cases, PhAROS can be used to design new polypharmaceutical approaches for treating migraine (see, e.g., Example 6). In some embodiments, outputting the processed data returned by the query comprises outputting: a list of compounds associated with the user selected clinical indication, a list of prescription formulae for a given TMS associated with the user selected clinical indication, or a combination thereof. In certain embodiments, the list of compounds is ranked by efficacy with statistical significance. See, for example, FIGS. 50A-C for exemplary outputs produced when clinical indication inputted into PhAROS is migraine.

In some embodiments, the outputting further comprises outputting molecular targets for the list of compounds that are clinically indicated for migraine across one or more TMS.

In some embodiments, the molecular targets comprise: Prelamin-A/C; Lysine-specific demethylase 4D-like; Microtubule-associated protein tau; Microtubule-associated protein tau; Endonuclease 4; Peripheral myelin protein 22; Nonstructural protein 1; Bloom syndrome protein; Bloom syndrome protein; Neuropeptide S receptor; Geminin; Histone-lysine N-methyltransferase, H3 lysine-9 specific 3; Geminin; Thioredoxin reductase 1, cytoplasmic; Acetylcholinesterase; Cholinesterase; Solute carrier organic anion transporter family member 1B1; Solute carrier organic anion transporter family member 1B3 Nuclear factor NF-kappa-B p65 subunit; p53-binding protein Mdm-2; Huntingtin; Ras-related protein Rab-9A; Survival motor neuron protein; Tyrosyl-DNA phosphodiesterase 1; Microtubule-associated protein tau; Microtubule-associated protein tau; Microtubule-associated protein tau; Nuclear receptor ROR-gamma; Aldehyde dehydrogenase 1A1; Thioredoxin glutathione reductase; 4′-phosphopantetheinyl transferase ffp; 4′-phosphopantetheinyl transferase ffp; Nonstructural protein 1; Microtubule-associated protein tau; Microtubule-associated protein tau; Type-1 angiotensin II receptor; Niemann-Pick C1 protein; MAP kinase ERK2; Nuclear receptor ROR-gamma; Alpha-galactosidase A; DNA polymerase beta; Beta-glucocerebrosidase; Nuclear factor erythroid 2-related factor 2; X-box-binding protein 1; Histone acetyltransferase GCN5; G-protein coupled receptor 55; Histone-lysine N-methyltransferase, H3 lysine-9 specific 3; DNA damage-inducible transcript 3 protein; ATPase family AAA domain-containing protein 5; Vitamin D receptor; Vitamin D receptor; Chromobox protein homolog 1; Thioredoxin reductase 1, cytoplasmic; DNA polymerase iota; DNA polymerase eta; Regulator of G-protein signaling 4; Beta-galactosidase; Regulator of G-protein signaling 4; Mothers against decapentaplegic homolog 3; Geminin; Alpha trans-inducing protein (VP16); ATPase family AAA domain-containing protein 5; ATPase family AAA domain-containing protein 5; ATPase family AAA domain-containing protein 5; DNA dC->dU-editing enzyme APOBEC-3G; Photoreceptor-specific nuclear receptor; Geminin; Ataxin-2; Glucagon-like peptide 1 receptor; ATPase family AAA domain-containing protein 5; ATPase family AAA domain-containing protein 5; ATPase family AAA domain-containing protein 5; ATPase family AAA domain-containing protein 5; Tyrosyl-DNA phosphodiesterase 1; Isocitrate dehydrogenase [NADP] cytoplasmic; Tyrosyl-DNA phosphodiesterase 1; Transcriptional activator Myb; Transcriptional activator Myb; Ubiquitin carboxyl-terminal hydrolase 1; Parathyroid hormone receptor; ATPase family AAA domain-containing protein 5; ATPase family AAA domain-containing protein 5; Telomerase reverse transcriptase; Telomerase reverse transcriptase Survival motor neuron protein; Thyroid hormone receptor beta-1; Arachidonate 15-lipoxygenase; Chromobox protein homolog 1; Geminin; Guanine nucleotide-binding protein G(s), subunit alpha; Pregnane X receptor; Nuclear receptor subfamily 1 group I member 2; Nuclear receptor subfamily 1 group I member 3; Pregnane X receptor; Pregnane X receptor; Pregnane X receptor; Pregnane X receptor; Nuclear receptor subfamily 1 group I member 2; Nuclear receptor subfamily 1 group I member 2; Pregnane X receptor; Pregnane X receptor; Nuclear receptor subfamily 1 group I member 2; Nuclear receptor subfamily 1 group I member 2; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; Nuclear receptor subfamily 1 group I member 3; and Nuclear receptor subfamily 1 group I member 3.

In some embodiments, the second user query input comprises the list of compounds.

In certain embodiments, further analysis initiated by the second user query input comprising the list of compounds comprises post-hoc screening for toxicity, chemical activity, or toxicity and chemical activity of the list of compounds. In certain embodiments, analysis comprises using the second user query input to search the data from the plurality of TMS associated with the second user query input.

In some embodiments, further analysis comprises processing the data associated with the second user query input to create a second processed data returned by the second query user input, and retrieving the second processed data based on the second query input for review by the user.

In some embodiments, the second processed data comprises a ranked list of potential minimal essential compounds required for efficacy of the new and/or optimized polypharmaceutical compositions.

In some embodiments, the list of compounds is categorized by class, identified as migraine dictionary search results, and are convergent between a plurality of TMS.

In some embodiments, the method further comprises further analysis initiated by a third user query input to identify the new polypharmaceutical and/or optimized polypharmaceutical compositions.

In some embodiments, further analysis comprises processing the data associated with the third user query input to create a third processed data returned by the query, and retrieving and outputting the third processed data based on the third user query input for review by the user.

In some embodiments, the third user query input comprises a query of neurotropic fungi associated with migraines in the plurality of TMS.

In some embodiments, the third processed data comprises one or more convergent compounds considered as alternative compounds of an existing transcultural compound with convergence between a plurality of TMS.

Pain Therapies Including Opioid-Alternative Strategies

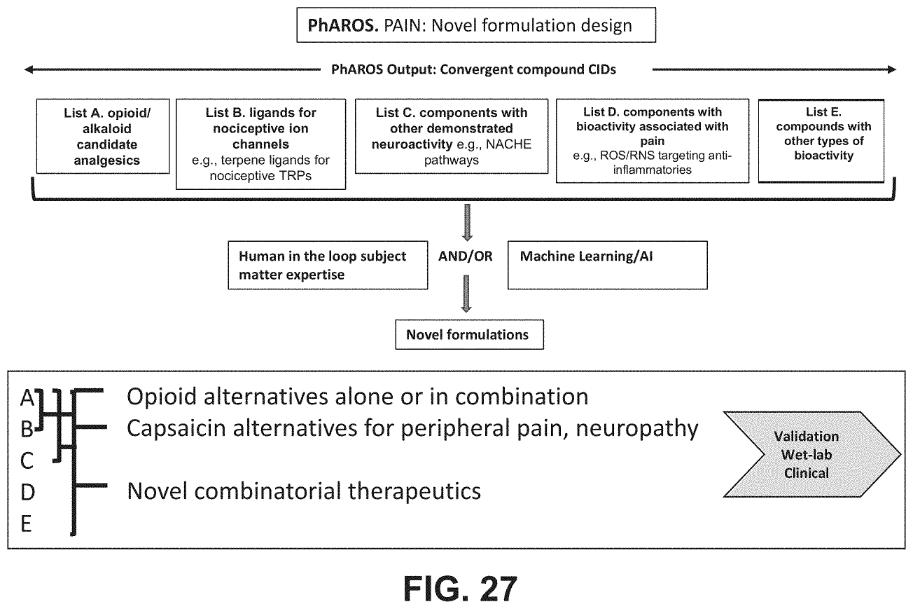

In some embodiments, the user selected clinical indication is pain. In such cases, PhAROS can be used to design new polypharmaceutical approaches for treating pain (see, e.g., Example 1). In some embodiments, PhAROS can be used to identify novel convergent formulation components for pain (see, e.g., Example 1). A non-limiting example for identifying and/or designing novel pain formulations includes the workflow as shown in FIG. 27.

In some embodiments, the processed data returned by the query comprises: a list of compounds associated with pain, a list of prescription formulae associated with pain, a list of organisms associated with pain, a list of chemicals associated with pain, or a combination thereof. In certain embodiments, the list of compounds, prescription formulae, organisms, and chemicals are indicated for pain across one or more TMS. See, for example, FIG. 22D for exemplary outputs.

In certain embodiments, the processed data further comprises: the identity of each TMS identified by an in silico convergent analysis, each TMS linked to one or more of: a number of compounds within the list of compounds associated with pain, a number of prescription formulae within the list of prescription formulae associated with pain, a number of organisms within the list of organisms associated with pain, and a number of chemicals within the list of chemicals associated with pain. See, for example, FIGS. 22A-22D for exemplary outputs for an in silico convergent analysis.

In some embodiments, the list of compounds comprises a list of alkaloids or terpenes.

In some embodiments, the list of compounds comprises: a list of opioids and/or alkaloid candidate analgesics, a list of ligands for nociceptive ion channels, a list of compounds with demonstrated neuroactivity, a list of compounds with bioactivity, and a list of compounds with bioactivity associated with pain.

In some embodiments, the second user query input comprises the list of compounds.

In some embodiments, further analysis initiated by the second user query input comprising the list of compounds comprises post-hoc screening for toxicity, chemical activity, or toxicity and chemical activity of the list of compounds.

In some embodiments, further analysis comprises using the second user query input to search the data from the plurality of TMS, the data from the plurality of TMS associated with the second user query input.

In some embodiments, further analysis comprises processing the data associated with the second user query input to create a second processed data returned by the second query user input, and retrieving the second processed data based on the second query input for review by the user.

In some embodiments, the second processed data comprises a ranked list of potential minimal essential compounds required for efficacy of the new and/or optimized polypharmaceutical compositions for treating pain. In certain embodiments, the second processed data comprises a second list of compounds ranked by one or more of: class, target, pathway, and coincidence or convergence of each of the compounds across specific TMS.

In some embodiments, the second processed data comprises a list of convergent compounds within the list of compounds between one or more TMS.

In some embodiments, the second processed data comprises a list of divergent compounds within the list of compounds

In some embodiments, the second processed data comprises a list of convergent compounds within the list of compounds that is considered as alternative compounds of an existing transcultural compound convergent between or more TMS.

In some embodiments, the list of compounds comprises a list of alkaloids, convergent between two or more TMS and associated with pain.

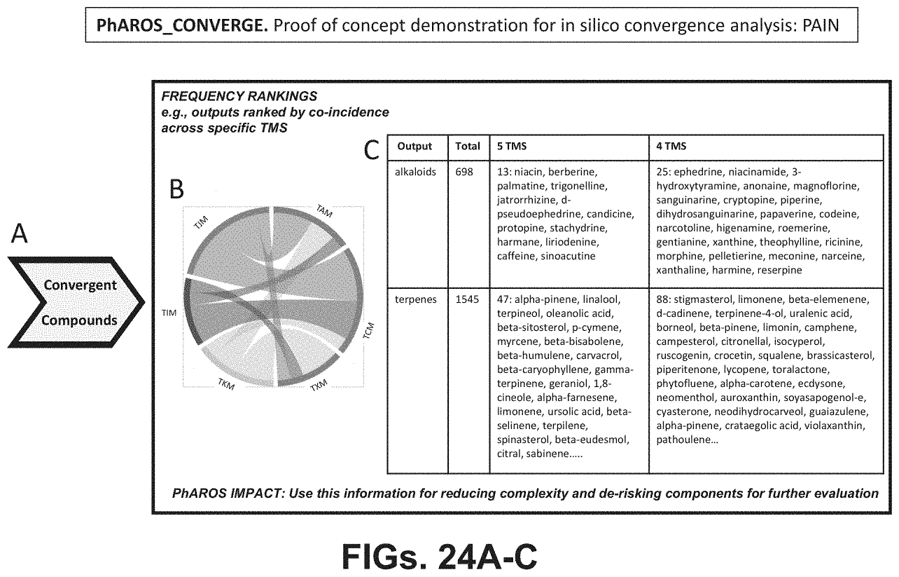

In certain embodiments, the list of alkaloids comprises: niacin, berberine, palmatine, trigonelline, jatrorrhizine, d-pseudoephedrine, candicine, protopine, stachydrine, harmane, liriodenine, caffeine, sinoacutine, ephedrine, niacinamide, 3-hydroxytyramine, anonaine, magnoflorine, sanguinarine, cryptopine, piperine, dihydrosanguinarine, papaverine, codeine, narcotoline, higenamine, roemerine, gentianine, xanthine, theophylline, ricinine, morphine, pelletierine, meconine, narceine, xanthaline, harmine, and reserpine (see, e.g., FIG. 24C).

In certain embodiments, the list of compounds comprises a list of terpenes convergent between one or more TMS and associated with pain.

In certain embodiments, the list of terpenes comprise: alpha-pinene, linalool, terpineol, oleanolic acid, beta-sitosterol, p-cymene, myrcene, beta-bisabolene, beta-humulene, carvacrol, beta-caryophyllene, gamma-terpinene, geraniol, 1,8-cineole, alpha-farnesene, limonene, ursolic acid, beta-selinene, terpilene, spinasterol, beta-eudesmol, citral, sabinene, stigmasterol, limonene, beta-elemenene, d-cadinene, terpinene-4-ol, uralenic acid, borneol, beta-pinene, limonin, camphene, campesterol, citronellal, isocyperol, ruscogenin, crocetin, squalene, brassicasterol, piperitenone, lycopene, toralactone, phytofluene, alpha-carotene, ecdysone, neomenthol, auroxanthin, soyasapogenol-e, cyasterone, neodihydrocarveol, guaiazulene, alpha-pinene, crataegolic acid, violaxanthin, and pathoulene (see, e.g., FIG. 24C).

Pain Type

In some embodiments, the user input query is pain type. In such cases, PhAROS can be used to identify new polypharmaceutical compositions targeted to specific pain subtypes (see, e.g., Example 2).

In some embodiments, the processed data returned by the query comprises: a list of pain types across one or more TMS.

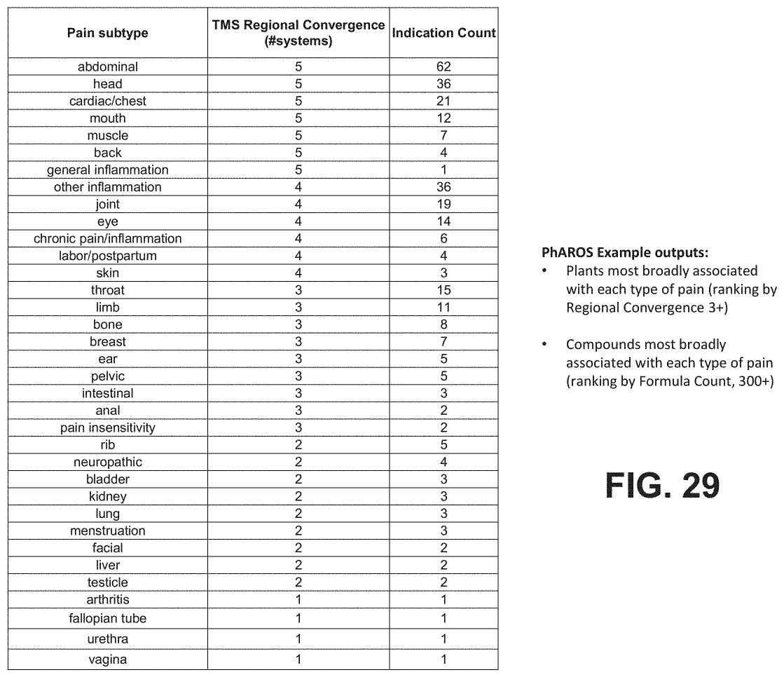

In some embodiments, the list of pain types comprises: abdominal, cardiac/chest, mouth, muscle, back, inflammation, joint, eye, chronic pain/inflammation, labor/postpartum, skin, throat, limb, bone, breast, ear, pelvic, intestinal, anal, pain sensitivity, rib, neuropathic, bladder, kidney, lung, menstruation, facial, liver, arthritis, fallopian tube, urethra, and vaginal, pain. See, for example, FIG. 29 and Tables 2 and 3 for exemplary analysis and output.

In some embodiments, for each pain type, the processed data comprises a list of TMS referenced from the plurality of TMS, associated with the pain type.

In some embodiments, the processed data returned by the query comprises a list of compounds associated with each pain type.

In some embodiments, the processed data further comprises a list of organisms for which the compounds within the list of compounds is derived.

In some embodiments, the processed data comprises the list of pain types and a list of organisms, wherein one or more pain types is associated with one or more organisms.

In some embodiments, the processed data comprises the list of pain types and a list of compounds, wherein one or more pain types is associated with one or more compounds.

In some embodiments, for each pain type, the processed data comprises identity of a plurality of TMS linked to one or more selected from: the pain type, one or more compounds associated with the pain type, and one or more organisms associated with the pain type.

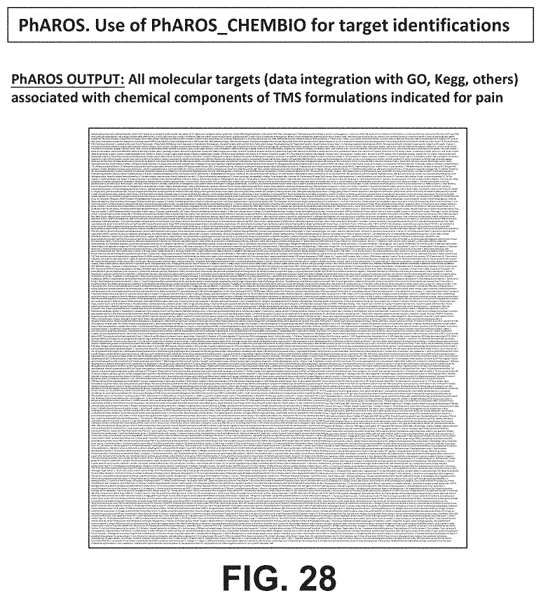

In some embodiments, an example PhAROS OUTPUT can include all molecular targets (data integration with GO, KEGG, others) associated with chemical components of TMS formulations indicated for pain. As shown in FIG. 28, the molecular targets include, but are not limited: Replicase polyprotein lab, Acetylcholinesterase, Solute carrier organic anion transporter family member 1B1, Solute carrier organic anion transporter family member 1B3, Tyrosyl-DNA phosphodiesterase 1, Cytochrome P450 3A4, Cyclooxygenase-2, Cholinesterase, Aldose reductase, Geminin, Cyclooxygenase-1, Cytochrome P450 2D6, Nuclear factor erythroid 2-related factor 2, Cytochrome P450 1A2, Cytochrome P450 2C9, Cytochrome P450 2C19, Aldehyde dehydrogenase 1A1, Estrogen receptor alpha, DNA-(apurinic or apyrimidinic site) lyase, Carbonic anhydrase II, MAP kinase ERK2, Glucocorticoid receptor, Androgen Receptor, Prelamin-A/C, Arachidonate 15-lipoxygenase, Nuclear receptor ROR-gamma, Epidermal growth factor receptor erbB1, Microtubule-associated protein tau, Histone-lysine N-methyltransferase, H3 lysine-9 specific 3, Isocitrate dehydrogenase [NADP] cytoplasmic, Monoamine oxidase A, Adenosine A1 receptor, Nitric oxide synthase, inducible, Chromobox protein homolog 1, Protein-tyrosine phosphatase 1B, Tyrosinase, P-glycoprotein 1, Tyrosine-protein kinase LCK, HERG, DNA polymerase beta, Ubiquitin carboxyl-terminal hydrolase 1, Protein kinase C alpha, Lysine-specific demethylase 4D-like, Leukocyte elastase, DNA polymerase iota, Matrix metalloproteinase 9, Dopamine D1 receptor, Muscarinic acetylcholine receptor M1, Angiotensin-converting enzyme, MAP kinase p38 alpha, Matrix metalloproteinase-1, MAP kinase ERK1, DNA polymerase kappa, Adenosine A3 receptor, Thyroid stimulating hormone receptor, Beta amyloid A4 protein, Adenosine A2a receptor, Endoplasmic reticulum-associated amyloid beta-peptide-binding protein, 4′-phosphopantetheinyl transferase ffp, Peripheral myelin protein 22, Bile acid receptor FXR, Thioredoxin reductase 1, cytoplasmic, Serotonin 1a (5-HT1a) receptor, ATPase family AAA domain-containing protein 5, Arachidonate 5-lipoxygenase, Mu opioid receptor, Anthrax lethal factor, Delta opioid receptor, Phosphodiesterase 5A, Kappa opioid receptor, Thyroid hormone receptor beta-1, 15-hydroxyprostaglandin dehydrogenase [NAD+], Peroxisome proliferator-activated receptor gamma, Dopamine D4 receptor, Caspase-1, Peroxisome proliferator-activated receptor delta, Leukocyte common antigen, Insulin receptor, Estrogen receptor beta, Interleukin-8 receptor A, C—C chemokine receptor type 4, Dopamine transporter, Xanthine dehydrogenase, Cannabinoid CB1 receptor, Receptor protein-tyrosine kinase erbB-2, Serotonin 2c (5-HT2c) receptor, Beta-2 adrenergic receptor, Cytochrome P450 2A6, Dopamine D2 receptor, Cathepsin G, Tyrosine-protein kinase FYN, HMG-CoA reductase, Glycogen synthase kinase-3 beta, Histone acetyltransferase GCN5, Serotonin transporter, Alpha-2a adrenergic receptor, Carbonic anhydrase I, Alpha-2c adrenergic receptor, Serotonin 2a (5-HT2a) receptor, Progesterone receptor, 6-phospho-1-fructokinase, Nitric-oxide synthase, brain, Cytochrome P450 2E1, UDP-glucuronosyltransferase 1-1, Beta-lactamase AmpC, Norepinephrine transporter, Flap endonuclease 1, Dopamine D3 receptor, Cytochrome P450 19A1, Alpha-1b adrenergic receptor, Beta-1 adrenergic receptor, Muscarinic acetylcholine receptor M3, Alpha-1a adrenergic receptor, Serotonin 2b (5-HT2b) receptor, Muscarinic acetylcholine receptor M5, Muscarinic acetylcholine receptor M4, Muscarinic acetylcholine receptor M2, Histamine H1 receptor, Serotonin 6 (5-HT6) receptor, Alpha-1d adrenergic receptor, Serotonin 1b (5-HT1b) receptor, Alpha-2b adrenergic receptor, Vascular endothelial growth factor receptor 1, Vitamin D receptor, Sigma opioid receptor, Platelet activating factor receptor, UDP-glucuronosyltransferase 1A4, Histamine H2 receptor, Endothelin receptor ET-A, Thromboxane-A synthase, Neuropeptide Y receptor type 2, Neuropeptide Y receptor type 1, Serotonin 4 (5-HT4) receptor, Beta-3 adrenergic receptor, Vasopressin V1a receptor, Vasoactive intestinal polypeptide receptor 1, Serine/threonine protein phosphatase 2B catalytic subunit, alpha isoform, Neurokinin 2 receptor, Neurokinin 1 receptor, Melanocortin receptor 5, Melanocortin receptor 4, Melanocortin receptor 3, Leukotriene C4 synthase, Interleukin-8 receptor B, Cysteinyl leukotriene receptor 1, Cholecystokinin A receptor, Calcitonin receptor, C—C chemokine receptor type 5, C—C chemokine receptor type 2, Bradykinin B2 receptor, Angiotensin II type 2 (AT-2) receptor, Survival motor neuron protein, Serum albumin, Carbonic anhydrase XII, Cellular tumor antigen p53, Carbonic anhydrase VII, Glutaminase kidney isoform, mitochondrial, Parathyroid hormone receptor, ATP-dependent DNA helicase Q1, Lysine-specific demethylase 4A, Thrombin, Luciferin 4-monooxygenase, Cruzipain, Carbonic anhydrase IV, Carbonic anhydrase IX, Mothers against decapentaplegic homolog 3, Nuclear factor NF-kappa-B p105 subunit, Bromodomain adjacent to zinc finger domain protein 2B, DNA topoisomerase I, Lysosomal alpha-glucosidase, Arachidonate 15-lipoxygenase, type II, Putative fructose-1,6-bisphosphate aldolase, Pancreatic triacylglycerol lipase, ATP-binding cassette sub-family G member 2, Neuraminidase, Aldo-keto reductase family 1 member B10, Fatty acid synthase, DNA topoisomerase II alpha, Butyrylcholinesterase, Bloom syndrome protein, UDP-glucuronosyltransferase 1-10, Rap guanine nucleotide exchange factor 3, Regulator of G-protein signaling 4, Dipeptidyl peptidase IV, Serine/threonine-protein kinase PLK1, Beta-glucocerebrosidase, Peptidyl-prolyl cis-trans isomerase NIMA-interacting 1, Bile salt export pump, Solute carrier organic anion transporter family member 2B1, Cerebroside-sulfatase, UDP-glucuronosyltransferase 1-9, UDP-glucuronosyltransferase 1-8, Monoamine oxidase B, Retinoid X receptor alpha, Reverse transcriptase, UDP-glucuronosyltransferase 2B15, Transcription factor Sp1, Peroxisome proliferator-activated receptor alpha, Muscleblind-like protein 1, 3-oxoacyl-acyl-carrier protein reductase, Alpha-glucosidase MAL62, Hypoxia-inducible factor 1 alpha, Ataxin-2, Beta-secretase 1, DNA polymerase eta, Carbonic anhydrase XIII, Glucagon-like peptide 1 receptor, Multidrug resistance-associated protein 1, Inositol monophosphatase 1, Cytochrome P450 1B1, Carbonic anhydrase VI, Carbonic anhydrase XIV, Cytochrome P450 1A1, Ferritin light chain, Carbonic anhydrase VA, Human immunodeficiency virus type 1 integrase, UDP-glucuronosyltransferase 1-3, TAR DNA-binding protein 43, UDP-glucuronosyltransferase 1-6, Rap guanine nucleotide exchange factor 4, Carbonic anhydrase VB, Quinone oxidoreductase, Carbonic anhydrase III, Canalicular multispecific organic anion transporter 1, Dihydroorotate dehydrogenase (fumarate), Seed lipoxygenase-1, Interleukin-8, Dual specificity protein phosphatase 3, Protein-tyrosine phosphatase LC-PTP, Tyrosine-protein kinase SYK, Integrase, Alpha-galactosidase A, Proteasome Macropain subunit MB1, Enoyl-acyl-carrier protein reductase, Estradiol 17-beta-dehydrogenase 2, Thioredoxin glutathione reductase, Matrix metalloproteinase-2, Guanine nucleotide-binding protein G(s), subunit alpha, Solute carrier family 2, facilitated glucose transporter member 4, Tyrosine-protein kinase SRC, Serotonin 7 (5-HT7) receptor, GABA receptor subunit, Serine/threonine-protein kinase PIM1, Serine/threonine-protein kinase AKT, Myeloperoxidase, UDP-glucuronosyltransferase 2A1, LDL-associated phospholipase A2, Acidic alpha-glucosidase, Ubiquitin carboxyl-terminal hydrolase 2, Transient receptor potential cation channel subfamily A member 1, 11-beta-hydroxysteroid dehydrogenase 2, Sucrase-isomaltase, Neuropeptide S receptor, Taste receptor type 2 member 39, Nuclear factor NF-kappa-B p65 subunit, Matrix metalloproteinase 3, Lethal(3)malignant brain tumor-like protein 1, Pancreatic alpha-amylase, Protein-tyrosine phosphatase 2C, Toll-like receptor 2, Hydroxycarboxylic acid receptor 2, Breast cancer type 1 susceptibility protein, Epoxide hydratase, Carbonic anhydrase, Anthrax toxin receptor 2, Voltage-gated L-type calcium channel alpha-1C subunit, Nonstructural protein 1, Signal transducer and activator of transcription 3, Estradiol 17-beta-dehydrogenase 1, Cyclin-dependent kinase 2, Quinolone resistance protein norA, Salivary alpha-amylase, Arginase, Low molecular weight phosphotyrosine protein phosphatase, Glyoxalase I, 78 kDa glucose-regulated protein, Sialidase, Beta-lactamase, Tyrosine-protein kinase receptor FLT3, Aryl hydrocarbon receptor, Egl nine homolog 1, Histone-lysine N-methyltransferase MLL, Genome polyprotein, Death-associated protein kinase 1, Lactoperoxidase, Prolyl endopeptidase, Enoyl-ACP reductase, Solute carrier family 22 member 1, Free fatty acid receptor 3, M-phase phosphoprotein 8, Serotonin 3a (5-HT3a) receptor, LXR-alpha, Toll-like receptor 4, LXR-beta, Arachidonate 12-lipoxygenase, Lysine-specific histone demethylase 1, Glycogen phosphorylase, muscle form, Neuronal acetylcholine receptor protein alpha-7 subunit, Anandamide amidohydrolase, T-cell protein-tyrosine phosphatase, 11-beta-hydroxysteroid dehydrogenase 1, Protein-tyrosine phosphatase 1C, GABA transporter 1, Dual specificity tyrosine-phosphorylation-regulated kinase 1A, Diacylglycerol O-acyltransferase 1, Large T antigen, Aldehyde oxidase, Fatty acid binding protein adipocyte, Niemann-Pick C1 protein, Hepatocyte growth factor receptor, Glyceraldehyde-3-phosphate dehydrogenase liver, Monocarboxylate transporter 1, Solute carrier family 22 member 2, Canalicular multispecific organic anion transporter 2, Solute carrier family 22 member 5, Putative uncharacterized protein, Pyruvate dehydrogenase kinase isoform 1, Sphingomyelin phosphodiesterase, Ras-related protein Rab-9A, Lecithin retinol acyltransferase, Plasma retinol-binding protein, Transient receptor potential cation channel subfamily V member 2, DNA-3-methyladenine glycosylase, G-protein coupled bile acid receptor 1, Fatty acid binding protein muscle, Casein kinase II alpha, Transthyretin, Solute carrier family 22 member 6, 2-heptyl-4(1H)-quinolone synthase PqsD, Transient receptor potential cation channel subfamily M member 8, Fatty acid binding protein epidermal, Solute carrier organic anion transporter family member 1A5, Sialidase 2, Olfactory receptor 51E2, MAP kinase-activated protein kinase 2, Voltage-gated potassium channel subunit Kv1.5, RAC-alpha serine/threonine-protein kinase, Solute carrier family 22 member 8, DNA polymerase lambda, 5′-AMP-activated protein kinase catalytic subunit alpha-2, ATP-dependent Clp protease proteolytic subunit, Inositol polyphosphate multikinase, Inositol hexakisphosphate kinase 2, Endonuclease 4, Avian myoblastosis virus polyprotein II, Matrix metalloproteinase 8, Alpha-chymotrypsin, Carbonic anhydrase 15, Multidrug resistance-associated protein 4, Cannabinoid CB2 receptor, Telomerase reverse transcriptase, PI3-kinase p110-alpha subunit, Cathepsin D, Dual specificity mitogen-activated protein kinase kinase 1, Solute carrier family 22 member 4, Solute carrier family 22 member 3, Mitogen-activated protein kinase kinase kinase 5, Cyclin-dependent kinase 6, Indoleamine 2,3-dioxygenase, Catalase, Serine/threonine-protein kinase Sgk1, Alpha-synuclein, Glyceraldehyde-3-phosphate dehydrogenase, glycosomal, DNA dC->dU-editing enzyme APOBEC-3G, Phospholipase A2 group 1B, Calmodulin, Rhodopsin, NADPH oxidase 4, Phosphoglycerate kinase, glycosomal, Serum paraoxonase/arylesterase 1, Fatty acid binding protein intestinal, Olfactory receptor 5K1, Caspase-7, UDP-glucuronosyltransferase 2B17, Hyaluronidase-1, Trypsin I, Serine/threonine-protein kinase mTOR, Sortase A, Gamma-amino-N-butyrate transaminase, Alkaline phosphatase, tissue-nonspecific isozyme, Sorbitol dehydrogenase, Intestinal alkaline phosphatase, Choline acetylase, Plasminogen, Fibroblast growth factor receptor 1, Protease, Fibroblast growth factor receptor 2, Aberrant vpr protein, Cell division protein kinase 5, Transcriptional regulator ERG, Thrombopoietin, Short transient receptor potential channel 5, Streptokinase A, c-Jun N-terminal kinase 2, Tartrate-resistant acid phosphatase type 5, Enoyl-[acyl-carrier-protein] reductase, Alanine aminotransferase 1, c-Jun N-terminal kinase 1, Choline transporter, Monoglyceride lipase, Dual specificity phosphatase Cdc25B, 1-phosphatidylinositol-4,5-bisphosphate phosphodiesterase gamma-1, Creatine transporter, Glucose transporter, Serine/threonine-protein kinase Aurora-B, Tyrosine-protein kinase JAK1, Receptor-type tyrosine-protein phosphatase F (LAR), Alkaline phosphatase placental-like, Coagulation factor X, Protein E6, Nuclear receptor subfamily 1 group I member 2, Serine/threonine-protein kinase B-raf, Serine-protein kinase ATM, DNA ligase 1, Vanilloid receptor, Coagulation factor III, Aldo-keto-reductase family 1 member C3, Cytochrome P450 2B6, P-selectin, Selectin E, Receptor-type tyrosine-protein phosphatase alpha, NAD-dependent deacetylase sirtuin 1, Solute carrier family 22 member 20, Signal transducer and activator of transcription 6, UDP-glucuronosyltransferase 1-7, Cholesteryl ester transfer protein, 3-phosphoinositide dependent protein kinase-1, Polymerase acidic protein, Leukocyte adhesion molecule-1, Protein kinase C beta, D-amino-acid oxidase, MAP kinase p38 beta, Streptavidin, Serine/threonine-protein kinase Chk1, Ribosomal protein S6 kinase 1, GP41, Focal adhesion kinase 1, Serine/threonine-protein kinase PIM2, Beta-glucuronidase, Receptor-type tyrosine-protein phosphatase epsilon, Free fatty acid receptor 1, cAMP-dependent protein kinase alpha-catalytic subunit, G-protein coupled receptor 120, Trypsin, Vascular endothelial growth factor receptor 2, Aldo-keto reductase family 1 member C2, Insulin-like growth factor I receptor, Human immunodeficiency virus type 1 reverse transcriptase, AMP-activated protein kinase, alpha-2 subunit, G-protein coupled receptor 35, Histamine H3 receptor, Fibrinogen C domain-containing protein 1, Serine/threonine-protein kinase PAK 4, Serine/threonine-protein kinase NEK2, Ribosomal protein S6 kinase alpha 3, Acyl coenzyme A:cholesterol acyltransferase 1, Heat shock protein HSP 90-beta, Serine/threonine-protein kinase PIM3, Serine/threonine-protein kinase PAK6, D-aspartate oxidase, Serine/threonine-protein kinase PAK7, Serine/threonine-protein kinase NEK6, Serine/threonine-protein kinase Chk2, CaM-kinase kinase beta, Beta-amylase, Alpha-amylase, MAP kinase-activated protein kinase 5, DNA topoisomerase II beta, Aldehyde reductase, Ribosomal protein S6 kinase alpha 5, Rho-associated protein kinase 2, Cathepsin L, Heat shock factor protein 1, Rac GTPase-activating protein 1, Aldehyde dehydrogenase, 14-3-3 protein epsilon, Phosphotyrosine protein phosphatase, Proto-oncogene c-JUN, Cholesterol 24-hydroxylase, Prolyl 4-hydroxylase, Cyclin-dependent kinase 1, MAP kinase p38 gamma, MAP kinase p38 delta, Serine/threonine-protein kinase PLK4, Chymotrypsin C, Dual specificty protein kinase CLK1, PI3-kinase p110-gamma subunit, G-protein coupled receptor 84, Glycine receptor subunit alpha-1, 2,3-bisphosphoglycerate-independent phosphoglycerate mutase, Acetyl-CoA acetyltransferase, mitochondrial, Casein kinase I gamma 2, Carboxy-terminal domain RNA polymerase II polypeptide A small phosphatase 1, Dual specificity mitogen-activated protein kinase kinase 6, Casein kinase I gamma 1, Serine/threonine-protein kinase 10, Serine/threonine-protein kinase MST1, Casein kinase I isoform gamma-3, CaM kinase IV, CaM kinase II gamma, CaM kinase II delta, Solute carrier family 28 member 3, Huntingtin, Carbonic anhydrase 2, Dual specificity protein kinase CLK3, Dual specificity protein kinase CLK2, Death-associated protein kinase 3, Serine/threonine-protein kinase VRK2, Serine/threonine-protein kinase MST4, Serine/threonine-protein kinase 2, Serine/threonine-protein kinase 17A, Serine/threonine-protein kinase 16, Myotonin-protein kinase, Dual specificity mitogen-activated protein kinase kinase 2, CaM kinase II beta, CaM kinase I delta, TRAF2- and NCK-interacting kinase, Serine/threonine-protein kinase PCTAIRE-1, Serine/threonine-protein kinase 38, Alpha 1,4 galactosyltransferase, M18 aspartyl aminopeptidase, Lymphocyte differentiation antigen CD38, Werner syndrome ATP-dependent helicase, Transcription factor p65, Pyruvate kinase isozymes M1/M2, Liver glycogen phosphorylase, Serine/threonine-protein kinase OSR1, Mitogen-activated protein kinase 6, CaM kinase II alpha, Serine/threonine-protein kinase VRK1, Serine/threonine-protein kinase RIO2, Serine/threonine-protein kinase 25, PDZ-binding kinase, Inactive serine/threonine-protein kinase VRK3, Cyclin-dependent kinase-like 1, CaM kinase I gamma, Proton-coupled amino acid transporter 1, Protein disulfide-isomerase, Catechol O-methyltransferase, Maltase-glucoamylase, Human immunodeficiency virus type 1 protease, SLC16A10 protein, Pendrin, Galactocerebrosidase, Receptor-type tyrosine-protein phosphatase S, Cytochrome P450 2A5, NACHT, LRR and PYD domains-containing protein 3, Acrosin, NADP-dependent malic enzyme, mitochondrial, DNA polymerase III, Carbonyl reductase [NADPH] 3, Carbonyl reductase [NADPH] 1, Calcium-activated potassium channel subunit alpha-1, Tumor susceptibility gene 101 protein, Aldo-keto reductase family 1 member C4, Aldo-keto reductase family 1 member C1, p53-binding protein Mdm-2, Cytochrome P450 2C8, DNA repair protein RAD52 homolog, Succinate semialdehyde dehydrogenase, Eyes absent homolog 2, Polyphenol oxidase, Neuromedin-U receptor 2, Endoplasmic reticulum aminopeptidase 1, G-protein coupled receptor 81, Matrix metalloproteinase 13, Matrix metalloproteinase 12, Squalene monooxygenase, Inhibitor of apoptosis protein 3, 1-phosphatidylinositol-4,5-bisphosphate phosphodiesterase gamma-2, Nitric-oxide synthase, endothelial, Inhibitor of nuclear factor kappa B kinase beta subunit, Hypoxia-inducible factor 1-alpha inhibitor, Aryl sulfotransferase, Multidrug and toxin extrusion protein 1, Tyrosine-protein kinase CSK, Equilibrative nucleoside transporter 1, Sodium/nucleoside cotransporter 2, Sodium/nucleoside cotransporter 1, Equilibrative nucleoside transporter 2, Signal transducer and activator of transcription 1-alpha/beta, UDP-glucuronosyltransferase 2B7, Signal transducer and activator of transcription 5B, Platelet-derived growth factor receptor beta, Aminopeptidase N, L-xylulose reductase, P-glycoprotein 3, Estrogen-related receptor alpha, Potassium channel subfamily K member 2, 5-lipoxygenase, Histone deacetylase 1, High-affinity choline transporter, BiP isoform A, Solute carrier family 22 member 11, Dihydroorotate dehydrogenase, Galactokinase, Cytosol aminopeptidase, Papain, Tyrosine-protein kinase Lyn, Aldo-keto reductase family 1 member C21, Neprilysin, Heat shock cognate 71 kDa protein, Acyl coenzyme A:cholesterol acyltransferase, CaM kinase I alpha, Sterol regulatory element-binding protein 2, HM74 nicotinic acid GPCR, Adenosine kinase, Thiopurine S-methyltransferase, Dynamin-1, CDGSH iron-sulfur domain-containing protein 1, FAD-linked sulfhydryl oxidase ALR, Sulfotransferase 1A1, Glutathione reductase, Serine/threonine-protein kinase Aurora-A, Apoptosis regulator Bcl-2, Oleandomycin glycosyltransferase, L-lactate dehydrogenase A chain, D-alanylalanine synthetase, D-alanine-D-alanine ligase, Mitogen-activated protein kinase kinase kinase 7, Poly [ADP-ribose] polymerase-1, Glucose-6-phosphate 1-dehydrogenase, Lysine-specific demethylase 5A, ELAV-like protein 3, Adenosine A2b receptor, Alpha-ketoglutarate-dependent dioxygenase FTO, High mobility group protein B1, Steroid 5-alpha-reductase 1, Adenylate cyclase type V, Purine nucleoside phosphorylase, Adenosine deaminase-like protein, Adenylate kinase 2, Adenosylhomocysteinase, Adenylate kinase 1, Major pollen allergen Bet v 1-A, Fluoroquinolone resistance protein, Endoplasmic reticulum aminopeptidase 2, Inosine-5′-monophosphate dehydrogenase 2, Adenosine deaminase, 5-methylthioadenosine/S-adenosylhomocysteine deaminase, 3-dehydroquinate synthase, Inosine-5′-monophosphate dehydrogenase 1, Histone-lysine N-methyltransferase, H3 lysine-79 specific, ATP-citrate synthase, Spermidine synthase, S-methyl-5-thioadenosine phosphorylase, S-adenosylhomocysteine nucleosidase, Avidin, Adenosine transporter 1, Solute carrier family 22 member 7, Ribonuclease pancreatic, UDP-glucuronosyltransferase 2B4, Taste receptor type 1 member 3, Zn finger protein, GABA transporter 3, Purinergic receptor P2Y12, Oligo-1,6-glucosidase, GABA transporter 4, ADAM17, DNA-dependent protein kinase, Serine/threonine-protein kinase AKT2, Monocarboxylate transporter 10, Interleukin-2, TNF-alpha, c-Jun N-terminal kinase 3, Ras-related C3 botulinum toxin substrate 1, Autoinducer 2-binding periplasmic protein luxP, Cell division control protein 42 homolog, Rho-associated protein kinase 1, Solute carrier organic anion transporter family member 1A2, Acetylcholine receptor subunit beta-like 2, Testis-specific androgen-binding protein, Solute carrier organic anion transporter family member 1A1, ALK tyrosine kinase receptor, Monocarboxylate transporter 2, Sarcoplasmic/endoplasmic reticulum calcium ATP-ase, Superoxide dismutase, Tyrosine-protein kinase YES, Dual-specificity tyrosine-phosphorylation regulated kinase 1A, GABA transporter 2, Phenylethanolamine N-methyltransferase, Solute carrier organic anion transporter family member 1A4, Tumor necrosis factor receptor superfamily member 10B, Histone deacetylase 6, GABA receptor rho-1 subunit, GABA receptor alpha-1 subunit, GABA receptor gamma-1 subunit, Tyrosine-protein kinase receptor UFO, Ribonuclease HI, 3-keto-steroid reductase, Transcription intermediary factor 1-alpha, E3 ubiquitin-protein ligase TRIM33, Tankyrase-2, Tankyrase-1, Dengue virus type 2 NS3 protein, Voltage-gated potassium channel subunit Kv1.1, Pyruvate kinase, Cytochrome b-245 heavy chain, Translin-associated protein X, NUAK family SNF1-like kinase 1, Lysozyme, Ornithine decarboxylase, Proteasome component C5, Proteasome Macropain subunit, Tyrosine-protein kinase receptor RET, Glutamate decarboxylase 67 kDa isoform, Beta-chymotrypsin, Ribosomal protein S6 kinase alpha 1, Betaine transporter, Polypeptide N-acetylgalactosaminyltransferase 2, Fructose-bisphosphate aldolase A, Calcium release-activated calcium channel protein 1, Carbonic anhydrase-like protein, putative, Alpha-(1,3)-fucosyltransferase 7, Fucosyltransferase 4, Heat shock protein beta-1, Collagen, Serine racemase, Gamma-hydroxybutyrate receptor, GABA receptor alpha-4 subunit, Botulinum neurotoxin type A, Hemoglobin beta chain, Voltage-gated potassium channel subunit Kv1.3, GABA-B receptor 1, GABA receptor alpha-6 subunit, GABA receptor alpha-3 subunit, GABA receptor alpha-2 subunit, UDP-glucuronosyltransferase 2B10, Uncharacterized protein Rv1284/MT1322, PROBABLE TRANSMEMBRANE CARBONIC ANHYDRASE (CARBONATE DEHYDRATASE) (CARBONIC DEHYDRATASE), Alpha-L-fucosidase I, Carboxylesterase 2, Tyrosine-protein kinase JAK3, Glycoprotein hormones alpha chain, Protein kinase N1, Tyrosine-protein kinase FES, Serine/threonine-protein kinase RIPK2, Serine/threonine-protein kinase PAK 2, Solute carrier family 2, facilitated glucose transporter member 2, Squalene synthetase, Estrogen sulfotransferase, Phosphodiesterase 2A, Prenyltransferase homolog, Tyrosine-protein kinase FGR, Cytochrome P450 2J2, Histone deacetylase 3, NF-kappa-B inhibitor alpha, Zinc finger protein mex-5, Cytoplasmic zinc-finger protein, Voltage-gated potassium channel subunit Kv1.2, DNA dC->dU-editing enzyme APOBEC-3F, General amino-acid permease GAP1, Urokinase-type plasminogen activator, Replicative DNA helicase, Protein RecA, Malate dehydrogenase, 5′-nucleotidase, Protein kinase C delta, Vasopressin V2 receptor, PI3-kinase p85-alpha subunit, Hexokinase, ELAV-like protein 1, Aflatoxin B1 aldehyde reductase, Myelin basic protein, Serine/threonine-protein kinase RAF, Nicotinate phosphoribosyltransferase, Elastase 2A, Receptor protein-tyrosine kinase erbB-4, Tyrosine-protein kinase ITK/TSK, Cystic fibrosis transmembrane conductance regulator, Thioredoxin reductase 2, mitochondrial, PI3-kinase p110-delta subunit, Histone deacetylase 5, Histone deacetylase 4, Ubiquitin carboxyl-terminal hydrolase 7, Dihydrofolate reductase, DNA polymerase alpha subunit, Cytosolic purine 5′-nucleotidase, Alanine aminotransferase, Voltage-gated potassium channel subunit Kv3.1, Voltage-gated potassium channel subunit Kv1.6, Mitogen-activated protein kinase kinase kinase kinase 5, Glucose-6-phosphate translocase, Cell division protein ftsZ, Stem cell growth factor receptor, Fucosyltransferase 10, Histidine-rich protein, Tryptophan 5-monooxygenase 1, Beta-1,3-glucan synthase, Cytochrome P450 51, Pregnane X receptor, Phenol oxidase, Dihydrodipicolinate synthase, Hepatitis C virus NS3 protease/helicase, L-type amino acid transporter 1, Ubiquitin carboxyl-terminal hydrolase 47, Phospholipase A2 isozyme PLA-A, Phospholipase A2 isozyme DE-I, Phospholipase A2, Hemagglutinin, Glycogen [starch] synthase, liver, Glucose-6-phosphatase, Ghrelin O-acyltransferase, Serine/threonine-protein kinase BUD32, Protein kinase C epsilon, 3-oxo-5-beta-steroid 4-dehydrogenase, Protein kinase C eta, Putative cytochrome P450 125, Tumor necrosis factor ligand superfamily member 11, Induced myeloid leukemia cell differentiation protein Mcl-1, Heparanase, T-complex protein 1 subunit beta, Sodium/glucose cotransporter 2, Sodium/glucose cotransporter 1, Solute carrier family 2, facilitated glucose transporter member 1, Mucin-1, Transforming protein RhoA, Sulfotransferase family cytosolic 2B member 1, Muscle glycogen phosphorylase, Brain glycogen phosphorylase, Fatty acid-binding protein, liver, Thymidine kinase, Acetylcholine receptor protein delta chain, Envelope glycoprotein, mRNA interferase MazF, Solute carrier organic anion transporter family member 1B2, Niemann-Pick C1-like protein 1, Cytochrome P450 11A1, Malate dehydrogenase cytoplasmic, 6-phosphogluconate dehydrogenase, Lipopolysaccharide heptosyltransferase 1, Trypanothione reductase, Prostaglandin E synthase, Retinoic acid receptor alpha, Heat shock protein HSP 90-alpha, Citrate synthase, mitochondrial, DNA (cytosine-5)-methyltransferase 1, Thiosulfate sulfurtransferase, 60 kDa chaperonin, Putative organic anion transporter 5, P2X purinoceptor 7, Zinc finger protein GLI1, Fructose-1,6-bisphosphatase, Caspase-3, Proteasome Macropain subunit PRE2, Protein kinase Pfmrk, Collagenase, Beta-lactoglobulin, Tryptase beta-1, 72 kDa type IV collagenase, Thymidine phosphorylase, Apoptosis regulator Bcl-X, Bifunctional protein glmU, Ubiquitin-like domain-containing CTD phosphatase 1, Tyrosine-protein phosphatase yopH, Hydroxyacid oxidase 1, Cytochrome P450 2A13, Dihydroorotase, 3-oxoacyl-[acyl-carrier-protein] synthase 3, Quinone reductase 1), NAD-dependent deacetylase sirtuin 2, Arginase-1, 4-hydroxyphenylpyruvate dioxygenase, Melatonin receptor 1A, Amino acid transporter, Monocarboxylate transporter 8, Nuclear receptor subfamily 0 group B member 1, Phospholipase A-2-activating protein, NAD-dependent protein deacylase sirtuin-5, mitochondrial, Amine oxidase, copper containing, S-adenosylmethionine synthetase gamma form, S-adenosylmethionine synthetase alpha and beta forms, Alpha-mannosidase 2C1, Glucosyltransferase-SI, M1-family aminopeptidase, Lectin, Fucose-binding lectin PA-IIL, CD209 antigen, Adhesin protein fimH, Pulmonary surfactant-associated protein D, Mannosyl-oligosaccharide alpha-1,2-mannosidase isoform B, Mannose-binding protein C, Macrophage mannose receptor 1, C-type lectin domain family 6 member A, C-type lectin domain family 4 member M, C-type lectin domain family 4 member K, C-type lectin domain family 4 member C, Beta-galactosidase, DNA topoisomerase 1, Transcription factor E3, Sensor protein kinase WalK family protein, Protein polybromo-1, Chemotaxis protein CheA, Taq polymerase 1, CD81 antigen, UDP-3-O-[3-hydroxymyristoyl] N-acetylglucosamine deacetylase, Kynurenine-oxoglutarate transaminase I, Metabotropic glutamate receptor 6, Coagulation factor XI, Metabotropic glutamate receptor 2, Metabotropic glutamate receptor 1, Ribonuclease T, RNA demethylase ALKBH5, Perilipin-1, N(G),N(G)-dimethylarginine dimethylaminohydrolase 1, Hepatocyte nuclear factor 4-alpha, G-protein coupled receptor family C group 6 member A, Cell death-related nuclease 4, 3-dehydroquinate dehydratase, Glutamate transporter homolog, Histone acetyltransferase p300, Malate dehydrogenase mitochondrial, Estradiol 17-beta-dehydrogenase 3, Brevianamide F prenyltransferase, Tyrosine-protein kinase ABL, Glucose-6-phosphate dehydrogenase-6-phosphogluconolactonase, Cathepsin B, Metabotropic glutamate receptor 5, Uracil nucleotide/cysteinyl leukotriene receptor, Melatonin receptor 1B, Aldehyde dehydrogenase dimeric NADP-preferring, Lysozyme C, Corticosteroid binding globulin, Excitatory amino acid transporter 2, SARS coronavirus 3C-like proteinase, Glutamate (NMDA) receptor subunit zeta 1, Excitatory amino acid transporter 3, Excitatory amino acid transporter 1, Bcl-2-related protein A1, Ubiquitin-conjugating enzyme E2 N, Alcohol sulfotransferase, Glutaminyl-peptide cyclotransferase, Carnitine/acylcarnitine translocase, Taste receptor type 2 member 7, Myocilin, Excitatory amino acid transporter 4, Myosin light chain kinase, smooth muscle, Uridine-cytidine kinase 1, Thymidine kinase 2, Cytidine deaminase, Cell death protein 3, Beta-1,4-galactosyltransferase 1, Neutral amino acid transporter B(0), Neutral amino acid transporter A, Asc-type amino acid transporter 1, ATP-dependent molecular chaperone HSP82, DOPA decarboxylase, Taste receptor type 2 member 16, G protein-coupled receptor kinase 6, Transient receptor potential cation channel subfamily M member 7, BDNF/NT-3 growth factors receptor, Nonstructural protein 5, Human rhinovirus A protease, Glutathione S-transferase Pi, Free fatty acid receptor 2, C-terminal-binding protein 2, NAD(P)H dehydrogenase [quinone] 1, Estrogen-related receptor beta, Leukotriene A4 hydrolase, Sulfotransferase 4A1, Trace amine-associated receptor 5, Cyclic AMP-responsive element-binding protein 1, Transketolase, Thiamine transporter ThiT, Thiamin pyrophosphokinase 1, Ketopantoate reductase, Phospholipase A2, acidic, Nicotinic acetylcholine receptor alpha 5 subunit, Paired box protein Pax-8, Urease, Glutamate racemase, Eukaryotic peptide chain release factor GTP-binding subunit, Voltage-gated calcium channel alpha2/delta subunit 1, UDP-glucuronosyltransferase 2B28, Metabotropic glutamate receptor 7, Metabotropic glutamate receptor 4, Metabotropic glutamate receptor 3, Tryptophan 2,3-dioxygenase, Steryl-sulfatase, Sentrin-specific protease 8, Riboflavin-binding protein, 17-beta-hydroxysteroid dehydrogenase 14, Sulfonylurea receptor 1, Cytochrome c oxidase subunit 2, Cholesterol esterase, Sialidase 3, C—C chemokine receptor type 3, Sialidase A, NAD-dependent histone deacetylase SIR2, Glutathione S-transferase Mu 1, Snake venom metalloproteinase Bap1, Accessory gene regulator protein A, Mitochondrial peptide methionine sulfoxide reductase, Gamma-glutamyltranspeptidase 1, Protein-tyrosine phosphatase 4A3, Ezrin, Insulin-degrading enzyme, Exportin-1, Forkhead box protein 03, Solute carrier organic anion transporter family member 2A1, Multidrug resistance-associated protein 7, Renal sodium-dependent phosphate transport protein 1, Glutamate receptor ionotropic, AMPA 4, Glutamate receptor ionotropic, AMPA 3, Glutamate receptor ionotropic, AMPA 2, Glutamate receptor ionotropic, AMPA 1, Glutamate receptor ionotropic kainate 5, Glutamate receptor ionotropic kainate 3, Glutamate receptor ionotropic kainate 2, Glutamate receptor ionotropic kainate 1, Tetanus toxin, Metabotropic glutamate receptor 8, Glutamate carboxypeptidase II, Alpha-fetoglobulin, Monocarboxylate transporter 4, Heat sensitive channel TRPV3, Thymidine kinase, cytosolic, Scavenger receptor type A, Plectin, Beta-glucosidase A, Beta-mannosidase, Solute carrier organic anion transporter family member 1A3, Phosphodiesterase 4D, Alpha-tocopherol transfer protein, Mineralocorticoid receptor, Succinate dehydrogenase [ubiquinone] flavoprotein subunit, mitochondrial, Taste receptor type 2 member 46, Receptor-type tyrosine-protein phosphatase beta, S-ribosylhomocysteine lyase, Dopamine beta-hydroxylase, Synaptojanin-2, Synaptojanin-1, Mitochondrial import inner membrane translocase subunit TIM10, Phosphatidylinositol synthase, X-box-binding protein 1, NF-kappaB inhibitor alpha, Dopamine D5 receptor, Heat shock 70 kDa protein 1, Mothers against decapentaplegic homolog 2, HSP40, subfamily A, putative, Carbonic anhydrase 3, Eukaryotic initiation factor 4A-II, Cytochrome P450 3A5, Eukaryotic initiation factor 4A-I, Cellular retinoic acid-binding protein II, Taste receptor type 2 member 14, Transcriptional activator Myb, Sulfotransferase 1C4, T-cell surface antigen CD4, Estradiol 17-beta-dehydrogenase 12, Putative hexokinase HKDC1, NAD-dependent deacetylase sirtuin 3, UDP-N-acetylglucosamine 1-carboxyvinyltransferase, Human immunodeficiency virus type 2 integrase, Neuronal acetylcholine receptor subunit alpha-4, Neuronal acetylcholine receptor subunit alpha-3, Acetylcholine-binding protein, Aldehyde dehydrogenase, cytosolic 1, Cysteine protease ATG4B, L-lactate dehydrogenase B chain, Mannose-6-phosphate isomerase, Acetylcholine receptor protein alpha chain, Luciferase, CpG DNA methylase, 3-alpha-hydroxysteroid dehydrogenase, Early growth response protein 1, Histamine H4 receptor, Serotonin 1d (5-HT1d) receptor, Trace amine-associated receptor 1, Equilibrative nucleoside transporter 4, Tyrosine-protein kinase JAK2, Hexose transporter 1, Estrogen-related receptor gamma, Peroxisomal sarcosine oxidase, G-protein coupled estrogen receptor 1, Estrogen receptor, 3-hydroxyacyl-CoA dehydrogenase type-2, S-adenosylmethionine decarboxylase 1, Homoisocitrate dehydrogenase, mitochondrial, Alanine racemase, Constitutive androstane receptor, Calpain 1, Hormone-sensitive lipase, Beta-glucosidase, Sarcoplasmic/endoplasmic reticulum calcium ATPase 2, OXA-48, Phosphatidylinositol-3,4,5-trisphosphate 5-phosphatase 1, Phosphatidylinositol 3,4,5-trisphosphate 5-phosphatase 2, Voltage-dependent L-type calcium channel subunit alpha-1C, Lycopene cyclase, Alpha carbonic anhydrase, Bile acid transporter, Neuronal acetylcholine receptor protein alpha-4 subunit, Soluble acetylcholine receptor, Neuronal acetylcholine receptor subunit beta-4, Neuronal acetylcholine receptor protein alpha-9 subunit, Neuronal acetylcholine receptor protein alpha-2 subunit, Ephrin type-B receptor 4, Serine hydroxymethyltransferase, mitochondrial, Ecdysone receptor, Thymidylate synthase, Protein tyrosine phosphatase receptor type C-associated protein, Phosphodiesterase isozyme 4, Serine/threonine-protein kinase MST2, Casein kinase I delta, HSP90, Testis-specific serine/threonine-protein kinase 1, Protein kinase N2, Maternal embryonic leucine zipper kinase, Catenin beta-1, Regulator of G-protein signaling 12, Cytochrome P450 monooxygenase, Jacalin, Histone deacetylase 7, Chitinase, Polyamine oxidase, Putative silent information regulator 2, NAD-dependent protein deacetylase sirtuin-6, NAD-dependent protein deacetylase, NAD-dependent deacetylase HST2, NAD(+) hydrolase SARM1, Dimethylaniline monooxygenase [N-oxide-forming] 3, Sulfotransferase 1A3/1A4, Hydroxyproline dehydrogenase, Multidrug resistance protein 1a, P2X purinoceptor 3, 3-hydroxy-3-methylglutaryl-coenzyme A reductase, Calcium dependent protein kinase, Sarcoplasmic/endoplasmic reticulum calcium ATPase 1, Cyclin-dependent kinase 5, Thioredoxin reductase 1, Hepatitis C virus NS5B RNA-dependent RNA polymerase, Pantothenate synthetase, Tyrosine-protein kinase TEC, C—X—C chemokine receptor type 4, Oxidation resistance protein 1, Microphthalmia-associated transcription factor, Purinergic receptor P2Y2, Glutamate [NMDA] receptor subunit epsilon 2, P2X purinoceptor 4, cyclic AMP phosphoprotein, Sulfotransferase family cytosolic 1B member 1, Spermine oxidase, Spermidine/spermine N(1)-acetyltransferase 1, Ornithine decarboxylase antizyme 1, Caspase-2, CREB-binding protein, Serine/threonine-protein kinase PAK 1, Pyrimidinergic receptor P2Y6, Dual-specificity tyrosine-phosphorylation regulated kinase 2, Pyrimidinergic receptor P2Y4, Purinergic receptor P2Y11, Purinergic receptor P2Y1, P2Y purinoceptor 1, Kelch-like ECH-associated protein 1, Human immunodeficiency virus type 1 Tat protein, Serine/threonine-protein kinase c-TAK1, Nuclear receptor ROR-beta, Nuclear receptor ROR-alpha, Mitogen-activated protein kinase 15, Cyclin-dependent kinase 9, Cyclic GMP-AMP synthase, P2X purinoceptor 1, Glycerol kinase, G-protein coupled receptor 55, P2X purinoceptor 6, P2X purinoceptor 5, Heat shock protein 90 beta, Heat shock protein 75 kDa, mitochondrial, Endoplasmin, Ectonucleotide pyrophosphatase/phosphodiesterase family member 3, Ectonucleotide pyrophosphatase/phosphodiesterase family member 1, Ectonucleoside triphosphate diphosphohydrolase 1, Tyrosine-protein kinase TIE-2, Vascular endothelial growth factor receptor 3, Dual specificity protein phosphatase 6, Tyrosine-protein kinase BMX, NADH-ubiquinone oxidoreductase chain 1, Solute carrier family 22 member 21, ORF 73, Bromodomain-containing protein 9, Serine/threonine-protein kinase SRPK1, Serine/threonine-protein kinase EEF2K, Protein kinase C zeta, Protein kinase C mu, Nerve growth factor receptor Trk-A, MAP kinase-activated protein kinase 3, MAP kinase signal-integrating kinase 2, Homeodomain-interacting protein kinase 3, Homeodomain-interacting protein kinase 2, BR serine/threonine-protein kinase 2, Tyrosyl-tRNA synthetase, Serine/threonine-protein kinase NEK7, Serine/threonine-protein kinase Aurora-C, Nuclear factor of activated T-cells, cytoplasmic 1, Histone acetyltransferase PCAF, Prion protein, Mitogen-activated protein kinase kinase kinase 8, DNA repair and recombination protein RAD54-like, ATPase family AAA domain-containing protein 2, Caspase-8, Presenilin 1, Macrophage migration inhibitory factor, Cell division protein kinase 8, Inhibitor of nuclear factor kappa B kinase alpha subunit, Cytochrome P450 2B1, MAP kinase-interacting serine/threonine-protein kinase MNK1, Dual-specificity tyrosine-phosphorylation regulated kinase 3, Lysyl oxidase homolog 2, Killer cell lectin-like receptor subfamily B member 1A, CaM-kinase kinase alpha, Type-1A angiotensin II receptor, Galectin-3, Galectin-1, Voltage-gated potassium channel subunit Kv4.3, Flavodoxin, Histone deacetylase 2, Creatine kinase M, Phosphoglycerate kinase, DNA polymerase I, DNA nucleotidylexotransferase, Acyl-CoA synthase, Neurotrophic tyrosine kinase receptor type 2, Dual specificity phosphatase Cdc25A, C-8 sterol isomerase, 3-beta-hydroxysteroid-delta(8),delta(7)-isomerase, Apoptotic protease-activating factor 1, 7,8-dihydro-8-oxoguanine triphosphatase, Transient receptor potential cation channel subfamily V member 6, Quinone reductase 2, Transient receptor potential cation channel subfamily V member 1, Adenosylmethionine-8-amino-7-oxononanoate aminotransferase, Serotonin 5a (5-HT5a) receptor, Histone-lysine N-methyltransferase EZH2, Prostanoid EP2 receptor, Neutrophil cytosol factor 1, DNA damage-inducible transcript 3 protein, Glycogen synthase kinase-3 alpha, TGF-beta receptor type II, Lymphocyte antigen 96, L-lactate dehydrogenase, Sigma-1 receptor, Type-1 angiotensin II receptor, Serine-protein kinase ATR, Putative glycosyltransferase, Early activation antigen CD69, Sulfate anion transporter 1, Retinal dehydrogenase 2, Aldehyde dehydrogenase X, Aldehyde dehydrogenase 1A3, Indoleamine 2,3-dioxygenase 2, Multidrug and toxin extrusion protein 2, Tyrosine-protein kinase BTK, Hematopoietic cell protein-tyrosine phosphatase 70Z-PEP, Protein kinase C theta, Serine/threonine-protein phosphatase, Type III pantothenate kinase, Type II pantothenate kinase, Galectin-9, Galectin-8, Galectin-7, Sterol 14-alpha demethylase, Eukaryotic translation initiation factor 4E, Beta-galactoside-binding lectin, Serotonin 1e (5-HT1e) receptor, Peptide deformylase mitochondrial, Peptide deformylase 1A, chloroplastic, Peptide deformylase, Pancreatic lipase, Prolyl 4-hydroxylase subunit alpha-1, Multidrug resistance-associated protein 5, Hypoxia-inducible factor prolyl hydroxylase 1, Egl nine homolog 3, Strictosidine synthase, MAP/microtubule affinity-regulating kinase 4, Cytosolic 10-formyltetrahydrofolate dehydrogenase, Histone acetyltransferase KAT8, M17 leucyl aminopeptidase, Protein-tyrosine phosphatase G1, Probable low molecular weight protein-tyrosine-phosphatase, Histone-arginine methyltransferase CARM1, Testosterone 17-beta-dehydrogenase 3, Pyridoxal kinase, Likely tRNA 2′-phosphotransferase, Guanyl-specific ribonuclease T1, Glutathione S-transferase, Endoribonuclease Dicer, Acetyl-CoA acetyltransferase/HMG-CoA reductase, Peripheral-type benzodiazepine receptor, Cathepsin K, PI3-kinase p110-beta subunit, Proline racemase, Beta Lactamase, Large neutral amino acids transporter small subunit 1, Toll-like receptor 9, Sarcoplasmic/endoplasmic reticulum calcium ATPase 3, Probable nicotinate-nucleotide adenylyltransferase, Proto-oncogene C-crk, Growth factor receptor-bound protein 2, Small ubiquitin-related modifier 1, Tyrosine-protein kinase ZAP-70, Nuclear receptor subfamily 1 group I member 3, Oligopeptide transporter small intestine isoform, Histidase, DNA polymerase delta subunit 1, Histone acetyltransferase KAT5, Glycine transporter 1, Solute carrier organic anion transporter family member 3A1, Caspase-6, Regulator of G-protein signaling 17, Fibrinogen beta chain, Bcl2-antagonist of cell death (BAD), Vascular endothelial growth factor A, Placenta growth factor, Pteridine reductase 1, Mitogen-activated protein kinase 8, Interleukin-1 receptor-associated kinase 1, Ribosomal protein S6 kinase alpha 4, Monocyte differentiation antigen CD14, Mitogen-activated protein kinase 3, Casein kinase II alpha (prime), Solute carrier family 2, facilitated glucose transporter member 3, Protein tyrosine kinase 2 beta, N1L, Myc proto-oncogene protein, Forkhead box protein 01, Alkaline phosphatase, Sphingosine kinase 2, Sphingosine kinase 1, Endoglycoceramidase II, C—X—C chemokine receptor type 5, C—C chemokine receptor type 6, Apelin receptor, Endochitinase, Ephrin type-B receptor 2, Retinoid X receptor gamma, Retinoid X receptor beta, LIM domain kinase 1, Tyrosine-protein kinase BRK, Serine/threonine-protein kinase TBK1, Serine/threonine-protein kinase TAO1, Serine/threonine-protein kinase 24, Serine/threonine-protein kinase 11, MAP/microtubule affinity-regulating kinase 2, Interleukin-1 receptor-associated kinase 4, Ephrin type-B receptor 3, Ephrin type-A receptor 4, Ephrin type-A receptor 2, Discoidin domain-containing receptor 2, cGMP-dependent protein kinase 1 beta, Tyrosine-protein kinase HCK, Tyrosine-protein kinase FRK, Tyrosine-protein kinase FER, Tyrosine-protein kinase ABL2, Tyrosine kinase non-receptor protein 2, TGF-beta receptor type I, Serine/threonine-protein kinase ULK3, Serine/threonine-protein kinase TAO3, Serine/threonine-protein kinase TAO2, Serine/threonine-protein kinase Nek3, Serine/threonine-protein kinase MRCK-A, Serine/threonine-protein kinase MRCK beta, Serine/threonine-protein kinase D2, Serine/threonine-protein kinase AKT3, Serine/threonine protein kinase NLK, Ribosomal protein S6 kinase alpha 6, Protein kinase C iota, Prostanoid IP receptor, Phosphorylase kinase gamma subunit 2, Mitogen-activated protein kinase kinase kinase kinase 2, Macrophage-stimulating protein receptor, Ephrin type-A receptor 7, Ephrin type-A receptor 5, Ephrin type-A receptor 1, Activin receptor type-1B, Apolipoprotein A-I, Prostanoid EP4 receptor, Prostanoid EP3 receptor, Prostanoid EP1 receptor, Gamma-glutamyltranspeptidase, Phosphodiesterase 3B, Macrophage-expressed gene 1 protein, Glutathione S-transferase kappa 1, Cytosolic phospholipase A2 gamma, 5-lipoxygenase activating protein, Fibroblast growth factor 22, Dual specificity protein phosphatase 1, Proto-oncogene c-Fos, Methionyl-tRNA synthetase, putative, Cystathionine gamma-lyase, Cystathionine beta-synthase, ATP-binding cassette sub-family C member 11, Guanine deaminase, Tyrosine-protein kinase transforming protein FPS, Probable DNA dC->dU-editing enzyme APOBEC-3A, DNA primase traC, Chaperone protein dnaK, Casein kinase I alpha, Retinoic acid receptor beta, Histone deacetylase 9, Histone deacetylase 8, Lysyl-tRNA synthetase, Lysine-specific demethylase 4C, Serine/threonine-protein kinase/endoribonuclease IRE1, Nicotinic acetylcholine receptor alpha8 subunit, Lysine-specific demethylase 7, Lysine-specific demethylase 6B, Lysine-specific demethylase 6A, Lysine-specific demethylase 5C, Lysine-specific demethylase 2A, Histone lysine demethylase PHF8, Gamma-butyrobetaine dioxygenase, Serine/threonine-protein kinase PINK1, mitochondrial, Cofilin-1, Acidic mammalian chitinase, NADH-ubiquinone oxidoreductase chain 4, Intercellular adhesion molecule-1, Cytochrome b-245 light chain, Acetolactate synthase, Serine/threonine-protein kinase haspin, Protein-glutamine gamma-glutamyltransferase, Mitochondrial import inner membrane translocase subunit TIM23, Histone-lysine N-methyltransferase, H3 lysine-9 specific 5, Falcipain 2, G-protein coupled receptor kinase 2, Phosphatidylinositol-5-phosphate 4-kinase type-2 gamma, Interleukin-1 receptor-associated kinase 3, Cyclin-dependent kinase 7, Citron Rho-interacting kinase, Casein kinase I epsilon, Vitamin D-binding protein, Smoothened homolog, Serine/threonine-protein kinase GAK, Multidrug resistance associated protein, Heme oxygenase 2, 17-beta-hydroxysteroid-dehydrogenase, dCTP pyrophosphatase 1, Nicotinic acetylcholine receptor alpha1 subunit, Delta(24)-sterol reductase, Advanced glycosylation end product-specific receptor, Serine/threonine-protein kinase SIK3, Serine/threonine-protein kinase SIK2, Oxidized low-density lipoprotein receptor 1, Mitogen-activated protein kinase kinase kinase 11, Mitogen-activated protein kinase kinase kinase 1, Inhibitor of nuclear factor kappa B kinase epsilon subunit, Dual specificity testis-specific protein kinase 1, Dual specificity protein kinase TTK, cAMP-dependent protein kinase beta-1 catalytic subunit, UMP-CMP kinase, Tyrosine-protein kinase TYK2, Tyrosine- and threonine-specific cdc2-inhibitory kinase, TGF-beta receptor type-1, Serine/threonine-protein kinase WEEi, Serine/threonine-protein kinase PCTAIRE-2, Serine/threonine-protein kinase Nekl, Serine/threonine-protein kinase NEK9, Serine/threonine-protein kinase MRCK gamma, Serine/threonine-protein kinase LATS1, Serine/threonine-protein kinase ICK, Protein kinase C nu, Non-receptor tyrosine-protein kinase TNK1, NUAK family SNF1-like kinase 2, Myosin light chain kinase, Mixed lineage kinase 7, Mitogen-activated protein kinase kinase kinase kinase 4, Mitogen-activated protein kinase kinase kinase kinase 3, Mitogen-activated protein kinase kinase kinase kinase 1, Mitogen-activated protein kinase kinase kinase 6, Mitogen-activated protein kinase kinase kinase 4, Mitogen-activated protein kinase kinase kinase 3, Mitogen-activated protein kinase kinase kinase 2, Mitogen-activated protein kinase 7, LIM domain kinase 2, GTP-binding nuclear protein Ran, Eukaryotic translation initiation factor 2-alpha kinase 1, Epithelial discoidin domain-containing receptor 1, Ephrin type-B receptor 6, Dual specificity mitogen-activated protein kinase kinase 5, Dual specificity mitogen-activated protein kinase kinase 3, Deoxycytidine kinase, Cyclin-dependent kinase 4, Cyclin-dependent kinase 3, Chaperone activity of bel complex-like, mitochondrial, Cell division cycle 7-related protein kinase, Bone morphogenetic protein receptor type-2, Bone morphogenetic protein receptor type-1B, Bone morphogenetic protein receptor type-1A, BMP-2-inducible protein kinase, Adaptor-associated kinase, Activin receptor type-2B, Activin receptor type-1, AMP-activated protein kinase, alpha-1 subunit, cAMP-dependent protein kinase, gamma catalytic subunit, cAMP-dependent protein kinase type II-alpha regulatory subunit, Very long-chain specific acyl-CoA dehydrogenase, mitochondrial, Uncharacterized protein FLJ45252, Uncharacterized aarF domain-containing protein kinase 1, U5 small nuclear ribonucleoprotein 200 kDa helicase, TP53-regulating kinase, Succinate-CoA ligase [ADP-forming] subunit beta, mitochondrial, Structural maintenance of chromosomes protein 2, Signal recognition particle receptor subunit alpha, Serine/threonine-protein kinase/endoribonuclease IRE2, Serine/threonine-protein kinase ILK-1, Serine/threonine-protein kinase A-Raf, Septin-9, STE20-related kinase adapter protein alpha, S-adenosylmethionine synthase isoform type-2, Receptor-interacting serine/threonine-protein kinase 3, Ras-related protein Rab-6A, Ras-related protein Rab-27A, Ras-related protein Rab-10, RNA cytidine acetyltransferase, Probable ATP-dependent RNA helicase DDX6, Phosphofructokinase platelet type, Phosphatidylinositol-5-phosphate 4-kinase type-2 alpha, Phosphatidylethanolamine-binding protein 1, Phenylalanine-tRNA ligase beta subunit, Peroxisomal acyl-coenzyme A oxidase 3, Peroxisomal acyl-coenzyme A oxidase 1, Obg-like ATPase 1, Nucleolar GTP-binding protein 1, NADH dehydrogenase [ubiquinone] 1 alpha subcomplex subunit 13, Myosin-14, Myosin-10, Multifunctional protein ADE2, Mitotic checkpoint serine/threonine-protein kinase BUB1, Midasin, Membrane-associated progesterone receptor component 1, Long-chain-fatty-acid-CoA ligase 5, Guanine nucleotide-binding protein G(i) subunit alpha-2, Glycine-tRNA ligase, General transcription and DNA repair factor IIH helicase subunit XPD, Ferrochelatase, mitochondrial, Exosome RNA helicase MTR4, Elongation factor Tu, mitochondrial, Electron transfer flavoprotein subunit beta, Dynamin-like 120 kDa protein, mitochondrial, DnaJ homolog subfamily A member 1, DNA replication licensing factor MCM4, Cytochrome cI, heme protein, mitochondrial, Cysteine-tRNA ligase, cytoplasmic, Cyclin-dependent kinase 12, Cyclin-dependent kinase 10, Chromodomain-helicase-DNA-binding protein 4, Breakpoint cluster region protein, Adenine phosphoribosyltransferase, Actin-related protein 3, Actin-related protein 2, ATP-dependent RNA helicase DDX3X, ATP-dependent RNA helicase DDX1, AMP-activated protein kinase, gamma-2 subunit, AMP-activated protein kinase, gamma-1 subunit, ADP/ATP translocase 3, ADP/ATP translocase 2, 26S protease regulatory subunit 6B, Structural maintenance of chromosomes protein 1A, Rab-like protein 3, Putative heat shock protein HSP 90-beta 2, Isoleucine-tRNA ligase, mitochondrial, ATP-dependent RNA helicase DDX42, Alpha-1B adrenergic receptor, Probable ubiquitin carboxyl-terminal hydrolase FAF-X, Complement C5, Taste receptor type 2 member 10, Asialoglycoprotein receptor 1, Deoxyhypusine synthase, Tubulin beta chain, Transporter, Beta tubulin, Pyruvate dehydrogenase kinase isoform 4, Phosphatidylcholine:ceramide cholinephosphotransferase 2, Phosphatidylcholine:ceramide cholinephosphotransferase 1, Formyl peptide receptor 1, Carbonic anhydrase, alpha family, Cytochrome P450 2D3, Cytochrome P450 2D2, Cytochrome P450 2D18, Cytochrome P450 2D1, Tubulin alpha chain, Transient receptor potential cation channel subfamily V member 3, RNA-editing ligase 1, mitochondrial, Multidrug resistance protein 3, G protein-coupled receptor kinase 5, Serine/threonine-protein kinase PLK3, Serine/threonine-protein kinase PLK2, Protein kinase C gamma, Homeodomain-interacting protein kinase 1, Dual specificity mitogen-activated protein kinase kinase 7, Death-associated protein kinase 2, Phosphodiesterase 10A, Synapsin-1, Squalene-hopene cyclase, Phosphodiesterase 4A, Phosphodiesterase 3A, Telomere resolvase resT, Shiga toxin 1 variant A subunit, Proto-oncogene tyrosine-protein kinase MER, Beta-glucosidase cytosolic, Kinesin-like protein KIF20A, cAMP and cAMP-inhibited cGMP 3′,5′-cyclic phosphodiesterase 10A, Metallo-beta-lactamase type 2, Tubulin beta-5 chain, Serine/threonine-protein kinase MARK1, Serine/threonine-protein kinase 33, Ribosomal protein S6 kinase alpha 2, Misshapen-like kinase 1, Insulin receptor-related protein, Hypoxanthine-guanine phosphoribosyltransferase, Ephrin type-B receptor 1, BR serine/threonine-protein kinase 1, Voltage-gated T-type calcium channel alpha-1H subunit, Voltage-gated T-type calcium channel alpha-1G subunit, Tyrosine-protein kinase receptor TYRO3, Tyrosine-protein kinase TXK, Tyrosine-protein kinase BLK, Tubulin alpha-1 chain, Testis-specific serine/threonine-protein kinase 2, Serine/threonine-protein kinase tousled-like 2, Serine/threonine-protein kinase ULK2, Serine/threonine-protein kinase Sgk3, Serine/threonine-protein kinase Sgk2, Serine/threonine-protein kinase SRPK3, Serine/threonine-protein kinase SRPK2, Serine/threonine-protein kinase SIK1, Serine/threonine-protein kinase PRKX, Serine/threonine-protein kinase PAK 3, Serine/threonine-protein kinase Nek11, Serine/threonine-protein kinase DCLK2, Proto-oncogene tyrosine-protein kinase ROS, Platelet-derived growth factor receptor alpha, PAS domain-containing serine/threonine-protein kinase, Muscle, skeletal receptor tyrosine protein kinase, Mitogen-activated protein kinase kinase kinase 9, Macrophage colony stimulating factor receptor, Kinesin-like protein 1, G protein-coupled receptor kinase 7, Fibroblast growth factor receptor 4, Fibroblast growth factor receptor 3, Ephrin type-A receptor 8, Ephrin type-A receptor 3, Bcl-2-like protein 1, 5-enolpyruvylshikimate-3-phosphate synthase, Tubulin polymerization-promoting protein, Serine/threonine-protein kinase WNK3, Serine/threonine-protein kinase WNK2, Nischarin, Cytochrome P450 2B4, Transcription factor AP-1, Bromodomain-containing protein 4, Phosphodiesterase 8A, Folylpoly-gamma-glutamate synthetase, Folate receptor beta, Folate receptor alpha, Bifunctional protein FolC, Short transient receptor potential channel 4, Cyclophilin A, Interleukin-5 receptor subunit alpha, Interleukin-5, dTDP-4-dehydrorhamnose reductase, cAMP-specific 3′,5′-cyclic phosphodiesterase 4B, Serine palmitoyltransferase, Cystine/glutamate transporter, Carboxypeptidase A1, Adenylate cyclase, Histidine kinase, Cytokinin dehydrogenase 2, Cholecystokinin B receptor, Syk protein, Alpha-crystallin B chain, 1-phosphatidylinositol 4,5-bisphosphate phosphodiesterase gamma-2, Sodium channel protein type V alpha subunit, Tubulin beta-6 chain, 2-amino-4-hydroxy-6-hydroxymethyldihydropteridine pyrophosphokinase, DNA polymerase, Phosphoglycerate mutase 1, Dihydroorotate dehydrogenase (quinone), mitochondrial, Alpha-glucosidase, Brain adenylate cyclase 1, Aromatic peroxygenase, NADPH oxidase 1, Mitogen-activated protein kinase 1, Bile acid receptor, Solute carrier organic anion transporter family member 1C1, Diamine oxidase, Trehalase, Ryanodine receptor 2, Neutral alpha-glucosidase C, Neutral alpha-glucosidase AB, Legumain, Lactase-glycosylceramidase, Glycogen debranching enzyme, Glucosylceramidase, Ceramide glucosyltransferase, Alpha-L-fucosidase 1, Uncharacterized protein, TyRI, Phosphodiesterase 4B, Organic solute transporter subunit alpha, Mannosidase 2, alpha B1, Mannosidase 2 alpha 1, Lysosomal alpha-mannosidase, Lactase-phlorizin hydrolase, Epididymis-specific alpha-mannosidase, Astrosclerin-3, Alpha-mannosidase, Alpha-galactosidase C, Alpha-galactosidase B, Alpha-galactosidase, Solute carrier organic anion transporter family member 4A1, N-acylethanolamine-hydrolyzing acid amidase, Sodium/bile acid cotransporter, Matrix metalloproteinase 14, Ileal sodium/bile acid cotransporter, Glucoamylase, intracellular sporulation-specific, Autotaxin, Von Hippel-Lindau disease tumor suppressor, SUMO-activating enzyme subunit 1, Ricin, Methionyl-tRNA synthetase, Ileal bile acid transporter, Gamma-crystallin D, Gamma-crystallin C, Fructose-bisphosphate aldolase, Beta-crystallin B2, Alpha-crystallin A chain, 14-alpha sterol demethylase, Vesicular acetylcholine transporter, Probable linoleate 9S-lipoxygenase 5, Solute carrier organic anion transporter family member, Phosphodiesterase 1B, Methionine aminopeptidase 2, Transmembrane domain-containing protein TMIGD3, Phosphodiesterase 7A, Phosphodiesterase 1C, Phosphodiesterase 1A, PDE7B protein, Platelet glycoprotein VI (GPVI), Cytochrome c, Synaptic vesicular amine transporter, Signal transduction protein TRAP, Protein-arginine N-methyltransferase 1, Glutathione S-transferase theta 1, RmtA, Kappa-type opioid receptor, Isocitrate lyase, Glutamate decarboxylase 65 kDa isoform, Gag-Pol polyprotein, Delta-type opioid receptor, Triosephosphate isomerase, glycosomal, N-acylsphingosine-amidohydrolase, DNA topoisomerase 2, Transient receptor potential cation channel, subfamily V, member 3, Transient receptor potential cation channel subfamily V member 4, Taste receptor type 2 member 31, Sn1-specific diacylglycerol lipase alpha, N-arachidonyl glycine receptor, Matrix metalloproteinase 7, Heat shock 70 kDa protein 6, Interleukin-6, Inhibitor of nuclear factor kappa-B kinase subunit beta, 40S ribosomal protein SA, 3-oxoacyl-(Acyl-carrier protein) reductase, Subtilisin/kexin type 7, D-3-phosphoglycerate dehydrogenase, Rhodesain, Opioid receptor, delta 1b, Opioid receptor homologue, Nociceptin receptor, Mu opioid receptor-like OR2, Serotonin if (5-HTlf) receptor, Rhodopsin kinase, Vitamin k epoxide reductase complex subunit 1 isoform 1, Transcriptional activator protein lasR, Serine/threonine-protein kinase 17B, Regulatory protein RhlR, Dual specificity protein kinase CLK1, Cytochrome P450 17A1, Serine/threonine protein phosphatase PP1-alpha catalytic subunit, Sentrin-specific protease 7, Sentrin-specific protease 6, P14-kinase beta subunit, Histone-lysine N-methyltransferase NSD2, Dual specificity tyrosine-phosphorylation-regulated kinase 4, Dual specificity tyrosine-phosphorylation-regulated kinase 1B, Dual specificity protein kinase CLK4, Cyclin-dependent kinase-like 5, Cell division cycle 2-like protein kinase 6, Beta-adrenergic receptor kinase 1, Tubulin beta-1 chain, Protein-lysine 6-oxidase, Ornithine carbamoyltransferase, Fe(3+)-Zn(2+) purple acid phosphatase, Amiloride-sensitive amine oxidase [copper-containing], Protein cereblon, Protein Rev, Methionine aminopeptidase 1, Long-wave-sensitive opsin 1, Female germline-specific tumor suppressor gld-1, Farnesyl diphosphate synthase, Cereblon isoform 4, Carboxylesterase, 40S ribosomal protein S6, Steroid 5-alpha-reductase 2, Solute carrier family 15 member 1, RAS guanyl-releasing protein 1, RAS guanyl releasing protein 3, Purine-nucleoside phosphorylase, Protoporphyrinogen IX oxidase, Phosphatidylinositol 3-kinase catalytic subunit type 3, Oligopeptide transporter, kidney isoform, Nuclear receptor subfamily 4 group A member 2, Nuclear factor of activated T-cells cytoplasmic 1, Methionine aminopeptidase, Heme oxygenase 1, Glycoprotein, Geranylgeranyl pyrophosphate synthetase, Eukaryotic translation initiation factor 4E-binding protein 1, Purinergic receptor P2Y14, Melatonin receptor 1C, Inosine-5′-monophosphate dehydrogenase, Gasdermin-D, Chloroquine resistance transporter, Tubulin beta-4 chain, Tubulin beta-3 chain, Trehalose-phosphatase, Transient receptor potential cation channel subfamily M member 2, Steroidogenic acute regulatory protein, mitochondrial, Proteinase-activated receptor 2, Phosphodiesterase 9A, Nucleotide-binding oligomerization domain-containing protein 2, Mitogen-activated protein kinase kinase kinase 14, Lanosterol 14-alpha demethylase, Lactate dehydrogenase, H(+)/Cl(−) exchange transporter 3, Dual specificity phosphatase Cdc25C, Dihydrolipoamide dehydrogenase, Apoptosis regulator Bcl-W, AmpC, ADP-ribose glycohydrolase MACROD1, 4-diphosphocytidyl-2-C-methyl-D-erythritol kinase, chloroplastic, 4-diphosphocytidyl-2-C-methyl-D-erythritol kinase, Transcription factor SKN7, Signal transducer and activator of transcription 1, Serine/threonine protein phosphatase PP1-gamma catalytic subunit, Serine/threonine protein phosphatase 2A, catalytic subunit, alpha isoform, Serine-threonine protein phosphatase 2A regulatory subunit, Seed linoleate 9S-lipoxygenase, Protein phosphatase 2C beta, Proteasome subunit beta type-5, Prosaposin, Prolactin-releasing peptide receptor, Orexin receptor 2, Islet amyloid polypeptide, Human papillomavirus regulatory protein E2, HTH-type transcriptional regulator EthR, Glucose-dependent insulinotropic receptor, Dihydropteroate synthase, C—C chemokine receptor type 8, Betaine-homocysteine S-methyltransferase 1, Acetylcholine receptor protein epsilon chain, UDP-N-acetylglucosamine-peptide N-acetylglucosaminyltransferase 110 kDa subunit, Tumor necrosis factor receptor R1, Squalene synthase, Regulatory protein E2, Protein prune homolog, Prostanoid FP receptor, Phosphodiesterase 7B, Phosphodiesterase 11A, Neurogenic locus notch homolog protein 1, Mesoderm-specific transcript homolog protein, MBT domain-containing protein 1, Lethal(3)malignant brain tumor-like protein 4, Lethal(3)malignant brain tumor-like protein 3, Indoleamine 2,3-dioxygenase 1, Histone-lysine N-methyltransferase SETD7, Glycerol-3-phosphate acyltransferase 4, Glycerol-3-phosphate acyltransferase 3, Glycerol-3-phosphate acyltransferase 1, mitochondrial, Glutathione-S-transferase, Glutathione transferase omega 1, Dehydrosqualene desaturase, Cytokinin dehydrogenase 1, 4,4′-diapophytoene desaturase (4,4′-diaponeurosporene-forming), Xaa-Pro dipeptidase, Type IV secretion-like conjugative transfer relaxase protein TraI, Thromboxane A2 receptor, Solute carrier organic anion transporter family member 4C1, Sodium/iodide cotransporter, Snq2p, Shiga toxin subunit A, Serine/threonine-protein kinase tousled-like 1, Prostanoid DP receptor, Potassium voltage-gated channel subfamily H member 2, Potassium voltage-gated channel subfamily E member 1, Pleiotropic ABC efflux transporter of multiple drugs, Kallikrein 7, Isocitrate lyase 1, Insulin-like growth factor binding protein 5, Inositol-1(or 4)-monophosphatase 1, Glutamine synthetase, Geranylgeranyl pyrophosphate synthase, General transcription and DNA repair factor IIH helicase subunit XPB, CG8425-PA [Drosophila melanogaster], Beta-lactamase OXA-9, Acyl-CoA desaturase, ATP binding cassette transporter Abc1p, myosin light chain kinase 2, cGMP-dependent protein kinase 2, Wee1-like protein kinase 2, Voltage-gated L-type calcium channel alpha-1S subunit, Uncharacterized aarF domain-containing protein kinase 4, ULK3 kinase, UDP-glucose 4-epimerase, Tyrosyl-DNA phosphodiesterase 2, Tyrosine-protein kinase receptor Tie-1, Tyrosine-protein kinase Srms, Tyrosine-protein kinase CTK, Transient receptor potential cation channel subfamily M member 6, Serine/threonine-protein kinase receptor R3, Serine/threonine-protein kinase pknB, Serine/threonine-protein kinase ULK1, Serine/threonine-protein kinase TNNI3K, Serine/threonine-protein kinase SBK1, Serine/threonine-protein kinase RI03, Serine/threonine-protein kinase RIO1, Serine/threonine-protein kinase PRP4 homolog, Serine/threonine-protein kinase PFTAIRE-2, Serine/threonine-protein kinase PFTAIRE-1, Serine/threonine-protein kinase PCTAIRE-3, Serine/threonine-protein kinase Nek5, Serine/threonine-protein kinase Nek4, Serine/threonine-protein kinase NIM1, Serine/threonine-protein kinase MAK, Serine/threonine-protein kinase LATS2, Serine/threonine-protein kinase DCLK3, Serine/threonine-protein kinase DCLK1, Serine/threonine-protein kinase 38-like, Serine/threonine-protein kinase 36, Serine/threonine-protein kinase 35, Serine/threonine-protein kinase 32C, Serine/threonine-protein kinase 32B, Serine/threonine-protein kinase 32A, Sclerostin, STE20/SPS1-related proline-alanine-rich protein kinase, SPS1/STE20-related protein kinase YSK4, SNF-related serine/threonine-protein kinase, Receptor-interacting serine/threonine-protein kinase 4, Receptor-interacting serine/threonine-protein kinase 1, Receptor tyrosine-protein kinase erbB-3, Putative uncharacterized serine/threonine-protein kinase SgK110, Potassium channel subfamily K member 3, Phosphorylase kinase gamma subunit 1, Phosphatidylinositol-5-phosphate 4-kinase type-2 beta, Phosphatidylinositol-4-phosphate 5-kinase type-1 gamma, Phosphatidylinositol-4-phosphate 5-kinase type-1 alpha, Phosphatidylinositol-4-phosphate 3-kinase C2 domain-containing subunit gamma, Phosphatidylinositol-4-phosphate 3-kinase C2 domain-containing beta polypeptide, Peripheral plasma membrane protein CASK, Paxillin, PITSLRE serine/threonine-protein kinase CDC2L2, PITSLRE serine/threonine-protein kinase CDC2L1, NT-3 growth factor receptor, NADPH-cytochrome P450 reductase, Myosin-IIIB, Myosin light chain kinase family member 4, Myosin IIIA, Myelin transcription factor 1, Multidrug resistance protein 1, Multidrug efflux pump LfrA, Mitogen-activated protein kinase kinase kinase 15, Mitogen-activated protein kinase kinase kinase 13, Mitogen-activated protein kinase kinase kinase 12, Mitogen-activated protein kinase kinase kinase 10, Mitogen-activated protein kinase 4, Microtubule-associated serine/threonine-protein kinase 1, Leukocyte tyrosine kinase receptor, Leucine-rich repeat serine/threonine-protein kinase 2, L-type amino acid transporter 3, Interferon-induced, double-stranded RNA-activated protein kinase, Hormonally up-regulated neu tumor-associated kinase, Homeodomain-interacting protein kinase 4, G protein-coupled receptor kinase 4, Eukaryotic translation initiation factor 2-alpha kinase 4, Ephrin type-A receptor 6, Enoyl-[acyl-carrier-protein]reductase [NADH], Endothelin receptor ET-B, Emopamil-binding protein-like, Dual specificity mitogen-activated protein kinase kinase 4, Dual serine/threonine and tyrosine protein kinase, Cytochrome P450 11B2, Cytochrome P450 11B1, Cyclin-dependent kinase-like 3, Cyclin-dependent kinase-like 2, Cyclin-dependent kinase 13, Coagulation factor XII, Chorismate synthase, Cell division control protein 2 homolog, Casein kinase I isoform alpha-like, Calcium/calmodulin-dependent protein kinase kinase 2, Calcium-dependent protein kinase 4, Calcium-dependent protein kinase 1, Ankyrin repeat and protein kinase domain-containing protein 1, Activin receptor type-2A, ATP phosphoribosyltransferase, 5′-AMP-activated protein kinase catalytic subunit alpha-1, Zinc finger protein GLI2, Uncharacterized aarF domain-containing protein kinase 5, Type 1 InsP3 receptor isoform S2, Translocator protein, Transitional endoplasmic reticulum ATPase, Stimulator of interferon genes protein, Solute carrier family 15 member 2, Sodium/potassium-transporting ATPase alpha-1 chain, Serine/threonine-protein kinase N3, Sensor histidine kinase yycG, Rho guanine nucleotide exchange factor 1, Retinoic acid receptor gamma, Relaxin receptor 2, Relaxin receptor 1, Protoporphyrinogen oxidase, chloroplastic/mitochondrial, Potassium channel subfamily K member 9, Phosphoglycerate kinase 1, Peroxisome proliferator-activated receptor gamma coactivator 1-alpha, Octopamine receptor, Neutral sphingomyelinase, Neuroepithelial cell-transforming gene 1 protein, Multidrug resistance-associated protein 6, Multidrug resistance protein CDR1, Metallo beta-lactamase, Membrane-associated phosphatidylinositol transfer protein 1, Mast/stem cell growth factor receptor Kit, Macrophage migration inhibitory factor homologue, Lipoprotein lipase, Histamine N-methyltransferase, Glutamate receptor ionotropic kainate 4, Glutamate [NMDA] receptor subunit epsilon 3, GABA receptor alpha-5 subunit, Eukaryotic peptide chain release factor GTP-binding subunit ERF3B, Epoxide hydrolase 1, Endothelial lipase, Cytochrome P450 71B1, Cytochrome P450 3A7, Choline-phosphate cytidylyltransferase A, Beta-lactamase VIM-2, Beta-lactamase NDM-1, Beta-lactamase L1, BCR/ABL p210 fusion protein, Aquaporin-2, Aldehyde oxidase 1, Adenylate cyclase type II, Adenylate cyclase type I, Acyl-CoA dehydrogenase family member 11, Acyl-CoA dehydrogenase family member 10, 2-dehydro-3-deoxyphosphooctonate aldolase, 14-3-3 protein sigma, tRNA-guanine transglycosylase, Vimentin, Vesicular glutamate transporter 3, Uridine 5′-monophosphate synthase, UDP-N-acetylmuramoyl-tripeptide-D-alanyl-D-alanine ligase, Tubulin alpha-1B chain, Tryptophan dimethylallyltransferase, Transmembrane 4 L6 family member 5, Trans-sialidase, Trans-cinnamate 4-monooxygenase, Topoisomerase I, Thermolysin, Tau-tubulin kinase 1, Sulfotransferase 1C2, Sulfotransferase 1A2, Strictosidine beta-glucosidase, Stress-70 protein, mitochondrial, Serine/threonine-protein kinase WNK1, Sepiapterin reductase, Sensor protein kinase WalK, Rho-related GTP-binding protein RhoQ, Ras-related protein Rab-7a, Putative tubulin-like protein alpha-4B, Putative annexin A2-like protein, Protein-tyrosine phosphatase 1, Protein skinhead-1, Prostaglandin 12 synthase, Programmed cell death protein 6, Potassium-transporting ATPase alpha chain 2, Polyadenylate-binding protein 1, Poly(rC)-binding protein 2, Photoreceptor-specific nuclear receptor, Phosphate carrier protein, mitochondrial, Peroxiredoxin-5, mitochondrial, Peroxiredoxin-1, PA-I galactophilic lectin, P2X purinoceptor 2, Oxoeicosanoid receptor 1, Orotidine phosphate decarboxylase, Orotidine 5′-phosphate decarboxylase, Muscarinic receptor 2, Low affinity sodium-glucose cotransporter, Leukocyte adhesion glycoprotein LFA-1 alpha, Kruppel-like factor 5, Kinesin-like protein KIFC3, Kinesin-like protein KIF3C, Kinesin-like protein KIF23, Kinesin-1 heavy chain, IAG-nucleoside hydrolase, Histone H1.0, Hexokinase type IV, Heat shock protein HSP 60, Growth hormone-releasing hormone receptor, Glyceraldehyde-3-phosphate dehydrogenase, Glucagon receptor, Gastrotropin, Frizzled-8, Eukaryotic translation initiation factor 2-alpha kinase 3, Elongation factor 2, Elongation factor 1-gamma, Elongation factor 1-delta, Elongation factor 1-beta, Elongation factor 1-alpha 1, Dipeptidyl peptidase 3, Dihydrolipoyllysine-residue acetyltransferase component of pyruvate dehydrogenase complex, Diacylglycerol kinase alpha, DNA topoisomerase type IB small subunit, Cytochrome P450 3A11, Concanavalin-A, Chromosome-associated kinesin KIF4A, Centromere-associated protein E, Cathepsin L2, Calpain 2, CAAX prenyl protease 1, Butyrophilin subfamily 3 member A1, Bromodomain-containing protein 3, Bromodomain-containing protein 2, Bromodomain testis-specific protein, Bacterial leucyl aminopeptidase, Alpha enolase, Alkyldihydroxyacetonephosphate synthase, peroxisomal, Acyl coenzyme A:cholesterol acyltransferase 2, Actin, cytoplasmic 1, AICAR transformylase, 60S acidic ribosomal protein P2, 40S ribosomal protein S27, and 26S proteasome non-ATPase regulatory subunit 14, 14-3-3 protein zeta/delta.

Piper Species-Containing Phytomedicines

In some embodiments, at least one transcultural dictionary of the transcultural dictionaries comprises a search dictionary that collates Western and non-Western epistemological understanding of Piper species associated with a therapeutic indication. See, for example, non-limiting methods described in Example 3.

In some embodiments, PhAROS is sued to identify alternatives to Piper species for anxiety, pain, relaxation, and epilepsy.

In some embodiments, populating the transcultural dictionaries with additional data developed by the machine learning algorithm comprises generating a dictionary for Piper species.

In some embodiments, the therapeutic indication is selected from pain, sedation, anxiety, depression, epilepsy, mood, and sleep.

In some embodiments, the therapeutic indication is selected from: hydropisy, gout, acne, coma, generalized hypopigmentation of hair, abnormal intrinsic pathway, abnormal female internal genitalia, pterygium, pain, gout, apoplexy, atony, headache, cancer giddiness, ring worm, epilepsy, otalgia, sciatica, hallucinations, alopecia, leucoderma/vitiligo, paralysis/hemiplegia, quartan fever ichthyosis, arthralgia, ptyriasis alba, congenital deafness alopecia furfuracea, hepatic obstruction, psychosis/insanity/mania, diseases of head and neck, bronchial asthma scrofula/cervical lymphadenitis, paroxysmal fever/intermittent fever bellas palsy, cramp/convulsion/spasm, strangury/dribbling of urine flaccidity, dyspnea, tremor, vertigo, tenesmus, poisoning flatulence, jaundice, toothache, hemorrhage, arthritis, lumbago backache, urinary incontinence, colic, weakness of stomach, sexual debility/anaphrodisia, palpitation, delerium, ptyriasis nigra, gastric dyscrasia, piles/ano rectal mass/haemorrhoids, fever with vata predominance, fatigue, insect bite, phlegmetic cough, splenic obstruction, blurring of vision, night blindness, corneal opacity, indigestion, vata-kaphaja, oedema/inflammation, anemia, chronic obstructive jaundice/chlorosis, cough/bronchitis, emaciation/cachexia, seminal disorders, pulmonary cavitation, gaseous/flatulence, disease with kapha predominance, tubercular cough/cough due to weakness or emaciation, pyrexia, diseases of spleen, dyspepsia/loss of appetite sprue/malabsorption syndrome, urinary disorders/polyuria curable disease of severe nature, obesity, cholera, asthma insomnia, sedative, diarrhea, anorexia, dysentery, dyspepsia, gonorrhea, rheumatism, bronchitis, cholagogue, emmenagogue, abdominal lump, angina pectoris, pleurodynia and intercostal neuralgia, stiffness, dryness of mouth, diseases of the mouth, diseases of head, and disease with vata predominance.

In some embodiments, the user input query comprises a list of Piper species of the family Piperaceae.

In some embodiments, said outputting the processed data returned by the query comprises outputting: a list of Piper species associated with one or more therapeutic indications.

In some embodiments, the one or more therapeutic indications is selected from pain, sedation, anxiety, depression, epilepsy, mood, and sleep.

In some embodiments, the therapeutic indication is selected from: hydropisy, gout, acne, coma, generalized hypopigmentation of hair, abnormal intrinsic pathway, abnormal female internal genitalia, pterygium, pain, gout, apoplexy, atony, headache, cancer giddiness, ring worm, epilepsy, otalgia, sciatica, hallucinations, alopecia, leucoderma/vitiligo, paralysis/hemiplegia, quartan fever ichthyosis, arthralgia, ptyriasis alba, congenital deafness alopecia furfuracea, hepatic obstruction, psychosis/insanity/mania, diseases of head and neck, bronchial asthma scrofula/cervical lymphadenitis, paroxysmal fever/intermittent fever bellas palsy, cramp/convulsion/spasm, strangury/dribbling of urine flaccidity, dyspnea, tremor, vertigo, tenesmus, poisoning flatulence, jaundice, toothache, hemorrhage, arthritis, lumbago backache, urinary incontinence, colic, weakness of stomach, sexual debility/anaphrodisia, palpitation, delerium, ptyriasis nigra, gastric dyscrasia, piles/ano rectal mass/haemorrhoids, fever with vata predominance, fatigue, insect bite, phlegmetic cough, splenic obstruction, blurring of vision, night blindness, corneal opacity, indigestion, vata-kaphaja, oedema/inflammation, anemia, chronic obstructive jaundice/chlorosis, cough/bronchitis, emaciation/cachexia, seminal disorders, pulmonary cavitation, gaseous/flatulence, disease with kapha predominance, tubercular cough/cough due to weakness or emaciation, pyrexia, diseases of spleen, dyspepsia/loss of appetite sprue/malabsorption syndrome, urinary disorders/polyuria curable disease of severe nature, obesity, cholera, asthma insomnia, sedative, diarrhea, anorexia, dysentery, dyspepsia, gonorrhea, rheumatism, bronchitis, cholagogue, emmenagogue, abdominal lump, angina pectoris, pleurodynia and intercostal neuralgia, stiffness, dryness of mouth, diseases of the mouth, diseases of head, and disease with vata predominance.

In some embodiments, outputting the processed data returned by the query comprises outputting: the list of piper species that are convergent across one or more TMS using the in silico convergent analysis.

In some embodiments, the list of Piper species comprises Piper attenuatum, Piper betle, Piper betle, Piper boehmeriaefolium, Piper borbonense, Piper capense, Piper chaba, Piper cubeba, Piper cubeba, Piper cubeba, Piper cubeba, Piper futokadsura, Piper futo-kadzura, Piper guineense, Piper hamiltonii, Piper kadsura, Piper kadsura, Piper laetispicum, Piper longum, Piper longum, Piper longum, Piper longum, Piper mullesua, Piper nigrum, Piper nigrum, Piper nigrum, Piper nigrum, Piper nigrurml., Piper puberulum, Piper pyrifolium, Piper retrofractum, Piper retrofractum, Piper retrofractum, Piper schmidtii, Piper sylvaticum, Piper sylvestre, and Piper umbellatum.

In some embodiments, each Piper species within the list of Piper species is associated with one or more TMS, therapeutic indications within the one or more TMS, sets of chemical components linked to each Pipers species and associated with the therapeutic indication, or a combination thereof.

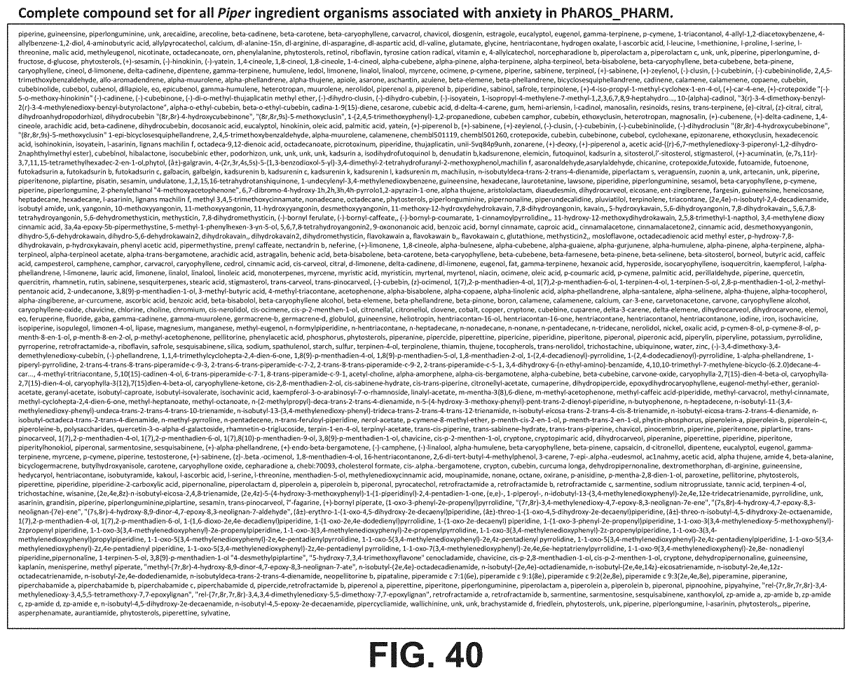

In some embodiments, the list of chemical components for the list of Piper species associated with the therapeutic indication, anxiety, comprises piperine, guineensine, piperlonguminine, arecaidine, arecoline, beta-cadinene, beta-carotene, beta-caryophyllene, carvacrol, chavicol, diosgenin, estragole, eucalyptol, eugenol, gamma-terpinene, p-cymene, 1-triacontanol, 4-allyl-1,2-diacetoxybenzene, 4-allylbenzene-1,2-diol, 4-aminobutyric acid, allylpyrocatechol, calcium, dl-alanine-15n, dl-arginine, dl-asparagine, dl-aspartic acid, dl-valine, glutamate, glycine, hentriacontane, hydrogen oxalate, 1-ascorbic acid, 1-leucine, 1-methionine, l-proline, 1-serine, 1-threonine, malic acid, methyleugenol, nicotinate, octadecanoate, orn, phenylalanine, phytosterols, retinol, riboflavin, tyrosine cation radical, vitamin e, 4-allylcatechol, norcepharadione b, piperolactam a, piperolactam c, piperine, piperlongumine, d-fructose, d-glucose, phytosterols, (+)-sesamin, (−)-hinokinin, (−)-yatein, 1,4-cineole, 1,8-cineol, 1,8-cineole, 1-4-cineol, alpha-cubebene, alpha-pinene, alpha-terpinene, alpha-terpineol, beta-bisabolene, beta-caryophyllene, beta-cubebene, beta-pinene, caryophyllene, cineol, d-limonene, delta-cadinene, dipentene, gamma-terpinene, humulene, ledol, limonene, linalol, linalool, myrcene, ocimene, p-cymene, piperine, sabinene, terpineol, (+)-sabinene, (+)-zeylenol, (−)-clusin, (−)-cubebinin, (−)-cubebininolide, 2,4,5-trimethoxybenzaldehyde, allo-aromadendrene, alpha-muurolene, alpha-phellandrene, alpha-thujene, apiole, asarone, aschantin, azulene, beta-elemene, beta-phellandrene, bicyclosesquiphellandrene, cadinene, calamene, calamenene, copaene, cubebin, cubebinolide, cubebol, cubenol, dillapiole, eo, epicubenol, gamma-humulene, heterotropan, muurolene, nerolidol, piperenol a, piperenol b, piperidine, sabinol, safrole, terpinolene, (+)-4-iso-propyl-1-methyl-cyclohex-1-en-4-ol, (+)-car-4-ene, (+)-crotepoxide “(−)-5-o-methoxy-hinokinin” (−)-cadinene, (−)-cubebinone, (−)-di-o-methyl-thujaplicatin methyl ether, (−)-dihydro-clusin, (−)-dihydro-cubebin, (−)-isoyatein, 1-isopropyl-4-methylene-7-methyl-1,2,3,6,7,8,9-heptahydro, 10-(alpha)-cadinol, “3(r)-3-4-dimethoxy-benzyl-2(r)-3-4-methylenedioxy-benzyl-butyrolactone”, alpha-o-ethyl-cubebin, beta-o-ethyl-cubebin, cadina-1-9(15)-diene, cesarone, cubebic acid, d-delta-4-carene, gum, hemi-ariensin, 1-cadinol, manosalin, resinoids, resins, trans-terpinene, (e)-citral, (z)-citral, citral, dihydroanhydropodorhizol, dihydrocubebin “(8r,8r)-4-hydroxycubebinone”, “(8r,8r,9s)-5-methoxyclusin”, 1-(2,4,5-trimethoxyphenyl)-1,2-propanedione, cubeben camphor, cubebin, ethoxyclusin, heterotropan, magnosalin, (+)-cubenene, (+)-delta-cadinene, 1,4-cineole, arachidic acid, beta-cadinene, dihydrocubebin, docosanoic acid, eucalyptol, hinokinin, oleic acid, palmitic acid, yatein, (+)-piperenol b, (+)-sabinene, (+)-zeylenol, (−)-clusin, (−)-cubebinin, (−)-cubebininolide, (−)-dihydroclusin “(8r,8r)-4-hydroxycubebinone”, “(8r,8r,9s)-5-methoxyclusin” 1-epi-bicyclosesquiphellandrene, 2,4,5-trimethoxybenzaldehyde, alpha-muurolene, calamenene, chembl501119, chembl501260, crotepoxide, cubebin, cubebinone, cubebol, cyclohexane, epizonarene, ethoxyclusin, hexadecenoic acid, isohinokinin, isoyatein, 1-asarinin, lignans machilin f, octadeca-9,12-dienoic acid, octadecanoate, picrotoxinum, piperidine, thujaplicatin, unii-5vg84p9unh, zonarene, (+)-deoxy, (+)-piperenol a, acetic acid-((r)-6,7-methylenedioxy-3-piperonyl-1,2-dihydro-2naphthylmethyl ester), cubebinol, hibalactone, isocubebinic ether, podorhizon, kadsurin a, isodihydrofutoquinol b, denudatin b, kadsurenone, elemicin, futoquinol, kadsurin a, sitosterol, î′-sitosterol, stigmasterol, (+)-acuminatin, (e,7s,11r)-3,7,11,15-tetramethylhexadec-2-en-1-ol, phytol, (â±)-galgravin, 4-(2r,3r,4s,5s)-5-(1,3-benzodioxol-5-yl)-3,4-dimethyl-2-tetrahydrofuranyl-2-methoxyphenol, machilin f, asaronaldehyde, asarylaldehyde, chicanine, crotepoxide, futoxide, futoamide, futoenone, futokadsurin a, futokadsurin b, futokadsurin c, galbacin, galbelgin, kadsurenin b, kadsurenin c, kadsurenin k, kadsurenin l, kadsurenin m, machilusin, n-isobutyldeca-trans-2-trans-4-dienamide, piperlactam s, veraguensin, zuonin a, artecanin, piperine, piperitenone, piplartine, pisatin, sesamin, undulatone, 1,2,15,16-tetrahydrotanshiquinone, 1-undecylenyl-3,4-methylenedioxybenzene, guineensine, hexadecane, laurotetanine, lawsone, piperidine, piperlonguminine, sesamol, beta-caryophyllene, p-cymene, piperine, piperlongumine, 2-phenylethanol “4-methoxyacetophenone”, 6,7-dibromo-4-hydroxy-1h,2h,3h,4h-pyrrolo1,2-apyrazin-1-one, alpha thujene, aristololactam, diaeudesmin, dihydrocarveol, eicosane, ent-zingiberene, fargesin, guineensine, heneicosane, heptadecane, hexadecane, 1-asarinin, lignans machilin f, methyl 3,4,5-trimethoxycinnamate, nonadecane, octadecane, phytosterols, piperlonguminine, pipernonaline, piperundecalidine, pluviatilol, terpinolene, triacontane, (2e,4e)-n-isobutyl-2,4-decadienamide, isobutyl amide, yangonin, 10-methoxyyangonin, 11-methoxyyangonin, 11-hydroxyyangonin, desmethoxyyangonin, 11-methoxy-12-hydroxydehydrokavain, 7,8-dihydroyangonin, kavain, 5-hydroxykavain, 5,6-dihydroyangonin, 7,8-dihydrokavain, 5,6,7,8-tetrahydroyangonin, 5,6-dehydromethysticin, methysticin, 7,8-dihydromethysticin, (−)-bornyl ferulate, (−)-bornyl-caffeate, (−)-bornyl-p-coumarate, 1-cinnamoylpyrrolidine, 11-hydroxy-12-methoxydihydrokawain, 2,5,8-trimethyl-1-napthol, 3,4-methylene dioxy cinnamic acid, 3a,4a-epoxy-5b-pipermethystine, 5-methyl-1-phenylhexen-3-yn-5-ol, 5,6,7,8-tetrahydroyangonin2, 9-oxononanoic acid, benzoic acid, bornyl cinnamate, caproic acid, cinnamalacetone, cinnamalacetone2, cinnamic acid, desmethoxyyangonin, dihydro-5,6-dehydrokawain, dihydro-5,6-dehydrokawain2, dihydrokavain, dihydrokavain2, dihydromethysticin, flavokawain a, flavokawain b, flavokawain c, glutathione, methysticin2, mosloflavone, octadecadienoic acid methyl ester, p-hydroxy-7,8-dihydrokavain, p-hydroxykavain, phenyl acetic acid, pipermethystine, prenyl caffeate, nectandrin b, neferine, (+)-limonene, 1,8-cineole, alpha-bulnesene, alpha-cubebene, alpha-guaiene, alpha-gurjunene, alpha-humulene, alpha-pinene, alpha-terpinene, alpha-terpineol, alpha-terpineol acetate, alpha-trans-bergamotene, arachidic acid, astragalin, behenic acid, beta-bisabolene, beta-carotene, beta-caryophyllene, beta-cubebene, beta-farnesene, beta-pinene, beta-selinene, beta-sitosterol, borneol, butyric acid, caffeic acid, campesterol, camphene, camphor, carvacrol, caryophyllene, cedrol, cinnamic acid, cis-carveol, citral, d-limonene, delta-cadinene, dl-limonene, eugenol, fat, gamma-terpinene, hexanoic acid, hyperoside, isocaryophyllene, isoquercitrin, kaempferol, 1-alpha-phellandrene, 1-limonene, lauric acid, limonene, linalol, linalool, linoleic acid, monoterpenes, myrcene, myristic acid, myristicin, myrtenal, myrtenol, niacin, ocimene, oleic acid, p-coumaric acid, p-cymene, palmitic acid, perillaldehyde, piperine, quercetin, quercitrin, rhamnetin, rutin, sabinene, sesquiterpenes, stearic acid, stigmasterol, trans-carveol, trans-pinocarveol, (−)-cubebin, (z)-ocimenol, 1(7),2-p-menthadien-4-ol, 1(7),2-p-menthadien-6-ol, 1-terpinen-4-ol, 1-terpinen-5-ol, 2,8-p-menthadien-1-ol, 2-methyl-pentanoic acid, 2-undecanone, 3,8(9)-p-menthadien-1-ol, 3-methyl-butyric acid, 4-methyl-triacontane, acetophenone, alpha-bisabolene, alpha-copaene, alpha-linolenic acid, alpha-phellandrene, alpha-santalene, alpha-selinene, alpha-thujene, alpha-tocopherol, alpha-zingiberene, ar-curcumene, ascorbic acid, benzoic acid, beta-bisabolol, beta-caryophyllene alcohol, beta-elemene, beta-phellandrene, beta-pinone, boron, calamene, calamenene, calcium, car-3-ene, carvetonacetone, carvone, caryophyllene alcohol, caryophyllene-oxide, chavicine, chlorine, choline, chromium, cis-nerolidol, cis-ocimene, cis-p-2-menthen-1-ol, citronellal, citronellol, clovene, cobalt, copper, cryptone, cubebine, cuparene, delta-3-carene, delta-elemene, dihydrocarveol, dihydrocarvone, elemol, eo, feruperine, fluoride, gaba, gamma-cadinene, gamma-muurolene, germacrene-b, germacrene-d, globulol, guineensine, heliotropin, hentriacontan-16-ol, hentriacontan-16-one, hentriacontane, hentriacontanol, hentriacontanone, iodine, iron, isochavicine, isopiperine, isopulegol, limonen-4-ol, lipase, magnesium, manganese, methyl-eugenol, n-formylpiperidine, n-hentriacontane, n-heptadecane, n-nonadecane, n-nonane, n-pentadecane, n-tridecane, nerolidol, nickel, oxalic acid, p-cymen-8-ol, p-cymene-8-ol, p-menth-8-en-1-ol, p-menth-8-en-2-ol, p-methyl-acetophenone, pellitorine, phenylacetic acid, phosphorus, phytosterols, piperanine, pipercide, piperettine, pipericine, piperidine, piperitone, piperonal, piperonic acid, piperylin, piperyline, potassium, pyrrolidine, pyrroperine, retrofractamide-a, riboflavin, safrole, sesquisabinene, silica, sodium, spathulenol, starch, sulfur, terpinen-4-ol, terpinolene, thiamin, thujene, tocopherols, trans-nerolidol, trichostachine, ubiquinone, water, zinc, (−)-3,4-dimethoxy-3,4-demethylenedioxy-cubebin, (−)-phellandrene, 1,1,4-trimethylcyclohepta-2,4-dien-6-one, 1,8(9)-p-menthadien-4-ol, 1,8(9)-p-menthadien-5-ol, 1,8-menthadien-2-ol, 1-(2,4-decadienoyl)-pyrrolidine, 1-(2,4-dodecadienoyl)-pyrrolidine, 1-alpha-phellandrene, 1-piperyl-pyrrolidine, 2-trans-4-trans-8-trans-piperamide-c-9-3, 2-trans-6-trans-piperamide-c-7-2, 2-trans-8-trans-piperamide-c-9-2, 2-trans-piperamide-c-5-1, 3,4-dihydroxy-6-(n-ethyl-amino)-benzamide, 4,10,10-trimethyl-7-methylene-bicyclo-(6.2.0)decane-4-car . . . , 4-methyl-tritriacontane, 5,10(15)-cadinen-4-ol, 6-trans-piperamide-c-7-1, 8-trans-piperamide-c-9-1, acetyl-choline, alpha-amorphene, alpha-cis-bergamotene, alpha-cubebine, beta-cubebine, carvone-oxide, caryophylla-2,7(15)-dien-4-beta-ol, caryophylla-2,7(15)-dien-4-ol, caryophylla-3(12),7(15)dien-4-beta-ol, caryophyllene-ketone, cis-2,8-menthadien-2-ol, cis-sabinene-hydrate, cis-trans-piperine, citronellyl-acetate, cumaperine, dihydropipercide, epoxydihydrocaryophyllene, eugenol-methyl-ether, geraniol-acetate, geranyl-acetate, isobutyl-caproate, isobutyl-isovalerate, isochavinic acid, kaempferol-3-o-arabinosyl-7-o-rhamnoside, linalyl-acetate, m-mentha-3(8),6-diene, m-methyl-acetophenone, methyl-caffeic acid-piperidide, methyl-carvacrol, methyl-cinnamate, methyl-cyclohepta-2,4-dien-6-one, methyl-heptanoate, methyl-octanoate, n-(2-methylpropyl)-deca-trans-2-trans-4-dienamide, n-5-(4-hydroxy-3-methoxy-phenyl)-pent-trans-2-dienoyl-piperidine, n-butyophenone, n-heptadecene, n-isobutyl-11-(3,4-methylenedioxy-phenyl)-undeca-trans-2-trans-4-trans-10-trienamide, n-isobutyl-13-(3,4-methylenedioxy-phenyl)-trideca-trans-2-trans-4-trans-12-trienamide, n-isobutyl-eicosa-trans-2-trans-4-cis-8-trienamide, n-isobutyl-eicosa-trans-2-trans-4-dienamide, n-isobutyl-octadeca-trans-2-trans-4-dienamide, n-methyl-pyrroline, n-pentadecene, n-trans-feruloyl-piperidine, nerol-acetate, p-cymene-8-methyl-ether, p-menth-cis-2-en-1-ol, p-menth-trans-2-en-1-ol, phytin-phosphorus, piperolein-a, piperolein-b, piperolein-c, piperoleine-b, polysaccharides, quercetin-3-o-alpha-d-galactoside, rhamnetin-o-triglucoside, terpin-1-en-4-ol, terpinyl-acetate, trans-cis-piperine, trans-sabinene-hydrate, trans-trans-piperine, chavicol, pinocembrin, piperine, piperitenone, piplartine, trans-pinocarveol, 1(7),2-p-menthadien-4-ol, 1(7),2-p-menthadien-6-ol, 1(7),8(10)-p-menthadien-9-ol, 3,8(9)-p-menthadien-1-ol, chavicine, cis-p-2-menthen-1-ol, cryptone, cryptopimaric acid, dihydrocarveol, piperanine, piperettine, piperidine, piperitone, piperitylhonokiol, piperonal, sarmentosine, sesquisabinene, (+)-alpha-phellandrene, (+)-endo-beta-bergamotene, (−)-camphene, (−)-linalool, alpha-humulene, beta-caryophyllene, beta-pinene, capsaicin, d-citronellol, dipentene, eucalyptol, eugenol, gamma-terpinene, myrcene, p-cymene, piperine, testosterone, (+)-sabinene, (z)-.beta.-ocimenol, 1,8-menthadien-4-ol, 16-hentriacontanone, 2,6-di-tert-butyl-4-methylphenol, 3-carene, 7-epi-.alpha.-eudesmol, aclnahmy, acetic acid, alpha thujene, amide 4, beta-alanine, bicyclogermacrene, butylhydroxyanisole, carotene, caryophyllone oxide, cepharadione a, chebi:70093, cholesterol formate, cis-.alpha.-bergamotene, crypton, cubebin, Curcuma longa, dehydropipernonaline, dextromethorphan, dl-arginine, guineensine, hedycaryol, hentriacontane, isobutyramide, kakoul, 1-ascorbic acid, 1-serine, 1-threonine, menthadien-5-ol, methylenedioxycinnamic acid, moupinamide, nonane, octane, oxirane, p-anisidine, p-mentha-2,8-dien-1-ol, paroxetine, pellitorine, phytosterols, piperettine, piperidine, piperidine-2-carboxylic acid, pipernonaline, piperolactam d, piperolein a, piperolein b, piperonal, pyrocatechol, retrofractamide a, retrofractamide b, retrofractamide c, sarmentine, sodium nitroprussiate, tannic acid, terpinen-4-ol, trichostachine, wisanine, (2e,4e,8z)-n-isobutyl-eicosa-2,4,8-trienamide, (2e,4z)-5-(4-hydroxy-3-methoxyphenyl)-1-(1-piperidinyl)-2,4-pentadien-1-one, (e,e)-, 1-piperoyl-, n-idobutyl-13-(3,4-methylenedioxyphenyl)-2e,4e,12e-tridecatrienamide, pyrrolidine, asarinin, grandisin, piperine, piperlonguminine, piplartine, sesamin, trans-pinocarveol, î″-fagarine, (+)-bornyl piperate, (1-oxo-3-phenyl-2e-propenyl)pyrrolidine, “(7r,8r)-3,4-methylenedioxy-4,7-epoxy-8,3-neolignan-7e-ene”, “(7s,8r)-4-hydroxy-4,7-epoxy-8,3-neolignan-(7e)-ene”, “(7s,8r)-4-hydroxy-8,9-dinor-4,7-epoxy-8,3-neolignan-7-aldehyde”, (â±)-erythro-1-(1-oxo-4,5-dihydroxy-2e-decaenyl)piperidine, (a*)-threo-1-(1-oxo-4,5-dihydroxy-2e-decaenyl)piperidine, (â±)-threo-n-isobutyl-4,5-dihydroxy-2e-octaenamide, 1(7),2-p-menthadien-4-ol, 1(7),2-p-menthadien-6-ol, 1-(1,6-dioxo-2e,4e-decadienyl)piperidine, 1-(1-oxo-2e,4e-dodedienyl)pyrrolidine, 1-(1-oxo-2e-decaenyl) piperidine, 1-(1-oxo-3-phenyl-2e-propenyl)piperidine, 1-1-oxo-3(3,4-methylenedioxy-5-methoxyphenyl)-2zpropenyl piperidine, 1-1-oxo-3(3,4-methylenedioxyphenyl)-2e-propenylpiperidine, 1-1-oxo-3(3,4-methylenedioxyphenyl)-2e-propenylpyrrolidine, 1-1-oxo-3(3,4-methylenedioxyphenyl)-2z-propenylpiperidine, 1-1-oxo-3(3,4-methylenedioxyphenyl)propylpiperidine, 1-1-oxo-5(3,4-methylenedioxyphenyl)-2e,4e-pentadienylpyrrolidine, 1-1-oxo-5(3,4-methylenedioxyphenyl)-2e,4z-pentadienyl pyrrolidine, 1-1-oxo-5(3,4-methylenedioxyphenyl)-2e,4z-pentadienylpiperidine, 1-1-oxo-5(3,4-methylenedioxyphenyl)-2z,4e-pentadienyl piperidine, 1-1-oxo-5(3,4-methylenedioxyphenyl)-2z,4e-pentadienyl pyrrolidine, 1-1-oxo-7(3,4-methylenedioxyphenyl)-2e,4e,6e-heptatrienylpyrrolidine, 1-1-oxo-9(3,4-methylenedioxyphenyl)-2e,8e-nonadienyl piperidine, pipernonaline, 1-terpinen-5-ol, 3,8(9)-p-menthadien-1-ol “4-desmethylpiplartine”, “5-hydroxy-7,3,4-trimethoxyflavone” cenocladamide, chavicine, cis-p-2,8-menthadien-1-ol, cis-p-2-menthen-1-ol, cryptone, dehydropipernonaline, guineensine, kaplanin, menisperine, methyl piperate, “methyl-(7r,8r)-4-hydroxy-8,9-dinor-4,7-epoxy-8,3-neolignan-7-ate”, n-isobutyl-(2e,4e)-octadecadienamide, n-isobutyl-(2e,4e)-octadienamide, n-isobutyl-(2e,4e,14z)-eicosatrienamide, n-isobutyl-2e,4e,12z-octadecatrienamide, n-isobutyl-2e,4e-dodedienamide, n-isobutyldeca-trans-2-trans-4-dienamide, neopellitorine b, pipataline, piperamide c 7:1(6e), piperamide c 9:1(8e), piperamide c 9:2(2e,8e), piperamide c 9:3(2e,4e,8e), piperamine, piperanine, piperchabamide a, piperchabamide b, piperchabamide c, piperchabamide d, pipercide, retrofractamide b, piperenol a, piperettine, piperitone, piperlonguminine, piperolactam a, piperolein a, piperolein b, piperonal, pipnoohine, pipyahyine, “rel-(7r,8r,7r,8r)-3,4-methylenedioxy-3,4,5,5-tetramethoxy-7,7-epoxylignan”, “rel-(7r,8r,7r,8r)-3,4,3,4-dimethylenedioxy-5,5-dimethoxy-7,7-epoxylignan”, retrofractamide a, retrofractamide b, sarmentine, sarmentosine, sesquisabinene, xanthoxylol, zp-amide a, zp-amide b, zp-amide c, zp-amide d, zp-amide e, n-isobutyl-4,5-dihydroxy-2e-decaenamide, n-isobutyl-4,5-epoxy-2e-decaenamide, pipercycliamide, wallichinine, brachystamide d, friedlein, phytosterols, piperine, piperlongumine, 1-asarinin, phytosterols, piperine, asperphenamate, aurantiamide, phytosterols, piperettine, and sylvatine (See FIG. 40).

In some embodiments, the list of chemical components for at least one Piper species comprises bis-noryangonin, 11-methoxy-nor-yangonin, 5,6-dehydrokawain, dihydromethysticin, and yangonin.

In some embodiments, the at least one Piper species is Piper methysticum.

In particular, PhAROS was used to identify alternatives to P. methysticum for anxiety, pain, relaxation and epilepsy based on the restricted biogeography of P. methysticum and reports of compounds within P. methysticum with purported liver toxicity.

In some embodiments, the second user query input for further analysis initiated by the second user query input comprises the list of chemical components: bis-noryangonin, 11-methoxy-nor-yangonin, 5,6-dehydrokawain, dihydromethysticin, and yangonin.

In some embodiments, further analysis initiated by the second user query input comprising the list of chemical components comprises using the second user query input to search transcultural dictionaries, the data from the plurality of TMS associated with the second user query input.

In some embodiments, further analysis comprises processing the data associated with the second user query input to create a second processed data returned by the second query user input, and retrieving the second processed data based on the second query user input.

In some embodiments, the second processed data comprises a list of non-Piper species comprising the list of chemical components.

In some embodiments, the list of non-Piper species comprises Petroselinum crispum, Dioscorea collettii, Dioscorea hypoglauca, Gentiana algida, Rubia cordifolia, and Alpinia speciosa.

In some embodiments, processing the data associated with the second query user input comprises screening for non-Piper species comprising the list of chemical components.

In some embodiments, further analysis comprises processing the data associated with the second user query input to create a second processed data returned by the second query user input, and retrieving the second processed data based on the second query user input.

In some embodiments, the second user query input comprises a biogeography of P. methysticum and a list of therapeutic indications, wherein the list of therapeutic indications comprises anxiety, mood, and depression.

In some embodiments, the second processed data comprises a list of non-Piper species associated with anxiety, mood, depression, or a combination thereof found in non-piper species within the biogeography of P. methysticum.

In some embodiments, the list of non-Piper species comprises Glycyrhizza uralensis/radix, Paeonia lactiflora, Scutellaria baicalensis, Panax ginseng, Saposhnikovia divaicata, and Poria cocos.

Cancer

In some embodiments, the first user input query comprises one or more user selected clinical indications.

In some embodiments, the one or more user selected clinical indications is selected from cancer, cancer pain, and cancer and cancer pain. In such cases, PhAROS_CONVERGE convergence analysis and PhAROS_DIVERGE divergence analysis are used to identify potential cytotoxic agents that could become new cancer fighting drugs within complex TMS formulations for cancer and identify compound sets with potential for cancer pain over other pain subtypes. See, for example, Example 4.

In some embodiments, said outputting the processed data returned by the query comprises outputting: a list of compounds associated with the user selected clinical indication, a list of prescription formulae for a given TMS, a list of organisms associated with the user selected clinical indication, or a combination thereof.

In some embodiments, the outputting further comprises outputting cytotoxic agents within the list of compounds that are indicated for pain and cancer across one or more TMS.

In some embodiments, outputting further comprises outputting the list of organisms associated with cancer and pain across one or more TMS.

In some embodiments, the list of compounds is categorized by class, identified as migraine dictionary hits, and are convergent between two or more TMS.

In some embodiments, the outputting further comprises outputting a list of compounds that is associated with a first user selected clinical indication, wherein the list of compounds that is associated with the first user selected clinical indication does not overlap with a list of compounds that is associated with a second user selected indication.

In some embodiments, the first user selected clinical indication is cancer, and the second user selected indication is pain.

### PhAROS System

Aspects of the present disclosure include systems for carrying out the steps of the methods described herein.

An aspect of the present disclosure provides a phytomedicine analytics for research optimization at scale (PhAROS) system for analyzing a plurality of traditional medical systems in a single computational space, the PhAROS system comprising: a computer server configured to communicate with one or more user clients (PhAROS_USER) comprising: (a) a database (PhAROS_BASE) comprising a memory configured to store a collection of data, the collection of data comprising: raw and optionally pre-processed data from a plurality of traditional medicine data sets; and optionally one or more of: plant data sets; literature-based text documents (corpus); and machine learning data sets; (b) a computer core processor (PhAROS_CORE), wherein the PhAROS_CORE is configured to receive and process the collection of data from the PhAROS_BASE to generate processed data; (c) one or more searchable repositories having data and optionally pre-processed data, wherein each searchable repository comprises a memory configured to store data entries, wherein the PhAROS_CORE is configured to send the processed data to and receive data from each of the searchable repositories, wherein each of the searchable repositories is configured to receive processed data from the PhAROS_CORE and send data and optionally pre-processed data to the PhAROS_CORE; (d) a computer-readable storage medium storing executable instructions that, when executed by a hardware processor, cause the PhAROS_CORE to communicate with the PhAROS_BASE and one or more of the searchable repositories to analyze data from a plurality of the traditional medicine data sets to produce an output responsive to a user query input into the PhAROS system.

In some embodiments, the PhAROS_CORE is further configured to manage, direct, collect, parse, and filter the collection of data from the PhAROS_BASE to generate processed data.

In some embodiments, the PhAROS system further comprises one or more user clients (PhAROS_USER).

In some embodiments, at least one PhAROS_USER client has a graphical user interface (GUI). The interface such as a graphical user interface (GUI) may be the visual component of the application for a user to enter inputs, selects different data entries, and views results generated by the computing server. In some embodiments, the interface may not include visual elements but allow a user to interface with the computing server directly through code instructions, such as in the case of an API. The interface may display various visualizations of data and results. For example, the interface may display various charts and analytics that summarize the results of a data analysis. The interface may also display visual data geographically such as by showing the associated locations of various data entries in a digital map. The interface may include various interactive elements such as checklists, dialog boxes, dropdown menus, tabs, and other control elements.

In some embodiments, at least one PhAROS_USER client is configured to allow the user to communicate with the PhAROS_CORE.

In some embodiments, at least one PhAROS_USER client is configured to allow the user to communicate with at least one of the searchable repositories.

In some embodiments, at least one PhAROS_USER client is configured to allow the user to communicate with the PhAROS_CORE, PhAROS_BASE, and the searchable repositories.

In some embodiments, at least one searchable repository comprises: a first meta-pharmacopeia database (PhAROS_PHARM) comprising (i) data from PhAROS_BASE; and (ii) pre-processed data processed from data in the PhAROS_BASE related to at least one of: medical formulations; organisms; medical compound data sets; therapeutic indications; processed and normalized formalized pharmacopeias from one or more geographic regions associated with traditional medicines.

In some embodiments, the one or more geographic regions is selected from: Japan, China, India, Korea, South East Asia, Middle East, North America, South America, Russia, India, Africa, Europe, and Australia.

In some embodiments, the one or more processed and normalized formalized pharmacopeias comprises processed, translated normalized, individual published datasets or case reports in the scientific literature that document relationships between medicinal plants and disease indications.

In some embodiments, the one or more processed and normalized formalized pharmacopeias comprises processed, curated ethical partnerships, indigenous, cultural phytomedical formulations.

In some embodiments, the one or more processed and normalized formalized pharmacopeias comprises processed contemporary and historical herbologies that document relationships between medicinal plants and disease indications (e.g., Hildegard of Bingen, Causae et Curae, Physica).

In some embodiments, the one or more processed and normalized formalized pharmacopeias comprises processed, translation of resources from original languages processed using approaches selected from one or more of: machine literal translation, natural language processing, multilingual concept extraction or conventional translation, Optical character recognition (OCR) of historical materials, and artificial intelligence (AI)-driven intent translation.

In some embodiments, at least one searchable repository (PhAROS_CONVERGE) comprises data and pre-processed data that allow identification of commonalities in therapeutic approaches from biogeographically and culturally traditional medical systems (TMS).

In some embodiments, the data and pre-processed data of the PhAROS_CONVERGE is further configured to allow identification of efficacious medical components across traditional medicine systems.

In some embodiments, the data and pre-processed data of the PhAROS_CONVERGE is further configured to allow ranking optimization of de novo compound formulations and compound mixtures by utilizing transcultural components for subsequent preclinical and clinical testing for a given therapeutic indication.

In some embodiments, the data and pre-processed data of the PhAROS_CONVERGE comprises at least one of: therapeutic indication dictionaries related to traditional medical systems that reflect modern and historical terminology, and/or Western and non-Western epistemologies; medical formulation compositions related to traditional medical systems; compound data sets for a given therapeutic indication; and a proprietary digital composition index (n-dimensional vector and/or fingerprint).

In some embodiments, the computer-readable storage medium storing executable instructions, when executed by the hardware processor, cause the hardware processor to: develop training data sets for one or more machine learning algorithms to optimize the searchable repositories for a user; populate the one or more searchable repositories with additional data developed by the machine learning algorithm; and create, update, annotate, process, download, analyze, or manipulate the collection of data received by the Pharos_CORE.

In some embodiments, the computer-readable storage medium storing executable instructions, when executed by the hardware processor, cause the PhAROS_CORE to: initiate a user to provide the user query input on the PhAROS_USER client, wherein the PhAROS_USER client is configured to communicate with the PhAROS_CORE and optionally the searchable repositories; search the user query input within the PhAROS_CORE, the searchable repositories, or a combination thereof, retrieve the processed data based on the user's query input for review by the user in PhAROS_USER; optionally initiate further processing of the retrieved processed data, if inquired by the user.

In some embodiments, the PhAROS_USER client further comprises a graphical data processing environment (PhAROS_FLOW) configured to allow the user to process data without or with reduced amount of at least one of: coding, system modeling tools comprising machine learning, or artificial intelligence (AI) tools.

In some embodiments, the machine learning and AI tools are selected from one or more of: support vector machine, artificial neural networks, deep learning, Naïve Bayesian, K-nearest neighbors, random forest, AdaBoost wisdom of crowds and ensemble predictors, and others, validation (such as MonteCarlo cross-validation, Leave-One-Out cross validation, Bootstrap Resampling, and y-randomization).

FIG. 2A shows for illustrative purposes only an example of a schematic of major components of the PhAROS system of one embodiment. FIG. 2A shows a schematic of major components of the PhAROS system.

In some embodiments PhAROS contains a suite of informatics tools, data pipelines and data repositories allowing for user access and decision support tools for identifying a drug, a compound, a mixture, or an organism discovery.

The PhAROS system contains a suite of informatics tools, data pipelines and data repositories allowing for user access and decision support tools for drug discovery. Depending on the need of the user/stakeholder, data repositories, and pre-processed repositories, can be cross correlated, analyzed and assessed for particular questions, these subcomponents and data sets, include but are not limited to: PhAROS_USER, PhAROS_CORE, PhAROS_BRAIN, PhAROS_FLOW, PhAROS_PHARM, PhAROS_CONVERGE, PhAROS_DIVERGE, PhAROS_CHEMBIO, PhAROS_BIOGEO, PhAROS_METAB, PhAROS_MICRO, PhAROS_CURE, PhAROS_QUANT, PhAROS_POPGEN, PhAROS_TOX, PhAROS_BH, PhAROS_EPIST, and PhAROS_BASE.

The PhAROS system includes a computing server, in accordance with some embodiments. The example computing server may include one or more computers such as one or more server-side computing devices and cloud computing devices. The server-side computing device and the cloud computing devices each may include one or more processors and memory. The memory may store computer code that includes instructions. The instructions, when executed by one or more processors, cause the processors to perform one or more processes described herein, such as one or more processes or workflows defined by instructions. In some embodiments, the server-side computing device and the cloud computing devices may be implemented in a distributed manner. For example, the server-side computing device may communicate with the cloud computing devices via the network. The cloud computing devices may include multiple computers operated in a distributed fashion. The computing server may also take other forms. For example, instead of implementing cloud computing devices, the computing server may take the form of a non-cloud server. The computing devices may be one of the on-site servers that communicate with the server-side computing device locally. In some embodiments, the computing server may take the form of a personal computer that executes code instructions directly instead of using any additional computing devices. Other suitable implementations are also possible.

In some embodiments, the computing server may include data mining engine, data integration engine, prediction and machine learning engine, pharmacopeia database, convergence analysis engine, chemical and biological substance database, plant and organism database, metabolomics database, microbiome database, cure prediction engine, quantitative analysis engine, population genetics database, toxicological and side-effect prediction engine, causality engine, epistemology engine, and visualization engine. In various embodiments, the computing server may include fewer or additional components, depending on the functionalities of the computing server in various embodiments. The computing server also may include different components. The functions of various components in computing server may be distributed in a different manner than described below. This particular example computing server may be used for a phytomedicine analytics platform. For other types of federated databases, different components may be used. While the phytomedicine analytics platform is used as an example throughout this description, various techniques and processes discussed herein may be applied to other federated database, medicine related or not.

The components of the computing server may be embodied as software engines that include code (e.g., program code comprised of instructions, machine code, etc.) that is stored on an electronic medium (e.g., memory and/or disk) and executable by one or more processors (e.g., CPUs, GPUs, other general processors). The components also could be embodied in hardware, e.g., field-programmable gate arrays (FPGAs) and/or application-specific integrated circuits (ASICs), that may include circuits alone or circuits in combination with firmware and/or software. Each component may be a combination of software code instructions and hardware such as one or more processors that execute the code instructions to perform various processes. Each component may include all or part of the example structure and configuration of the computing machine described in FIGS. 2A-2D.

The computing server may take the form of a tool accessible within the company for research and development purposes. The computing server may provide a GUI, use mySQL or similar architecture, and enable API code linking to publicly available external databases. In some embodiments, the computing server may take the form of an online platform made available as a science gateway and virtual research environment for drug discovery to users (industry, academia, agencies, healthcare providers) as a fee for service. In some embodiments, the computing server may serve as an exploration tool for consumers and patients.

Data mining engine parses data from various sources, such as external data servers, various databases or subsystems that may be stored in data store, and other unstructured sources such as the Internet and documents. In some embodiments, the data mining engine may include a format converter that changes data formats to a standardized format used in the computing server. For example, a user may provide a search term related to a traditional medicine formulation. The computing server may generate a query to an external data server, such as a traditional Chinese medicine (TCM) database, through a call of the API of the external data server. In response, the external data server provides a data payload in a format defined by the external data server, such as JSON, XML, CSV, or another data-serialization format. The data mining engine may parse data in the payload based on keys and relevant fields and convert the parsed data to a standardized format used in the computing server. The data mining engine may also retrieve data entries from data store through a query language such as SQL. In some embodiments, the data mining engine may also conduct Internet search of key search terms specified by the users. The data mining engine may parse data actual data from the HTML files based of HMTL identifiers, HMTL dividers, CSS selectors, XPath, etc. using parsing tools such as BEAUTIFUL SOUP or NOKOGIRI. The data mining engine may also perform curation and other text mining processes such as scanning of images, OCR, and natural language processing to store data, particularly historical data such as documentations and books of traditional medicines, to various databases operated by the computing server.

The data integration engine consolidating various data entries from different data sources to generate a compiled dataset. The data integration process may occur on demand or a part of the routine process to build various databases in the computing server, such as the pharmacopeia database. In some embodiments, a user of the computing server, through the application, may specify one or more herb components and/or one or more traditional medicine formulas. The computing server, based on the user input, carries out queries to various databases to retrieve data entries that are related to the user inputs. The data entries may include various attributes that agree with or contradict other data entries. The data integration engine may identify the attributes that belong to the same field and aggregate the attributes together. The data integration engine may also identify and flag attributes from different entries that are potentially in conflict with each other. In some embodiments, the data integration engine may also retrieves data from various sources and convert the data in a structured format that has common attributes, metrics and metadata. The standardized data may be saved in the pharmacopeia database.

In some embodiments, the method of creating the PhAROS_PHARM, pre-processed repository, and computational space, generally comprising and including but not limited to, the first ‘meta-pharmacopeia’, processed and normalized formalized pharmacopeias, formulations, associated plant/organisms, associated available compound sets, and indications, temporal and geographical data, indicating historical, and contemporary geographical, cultural and epistemology origins. Efficacy-based research approaches have been proposed as more appropriate for traditional Chinese medicine than attempting to fit the TCM into a Western mechanism-based research framework.

Tang (writing in the BMJ in 2006) asked “is the current Western model of research-trying out unknown treatments in animals-suitable for studying treatments that have long been used in humans?” The PhAROS_PHARM pre-processed repository, and computational space, overcomes these issues syncretically, allowing a diversity of inputs and pathways to outputs that can start from efficacy-based a priori assumptions or mechanistic inquiry. The method includes data input from multiple sources, to become the content of this meta-pharmacopeia repository. Importing, cleaning, reducing and normalizing data and metadata for compounds, ingredient lists, formulations and their associated therapeutic indications. Including but not limited to formalized publicly-available pharmacopeias from Japan, China, India, Korea, South East Asia, Middle East, South America, Russia, India, Africa, Oceania and Europe. Associated metadata will be imported, cleaned, normalized and compressed, this includes historical and contemporary data sources that document linkages between medicinal formulation, ingredient compounds/chemical components and indications for therapeutic use, translations of resources from original languages processed using approaches such as machine translation, natural language processing, multilingual concept extraction or conventional translation; OCR of historical materials.

In some embodiments, an example of a constructed PhAROS_PHARM meta pharmacopeia was assembled in a single computational space containing 20,826 phytomedicine formulations, >31,000 component chemicals and their indications, currently accessible through a graphical dashboard for direct interrogation of this system component, independently of other PhAROS system components and modules. This example dataset contains assembled phytomedical intelligence/data from three continents, five contemporary and historical cultural medical systems, spanning over 5000 years of human medical endeavor and the biogeography of >16.9M square miles of medicinal plant growth.

In some embodiment and one example here, the method used to construct a PhAROS_PHARM meta-pharmacopeia repository and computational space, utilized discrimination data protocols as ‘in-group’ and ‘out-group’ data for inclusion in PhAROS_PHARM data structure. The method in this example utilized only formalized medical systems with established indication-formulation-regimen frameworks, while excluding approaches that rely upon animal medicine, mineral medicine, shamanism and humoral medicine.

FIG. 2B shows a table describing the major components of the PhAROS system, with icon key.

FIG. 2C shows for illustrative purposes only an example of a schematic of major components of the PhAROS system, with icon key of one embodiment. FIG. 2C shows a schematic of major components of the PhAROS system, with icon key. Major components of the PhAROS system are accessible by a user and admin user through the server containing the PhAROS system. The WWW provides access to an external user through a WWW ftp and external databases and data sources. The PhAROS system includes major components including the PhAROS_USER, PhAROS_CORE and PhAROS_BRAlN. Subcomponents are accessed through the major components and include the PhAROS_PHARM, PhAROS_CONVERGE, PhAROS-CHEMBIO, PhAROS_BlOGEO, PhAROS_METAB, PhAROS_MICRO, PhAROS_CURE, PhAROS_QUANT, PhAROS_POPGEN, PhAROS_TOX, PhAROS_BH, PhAROS_EPIST, and PhAROS_BASE K.

In some embodiments, the PhAROS system can, using subcomponents of the system, provide a method to rationalize phytomedicine design and cultivation pipelines for global health issues. Phytomedicines remain as major components of medical optionality for billions of individuals in rural, developing or impoverished locations worldwide. There exists continued advocacy for equitable distribution of Western medicines, and additionally there is not only an economic exigency but an ethical responsibility to optimize formulation and improve availability and access of low cost phytomedicine alternatives to comparatively expensive Western medicines, for global health populations and rationally leverage their potential benefits.

In some embodiments, the PhAROS system can, using subcomponents of the system, provide a method to aid in democratization of optimized phytomedicines, that can also serve populations by decreasing the influence of fraudulent practitioners and eliminating the perceived need for medically-irrelevant exploitative, and sometimes abhorrent, formulation components. PhAROS systems can inform global health solutions using methods in specific sub-systems, by (1) Identifying minimal essential formulations for efficacy and safety through combining data results from PhAROS_METAB, and PhAROS_CHEMBIO, Subsequently utilizing the PhAROS_BIOGEO subsystem to identify plant, mixture, component and/or compound sources, for desired formulations and matching them to growing locations, environments and seasons, to generate cultivation plans for practitioners and community members.

FIG. 2D shows for illustrative purposes only an example of a schematic of major components of the PhAROS system, with user interaction description of one embodiment. FIG. 2D shows a schematic of major components of the PhAROS system, with user interaction description. A user and admin user access the subsystem name: PhAROS_USER through a standalone software application, users can interface within this subsystem: Users can interact and query the system. Users choose options for processing, appropriate tools, components, and output format. This is relayed to the PhAROS_CORE system networked on a server. An external user accesses the Subsystem name: PhAROS_USER through a web browser. Users can interface within this subsystem from any computer on the internet. Users can interact and query the system. Users choose options for processing, appropriate tools, components, and output formats. This is relayed to the PhAROS_CORE system, which can be networked remotely on a server, through the internet. The user, admin user, and external user access the server that contains the PhAROS system either directly or through the WWW. A WWW ftp with external databases and data sources. The server that contains the PhAROS system and WWW ftp are connected to the subsystem name: PhAROS_CORE. The PhAROS_USER subsystem interface communicates with this PhAROS_CORE subsystem. This subsystem collects the user query with their chosen options, and retrieves and processes data, from appropriate subsystems, and coordinates with other subsystems to further analyze, assess and visualize the data. Returning the results back to the user through the PhAROS_USER subsystem.

The PhAROS_CORE subsystem is connected to the other subsystems including the PhAROS_BRAlN. Subcomponents are accessed through the major components and include the PhAROS_PHARM, PhAROS_CONVERGE, PhAROS-CHEMBIO, PhAROS_BlOGEO, PhAROS_METAB, PhAROS_MICRO, PhAROS_CURE, PhAROS_QUANT, PhAROS_POPGEN, PhAROS_TOX, PhAROS_BH, PhAROS_EPIST, and PhAROS_BASE K.

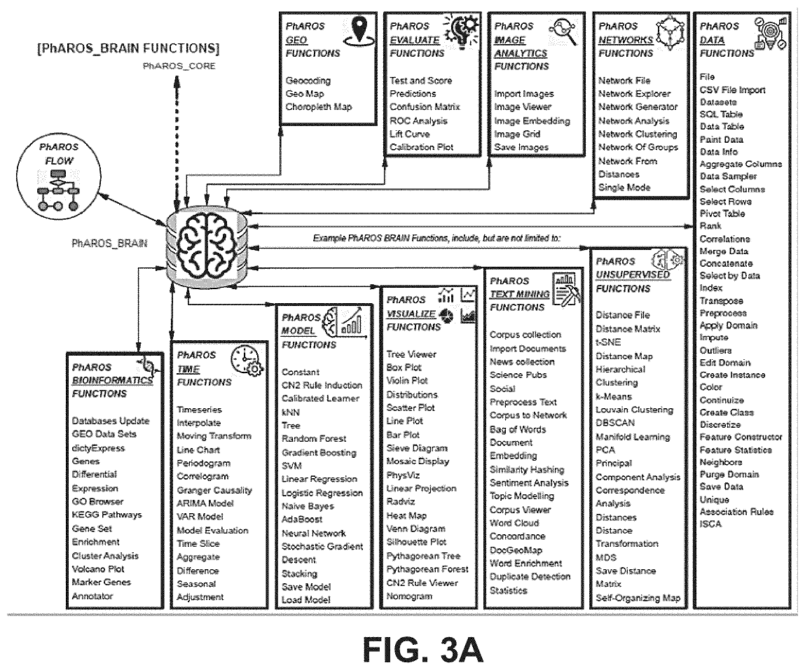

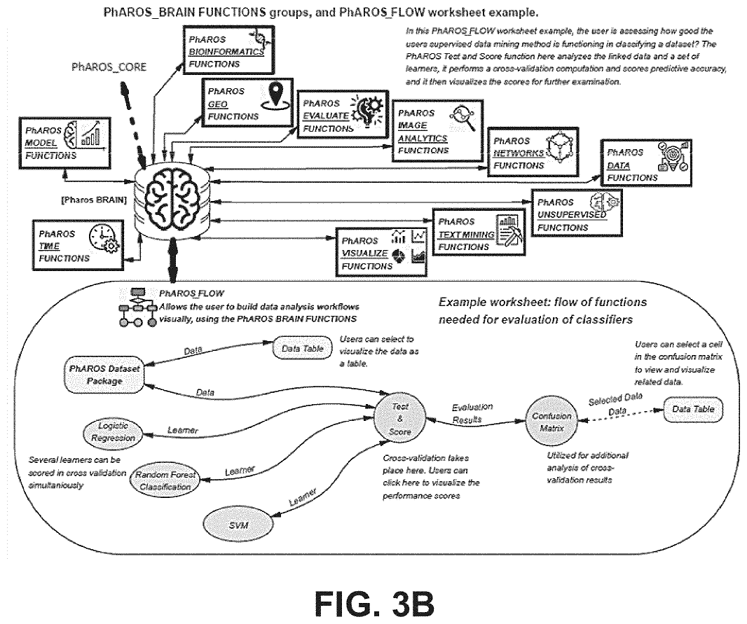

FIG. 3A shows for illustrative purposes only an example of a schematic of major components, and sub-functions of the PhAROS_BRAIN subsystem, indicating grouped PhAROS_BRAIN functions utilized by the PhAROS system and users, to create, update, annotate, process, download, analyze and manipulate data within the PhAROS system of one embodiment. FIG. 3A shows of a schematic of major components, and sub-functions of the PhAROS_BRAIN subsystem, indicating grouped PhAROS_BRAIN functions utilized by the PhAROS system and users, to create, update, annotate, process, download, analyze and manipulate data within the PhAROS system.

FIG. 3A shows PhAROS_BRAIN functions. PhAROS_BRAIN functions are processed into the PhAROS_CORE and are a bidirectional source of data with the PhAROS_FLOW. PhAROS_BRAIN functions include PhAROS_GEO Functions including Geocoding, Geo Map and Choropleth Map; PhAROS BIOINFORMATICS Functions with Databases Update, GEO Data Sets, dictyExpress, Genes, Differential, Expression, GO Browser, KEGG Pathways, Gene Set, Enrichment, Cluster Analysis, Volcano Plot, Marker Genes, and Annotator; PhAROS_EVALUATE Functions with Test and Score, Predictions, Confusion Matrix, ROC Analysis, Lift Curve, and Calibration Plot; PhAROS_IMAGE ANALYTICS Functions with Import Images, Image Viewer, Image Embedding, Image Grid, and Save Images; PhAROS_NETWORKS Functions with Network File, Network Explorer, Network Generator, Network Analysis, Network Clustering, Network of Groups, Network From, Distances, and Single Mode; PhAROS TIME Functions with Timeseries, Interpolate, Moving Transform, Line Chart, Periodogram, Correlogram, Granger Causality, ARIMA Model, VAR Model, Model Evaluation, Time Slice, Aggregate, Difference, Seasonal, and Adjustment; PhAROS_MODEL Functions with Constant, CN2 Rule Induction, Calibrated Learner, kNN, Tree, Random Forest, Gradient Boosting, SVM, Linear Regression, Logistic Regression, Naive Bayes, AdaBoost, Neural Network, Stochastic Gradient, Descent, Stacking, Save Model, and Load Model; PhAROS_VISUALIZE Functions with Tree Viewer, Box Plot, Violin Plot, Distributions, Scatter Plot, Line Plot, Bar Plot, Sieve Diagram, Mosaic Display, PhysViz, Linear Projection, Radviz, Heat Map, Venn Diagram, Silhouette Plot, Pythagorean Tree, Pythagorean Forest, CN2 Rule Viewer, and Nomogram; PhAROS_TEXT MINING Functions with Corpus collection, Import Documents, News collection, Science Pubs, Social, Preprocess Text, Corpus to Network, Bag of Words, Document, Embedding, Similarity Hashing, Sentiment Analysis, Topic Modeling, Corpus Viewer, Word Cloud, Concordance, DocGeoMap, Word Enrichment, Duplicate Detection, and Statistics; PhAROS_UNSUPERVISED Functions with Distance File, Distance Matrix, t-SNE, Distance Map, Hierarchical, Clustering, k-Means, Louvain Clustering, DBSCAN, Manifold Learning, PCA, Principal, Component analysis, Correspondence, Analysis, Distances, Distance, Transformation, MDS, Save Distance, Matrix, and Self-Organizing Map; and PhAROS_DATA Functions with File, CSV file import, Data sets, SOL Table, Data Table, Paint Data, Data Info, Aggregate Columns, Data Sampler, Select Columns, Select Rows, Pivot Table, Rank, Correlations, Merge Data, Concatenate, Select by Data, Index, Transpose, Preprocess, Apply Domain, Impute, Outliers, Edit Domain, Create Instance, Color, Continuize, Create Class, Discretize, Feature Constructor, Feature Statistics, Neighbors, Purge Domain, Save Data, Unique, Association Rules, and ISCA of one embodiment.

FIG. 3B shows for illustrative purposes only an example of a schematic of major components of the PhAROS_BRAIN subsystem, and the PhAROS_FLOW system utilized by the PhAROS system and users, to create, update, annotate, process, download, analyze and manipulate data within the PhAROS system, utilizing a graphical no-code/low code worksheet environment, without the need for coding of one embodiment. FIG. 3B shows a schematic of major components of the PhAROS_BRAIN subsystem, and the PhAROS_FLOW system utilized by the PhAROS system and users, to create, update, annotate, process, download, analyze and manipulate data within the PhAROS system, utilizing a graphical no-code/low code worksheet environment, without the need for coding.

FIG. 3B shows PhAROS_BRAIN Functions groups, and PhAROS_FLOW worksheet example. In this PhAROS_FLOW worksheet example, the user is assessing how good the users supervised data mining is functioning in classifying a data set. The PhAROS Test and Score function here analyses the linked data and a set of learners, it performs a cross-validation computation and scores predictive accuracy, and it then visualizes the scores for further examination. Bidirectional data transfers take place between the PhAROS BRAIN and the function modules.

The function modules being accessed include PhAROS_GEO Functions, PhAROS_BIOINFORMATICS Functions, PhAROS_EVALUATE Functions, PhAROS_IMAGE ANALYTICS Functions, PhAROS_NETWORKS Functions, PhAROS_TIME Functions, PhAROS_MODEL Functions, PhAROS_VISUALIZE Functions, PhAROS_TEXT MINING Functions, PhAROS_UNSUPERVISED Functions, and PhAROS_DATA Functions. As PhAROS_BRAIN Functions data is collected the data is transmitted to the PhAROS_FLOW. PhAROS_FLOW allows the user to build data analysis workflows visually, using the PhAROS_BRAIN Functions.

In this example worksheet flow of functions are needed for evaluation of classifiers. Users can select a cell in the confusion matrix to view and visualize related data. Selected data from a data table is displayed from the confusion matrix to the data table. The confusion matrix is utilized for additional analysis of cross validation results. Evaluation results are transferred to the test and score module. Cross-validation takes place in the test and score module. Users can click here to visualize the performance scores. Several learners can be scored in cross validation simultaneously.

In this example the learners include Logistic Regression, Random Forest Classification and SVM. Users can select to visualize the data as a table. That process transmits data back and forth from the test and score module to the PhAROS dataset package module as the user creates the desired data table of one embodiment.

In one embodiment, the PhAROS_BRAIN Subsystem functions include, but are not limited to the following functions accompanying uses in Table 1 below:

TABLE 1
PhAROS_BRAIN Functions

PhAROS_BRAIN
FUNCTIONS

| | |
|---|---|
| [symbol] | [symbol] [symbol] [symbol] |
| [symbol] | [symbol] [symbol] [symbol] |
| [symbol] | [symbol] [symbol] [symbol] |
| File | Reads attribute-value data from an input file. |
| | Output: |
| | Data: dataset from the file |
| CSV File Import | Allows the user to import a data table from a CSV formatted file. |
| | Output: |
| | Data: dataset from the .csv file: |
| | Data Frame: DataFrame object |
| Datasets | Allows the user to load a dataset from an online repository. |
| | Output: |
| | Data: output dataset |
| SQL Table | Allows the user to read data from an SQL database. |
| | Output: |
| | Data: dataset from the database |
| Data Table | Allows the user to displays attribute-value data in a spreadsheet table format. |
| | Input: |
| | Data: input dataset. |
| | Output: |
| | Selected Data: instances selected from the table |
| Paint Data | Allows the user to paint or select data on a 2D or 3D plane. Users can pick individual data points, or use |
| | a brush, or lasso to select larger datasets. |
| | Output: |
| | Data: dataset as painted in the plot |
| | Description: |
| | The PhAROS Paint Data function allows for users to interact with, and select specific areas of interest |
| | within a data set or sub-dataset. Once selected this data can be re-processed, assessed further or used to |
| | develop training sets for machine learning algorithms, or train human in the loop machine learning |
| | algorithms, in order to identify specific compounds, mixtures, indications or other uses of components |
| | within the PhAROS meta-pharmacopoeias. |
| Data Info | Allows the user to display information on a selected dataset. |
| | Input: |
| | Data: input dataset |
| Aggregate Columns | Allows the user to compute a sum, max, min . . . of selected columns. |
| | Input: |
| | Data: input dataset. |
| | Output: |
| | Data: extended dataset |
| Data Sampler | Allows the user to select a subset of data instances from an input dataset. |
| | Input: |
| | Data: input dataset: |
| | Output: |
| | Data Sample: sampled data instances. Remaining Data: out-of-sample data |
| Select Columns | Allows the user manual selection of data attributes and composition of data domain. |
| | Input: Data: input dataset: |
| | Output: Data: dataset with columns as set by the user |
| Select Rows | Allows the user to select data instances based on conditions over data features. |
| | Input: |
| | Data: input dataset. |
| | Output: |
| | Matching Data: data instances that match the user selected conditions. |
| | Non-Matching Data: data instances that do not match user selected conditions. |
| | Data: data with an additional column showing whether an instance is selected. |
| Pivot Table | Allows the user to reshape a data table based on column data. |
| | Input: Data: input data set. |
| | Output: |
| | Pivot Table: contingency matrix as indicated. |
| | Filtered Data: a subset, user selected from the plot. |
| | Grouped Data: aggregates over groups defined by row data. |
| Rank | Allows the user to rank attributes in classification or regression datasets. |
| | Input: |
| | Data: input dataset. |
| | Scorer: models for feature scoring. |
| | Output: |
| | Reduced Data: dataset with selected attributes. Scores: data table with feature scores. Features: list of |
| | attributes. |
| Correlations | Allows the user to process all pairwise attribute correlations. |
| | Input: |
| | Data: input dataset. |
| | Output: |
| | Data: input dataset. |
| | Features: selected pair of data features. |
| | Correlations: data table with correlation scores. |
| Merge Data | Allows the user to merge two user selected datasets, based on data of selected attributes. |
| | Input: |
| | Data: input dataset |
| | Extra Data: additional dataset |
| | Output: Data: dataset with added features and data from additional user selected dataset. |
| Concatenate | Allows the user to concatenate data from multiple user selected sources. |
| | Input: Primary Data: data set that defines the attribute set |
| | Additional Data: additional data set |
| | Output: Data: concatenated data |
| Select by Data Index | Allows the user to match data instances by the index from a user selected data subset. |
| | Input: |
| | Data: user selected reference data set |
| | Data Subset: user selected subset to match |
| | Output: |
| | Matching data: subset from reference data set that matches indices from subset data. |
| | Annotated data: reference data set with an additional column defining matches. |
| | Unmatched data: subset from reference data set that does not match indices from subset data. |
| Transpose | Allows the user to transpose a data table selected by the user. |
| | Input |
| | Data: input user selected dataset |
| | Output: |
| | Data: transposed dataset |
| Preprocess | Allows the user to preprocess data with user selected methods. |
| | Input: Data: input user selected dataset |
| | Output: Preprocessor: preprocessing method |
| | Preprocessed Data: data preprocessed with user selected methods |
| Apply Domain | Allows the user to transform a dataset based on a template dataset. |
| | Input: Data: input dataset |
| | Template Data: template for transforming the dataset |
| | Output: Transformed Data: transformed dataset |
| Impute | Allows the user to replace unknown values in the user selected dataset. |
| | Input: Data: user selected input dataset. |
| | Learner: learning algorithm for imputation. |
| | Output: Data: dataset with imputed values. |
| Outliers | Allows the user to detect outlying data, within a user selected dataset. |
| | Input: Data: user selected input dataset. |
| | Output: Outliers: instances scored as outliers. |
| | Inliers: instances not scored as outliers. |
| | Data: input dataset appended Outlier variable. |
| Edit Domain | Allows the user to edit/change a dataset's domain - rename features, rename or merge values of |
| | categorical features, add a categorical value, and assign labels. |
| | Input: Data: input dataset |
| | Output: Data: dataset with edited domain. |
| Create Instance | Allows the user to interactively create a new instance, based on the input data. |
| | Input: Data: input dataset |
| | Reference: reference dataset |
| | Output: Data: input dataset appended the created instance. |
| Color | Allows the user to select and set a color legend for variables. |
| | Input: Data: user selected input data set |
| | Output: Data: data set with a new color legend |
| Continuize | Allows the user to convert discrete variables (attributes) into numeric (“continuous”) dummy variables. |
| | Input: Data: input user selected data set. |
| | Output: Data: transformed data set. |
| Create Class | Allows the user to create a class attribute from a string attribute. |
| | Input: Data: input user selected data set. |
| | Output: Data: dataset with a new class variable. |
| Discretize | Allows the user to discretize continuous attributes from an input dataset. |
| | Input: Data: input user selected data set |
| | Output: Data: dataset with discretized values |
| Feature Constructor | Allows the user to manually add features (columns) into a dataset. The subsequent feature can be a |
| | computation of an existing one or a combination of several (addition, subtraction, etc.). |
| | Input: Data: input user selected data set |
| | Output: Data: dataset with additional features |
| Feature Statistics | Allows the user to show basic statistics for data features. Allows the user to a rapid and convenient way |
| | to inspect and find interesting features in a given data set. |
| | Input: Data: input user selected data set |
| | Output: Reduced data: table containing only selected features |
| | Statistics: table containing statistics of the selected features |
| Neighbors | Allows the user to compute nearest neighbors in data according to reference. |
| | Input: Data: input user selected dataset. |
| | Reference: A reference data for neighbor computation. |
| | Output: Neighbors: A data table of nearest neighbors according to reference. |
| Purge Domain | Allows the user to remove unused attribute values and useless attributes, and sort the remaining values. |
| | Input: Data: input user selected data set |
| | Data: input dataset |
| | Output: Data: filtered dataset |
| Save Data | Allows the user to save and export user selected data to a file. |
| | Input: Data: input user selected dataset. |
| | Output: A dataset saved as: |
| | a tab-delimited file (.tab) |
| | comma-separated file (.csv) |
| | pickle (.pkl), |
| | Excel spreadsheets (.xlsx) |
| | spectra ASCII (.dat) |
| | hyperspectral map ASCII (.xyz) |
| | compressed formats (.tab.gz, .csv.gz, .pkl.gz) |
| Unique | Allows the user to remove duplicated data instances. |
| | Input: Data: data table |
| | Output: Data: data table without duplicates |
| Association Rules | Allows users to induce association rules. |
| | Input: Data: Data set |
| | Output: Matching Data: Data instances matching the criteria. |
| | This PhAROS Association Rules function allows users to implement FP-growth frequent pattern mining |
| | algorithms with bucketing optimization or for conditional databases of few items. For inducing |
| | classification rules, it generates rules for the entire item set and skips the rules where the consequent |
| | does not match one of the class' values. |
| ISCA | Allows the user to perform In silico convergence analysis (ISCA). |
| | Input: Data: Data set |
| | Output: Matching Data: Data instances matching the criteria. |
| [symbol] | [symbol] [symbol] [symbol] |
| [symbol] | [symbol] [symbol] [symbol] |
| [symbol] | [symbol] [symbol] [symbol] |
| Tree Viewer | Allows the user to visualize classification and regression trees. |
| | Input: Tree: decision tree |
| | Output: Selected Data: instances selected from the tree node |
| | Data: data with an additional column showing whether a point is selected |
| Box Plot | Allows the user to visualize distribution of attribute values. |
| | Input: Data: input dataset |
| | Output: Selected Data: instances selected from the plot |
| | Data: data with an additional column showing whether a point is selected |
| Violin Plot | Allows the user to visualize the distribution of feature values in a violin plot. |
| | Input: Data: input dataset |
| | Output: Selected Data: instances selected from the plot |
| | Data: data with an additional column showing whether a point is selected |
| Distributions | Allows the user to display value distributions for a single attribute. |
| | Input: Data: input dataset |
| | Output: Selected Data: instances selected from the plot |
| | Data: data with an additional column showing whether an instance is selected |
| | Histogram Data: bins and instance counts from the histogram |
| Scatter Plot | Allows the user to visualize and explore data using a scatter plot method. |
| | Input: Data: input dataset |
| | Data Subset: subset of instances |
| | Features: list of attributes |
| | Output: Selected Data: instances selected from the plot |
| | Data: data with an additional column showing whether a point is selected |
| Line Plot | Allows the user to visualize and explore data using a line plot methods |
| | Input: Data: input dataset |
| | Data Subset: subset of instances |
| | Output: Selected Data: instances selected from the plot |
| | Data: data with an additional column showing whether a point is selected |
| Bar Plot | Allows the user to visualize and explore comparisons among discrete categories, using bar plot methods. |
| | Input: Data: input dataset |
| | Data Subset: subset of instances |
| | Output: Selected Data: instances selected from the plot |
| | Data: data with an additional column showing whether a point is selected |
| Sieve Diagram | Allows the user to plot and visualize a sieve diagram for a pair of attributes. |
| | Input: Data: input dataset |
| Mosaic Display | Allows the user to visualize and explore data in a mosaic plot. |
| | Input: Data: input dataset |
| | Data subset: subset of instances |
| | Output: Selected data: instances selected from the plot |
| PhysViz | Allows the user to display a PhysViz projection. This method utilizes particle physics: points in the same |
| | class attract each other, those from different class repel each other, and the resulting forces are exerted |
| | on the anchors of the attributes, that is, on unit vectors of each of the dimensional axis. The points |
| | cannot move (are projected in the projection space), but the attribute anchors can, so the optimization |
| | process is a hill-climbing optimization where at the end the anchors are placed such that forces are in |
| | equilibrium. The user can invoke the optimization process. |
| | Input: Data: input dataset |
| | Data Subset: subset of instances |
| | Output: Selected Data: instances selected from the plot |
| | Data: data with an additional column showing whether a point is selected |
| | Components: PhysViz vectors |
| Linear Projection | Allows the user to use linear projection method to visualize and explore datasets |
| | Input: |
| | Data: input dataset |
| | Data Subset: subset of instances |
| | Projection: custom projection vectors |
| | Output: Selected Data: instances selected from the plot |
| | Data: data with an additional column showing whether a point is selected |
| | Components: projection vectors |
| Radviz | Allows the user to visualize data using Radviz visualization, with exploratory data analysis and |
| | intelligent data visualization enhancements. This is a non-linear multi-dimensional visualization |
| | technique that can display data defined by three or more variables in a 2-dimensional projection. |
| | Input: |
| | Data: input dataset |
| | Data Subset: subset of instances |
| | Output: |
| | Selected Data: instances selected from the plot |
| | Data: data with an additional column showing whether a point is selected |
| | Components: Radviz vectors. |
| Heat Map | Allows the user to visualize data as a heat map. |
| | Input: Data: input dataset |
| | Output: Selected Data: instances selected from the plot |
| Venn Diagram | Allows the user to plots datasets as a venn diagram for two or more data subsets. |
| | Input: Data: input dataset |
| | Output: |
| | Selected Data: instances selected from the plot |
| | Data: entire data with a column indicating whether an instance was selected or not. |
| Silhouette Plot | Allows the user to generate a graphical representation of consistency within clusters of data. |
| | Input: Data: input dataset |
| | Output: |
| | Selected Data: instances selected from the plot |
| | Data: data with an additional column showing whether a point is selected |
| Pythagorean Tree | Allows the user to use Pythagorean tree visualization for classification or regression trees. |
| | Input: Tree: tree model |
| | Selected Data: instances selected from the tree |
| Pythagorean Forest | Allows the user to generate a Pythagorean forest for visualizing random forests. |
| | Pythagorean Forest visualizes all learned decision tree models from Random Forest |
| | Input: Random Forest: tree models from random forest |
| | Output: Tree: selected tree model |
| CN2 Rule Viewer | Allows the user to visualize a CN2 Rule. The CN2 induction algorithm is a learning algorithm for rule |
| | induction. It is designed to work even when the training data is imperfect. It is based on ideas from the |
| | AQ algorithm and the ID3 algorithm. As a consequence it creates a rule set like that created by AQ but is |
| | able to handle noisy data like ID3. |
| | Input: |
| | Data: dataset to filter |
| | CN2 Rule Classifier: CN2 Rule Classifier, including a list of induced rules |
| | Output: |
| | Filtered Data: data instances covered by all selected rules. |
| Nomogram | Allows the user to visualize nomograms of Naive Bayes and Logistic Regression classifiers. |
| | Input: |
| | Classifier: trained classifier |
| | Data: input dataset |
| | Output: Features: selected variables. |
| [symbol] | [symbol] [symbol] [symbol] |
| [symbol] | [symbol] [symbol] [symbol] |
| [symbol] | [symbol] |
| Constant | Allows the user to predict the most frequent class or mean value from the training set. |
| | Input: |
| | Data: input dataset |
| | Preprocessor: preprocessing method(s) |
| | Output: |
| | Learner: majority/mean learning algorithm |
| | Model: trained model |
| | Description: |
| | This PhAROS_Constant function is a learner that produces a model that always predicts the majority for |
| | classification tasks and means value for regression tasks. |
| | For classification, when predicting the class value with Predictions, the function will return relative |
| | frequencies of the classes in the training set. When there are two or more majority classes, the classifier |
| | chooses the predicted class randomly, but always returns the same class for a particular example. |
| | For regression, it learns the mean of the class variable and returns a predictor with the same mean value. |
| CN2 Rule Induction | Allows the user to induce rules from data using CN2 algorithm. |
| | Input: |
| | Data: input dataset |
| | Preprocessor: preprocessing method(s) |
| | Output: |
| | Learner: CN2 learning algorithm |
| | CN2 Rule Classifier: trained model |
| | Description: |
| | This PhAROS CN2 function and algorithm is a classification technique designed for the efficient |
| | induction of simple, comprehensible rules of form “if cond then predict class”, even in domains where |
| | noise may be present. The CN2 Rule Induction works only for classification. |
| Calibrated Learner | Allows the user to wrap another learner with probability calibration and decision threshold optimization. |
| | Input: |
| | Data: input dataset |
| | Preprocessor: preprocessing method(s) |
| | Base Learner: learner to calibrate |
| | Output: |
| | Learner: calibrated learning algorithm |
| | Model: trained model using the calibrated learner |
| | Description: |
| | This PhAROS Calibrated Learner function produces a model that calibrates the distribution of class |
| | probabilities and optimizes decision threshold, and works for binary classification tasks. |
| kNN | Allows the user to predict according to the nearest training instances. |
| | Input: |
| | Data: input dataset |
| | Preprocessor: preprocessing method(s) |
| | Output: |
| | Learner: kNN learning algorithm |
| | Model: trained model |
| | Description: |
| | This PhAROS kNN function uses the kNN algorithm that searches for k closest training examples in |
| | feature space and uses their average as prediction. In statistics, the k-nearest neighbor's algorithm (k- |
| | NN) is a non-parametric classification method first developed by Evelyn Fix and Joseph Hodges in |
| | 1951, and later expanded by Thomas Cover. It is used for classification and regression. In both cases, the |
| | input consists of the k closest training examples in data set. The output depends on whether k-NN is used |
| | for classification or regression: In k-NN classification, the output is a class membership. An object is |
| | classified by a plurality vote of its neighbors, with the object being assigned to the class most common |
| | among its k nearest neighbors (k is a positive integer, typically small). If k = 1, then the object is simply |
| | assigned to the class of that single nearest neighbor. In k-NN regression, the output is the property value |
| | for the object. This value is the average of the values of k nearest neighbors. |
| | k-NN is a type of classification where the function is only approximated locally and all computation is |
| | deferred until function evaluation. Since this algorithm relies on distance for classification, if the |
| | features represent different physical units or come in vastly different scales then normalizing the training |
| | data can improve its accuracy dramatically. Both for classification and regression, a useful technique can |
| | be to assign weights to the contributions of the neighbors, so that the nearer neighbors contribute more to |
| | the average than the more distant ones. For example, a common weighting scheme consists in giving |
| | each neighbor a weight of 1/d, where d is the distance to the neighbor. The neighbors are taken from a |
| | set of objects for which the class (for k-NN classification) or the object property value (for k-NN |
| | regression) is known. This can be thought of as the training set for the algorithm, though no explicit |
| | training step is required. |
| Tree | Allows the user to utilize a tree algorithm with forward pruning. |
| | Input: |
| | Data: input dataset. |
| | Preprocessor: preprocessing method(s). |
| | Output: |
| | Learner: decision tree learning algorithm. |
| | Model: trained model. |
| | Description: |
| | This PhAROS Tree functions acts as a method and algorithm that splits the data into nodes by class |
| | purity. It is a precursor to Random Forest. Here it is able to utilize both discrete and continuous datasets, |
| | and can also be used for both classification and regression tasks. |
| Random Forest | Allows the user to predict using an ensemble of decision trees. |
| | Input: |
| | Data: input dataset. |
| | Preprocessor: preprocessing method(s) |
| | Output: |
| | Learner: random forest learning algorithm. |
| | Model: trained model. |
| | Description: |
| | This PhAROS Random forest function is an ensemble learning method used for classification, regression |
| | and other tasks. Random Forest builds a set of decision trees. Each tree is developed from a bootstrap |
| | sample from the training data. When developing individual trees, an arbitrary subset of attributes is |
| | drawn (hence the term “Random”), from which the best attribute for the split is selected. The final model |
| | is based on the majority vote from individually developed trees in the forest. Random Forest works for |
| | both classification and regression tasks. |
| Gradient Boosting | Allows the user to predict using gradient boosting on decision trees. |
| | Input: |
| | Data: input dataset |
| | Preprocessor: preprocessing method(s) |
| | Output: |
| | Learner: gradient boosting learning algorithm |
| | Model: trained model |
| | Description: |
| | This PhAROS Gradient Boosting function is a machine learning module for regression and classification |
| | problems, which produces a prediction model in the form of an ensemble of weak prediction models, |
| | typically decision trees. |
| SVM | Allows the user to utilize Support Vector Machines in mapping Input to higher-dimensional feature |
| | spaces. |
| | Input: |
| | Data: input dataset |
| | Preprocessor: preprocessing method(s) |
| | Output: |
| | Learner: linear regression learning algorithm |
| | Model: trained model |
| | Support Vectors: instances used as support vectors |
| | Description: |
| | This PhAROS Support vector machine (SVM) function is a machine learning module that separates the |
| | attribute space with a hyperplane, thus maximizing the margin between the instances of different classes |
| | or class values. The technique often yields supreme predictive performance results. For regression tasks, |
| | SVM performs linear regression in a high dimension feature space using a ε-insensitive loss. Its |
| | estimation accuracy depends on a good setting of C, ε and kernel parameters. The function Output class |
| | predictions based on a SVM Regression, and works for both classification and regression tasks. |
| Linear Regression | Allows the user to utilize a linear regression algorithm with optional L1 (LASSO), L2 (ridge) or L1L2 |
| | (elastic net) regularization. |
| | Input: |
| | Data: input dataset |
| | Preprocessor: preprocessing method(s) |
| | Output: |
| | Learner: linear regression learning algorithm |
| | Model: trained model |
| | Coefficients: linear regression coefficients |
| | Description: |
| | This PhAROS Linear Regression function constructs a learner/predictor module that learns a linear |
| | function from its input data. The model can identify the relationship between a predictor xi and the |
| | response variable y. Additionally, Lasso and Ridge regularization parameters can be specified. Lasso |
| | regression minimizes a penalized version of the least squares loss function with L1-norm penalty and |
| | Ridge regularization with L2-norm penalty. This function works with regression tasks only. |
| Logistic Regression | Allows the user to utilize logistic regression classification algorithms with LASSO (L1) or ridge (L2) |
| | regularization. |
| | Input: |
| | Data: input dataset |
| | Preprocessor: preprocessing method(s) |
| | Output: |
| | Learner: logistic regression learning algorithm |
| | Model: trained model |
| | Coefficients: logistic regression coefficients |
| | Description: |
| | This PhAROS Logistic Regression function is generates a logistic regression model from the data, and |
| | works for classification tasks. |
| Naive Bayes | Allows the user to utilize the rapid and simple probabilistic classifier based on Bayes' theorem with the |
| | assumption of feature independence. |
| | Input: |
| | Data: input dataset |
| | Preprocessor: preprocessing method(s) |
| | Output: |
| | Learner: naive Bayes learning algorithm |
| | Model: trained model |
| | Naive Bayes learns a Naive Bayesian model from the data. It only works for classification tasks. |
| | Description: |
| | This PhAROS Naive Bayes function utilizes In Naive Bayes classifiers. These are a family of simple |
| | “probabilistic classifiers” based on applying Bayes' theorem with strong (naïve) independence |
| | assumptions between the features (see Bayes classifier). They are among the simplest Bayesian network |
| | models, but coupled with kernel density estimation, they can achieve higher accuracy levels. Naïve |
| | Bayes classifiers are highly scalable, requiring a number of parameters linear in the number of variables |
| | (features/predictors) in a learning problem. Maximum-likelihood training can be done by evaluating a |
| | closed-form expression, which takes linear time, rather than by expensive iterative approximation as |
| | used for many other types of classifiers. In the statistics and computer science literature, naive Bayes |
| | models are known under a variety of names, including simple Bayes and independence Bayes. All these |
| | names reference the use of Bayes' theorem in the classifier's decision rule, but naive Bayes is not |
| | (necessarily) a Bayesian method. |
| AdaBoost | Allows the user to utilize an ensemble meta-algorithm that combines weak learners and adapts to the |
| | ‘hardness’ of each training sample. |
| | Input: |
| | Data: input dataset |
| | Preprocessor: preprocessing method(s) |
| | Learner: learning algorithm |
| | Output: |
| | Learner: AdaBoost learning algorithm |
| | Model: trained model |
| | Description: |
| | This PhAROS AdaBoost function, (short for “Adaptive boosting”) is a machine-learning algorithm and |
| | function. It can be used with other learning algorithms to boost their performance. It does so by tweaking |
| | the weak learners, and works for both classification and regression. |
| | It can be used in conjunction with many other types of learning algorithms to improve performance. The |
| | output of the other learning algorithms (‘weak learners’) is combined into a weighted sum that represents |
| | the final output of the boosted classifier. AdaBoost is adaptive in the sense that subsequent weak learners |
| | are tweaked in favor of those instances misclassified by previous classifiers. In some problems it can be |
| | less susceptible to the over fitting problem than other learning algorithms. The individual learners can be |
| | weak, but as long as the performance of each one is slightly better than random guessing, the final model |
| | can be proven to converge to a strong learner. Every learning algorithm tends to suit some problem types |
| | better than others, and typically has many different parameters and configurations to adjust before it |
| | achieves optimal performance on a dataset. AdaBoost (with decision trees as the weak learners) is often |
| | referred to as the best out-of-the-box classifier. When used with decision tree learning, information |
| | gathered at each stage of the AdaBoost algorithm about the relative ‘hardness’ of each training sample is |
| | fed into the tree growing algorithm such that later trees tend to focus on harder-to-classify examples. |
| Neural Network | Allows the user to utilize a multi-layer perceptron (MLP) algorithm with back propagation. |
| | Input: |
| | Data: input dataset |
| | Preprocessor: preprocessing method(s) |
| | Output: |
| | Learner: multi-layer perceptron learning algorithm |
| | Model: trained model |
| | Description: |
| | This PhAROS Neural Network function and module uses sklearn's Multi-layer Perceptron algorithm |
| | that can learn non-linear models as well as linear. |
| Stochastic Gradient | Allows the user to minimize an objective function using a stochastic approximation of gradient descent. |
| Descent | Input: |
| | Data: input dataset |
| | Preprocessor: preprocessing method(s) |
| | Output: |
| | Learner: stochastic gradient descent learning algorithm |
| | Model: trained model |
| | Description: |
| | This PhAROS Stochastic Gradient Descent function uses a stochastic gradient descent that minimizes a |
| | chosen loss function with a linear function. The algorithm approximates a true gradient by considering |
| | one sample at a time, and simultaneously updates the model based on the gradient of the loss function. |
| | For regression, it returns predictors as minimizers of the sum, i.e. M-estimators, and is especially useful |
| | for large-scale and sparse datasets. |
| Stacking | Allows the user to stack multiple models. |
| | Input: |
| | Data: input dataset |
| | Preprocessor: preprocessing method(s) |
| | Learners: learning algorithm |
| | Aggregate: model aggregation method |
| | Output: |
| | Learner: aggregated (stacked) learning algorithm |
| | Model: trained model |
| | Description: |
| | This PhAROS Stacking function is an ensemble module and method that computes a meta model from |
| | several base models. The Stacking function has the Aggregate input, which provides a method for |
| | aggregating the input models. If no aggregation input is given the default methods are used. Those are |
| | Logistic Regression for classification and Ridge Regression for regression problems. |
| Save Model | Allows the user to save their trained model to an output file. |
| | Input: Model: trained model |
| | Output: Model file |
| Load Model | Allows the user to load a model from an input file. |
| | Output: Model: trained model |
| [symbol] | [symbol] [symbol] [symbol] |
| [symbol] | [symbol] [symbol] [symbol] |
| [symbol] | [symbol] |
| Test and Score | Allows the user to test learning algorithms on data. |
| | Input: |
| | Data: input dataset |
| | Test Data: separate data for testing |
| | Learner: learning algorithm(s) |
| | Output: |
| | Evaluation Results: results of testing classification algorithms |
| | Description: |
| | This PhAROS Test and Score function, tests learning algorithms. Different sampling schemes are |
| | available, including using separate test data. The function does two things. First, it shows a table with |
| | different classifier performance measures, such as classification accuracy and area under the curve. |
| | Second, it produces evaluation results, which can be used by other functions for analyzing the |
| | performance of classifiers, such as ROC Analysis or Confusion Matrix. The Learner signal has an |
| | uncommon property: it can be connected to more than one function to test multiple learners with the |
| | same procedures. |
| Predictions | Allows the user to observe models' predictions on the data. |
| | Input: |
| | Data: input dataset |
| | Predictors: predictors to be used on the data |
| | Output: |
| | Predictions: data with added predictions |
| | Evaluation Results: results of testing classification algorithms. |
| | Description: |
| | This PhAROS Predictions function receives a dataset and one or more predictors (predictive models); it |
| | then outputs the data and the predictions based on the model. |
| Confusion Matrix | Allows the user to observe proportions between the predicted and actual class. |
| | Input: |
| | Evaluation results: results of testing classification algorithms |
| | Output: |
| | Selected Data: data subset selected from confusion matrix |
| | Data: data with the additional information on whether a data instance was selected |
| | This PhAROS Confusion Matrix function gives the number/proportion of instances between the |
| | predicted and actual class. The selection of the elements in the matrix feeds the corresponding instances |
| | into the output signal. This way, one can observe which specific instances were misclassified and how. |
| ROC Analysis | Allows the user to graphically plot a true positive rate against a false positive rate of a test. |
| | Input: |
| | Evaluation Results: results of testing classification algorithms |
| | This PhAROS ROC Analysis function shows ROC curves for the tested models and the corresponding |
| | convex hull. It serves as a mean of comparison between classification models. The curve plots a false |
| | positive rate on an x-axis (1-specificity; probability that target = 1 when true value = 0) against a true |
| | positive rate on a y-axis (sensitivity; probability that target = 1 when true value = 1). The closer the curve |
| | follows the left-hand border and then the top border of the ROC space, the more accurate the classifier. |
| | Given the costs of false positives and false negatives, the function can also determine the optimal |
| | classifier and threshold. |
| Lift Curve | Allows the user to measure the performance of a chosen classifier against a random classifier. |
| | Input: |
| | Evaluation Results: results of testing classification algorithms. |
| | This PhAROS Lift curve function allows the user to observe the curves for analyzing the proportion of |
| | true positive data instances in relation to the classifier's threshold or the number of instances that we |
| | classify as positive. The user can visualize cumulative gains as a chart showing the proportion of true |
| | positive instances as a function of the number of positive instances, assuming the instances are ordered |
| | according to the model's probability of being positive. |
| Calibration Plot | Allows the user to visualize the match between classifiers' probability predictions and actual class |
| | probabilities. |
| | Input: |
| | Evaluation Results: results of testing classification algorithms |
| | This PhAROS Calibration Plot function graphically plots class probabilities against those predicted by |
| | the classifier(s). |
| Unsupervised | These PhAROS Unsupervised machine learning functions, allows the PhAROS systems to rapidly clean |
| | analyze data, train, and model and predict using a variety of user defined data sources. These can be used |
| | in the pre-processed PhAROS sub-systems, or for de-novo analysis, depending on the user's case use. |
| Distance File | Allows the user to load an existing distance matrix file. |
| | Output: Distance File: distance matrix |
| Distance Matrix | Allows the user to visualize distance measures in a distance matrix. |
| | Input: |
| | Distances: distance matrix |
| | Output: |
| | Distances: distance matrix |
| | Table: distance measures in a distance matrix |
| | This PhAROS Distance Matrix function creates a distance matrix, which is a two-dimensional array |
| | containing the distances, taken pairwise, between the elements of a set. The number of elements in the |
| | dataset defines the size of the matrix. Data matrices are essential for hierarchical clustering and they are |
| | extremely useful in bioinformatics as well, where they are used to represent protein structures in a |
| | coordinate-independent manner. |
| t-SNE | Allows the user to produce a two-dimensional data projection with t-SNE. |
| | Input: |
| | Data: input dataset |
| | Data Subset: subset of instances |
| | Output: |
| | Selected Data: instances selected from the plot |
| | Data: data with an additional column showing whether a point is selected |
| | Description: |
| | This PhAROS t-SNE function allows the user to graphically plot the data with a t-distributed stochastic |
| | neighbor embedding method. t-SNE is a dimensionality reduction technique, similar to MDS, where |
| | points are mapped to 2-D space by their probability distribution. |
| Distance Map | Allows the user to visualize distances between items. |
| | Input: |
| | Distances: distance matrix |
| | Output: |
| | Data: instances selected from the matrix |
| | Features: attributes selected from the matrix |
| | Description: |
| | This PhAROS Distance Map function allows the user to visualize distances between objects. The |
| | visualization replaces a table of numbers, with colored spots. Distances are most often those between |
| | instances (“rows” in the Distances function) or attributes (“columns” in Distances function). The only |
| | suitable input for Distance Map is the Distances function. For the output, the user can select a region of |
| | the map and the function will output the corresponding instances or attributes. Also note that the |
| | Distances function ignores discrete values and calculates distances only for continuous data, thus it can |
| | display distance map for discrete data if the user utilizes the PhAROS Continuize function first. |
| Hierarchical | Allows the user to group items using a hierarchical clustering algorithm. |
| Clustering | Input: |
| | Distances: distance matrix |
| | Output: |
| | Selected Data: instances selected from the plot |
| | Data: data with an additional column showing whether an instance is selected |
| | Description: |
| | This PhAROS Hierarchical Clustering function allows the user to compute hierarchical clustering of |
| | arbitrary types of objects from a matrix of distances and shows a corresponding dendrogram. |
| k-Means | Allows the user to group items using the k-Means clustering algorithm. |
| | Input: |
| | Data: input dataset |
| | Output: |
| | Data: dataset with cluster index as a class attribute. |
| | Description: |
| | This PhAROS k-Means function allows the user to apply the k-Means clustering algorithm to the data |
| | and Output a new dataset in which the cluster index is used as a class attribute. The original class |
| | attribute, if it exists, is moved to meta attributes. Scores of clustering results for various k are also shown |
| | in the function. |
| Louvain Clustering | Allows the user to group items using the Louvain clustering algorithm. |
| | Input: |
| | Data: input dataset |
| | Output: |
| | Data: dataset with cluster index as a class attribute |
| | Graph (with the Network add-on): the weighted k-nearest neighbor graph. |
| | Description: |
| | This PhAROS Louvain Clustering function allows the user to convert the input data into a k-nearest |
| | neighbor graph visualization. In order to preserve the notions of distance, the Jaccard index for the |
| | number of shared neighbors is used to weight the edges. Finally, a modularity optimization community |
| | detection algorithm is applied to the graph to retrieve clusters of highly interconnected nodes. The |
| | function Output a new dataset in which the cluster index is used as a meta attribute. |
| DBSCAN | Allows the user to group items using the DBSCAN clustering algorithm. |
| | Input: |
| | Data: input dataset |
| | Output: |
| | Data: dataset with cluster index as a class attribute |
| | Description: |
| | The function applies the DBSCAN clustering algorithm to the data and Output a new dataset with cluster |
| | indices as a meta attribute. The function also shows the sorted graph with distances to k-th nearest |
| | neighbors. With k values set to Core point neighbors as suggested in the methods article. This gives the |
| | user the idea of an ideal selection for Neighborhood distance setting. This parameter should be set to the |
| | first value in the first “valley” in the graph. |
| Manifold Learning | Allows the user to transform the data using a nonlinear dimensionality reduction. |
| | Input: |
| | Data: input dataset |
| | Output: |
| | Transformed Data: dataset with reduced coordinates |
| | Description: |
| | This PhAROS Manifold Learning function allows the user to find a non-linear manifold within the |
| | higher-dimensional space. The function then Output new coordinates which correspond to a two- |
| | dimensional space. Such data can be later visualized with the PhAROS Scatter Plot function or other |
| | PhAROS visualization functions. |
| PCA | Allows the user to apply Principal Component Analysis (PCA) linear transformation to their dataset. |
| Principal | Input: |
| Component Analysis | Data: input dataset |
| | Output: |
| | Transformed Data: PCA transformed data |
| | Components: Eigenvectors. |
| | Description: |
| | This PhAROS Principal Component Analysis (PCA) allows the euser to compute the PCA linear |
| | transformation of the input data. The user selects an output result of either a transformed dataset with |
| | weights of individual instances or an output result of weights of principal components. The principal |
| | components of a collection of points in a real coordinate space are a sequence of p unit vectors, where |
| | the i-th vector is the direction of a line that best fits the data while being orthogonal to the first i-1 |
| | vectors. Here, a best-fitting line is defined as one that minimizes the average squared distance from the |
| | points to the line. These directions constitute an orthonormal basis in which different individual |
| | dimensions of the data are linearly uncorrelated. Principal component analysis (PCA) is the process of |
| | computing the principal components and using them to perform a change of basis on the data, sometimes |
| | using only the first few principal components and ignoring the rest. PCA is used in exploratory data |
| | analysis and for making predictive models. It is predominantly used for dimensionality reduction by |
| | projecting each data point onto only the first few principal components to obtain lower-dimensional data |
| | while preserving as much of the data's variation as possible. The first principal component can |
| | equivalently be defined as a direction that maximizes the variance of the projected data. The i-th |
| | principal component can be taken as a direction orthogonal to the first i-1 principal components that |
| | maximize the variance of the projected data. |
| | It can be shown that the principal components are eigenvectors of the data's covariance matrix. Thus, the |
| | principal components are often computed by eigendecomposition of the data covariance matrix or |
| | singular value decomposition of the data matrix. PCA is the simplest of the true eigenvector-based |
| | multivariate analyses and is closely related to factor analysis. Factor analysis typically incorporates more |
| | domain specific assumptions about the underlying structure and solves eigenvectors of a slightly |
| | different matrix. PCA is also related to canonical correlation analysis (CCA). CCA defines coordinate |
| | systems that optimally describe the cross-covariance between two datasets while PCA defines a new |
| | orthogonal coordinate system that optimally describes variance in a single dataset. Robust and L1-norm- |
| | based variants of standard PCA have also been proposed. |
| Correspondence | Allows the user to utilize correspondence analysis for categorical multivariate data. |
| Analysis | Input: |
| | Data: input dataset |
| | Output: |
| | Coordinates: coordinates of all components |
| | Description: |
| | This PhAROS Correspondence Analysis (CA) function allows the user to compute the CA linear |
| | transformation of the input data. While it is similar to PCA, CA computes linear transformation on |
| | discrete rather than on continuous data. |
| Distances | Allows the user to compute distances between rows/columns in a dataset. |
| | Input: |
| | Data: input dataset |
| | Output: |
| | Distances: distance matrix |
| | Description: |
| | This PhAROS Distances function allows the user to compute distances between rows or columns in a |
| | dataset. By default, the data will be normalized to ensure equal treatment of individual features. |
| | Normalization is always done column-wise. Sparse data can only be used with Euclidean, Manhattan |
| | and Cosine metric. The resulting distance matrix can be fed further to the PhAROS Hierarchical |
| | Clustering function for uncovering groups in the data, to the PhAROS Distance Map function or the |
| | PhAROS Distance Matrix function for visualizing the distances (Distance Matrix can be quite slow for |
| | larger data sets), to the PhAROS MDS function for mapping the data instances using the distance matrix |
| | and finally, saved with the PhAROS Save Distance Matrix function. The Distance file can be loaded into |
| | the user area with the PhAROS Distance File function. |
| Distance | Allows the user to transform distances in a dataset. |
| Transformation | Input: |
| | Distances: distance matrix |
| | Output: |
| | Distances: transformed distance matrix |
| | Description: |
| | This PhAROS Distances Transformation function allows the user to compute the normalization and |
| | inversion of distance matrices. The normalization of data is necessary to bring all the variables into |
| | proportion with one another. |
| MDS | Allows the user to utilize multidimensional scaling (MDS) by projecting items onto a plane fitted to |
| | given distances between points. |
| | Input: |
| | Data: input dataset |
| | Distances: distance matrix |
| | Data Subset: subset of instances |
| | Output: |
| | Selected Data: instances selected from the plot |
| | Data: dataset with MDS coordinates. |
| | Description: |
| | This PhAROS MDS function (multidimensional scaling) allows to user to compute a low-dimensional |
| | projection of points, where it attempts to fit distances between points as well as possible. The perfect fit |
| | is typically impossible to obtain since the data is high-dimensional or the distances are not Euclidean. In |
| | the input, the function needs either a dataset or a matrix of distances. When visualizing distances |
| | between rows, you can also adjust the color of the points, change their shape, mark them, and output |
| | them upon selection. The algorithm in this function iteratively moves the points around in a kind of a |
| | simulation of a physical model: if two points are too close to each other (or too far away), there is a force |
| | pushing them apart (or together). The change of the point's position at each time interval corresponds to |
| | the sum of forces acting on it. |
| Save Distance | Allows the user to save a distance matrix. |
| Matrix | Input: |
| | Distances: distance matrix |
| Self-Organizing | Allows the user to compute and visualize a self-organizing graphic map. |
| Map | Input: |
| | Data: input dataset |
| | Output: |
| | Selected Data: instances selected from the plot |
| | Data: data with an additional column showing whether a point is selected |
| | Description: |
| | This PhAROS self-organizing map (SOM) function allows the user to utilize a type of artificial neural |
| | network (ANN) that is trained using unsupervised learning to produce a two-dimensional, discretized |
| | representation of the data. The function undertakes dimensionality reduction. The PhAROS self- |
| | organizing map function uses a neighborhood function to preserve the topological properties of the input |
| | space. Just like other visualization functions, the Self-Organizing Maps function also supports |
| | interactive selection of groups. To allow the user to extract, visualize and possibly re-process selected |
| | data of interest. |
| | The PhAROS Self-organizing maps differs from other artificial neural networks as they apply |
| | competitive learning as opposed to error-correction learning (such as back propagation with gradient |
| | descent), and in the sense that they use a neighborhood function to preserve the topological properties of |
| | the input space. This function is useful for visualization as it creates low-dimensional views of high- |
| | dimensional data, akin to multidimensional scaling. Useful extensions include using toroidal grids |
| | where opposite edges are connected and using large numbers of nodes. A U-Matrix can also be |
| | optionally used. The U-Matrix value of a particular node is the average distance between the node's |
| | weight vector and that of its closest neighbors. In a square grid, for instance, the closest 4 or 8 nodes |
| | might be considered (the Von Neumann and Moore neighborhoods, respectively), or six nodes in a |
| | hexagonal grid. If the SOM becomes large it will display emergent properties. In maps consisting of |
| | thousands of nodes, cluster operations on the map itself can be performed. |
| [symbol] | [symbol] [symbol] [symbol] |
| [symbol] | [symbol] [symbol] [symbol] |
| [symbol] | [symbol] [symbol] [symbol] [symbol] |
| | [symbol] [symbol] [symbol] |
| Corpus collection | Allows the user to load a corpus of text documents into the PhAROS BASE repository for subsequent |
| | data extraction into sub-systems, (optionally) tagged with categories, or change the data input signal to |
| | the corpus. |
| | Input: |
| | Data: Input data (optional) |
| | Output: |
| | Corpus: A collection of documents. |
| | Description: |
| | This PhAROS Corpus function allows the user to compute in two modes: |
| | When no data is found on input, it reads text corpora from files and sends a corpus instance to its output |
| | channel. History of the most recently opened files is maintained in the function. The function also |
| | includes a directory with sample corpora that come pre-installed with the add-on. The function reads |
| | data from Excel (.xlsx), comma-separated (.csv), native tab-delimited (.tab) files, xml, pdf, html, Json, |
| | and other file formats. When the user provides data to the input, it transforms data into the corpus. Users |
| | can select which features are used as text features. |
| Import Documents | Allows the user to import text documents from external folders, into the PhAROS_BASE corpus. |
| | Input: |
| | Text document |
| | Output |
| | Corpus: A collection of documents from the local machine. |
| | Description: |
| | This PhAROS Import Documents function retrieves text files from folders and creates a corpus. The |
| | function reads .txt, .docx, .odt, .pdf, html and .xml files. If a folder contains subfolders, they will be used |
| | as class labels. |
| News collection | Allows the user to fetch text and extract data from newspapers. |
| | Input: |
| | Newspaper text data |
| | None |
| | Output: to the Corpus: A collection of documents from the XYZ newspaper. |
| | Description: |
| | This PhAROS News Collection function allows the user retrieve articles from newspapers via their |
| | institutions API system. For the function to work, you need to provide the API key, which is available at |
| | their access platform. Although rarely used, keyword retrieval and text mining of information from these |
| | sources, can especially provide geo-temporal, epistemological, and information on clinical indications, |
| | drug, compound and mixture use, as well as sentiment towards specific drugs, plants, and traditional |
| | medicines. This is an aid toward market analysis for putative products. |
| Science Pubs | Allows the user to fetch data and text from NCBI repositories. |
| | Input: |
| | Papers and abstracts |
| | Output: |
| | Corpus: A collection of documents from the PubMed online service. |
| | Description: |
| | This PhAROS Science Pubs function provides direct access to resources like Pubmed and PMC, and |
| | other databases listed here: |
| | Assembly |
| | BioCollections |
| | BioProject |
| | BioSample |
| | BioSystems |
| | ClinicalTrials.gov |
| | ClinVar |
| | Consensus CDS (CCDS) |
| | Conserved Domain Database (CDD) |
| | abase of Genomic Structural Variation (dbVar) |
| | Database of Genotypes and Phenotypes (dbGaP) |
| | Database of Short Genetic Variations (dbSNP) |
| | GenBank |
| | Gene |
| | GeneReviews |
| | Genes and Disease |
| | Genetic Testing Registry (GTR) |
| | Genome |
| | Genome Reference Consortium (GRC) |
| | Glycans |
| | HIV-1, Human Protein Interaction Database |
| | Identical Protein Groups |
| | Journals in NCBI Databases |
| | MedGen |
| | MEDLINE (Leasing) |
| | MeSH Database |
| | National Library of Medicine (NLM) Catalog |
| | National Library of Medicine (NLM) DTDs |
| | PopSet |
| | Protein Clusters |
| | Protein Database |
| | Protein Family Models |
| | PubChem BioAssay |
| | PubChem Compound |
| | PubChem Download Service |
| | PubChem Substance |
| | PubChem Substance records contain substance information electronically submitted to PubChem by |
| | depositors. This includes any chemical structure information submitted, as well as chemical names, |
| | comments, and links to the depositor's web site. |
| | PubMed |
| | PubMed Central (PMC) |
| | Taxonomy |
| | Trace Archive |
| | This function allows you to query and retrieve entries and datasets from these sources. The user can |
| | utilize regular search or construct advanced queries, linked to the results from PhAROS_BRAIN |
| | Functions. Keyword retrieval and text mining of information from these sources, can provide temporal |
| | use information, indications for, epistemological data and sentiment towards specific drugs, plants, and |
| | traditional medicines, and provide detailed physical and chemical information needed for specifically |
| | identifying; precise patterns of interest, targets for subsequent processing, metadata groupings that |
| | correlate with indications across traditional medicines, identify of missing plants, components or |
| | compounds, identification of unknown indications for traditional medicines, identification of toxic and |
| | non-toxic components and compounds, identification of plant, component and compound mixtures with |
| | ranked therapeutic potential, identification of plant, component and compound combination that would |
| | not be obvious, and/or have greater therapeutic potential, than existing mixtures in isolated traditional |
| | medicines. |
| Social | Allows the user to fetch data and text social media platforms |
| | Fetching data from Facebook, Twitter using a search API. |
| | Input: Twitter and Facebook posts |
| | Output: Corpus: A collection of posts, and tweets from the Facebook and Twitter APIs. |
| | Description: |
| | This PhAROS Social function enables users to query text and data through the Facebook and Twitter |
| | APIs. You can query by content, author or both and accumulate results should you wish to create a |
| | larger data set. Data collected can be used similarly News collection, and Science Pubs functions, and |
| | also gives insight into epistemology of compounds mixtures, patient reported outcomes, drugs, and |
| | traditional medicines mentioned in social media posts. |
| Preprocess Text | Allows the user to reprocess corpus with user selected methods and options. |
| | Input: |
| | Corpus: A collection of documents. |
| | Output: |
| | Corpus: Preprocessed corpus. |
| | Description: |
| | This PhAROS Preprocess Text function, allows the user to split larger text data into smaller units |
| | (tokens), filter them, run normalization (stemming, lemmatization), create n-grams and tag tokens with |
| | part-of-speech labels. Steps in the analysis are applied sequentially and can be reordered. Click and drag |
| | options allow the user to change the order of the preprocessing. |
| Corpus to Network | Allows the user to create a network from a given corpus. Network nodes can be either documents or |
| | words (ngrams). |
| | Input: |
| | Corpus: A collection of documents. |
| | Output: |
| | Network: A network generated from input corpus. |
| | Node data: Additional data about nodes. |
| | Description: |
| | This PhAROS Corpus to Network function allows users the option to process either on documents or |
| | words (ngrams). If nodes are documents, there's an edge between two documents if the number of words |
| | (ngrams) that appears in both documents is at least Threshold. If nodes are words (ngrams), there's an |
| | edge between two words if the number of times they both appear inside of a window (of size 2 * |
| | Window size + 1) is at least Threshold. Only words that have frequency higher than Frequency |
| | Threshold will be included as nodes. This is a word co-occurrence network. Co-occurrence networks are |
| | generally used to provide a graphic visualization of potential relationships between people, traditional |
| | medicine, compounds, organisms or other data points represented within written material. The |
| | generation and visualization of co-occurrence networks has become practical with the advent of |
| | electronically stored text compliant to text mining. By way of definition, co-occurrence networks are the |
| | collective interconnection of terms based on their paired presence within a specified unit of text. |
| | Networks are generated by connecting pairs of terms using a set of criteria defining co-occurrence. For |
| | example, terms A and B may be said to “co-occur” if they both appear in a particular article. Another |
| | article may contain terms B and C. Linking A to B and B to C creates a co-occurrence network of these |
| | three terms. Rules to define co-occurrence within a text corpus can be set according to desired criteria. |
| | For example, more stringent criteria for co-occurrence may require a pair of terms to appear in the same |
| | sentence. |
| Bag of Words | Allows the user to generate a bag of words from the input corpus. |
| | Input: |
| | Corpus: A collection of documents. |
| | Output: |
| | Corpus: Corpus with bag of words features appended. |
| | Description: |
| | This PhAROS Bag of Words function allows the user to create a corpus with word counts for each data |
| | instance (document). The count can be either absolute, binary (contains or does not contain) or sublinear |
| | (logarithm of the term frequency). Bag of words model is required in combination with Word |
| | Enrichment and could be used for predictive modeling. |
| Document | Allows the user to embed documents from input corpus into vector space by using pretrained fastText |
| Embedding | models |
| | Input: |
| | Corpus: A collection of documents. |
| | Output: |
| | Corpus: Corpus with new features appended. |
| | Description: |
| | This PhAROS Document Embedding function allows the user to parse ngrams of each document in |
| | corpus, obtain embedding for each ngram using pretrained model for chosen language and obtains one |
| | vector for each document by aggregating ngram embeddings using one of offered aggregators. This can |
| | function on any ngrams but it will give best results if corpus is preprocessed such that ngrams are words |
| | (because model was trained to embed words). |
| Similarity Hashing | Allows the user to compute document hashes. |
| | Input: |
| | Corpus: A collection of documents. |
| | Output: |
| | Corpus: Corpus with simhash value as attributes. |
| | This PhAROS Similarity Hashing function allows the user to transform documents into similarity |
| | vectors. The function uses SimHash method. |
| Sentiment Analysis | Allows the user to predict sentiment from text. |
| | Input: |
| | Corpus: A collection of documents. |
| | Output: |
| | Corpus: A corpus with information on the sentiment of each document. |
| | Description: |
| | This PhAROS Sentiment Analysis function allows the user to predict sentiment for each document in a |
| | corpus. The function uses Liu & Hu and Vader sentiment modules from NLTK and multilingual |
| | sentiment lexicons from the Data Science Lab. All of them are lexicon-based. The Liu & Hu and Vader |
| | function options work on English. However, multilingual sentiment supports several languages; as such |
| | it will be useful in assessing foreign language in traditional medical texts from other cultures and |
| | countries. |
| Topic Modeling | Allows the user to topic model with Latent Dirichlet Allocation, Latent Semantic Indexing and/or |
| | Hierarchical Dirichlet Processing. |
| | Input: |
| | Corpus: A collection of documents. |
| | Output: |
| | Corpus: Corpus with topic weights appended. |
| | Topics: Selected topics with word weights. |
| | All Topics: Token weights per topic. |
| | Description: |
| | This PhAROS Topic Modeling function allows the user to discover abstract topics in a corpus based on |
| | clusters of words found in each document and their respective frequency. A document typically contains |
| | multiple topics in different proportions, thus the function also reports on the topic weight per document. |
| | The function wraps gensim's topic models (LSI, LDA, and HDP). The first, LSI, can return both positive |
| | and negative words (words that are in a topic and those that aren't) and concurrently topic weights, that |
| | can be positive or negative. LDA can be more easily interpreted, but is slower than LSI. HDP has many |
| | parameters - the parameter that corresponds to the number of topics is Top level truncation level (T). |
| Corpus Viewer | Allows the user to display corpus content. |
| | Input: Corpus: A collection of documents. |
| | Output: Corpus: Documents containing the queried word. |
| | Description: |
| | This PhAROS Corpus Viewer function allows users to view text files (instances of Corpus) within the |
| | PhAROS_CORPUS, or other pre-processed texts within the PhAROS subsystems. It will output an |
| | instance of corpus. |
| Word Cloud | Allows the user to generate a word cloud from corpus. |
| | Input: |
| | Topic: Selected topic. |
| | Corpus: A collection of documents. |
| | Output: |
| | Corpus: Documents that match the selection. |
| | Selected Word: Selected word that can be used as query in Concordance. |
| | Word Counts: Words and their weights. |
| | Description: |
| | This PhAROS Word Cloud function allows users to display tokens in the corpus, their size denoting the |
| | frequency of the word in corpus or an average bag of words count, when the PhAROS bag of words |
| | function is utilized in conjunction with this function. Words are listed by their frequency (weight) in the |
| | function. The function Output documents, containing selected tokens from the word cloud. |
| Concordance | Allows the user to display the context of the word. |
| | Input: |
| | Corpus: A collection of documents. |
| | Output: |
| | Selected Documents: Documents containing the queried word. |
| | Concordances: A table of concordances. |
| | Description |
| | This PhAROS Concordance function allows the user to find the queried word in a text and displays the |
| | context in which this word is used. Results in a single color come from the same document. The function |
| | can output selected documents for further analysis or a table of concordances for the queried word. |
| DocGeoMap | Allows the user to display geographic locations mentioned in the text. |
| | Input: |
| | Data: Data set. |
| | Output: |
| | Corpus: Documents containing mentions of selected geographical regions. |
| | Description: |
| | This PhAROS Document GEO Map function allows users to visualize geolocations from textual (string) |
| | data. It processes mentions of geographic names (countries and capitals) and displays distributions |
| | (frequency of mentions) of these names on a map. It works with any PhAROS function that produces a |
| | data table and that contains at least one string attribute. The function produces selected data instances |
| | that are all documents containing mentions of a selected country (or countries). |
| Word Enrichment | Allows the user to utilize word enrichment analysis for selected documents. |
| | Input: |
| | Corpus: A collection of documents. |
| | Selected Data: Selected instances from corpus. |
| | Output: |
| | Enrichment analysis |
| | Description: |
| | This PhAROS Word Enrichment function, allows the user to visualize a list of words with lower p- |
| | values (higher significance) for a selected subset compared to the entire corpus. Lower p-value indicates |
| | a higher likelihood that the word is significant for the selected subset (not randomly occurring in a text). |
| | FDR (False Discovery Rate) is linked to p-value and reports on the expected percent of false predictions |
| | in the set of predictions, meaning it account for false positives in list of low p-values. |
| Duplicate Detection | Allows the user to detect & remove duplicates from a corpus. |
| | Input: |
| | Distances: A distance matrix. |
| | Output: |
| | Corpus Without Duplicated: Corpus with duplicates removed. |
| | Duplicates Cluster: Documents belonging to selected cluster. |
| | Corpus: Corpus with appended cluster labels. |
| | Description: |
| | This PhAROS Duplicate Detection function, allows users to utilize clustering to find duplicates in the |
| | corpus. It works well with the Social, and PUBMED/PMC and other functions for removing duplicates |
| | and other similar documents. Within the function the level of similarity can be set, through the |
| | interactive visualization. |
| Statistics | Allows the user to create new statistical variables for documents. |
| | Input: |
| | Corpus: A collection of documents. |
| | Output: |
| | Corpus: Corpus with additional attributes. |
| | Description: |
| | This PhAROS Statistics function that allows the user to add and calculate simple document statistics to a |
| | corpus. It supports both standard statistical measures and user-defined variables. |
| PhAROS | These PhAROS BIOINFORMATIC functions, allow the PhAROS system to rapidly collect, store, parse |
| BIOINFORMATIC | and analyze bioinformatics based data from a variety of sources, for use in the pre-processed PhAROS |
| FUNCTIONS | subsystems, or for de-novo analysis, depending on the users case use. Raw data, as well as specific sets |
| | of data are predominantly stored in the PhAROS CORE subsystem, or processed and added to PhAROS |
| | subsystems, for access by various types of user, depending on their use case. |
| Databases Update | Allows users to manually, semi-automatically or automatically update, local PhAROS sub-systems |
| | databases, like gene ontologies, annotations, gene names, protein interaction networks, and similar, from |
| | external databases |
| | Description: |
| | This PhAROS Databases Update function allows users to access several databases directly from |
| | PhAROS. The function can also be used to update and manage locally stored sub-system databases. |
| GEO Data Sets | Allows users to access data sets from gene expression omnibus GEO DataSets. |
| | Output: |
| | Expression data: Data set selected in the function with genes or samples in rows. |
| | Description: |
| | This PhAROS_GEO DataSets function, allows direct access to the gene expression omnibus GEO |
| | DataSets. This is a database of gene expression curated profiles maintained by NCBI and included in the |
| | Gene Expression Omnibus. This Pharos sub-system function provides access to all its data sets and |
| | outputs a data set selected for further processing. For convenience, each downloaded data set is stored |
| | locally, with the PhAROS_BASE repository. |
| dictyExpress | Allows users to access to dictyExpress databases. |
| | Output: |
| | Data: Selected experiment (time-course gene expression data). |
| | Description: |
| | This PhAROS dictyExpress function gives PhAROS users direct access to the dictyExpress database. It |
| | allows users to download the data from selected experiments in Dictyostelium by Baylor College of |
| | Medicine. |
| Genes | Allows users to match input gene ID's with corresponding Entrez ID's. |
| | Input: |
| | Data: Data set. |
| | Output: |
| | Data: Instances with meta data that the user has manually selected in the function. |
| | Genes: All genes from the input with included gene info summary and matcher result. |
| | Description: |
| | This PhAROS Genes function is a useful PhAROS function that allows users to retrieve and visualize |
| | information and data on the genes from the NCBI Gene database and can output an annotated data table. |
| | Users can also select a subset and feed it to other functions. |
| Differential | Allows users to visually generate plots describing differential gene expression for selected experiments. |
| Expression | Input: |
| | Data: Data set. |
| | Output: |
| | Data Subset: Differentially expressed genes. |
| | Remaining Data Subset: Genes that were not differentially expressed. |
| | Selected Genes: Genes from the select data with scores appended. |
| | Description: |
| | This PhAROS Differential Expression function allows users to calculate and produce visual plots and |
| | graphs showing a differential gene expression graph for a sample target. It takes gene expression data as |
| | an input (from dictyExpress, GEO Data Sets, etc.) and outputs a selected data subset. |
| GO Browser | Allows users to access to Gene Ontology database. |
| | Input: |
| | Cluster Data: Data on clustered genes. |
| | Reference Data: Data with genes for the reference set (optional). |
| | Output: |
| | Data on Selected Genes: Data on genes from the selected GO node. |
| | Enrichment Report: Data on GO enrichment analysis. |
| | The PhAROS GO Browser function provides users direct access to the Gene Ontology database. Gene |
| | Ontology (GO) classifies genes and gene products to terms organized in a graph structure called |
| | ontology. The PhAROS GO Browser function takes any data on genes as an input (it is best to input |
| | statistically significant genes, for example from the output of the Differential Expression function) and |
| | shows a ranked list of GO terms with p-values. This is a great tool for finding biological processes that |
| | are over- or under-represented in a particular gene set. The user can filter input data by selecting terms in |
| | a list. |
| KEGG Pathways | Allows users to access diagrams of molecular interactions, reactions, and relationships. |
| | Input: Data: Data set. |
| | Reference: Referential data set. |
| | Output: |
| | Selected Data: Data subset. |
| | Unselected Data: Remaining data. |
| | Description: |
| | The PhAROS KEGG Pathways function displays diagrams of molecular interactions, reactions and |
| | relations from the KEGG Pathways Database. It takes user selected data on gene expression as an input, |
| | matches the genes to the biological processes and displays a list of corresponding pathways. To explore |
| | the pathway, the user can interact with and click on any process displayed or rank sort them by P-value |
| | to get the most relevant processes at the top. |
| Gene Set | Enrich gene sets. |
| Enrichment | Input: |
| | Data: Data set. |
| | Custom Gene Sets: Genes to compare. |
| | Reference Genes: Genes used as reference. |
| | Output: |
| | Matched Genes: Genes that match. |
| | Description: |
| | The PhAROS Gene Set Enrichment function allows users to process and visualize genes and genes sets |
| | that match each other. |
| Cluster Analysis | Allows users to display differentially expressed genes that characterize the cluster. |
| | Input: |
| | Data: Data set. |
| | Custom Gene Sets: Genes to compare. |
| | Output: |
| | Selected Data: Data selected by the user in the PhAROS Cluster Analysis function. |
| | Description: |
| | The PhAROS Cluster Analysis function displays differentially expressed genes that characterize the |
| | cluster, and corresponding gene terms that describe differentially expressed genes. |
| Volcano Plot | Allows users to generate visual plots indicating significance versus fold-change for gene expression |
| | rates. |
| | Input: |
| | Data: Input data set. |
| | Output: |
| | Selected Data: Data subset. |
| | Description: |
| | The PhAROS volcano plot function, allows users to compute and visualize changes in replicate data. |
| | The PhAROS Volcano Plot function plots a binary logarithm of fold-change on the x-axis versus |
| | statistical significance (negative base 10 logarithm of p-value) on the y-axis. |
| | The PhAROS Volcano Plot function is useful for a rapid visual identification of statistically significant |
| | data. Genes that are highly dysregulated are farther to the left and right, while highly significant fold |
| | changes appear higher on the plot. A combination of the two is those genes that are statistically |
| | significant. |
| Marker Genes | Allows users to access to a public database of marker genes. |
| | Input: |
| | Database sources: PanglaoDB, CellMarker |
| | Output: Genes |
| | Description: |
| | This PhAROS Marker Gene function, allows user direct access to internet attached public databases of |
| | marker genes. Retrieve data, and data sets, and visualize data using other PhAROS sub-systems and |
| | functions.. |
| Annotator | Allows users an option to annotate cells with cell types based on marker genes. |
| | Input: |
| | Reference Data: Data set with gene expression values. |
| | Secondary Data: Subset of instances (optional). |
| | Genes: Marker genes. |
| | Output: |
| | Selected Data: Instances selected from the plot. |
| | Data: Data with additional columns with annotations, clusters, and projection |
| | This PhAROS Annotator function allows user to retrieve, and process gene expression data together with |
| | mapping to a two-dimensional space and marker genes. It selects the most expressed genes for each cell |
| | with the Mann-Whitney U test and computes the p-value of each cell types for a cell based on the |
| | selected statistical test. This PhAROS Annotator function visualizes groups of cells and for each group; |
| | it shows the few most present cell types. |
| PhAROS_{—}IMAGE | These PhAROS IMAGE_{—}ANALYTICS functions, allow the PhAROS system and users, to rapidly collect, |
| ANALYTICS | store, parse and analyze image based data from a variety of sources, for use in the pre-processed |
| FUNCTIONS | PhAROS subsystems, or for de-novo analysis, depending on the user's case use. Raw data, as well as |
| | specific sets of data are predominantly stored in the PhAROS CORE subsystem, or processed and added |
| | to PhAROS subsystems, for access by various types of user, depending on their use case. |
| Import Images | Allows users to import images external from the PhAROS system. |
| | Output: To be deposited in the PhAROS_BASE or other subsystems as needed |
| | Data: Dataset describing one image in each row. |
| | Description: |
| | This PhAROS Import Images function assesses all images in a directory and returns one per row per |
| | located image. Columns include image name, path to image, width, height and image size, and other |
| | metadata, based on the source and the image header. |
| Image Viewer | Allows users to display images that come with, or are attached to a data set. |
| | Input: |
| | Data: A data set with images. |
| | Output: |
| | Data: Images that come with the data. |
| | Selected images: Images selected in the PhAROS Image Viewer function. |
| | Description: |
| | This PhAROS Image Viewer function can display images from a data set, which are stored locally, in |
| | any of the subsystems, or on the internet. The function will assess image attributes with type = image in |
| | the third header row. It can be used for image comparison, while looking for similarities or discrepancies |
| | between selected data instances. |
| Image Embedding | Allows users to embed images through deep neural networks. |
| | Input: |
| | Images: List of images. |
| | Output: |
| | Embeddings: Images represented with a vector of numbers. |
| | Skipped Images: List of images where embeddings were not calculated. |
| | Description: |
| | The PhAROS Image Embedding function reads images and uploads them to the PhAROS_BASE |
| | subsystem, or other subsystem. Deep learning models are used to calculate a feature vector for each |
| | image. It returns an enhanced data table with additional columns (image descriptors). Images can be |
| | imported with the PhAROS Image Embedding function. |
| Image Grid | Allows users to display images in a similarity grid. |
| | Input: |
| | Embeddings: Image embeddings from Image Embedding function. |
| | Data Subset: A subset of embeddings or images. |
| | Output: |
| | Images: Images from the dataset with an additional column specifying if the image is selected or the |
| | group, if there are several. |
| | Selected Images: Selected images with an additional column specifying the group. |
| | Description: |
| | The PhAROS Image Grid function can display images from a dataset in a similarity grid - images with |
| | similar content are placed closer to each other. It can be used for image comparison, while looking for |
| | similarities or discrepancies between selected data instances. |
| Save Images | Allows users to save images in the directory structure. |
| | Input: |
| | Data: images to save. |
| | Description: |
| | The PhAROS Save Images function saves images sent to its input. Images will be saved as separate files |
| | in their own directory, or deposited with the appropriate database of PhAROS_BASE, or other PhAROS |
| | subsystems. |
| PhAROS | These PhAROS NETWORKS functions, allow the PhAROS system and users, to rapidly generate, |
| NETWORKS | compute, store, parse and analyze network datasets, from PhAROS subsystem data, and imported or |
| FUNCTIONS | external data, for use in the pre-processed PhAROS subsystems, or for de-novo analysis, depending on |
| | the users case use. Raw data, as well as specific sets of data are predominantly stored in the PhAROS |
| | CORE subsystem, or processed and added to PhAROS subsystems, for access by various types of user, |
| | depending on their use case. |
| Network File | Allows users to read and write network files in all formats. |
| | Output: |
| | Network: An instance of Network Graph. |
| | Items: Properties of a network file. |
| | Description: |
| | The PhAROS Network File function can open and save network files and send the input data to its |
| | output channel i.e. A file or PhAROS subsystem. History of the most recently opened files in maintained |
| | in the function. The function opens and saves data formats such as .net and .pajek formats. A |
| | complimentary .tab, .tsv or .csv data set can be provided for node information. |
| Network Explorer | Allows users to visually explore the network and its properties. |
| | Input: |
| | Network: An instance of Network Graph. |
| | Node Subset: A subset of vertices. |
| | Node Data: Information on vertices. |
| | Node Distances: Data on distances between nodes. |
| | Output: |
| | Selected sub-network: A network of selected nodes. |
| | Distance Matrix: Distance matrix. |
| | Selected Items: Information on selected vertices. |
| | Highlighted Items: Information on highlighted vertices. |
| | Remaining Items: Information on remaining items (not selected or highlighted). |
| | Description: |
| | The PhAROS Network Explorer function is the primary PhAROS function for visualizing network |
| | graphics visual. It displays a graph with Fruchterman-Reingold layout optimization and enables setting |
| | the color, size and label of nodes. One can also highlight nodes of specific properties and output them. |
| | The visualization in Network Explorer works just like the one for Scatter Plot. To select a subset of |
| | nodes, draw a rectangle around the subset. Add to a new group, or add to the existing group. |
| | Force-directed graph drawing algorithms are a class of algorithms for drawing graphs in an aesthetically- |
| | pleasing way. Their purpose is to position the nodes of a graph in two-dimensional or three-dimensional |
| | space so that all the edges are of more or less equal length and there are as few crossing edges as |
| | possible, by assigning forces among the set of edges and the set of nodes, based on their relative |
| | positions, and then using these forces either to simulate the motion of the edges and nodes or to |
| | minimize their energy. |
| Network Generator | Allows users to construct example graphs. |
| | Output: |
| | Generated Network: An instance of Network Graph. |
| | Description: |
| | The PhAROS Network Generator function constructs exemplary networks. |
| | Graph options include but are not limited to: |
| | Path: a graph that can be drawn so that all of its vertices and edges lie on a single straight line. |
| | Cycle: a graph that consists of a single cycle, i.e. some number of vertices (at least 3) is connected in a |
| | closed chain. |
| | Complete: simple undirected graph in which every pair of distinct vertices is connected by a unique |
| | edge. |
| | Complete bipartite: a graph whose vertices can be divided into two disjoint and independent sets. |
| | Barbell: two complete graphs connected by a path. |
| | Ladder: planar undirected graph with 2n vertices and 3n-2 edges. |
| | Circular ladder: Cartesian product of two path graphs. |
| | Grid: a graph whose drawing, embedded in some Euclidean space, forms a regular tiling. |
| | Hypercube: a graph formed from the vertices and edges of an n-dimensional hypercube. |
| | Star: Return the Star graph with n + 1 nodes: one center node, connected to n outer nodes. |
| | Lollipop: a complete graph (clique) and a path graph, connected with a bridge. |
| | Geometric: an undirected graph constructed by randomly placing N nodes in some metric space. |
| Network Analysis | Allows users to undertake statistical analysis of network data. |
| | Input: |
| | Network: An instance of Network Graph. |
| | Items: Properties of a network file. |
| | Output: |
| | Network: An instance of Network Graph with appended information. |
| | Items: New properties of a network file. |
| | Description: |
| | The PhAROS Network Analysis function computes node-level and graph-level summary statistics for |
| | the network. It outputs a network with the new computed statistics and an extended item data table |
| | (node-level indices only). |
| Network Clustering | Allows users to detect clusters in a network. |
| | Input: |
| | Network: An instance of Network Graph. |
| | Output: |
| | Network: An instance of Network Graph with clustering information appended. |
| | Description: |
| | The PhAROS Network Clustering function finds clusters in a network. Clustering works with two |
| | algorithms, one uses label propagation to find appropriate clusters, and one which adds hop attenuation |
| | as parameters for cluster formation. |
| Network Of Groups | Allows users to group instances by feature and connect related groups. |
| | Input: |
| | Network: An instance of network graph. |
| | Data: Properties of a network graph. |
| | Output: |
| | Network: A grouped network graph. |
| | Data: Properties of the group network graph. |
| | Description: |
| | The PhAROS Network of Groups function is the network version of the group-by operation. Nodes with |
| | the same values of the attribute, selected in the dropdown, will be represented as a single node. |
| Network From | Allows users to construct a network from distances between instances. |
| Distances | Input: |
| | Distances: A distance matrix. |
| | Output: |
| | Network: An instance of Network Graph. |
| | Data: Attribute-valued data set. |
| | Distances: A distance matrix. |
| | The PhAROS Network from Distances function constructs a network graph visual from a given distance |
| | matrix. The graph is constructed by connecting nodes from the matrix where the distance between nodes |
| | is below the given threshold. In other words, all instances with a distance lower than the selected |
| | threshold will be connected. |
| Single Mode | Allows users to convert multimodal graphs to single modal. |
| | Input: |
| | Network: An instance of a bipartite network graph. |
| | Output: |
| | Network: An instance of single network graph. |
| | Description: |
| | The PhAROS Single Mode function works with bipartite (or multipartite) networks, where different |
| | parts are distinguished by values of the chosen discrete variable. A typical example would be a network |
| | that connects persons with events that they attended. The function creates a new network, which contains |
| | the nodes from the chosen group of original network's nodes (e.g. persons). Two nodes in the resulting |
| | network are connected if they share a common neighbor from the second chosen group (e.g. events). |
| PhAROS | These PhAROS_{—}GEO functions, allow the PhAROS system and users, to rapidly generate, compute, |
| GEO | store, parse, analyze and visualize GEO linked data and datasets, from PhAROS subsystem data, and/or |
| FUNCTIONS | imported or external data, for use in the pre-processed PhAROS subsystems, or for de-novo analysis, |
| | depending on the users case use. Raw data, as well as specific sets of data are predominantly stored in |
| | the PhAROS_{—}CORE subsystem, or processed and added to PhAROS subsystems, for access by various |
| | types of user, depending on their use case. |
| Geocoding | Allows users to encode region names into geographical coordinates, or reverse-geocode latitude and |
| | longitude pairs into regions. |
| | Input: |
| | Data: An input data set. |
| | Output: |
| | Coded Data: Data set with new meta attributes. |
| | Description: |
| | This PhAROS Geocoding function extracts latitude/longitude pairs from region names or synthesizes |
| | latitude/longitude to return region name. If the region is large, say a country, the encoder will return the |
| | latitude and longitude, and the geometric center. |
| Geo Map | Allows users to show data points, and datasets on a map. |
| | Input: |
| | Data: input dataset |
| | Data Subset: subset of instances |
| | Output: |
| | Selected Data: instances selected from the plot |
| | Data: data with an additional column showing whether a point is selected. |
| | Description: |
| | This PhAROS Geo Map function visualizes geo-spatial data on a map. It works on datasets containing |
| | latitude and longitude variables in WGS 84 (EPSG: 4326) format, and can be used interactively, much |
| | like the PhAROS Scatter Plot function. |
| Choropleth Map | Allows users to utilize a thematic map in which areas are shaded in proportion to the measurement of the |
| | statistical variable being displayed. |
| | Input: |
| | Data: input dataset |
| | Output: |
| | Selected Data: instances selected from the map. |
| | Data: data with an additional column showing whether a point is selected. |
| | Description: |
| | This PhAROS Choropleth function provides an easy way to visualize how a data varies across a |
| | geographic area, or indicates the level of variability within a region. There are several levels of |
| | granularity available, from countries to states, counties, or municipalities. |
| PhAROS | These PhAROS TIME functions, allow the PhAROS system and users, to rapidly generate, compute, |
| TIME | store, parse, analyze and visualize temporally linked data and datasets, from PhAROS subsystem data, |
| FUNCTIONS | and/or imported or external data, for use in the pre-processed PhAROS subsystems, or for de-novo |
| | analysis, depending on the users case use. Raw data, as well as specific sets of data are predominantly |
| | stored in the PhAROS_{—}CORE subsystem, or processed and added to PhAROS subsystems, for access by |
| | various types of user, depending on their use case. |
| Timeseries | Allows users to reinterpret a Table object as a Timeseries object. |
| | Input: |
| | Data: Any data table. |
| | Output: |
| | Time series: Data table reinterpreted as time series. |
| | Description: |
| | This PhAROS Timeseries function allows users to reinterpret and visualize any data table as a time |
| | series, so it can be used with the rest of the functions available to the user through the PhAROS system. |
| | In the function, you can set which data attribute represents the time variable. |
| Interpolate | Allows users to Induce missing values in the time series by interpolation. |
| | Input: |
| | Time series: Time series as output by As Timeseries function. |
| | Output: |
| | Time series: The input time series with the chosen default interpolation method for when the algorithms |
| | require interpolated time series (without missing values). |
| | Interpolated time series: The input time series with any missing values interpolated according to the |
| | chosen interpolation method. |
| | Description: |
| | This PhAROS Interpolate function allows users to assess and visualize data with missing time points. |
| | Here users can choose the interpolation method to impute the missing values with. By default, it's linear |
| | interpolation (fast and reasonable default). |
| Moving Transform | Allows users to apply rolling window functions to the time series. Use this function to get a series' |
| | mean. |
| | Input: |
| | Time series: Time series as output by As Timeseries function. |
| | Output: |
| | Time series: The input time series with the added series' transformations. |
| | Description: |
| | This PhAROS Moving Transform function allows users to define what aggregation functions to run over |
| | the time series and with what window sizes. |
| Line Chart | Allows users to visualize time series' sequence and progression in the most basic time series |
| | visualization imaginable. |
| | Input: |
| | Time series: Time series as output by PhAROS Timeseries function. |
| | Forecast: Time series forecast as output by one of the models (like VAR or ARIMA). |
| | Description: |
| | This PhAROS Line Chart function allows users to visualize the time series. |
| Periodogram | Allows users to visualize time series' cycles, seasonality, periodicity, and most significant periods. |
| | Input: |
| | Time series: Time series as output by PhAROS Timeseries function. |
| | Description: |
| | This PhAROS Periodogram function allows users to visualize the most significant periods of the time |
| | series. |
| Correlogram | Allows users to visualize variables' auto-correlation. |
| | Input: |
| | Time series: Time series as output by the PhAROS Timeseries function |
| | Description: |
| | This PhAROS Correlogram function allows users to visualize the autocorrelation coefficients for the |
| | selected time series. |
| Granger Causality | Allows users to test if one time series, or data set, Granger-causes (i.e. can be an indicator of) another |
| | time series. |
| | Input: |
| | Time series: Time series as output by the PhAROS Timeseries function. |
| | Description: |
| | These functions perform a series of statistical tests to determine the series that cause other series so we |
| | can use the former to forecast the latter. |
| ARIMA Model | Allows users to model the time series data and datasets using ARMA, ARIMA, or ARIMAX model. |
| | Input: |
| | Time series: Time series as output by PhAROS Timeseries function. |
| | Exogenous data: Time series of additional independent variables that can be used in an ARIMAX model. |
| | Output: |
| | Time series model: The ARIMA model fitted to input time series. |
| | Forecast: The forecast time series. |
| | Fitted values: The values that the model was actually fitted to, equals to original values - residuals. |
| | Residuals: The errors the model made at each step. |
| | Description: |
| | This PhAROS ARIMA Model function allows users to model the time series with an ARIMA model. |
| VAR Model | Allows users to model the time series data, and datasets using vector auto regression (VAR) model. |
| | Inputs |
| | Time series: Time series as output by PhAROS Timeseries function. |
| | Outputs |
| | Time series model: The VAR model fitted to input time series. |
| | Forecast: The forecast time series. |
| | Fitted values: The values that the model was actually fitted to, equals to original values - residuals. |
| | Residuals: The errors the model made at each step. |
| | Description: |
| | This PhAROS VAR model function allows the user to model the time series using the VAR model |
| | system. This Vector auto regression (VAR) system is a statistical model used to capture the relationship |
| | between multiple quantities as they change over time. VAR is a type of stochastic process model. VAR |
| | models generalize the single-variable (univariate) autoregressive model by allowing for multivariate |
| | time series. VAR models are often used in economics and the natural sciences. Like the autoregressive |
| | model, each variable has an equation modeling its evolution over time. This equation includes the |
| | variables lagged (past) values, the lagged values of the other variables in the model, and an error term. |
| | VAR models do not require as much knowledge about the forces influencing a variable as do structural |
| | models with simultaneous equations. The only prior knowledge required is a list of variables which can |
| | be hypothesized to affect each other over time. |
| Time Slice | Allows users to select a “slice” of measurements on a time interval. |
| | Input: |
| | Data: Time series as output by PhAROS Timeseries function. |
| | Output: |
| | Subset: Selected time slice from the time series. |
| | Description: |
| | This PhAROS Time Slice function allows users to select subsets of data, and is designed specifically for |
| | time series and for interactive visualizations. It enables users to select a subset of the data by date and/or |
| | hour. Moreover, it can output data from a sliding window with options for step size and speed of the |
| | output change. |
| Aggregate | Allows users to aggregate data by second, minute, hour, day, week, month, or year. |
| | Input: |
| | Time series: Time series as output by PhAROS Timeseries function. |
| | Output: |
| | Time series: Aggregated time series. |
| | Description: |
| | This PhAROS Aggregate function, allows users to join together instances at the same level of |
| | granularity. In other words, if aggregating by day, all instances from the same day will be merged into |
| | one. This Aggregation function can be defined separately based on the type of the attribute. |
| Difference | Allows users to make the time series stationary by replacing it with 1st or 2nd order discrete difference |
| | along its values. |
| | Input: |
| | Time series: Time series as output by PhAROS Timeseries function. |
| | Output: |
| | Time series: Differences of input time series. |
| Seasonal Adjustment | Allows users to decompose the time series into seasonal, trend, and residual components. |
| | Input: |
| | Time series: Time series as output by PhAROS Timeseries function. |
| | Output: |
| | Time series: Original time series with some additional columns: seasonal component, trend component, |
| | residual component, and seasonally adjusted time series. |

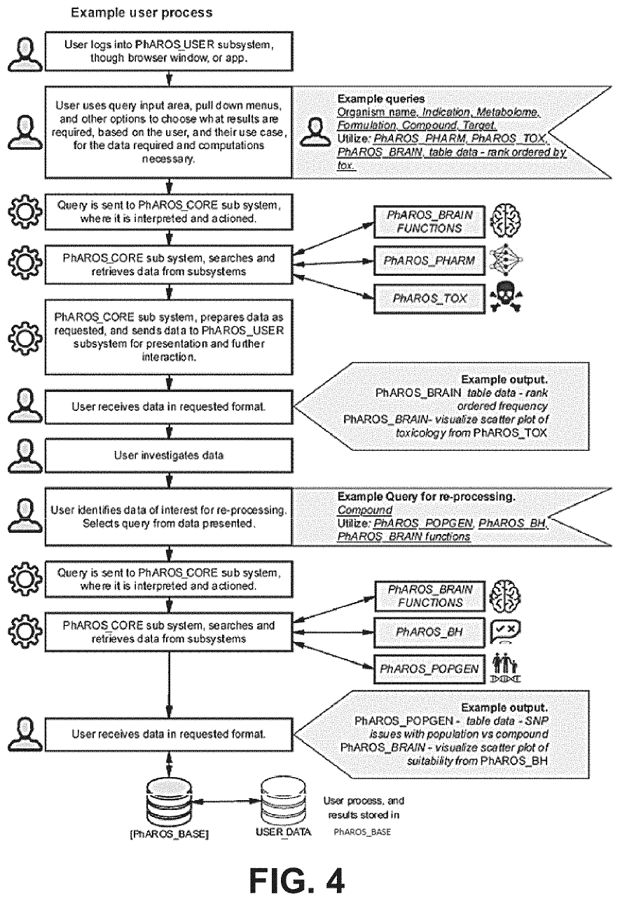

FIG. 4 shows for illustrative purposes only an example of a generalized example of user interaction with the PhAROS system and PhAROS subsystems of one embodiment. FIG. 4 shows a generalized example of user interaction with the PhAROS system and PhAROS Subsystems. FIG. 4 shows an example user process of the PhAROS SYSTEM. A user logs into PhAROS_USER subsystem, though a browser window, or app.

The user uses query input area, pull down menus, and other options to choose what results are required, based on the user and their use case for the data required and computations necessary. Example queries may include an Organism name, indication, Metabolome, formulation, compound and target. This example query will utilize the PhAROS_PHARM, PhAROS_TOX, PhAROS_BRAIN, and table data—rank ordered by tox. The query is sent to the PhAROS_CORE subsystem, where it is interpreted and actioned.

The PhAROS_CORE subsystem searches and retrieves data from subsystems. In this example the data is being retrieved from the PhAROS_BRAIN Functions, PhAROS_PHARM, and PhAROS_TOX. PhAROS_CORE sub system, prepares data as requested, and sends data to PhAROS_USER subsystem for presentation and further interaction. The user receives data in requested format.

An example of output includes PhAROS_BRAIN table data—rank ordered frequency PhAROS_BRAIN-visualize scatter plot of toxicology from PhAROS_TOX. The user investigates data. The user identifies data of interest for re-processing. Selects query from data presented.

In this example query for re-processing for a compound the user utilizes PhAROS_POPGEN, PhAROS_BH, and PhAROS_BRAIN Functions. The query is sent to the PhAROS_CORE subsystem, where it is interpreted and actioned. The PhAROS_CORE sub system searches and retrieves data from subsystems. PhAROS_POPGEN, PhAROS_BH, and PhAROS_BRAIN Functions. The user receives data in requested format. Example output. PhAROS_POPGEN—table data—SNP issues with population vs. compound PhAROS_BRAlN-visualize scatter plot of suitability from PhAROS_BH. This example of a user process is completed, and results are stored in the PhAROS_BASE in USER DATA of one embodiment.

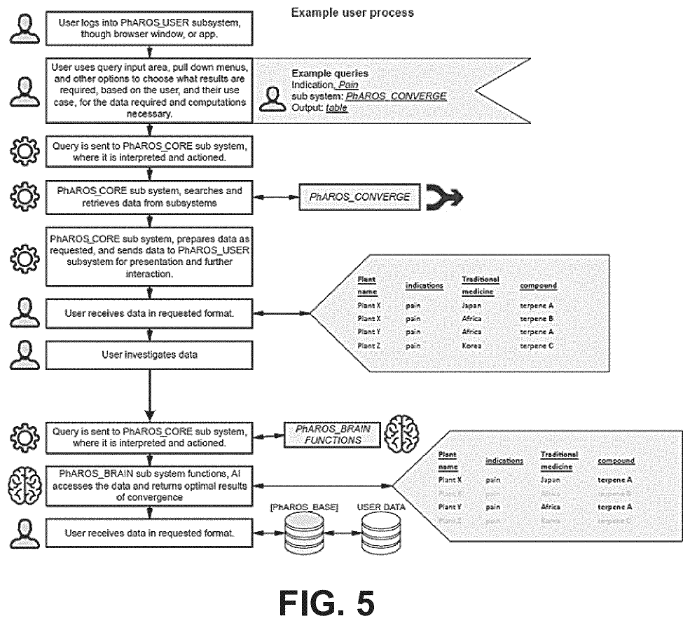

FIG. 5 shows for illustrative purposes only an example of a generalized example of user interaction with the PhAROS system and PhAROS subsystems of one embodiment. FIG. 5 shows a generalized example of user interaction with the PhAROS system and PhAROS subsystems. In an example user process a user logs into PhAROS_USER subsystem, though a browser window, or app. The user uses query input area, pull down menus, and other options to choose what results are required, based on the user and their use case for the data required and computations necessary.

Example queries may include an indication pain, Metabolome, formulation, compound and target. This example query will utilize the PhAROS_CONVERGE with output in a table. The query is sent to the PhAROS_CORE subsystem, where it is interpreted and actioned. The PhAROS_CORE sub system searches and retrieves data from subsystems. In this example the data is being retrieved from the PhAROS_CONVERGE Functions. A PhAROS_CORE sub system, prepares data as requested, and sends data to PhAROS_USER subsystem for presentation and further interaction. The user receives data in requested format. The user investigates data. The user actions AI interface with PhAROS_BRAIN to analyze data. The query is sent to the PhAROS_CORE subsystem, where it is interpreted and actioned with the PhAROS_BRAIN Functions. The PhAROS_BRAIN subsystem functions, AI accesses the data and returns optimal results of convergence.

TABLE 2
Example results table:

| Plant name, | indications, | Traditional Medicine | compound |
|---|---|---|---|
| Plant X, | pain, | Japan, | terpene A; |
| Plant X, | pain, | Africa, | terpene B; |
| Plant Y, | pain, | Africa, | terpene A; |
| Plant Z, | pain, | Korea, | terpene C.; |

The user receives data in requested format, and results are stored in the PhAROS_BASE USER DATA.

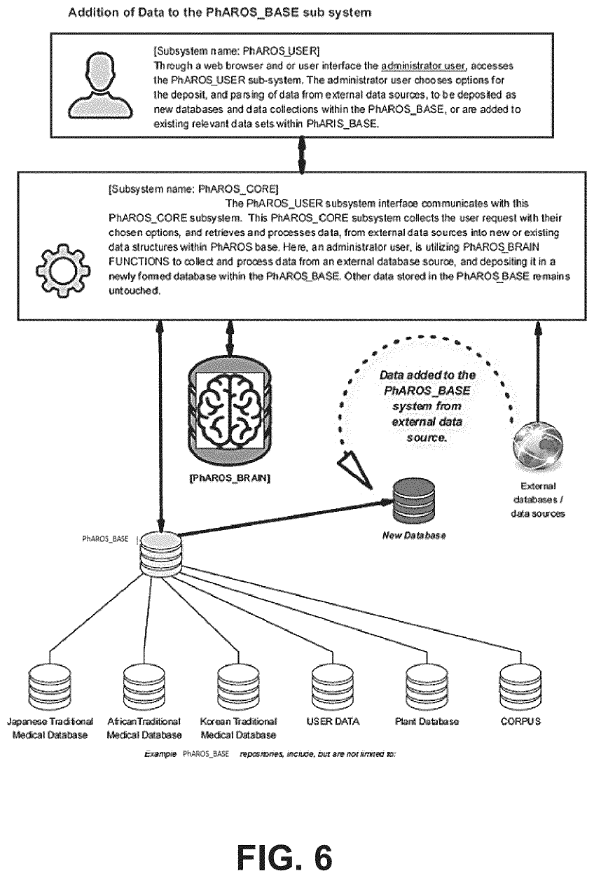

FIG. 6 shows for illustrative purposes only an example of a schematic of major components of the PhAROS system and subsystems, used in an example of importing data into the PhAROS BASE system, and creation of a new database to contain this data of one embodiment. FIG. 6 shows a schematic of major components of the PhAROS system and subsystems, used in an example of importing data into the PhAROS_BASE system, and creation of a new database to contain this data. This example shows how an addition of Data to the PhAROS_BASE sub system and Subsystem name: PhAROS_USER is processed.

Through a web browser and/or user interface the administrator user accesses the PhAROS_USER sub-system. The administrator user chooses options for the deposit and parsing of data from external data sources to be deposited as new databases and data collections within the PhAROS_BASE and alternatively is added to existing relevant data sets within the PhAROS_BASE. Another administrator user option for Subsystem name: PhAROS_CORE is the PhAROS_USER subsystem interface communicates with this PhAROS_CORE subsystem.

This PhAROS_CORE subsystem collects the user request with their chosen options, and retrieves and processes data, from external data sources into new or existing data structures within PhAROS_BASE. Here an administrator user is utilizing PhAROS_BRAIN FUNCTIONS to collect and process data from an external database source and depositing it in a newly formed database within the PhARO5 BASE. Other data stored in the PhAROS_BASE remains untouched.

External databases/data sources data mined for information is data added to the PhAROS_BASE system from external data source. The external data gathered is stored in a new database and distributed into the PhAROS_BRAIN and PhAROS_BASE repositories. An example of the distributions to the PhAROS_BASE repositories include, but are not limited to a Japanese Traditional medical database, African Traditional medical database, Korean Traditional medical database, USER DATA, Plant Database, and CORPUS of one embodiment.

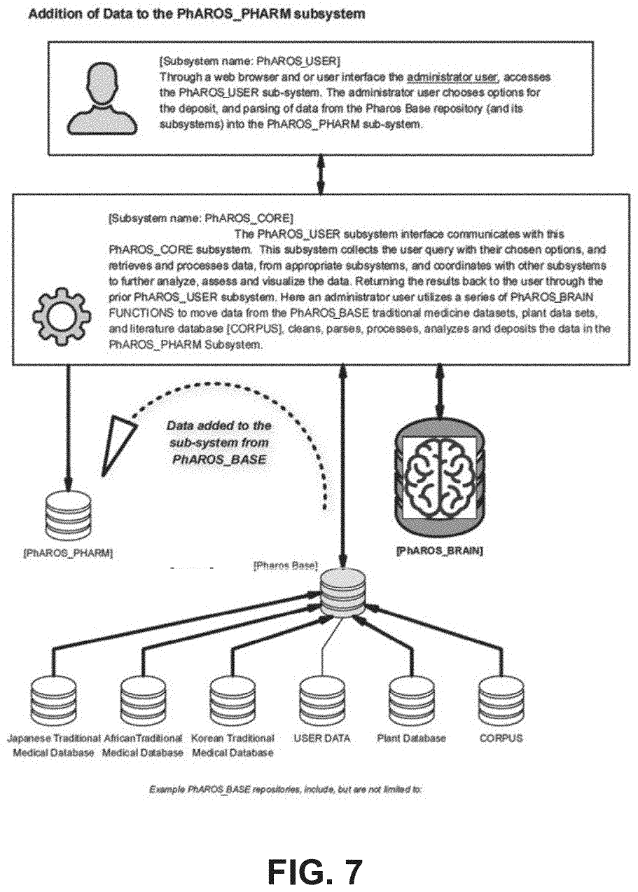

FIG. 7 shows for illustrative purposes only an example of a schematic of major components of the PhAROS system and subsystems, used in an example of processing, mining, and parsing specific data into the PhAROS_PHARM system, from multiple raw data sources in the PhAROS_BASE subsystem of one embodiment. FIG. 7 shows a schematic of major components of the PhAROS system and subsystems, used in an example of processing, mining, and parsing specific data into the PhAROS_PHARM system, from multiple raw data sources in the PhAROS_BASE subsystem.

In this example addition of data to the PhAROS_PHARM subsystem is shown with the Subsystem name: PhAROS_USER. Through a web browser and/or user interface the administrator user accesses the PhAROS_USER sub-system. The administrator user chooses options tor the deposit, and parsing of data from the Pharos Base repository (and its subsystems) into the PhAROS_PHARM sub-system. The Subsystem name: PhAROS_CORE directs the additions. The PhAROS_USER subsystem interface communicates with this PhAROS_CORE subsystem. This subsystem collects the user query with their chosen options, and retrieves and processes data, from appropriate subsystems and coordinates with other subsystems to further analyze, assess and visualize the data. Returning the results back to the user through the prior PhAROS_USER subsystem.

Here an administrator user utilizes a series of PhAROS_BRAIN Functions to move data from the PhAROS_BASE traditional medicine datasets, plant data sets, and literature database [CORPUS], cleans, parses, processes, analyzes and deposits the data in the PhAROS_PHARM Subsystem. PhAROS_BRAIN controls the processes for the additions. The PhAROS_BASE controls its subsystems. Data added to the sub-system from PhAROS_BASE subsystems include for example PhAROS_BASE repositories include, but are not limited to the Japanese Traditional medical database, African Traditional medical database, Korean Traditional medical database, USER DATA, Plant Database, and CORPUS. This data is added to the PhAROS_PHARM in one embodiment.

In one embodiment PhAROS includes a method for creation of the meta-pharmacopeia PhAROS_PHARM. In one embodiment PhAROS includes a user interaction dashboard for the PhAROS_PHARM component. In one embodiment PhAROS includes a method used to construct and assemble the PhAROS_PHARM meta-pharmacopeia repository and computational space.

In one embodiment, a PhAROS data process is utilized for in silico convergence analysis (ISCA). In one embodiment a PhAROS data process is utilized to deconvolve modes and mechanisms of action, inclusion priority and underlying epistemology to identify minimal essential formulations of phytochemicals for specific indications. In one embodiment a PhAROS data process is utilized to generate a method to diversify the supply chain of a user/stakeholder for phytomedicine plants, organisms, components and/or compounds.

In one embodiment PhAROS components can be utilized to provide a method to rationalize phytomedicine design and cultivation pipelines for global health issues.

In one embodiment PhAROS components can be utilized to provide a method to generate compositional benchmarking for quality control, assurance and fraud detection. In one embodiment PhAROS components can be utilized to provide a method to generate target-oriented rational design. In one embodiment PhAROS components can be utilized to provide a method to test the hypothesis that across the vast geographical, cultural and historical datasets encompassed by the meta-pharmacopeia, rare, non-obvious, curative combinations of phytomedicines will have emerged at intervals.

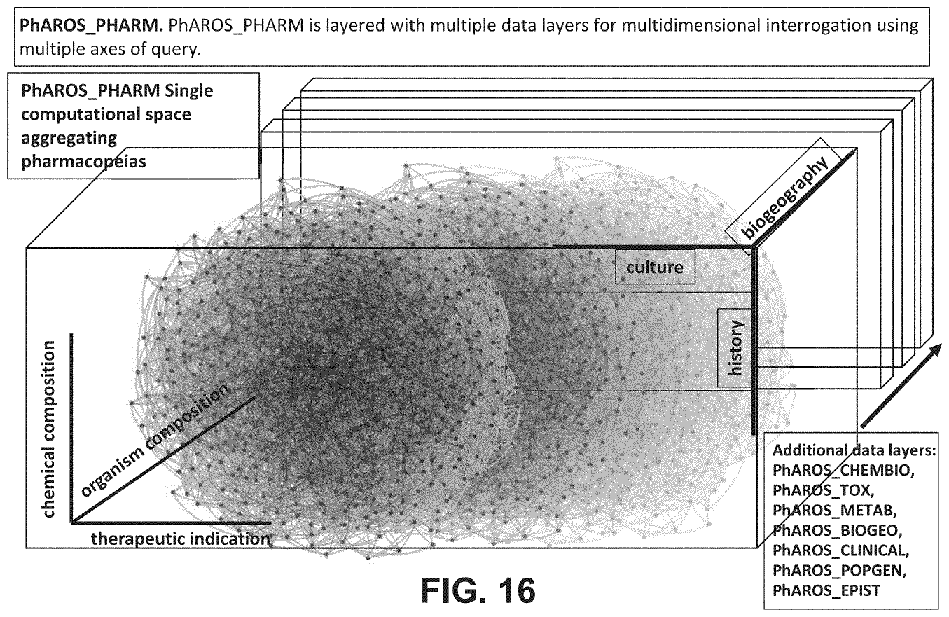

FIG. 16 shows for illustrative purposes only an example of PhAROS_PHARM of one embodiment. FIG. 16 shows a PhAROS_PHARM created from biogeography, culture, and history of non-western transcultural formulations and medical treatments for indications. The PhAROS_PHARM includes additional data layers PhAROS_CHEMBIO, PhAROS_TOX, PhAROS_METAB, PhAROS_BIOGEO, PhAROS_CLINICAL, PhAROS_POPGEN, and PhAROS_EPIST. The non-western transcultural formulations and medical treatments are processed into a PhAROS_PHARM single computational space aggregating pharmacopeias of the transcultural formulations. The PhAROS_PHARM includes for example chemical composition, plant composition, and therapeutic indication of the non-Western transcultural formulations and medical treatments for analysis in creating new formulations of one embodiment.

PhAROS in silico drug discovery engine has unique properties/claims. PhAROS includes multiple pharmacopeia in a single interrogatable space. PhAROS processes are for uncovering optimized therapeutic mixtures (OTM)/minimum essential mixtures (MEM). The PhAROS method is not looking for single ingredient-single target formulations or for whole plant medicine as in the traditional medical systems. The PhAROS method is using culturally-based epistemology to define the functional categories of necessary ingredients within these mixtures and salutogenesis: focusing on the promotion of health (rather than pathology).

PhAROS capabilities include identifying new drug-target-indication relationships for pre-clinical investigation and drug development; suggesting minimal essential phytomedical formulations for a given indication through filtering non-essential components; suggesting alternative, equivalent formulations for a given indication that provide for improved efficacy, decreased side effects or novel IP development; identifying alternate supply chain options for phytomedicine components; de-risking exploration of phytomedicines as therapeutic components by assessing their convergent emergence between geographically- and culturally-separated medical systems; de novo design of a new class of ‘transcultural’ medicines; and integrating phytomedical intelligence for a particular indication across geographically and culturally distinct pharmacopeias.

The embodiments show a method for creation of the meta-pharmacopeia PhAROS_PHARM. In some embodiments PhAROS contains a suite of informatics tools, data pipelines and data repositories allowing for user access and decision support tools for identifying a drug, a compound, a mixture, an organism discovery. Depending on the need of the user data repositories, and pre-processed repositories, can be cross correlated, analyzed and assessed for particular questions, these sub components and data sets, include but are not limited to.

PhAROS_USER. This is the user interactive system including but not limited to functional user tools designed to aid in coordinating user defined in silico analysis across multiple sub repositories and tools, coordinating with PhAROS_CORE, to utilize processes, connect and retrieve data and present user requested data, in an accessible manner. Basic and administrative levels of access limit possible disruption of data resources and tools.

PhAROS_CORE. This is the core system of functional system including but not limited to tools designed to collect, parse and maintain sub-systems, raw data repositories, pre-processed repositories, training data, data tools, automated and manual processing and task management.

PhAROS_PHARM. This is a proprietary pre-processed repository, and computational space, comprising and including but not limited to, the first ‘meta-pharmacopeia’, processed and normalized formalized pharmacopeias, formulations, associated plant/organisms, associated available compound sets, and indications, temporal and geographical data, indicating historical, and contemporary geographical, cultural and epistemology origins; Including but not limited to processed and normalized formalized pharmacopeias from Japan, China, India, Korea, South East Asia, Middle East, North/South America, Russia, India, Africa, Europe, Australia; Including but not limited to processed, translated normalized, individual relevant published datasets or case reports in the scientific literature that document relationships between medicinal plants and disease indications; Including, but not limited to processed, curated ethical partnerships, indigenous, cultural (e.g., African, Oceanic) phytomedical formulations; Including but not limited to processed contemporary and historical herbologies that document relationships between medicinal plants and disease indications (e.g., Hildegard of Bingen, Causae et Curae, Physica); Including but not limited to processed, translation of resources from original languages processed using approaches such as machine literal translation, natural language processing, multilingual concept extraction or conventional translation; OCR of historical materials. AI driven intent translation.

PhAROS_CONVERGE. This is a pre-processed repository including but not limited to, an un biased in silico convergence analysis of formulation composition explicitly between medical systems, predictions of minimal and/or essential compound sets for a given indication, a proprietary digital composition index (n-dimensional vector and/or fingerprint), identifying efficacy across traditional medicine systems, ranked optimized de novo formulations and mixtures utilizing transcultural components for subsequent preclinical and clinical testing in particular indications.

PhAROS_CHEMBIO. This is a pre-processed repository of chemical and biological data, including but not limited to chemical structure, physicochemical properties, known and/or algorithmically calculated or predicted PD/PK properties, putative biological effects, data informing of receptor binding, docking, regulation of signaling pathways, metabolism, drug-target relationships, and mechanism of action, CYP interactions, as well as published studies and clinical trials.

PhAROS_BIOGEO. This is a pre-processed repository of integrated data, including but not limited to the meta-pharmacopeia, associated temporally, geographical, botanical, climatological, environmental, genomic, metagenomic, and metabolomic data on originating plants, components or other organisms.

PhAROS_METAB. This is a pre-processed repository of integrated data of, including but not limited to, the meta-pharmacopeia with de novo metabolomic data for plants, and organisms that are not currently in medicinal use, supplemental metabolomic data secured for known medicinal plants and/or associated organisms.

PhAROS_MICRO. This is a pre-processed repository of integrated data of, including but not limited to, the meta-pharmacopeia with microbiome data on microorganisms associated with plants/organisms/components of interest, and their secondary metabolome compositions.

PhAROS_CURE. This is a pre-processed repository of integrated data, including but not limited to, the meta-pharmacopeia with documented spontaneous regression/remission events associated with botanical medicine or supplement usage, organized by organism, including plant, compound set and clinical manifestation/ICD codes.

PhAROS_QUANT. This is a pre-processed repository of integrated data of, including but not limited to, the meta-pharmacopeia with component weighting data based on either proportional components using standardized measurements and normalizations, for formulations and/or de novo quantitative analysis of formulated components.

PhAROS_POPGEN. This is a pre-processed repository of integrated data of, including but not limited to, the genetic admixtures, SNP characteristics and genetic/ethnic variability in populations in whom the formulations within the meta-pharmacopeia have been tested geographically and temporally.

PhAROS_TOX. This is a pre-processed repository of integrated data of, including but not limited to, the meta-pharmacopeia with toxicological and side-effect profile data, and/or de novo experimentally-derived data, and/or in silico predicted toxicological and side-effect data.

PhAROS_BH. This is a pre-processed repository of integrated data and a data processing/assessing tool, including but not limited to, contextualization data of meta-pharmacopeia datasets within a novel proprietary Bradford-Hill decision support framework, predicting data interpretation and assessing the evidence base for assertions of potential efficacy.

PhAROS_EPIST. This is a pre-processed repository of integrated data and a data processing/assessing tool, including but not limited to, parsed of formulation components data, plant, compound, a proprietary PhAROS correlation tool, that links composition to underlying epistemology for inclusion of a component (e.g., TCM/Kampo concept of JUN-CHEN-ZUO-SHI (‘Monarch, Minister, Assistant and Envoy’).

PhAROS_BRAIN. This is a repository of integrated data and a data processing/assessing tool, including but not limited to, a system that links the PhAROS_USER interactive system above to advanced analysis tools, PhAROS_BRAIN Functions which enable de novo analysis, as well as being able to populate PhAROS subsystems with data.

PhAROS_FLOW, a graphical data processing environment that allows users and administrators the ability to process data using the PhAROS_BRAIN functions without extensive coding, system modeling tools including machine learning and AI tools such as support vector machine, artificial neural networks, deep learning, Naïve Bayesian, K-nearest neighbors, random forest, AdaBoost wisdom of crowds and ensemble predictors, and others, validation (such as MonteCarlo cross-validation, Leave-One-Out cross validation, Bootstrap Resampling, and y-randomization).

The application relates generally to a method and system that can be used for the unbiased, user or artificial intelligence (AI) guided, identification of putative human and animal therapeutic targets, proof of mechanism, analysis of therapeutic potential of a compound, identification of complex mixtures for human on animal therapeutics, optimization of complex mixtures for human on animal therapeutics, supply chain.

This system utilizes the processing of large amounts of pharmacopeia data; data analysis, mathematical manipulation, machine learning identification, and other unique combinations of mathematical assessment of this data, through a user interface and user interaction to produce easily interpretable results and visualizations that inform the user of potential therapeutic targets for an indication, therapeutic potential of a compound, and formulations of new classes of transcultural medicines.

Lead identified compounds are subjected to further chemical modification processed to improve putative action, toxicity and availability, and are ultimately tested in human clinical trials. During the identification and testing of a new medicine for an indication, information and data on historical phytomedical approaches are generally ignored, overlooked, and/or over simplified, in favor of current computational analysis of fundamental single compound chemical analysis, based on structure, and comparison of said structure to other structures, and substructure components.

Pathways for potentially efficacious medicine to move from non-Western pharmacopeias to mainstream medicine are currently inadequate; relying on either painstaking, high cost, compound-by-compound testing in Western preclinical and clinical efficacy paradigms, or on ‘rediscovery’ of components during high-throughput screening in academic or pharmaceutical industry research settings. Moreover, since non-Western medical systems are inherently polypharmaceutical and Western approaches are typically ‘single drug-single target’, simple preclinical or clinical screening will miss compounds that only work when contextualized by other components. Non-Western pharmacopeias are also highly siloed along cultural dividing lines, and tend to be examined in isolation by scientists from the originating country. This misses opportunities to identify consonant approaches that are duplicated across pharmacopeias, which could help pre-validate drug-target-indication relationships. In addition, it misses a major opportunity to combine efficacious components across cultural lines to design optimal new polypharmaceutical medicines.

These historical phytomedical approaches have spanned all human geographies, cultures and civilizations, across thousands of years, and although most have not been tested in any formalized setting, they have most likely been tested on enormous numbers of individuals, to produce effective therapeutics without strict scientific method, but rather Monte Carlo methods, using empirical and observational evidence over significantly longer time periods than current de novo compounds are tested in clinical trials. Much of this historical phytomedical information has become formalized pharmacopeias and have evolved and coalesced in many geographically isolated societies.

The majority of historical phytomedical compositions are organized into multi-ingredient formulations, and are usually based on collections of whole plant components rather than single chemical compounds, as the means to purify and identify such components has only become available in the last few hundred years. At this level the composition is often hundreds or thousands of individual chemical components. Moreover, the underlying epistemologies for inclusion of some components may have no parallel in an evidence-based medicine approach, rather reflecting a response to a belief system grounded in regional religion, superstition or myth.

The systems and methods described here as the PhAROS discovery platform for computational phyto-pharmacology is designed not to assess solely the identified chemical components (many of which are missing), in each traditional medicine versus a symptom or indication, as would be usually found in contemporary assessment systems. Rather the PhAROS discovery platform is designed to assess and analyze the entire epistemological framework for a traditional medicine, the prescribing and development of indication-prescription relationships, and utilizes assumptions and anachronistic knowledge cross-correlated across other geographically and temporally evolved traditional medicines.

This knowledge given in isolation may appear to have no significant utility, interest or translatability in modern medico-pharmacological development; however analysis across these systems can present clear decision support frameworks that incorporate the epistemological basis for syndrome differentiation and design of formulations and uses an unbiased methodology for validation and inclusion/exclusion criteria of components in formulations.

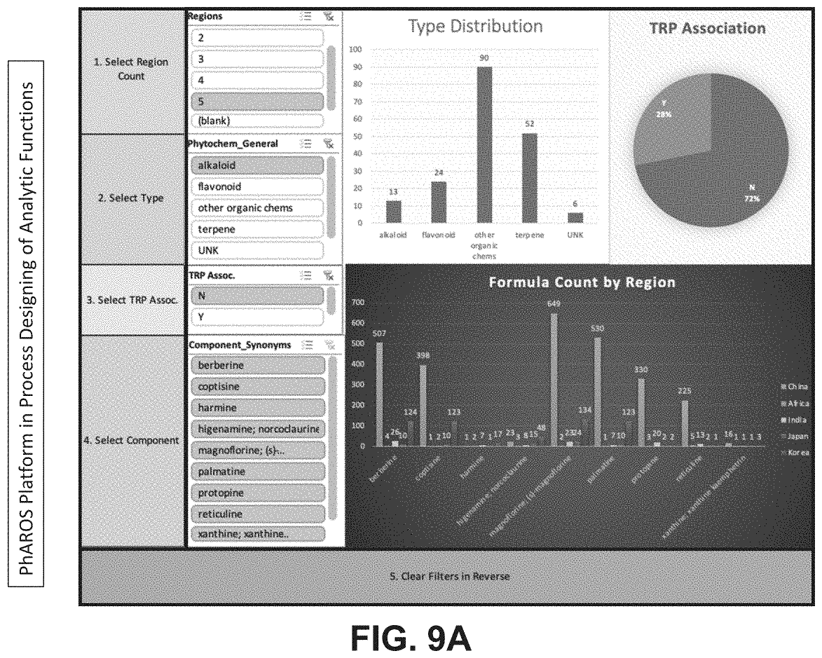

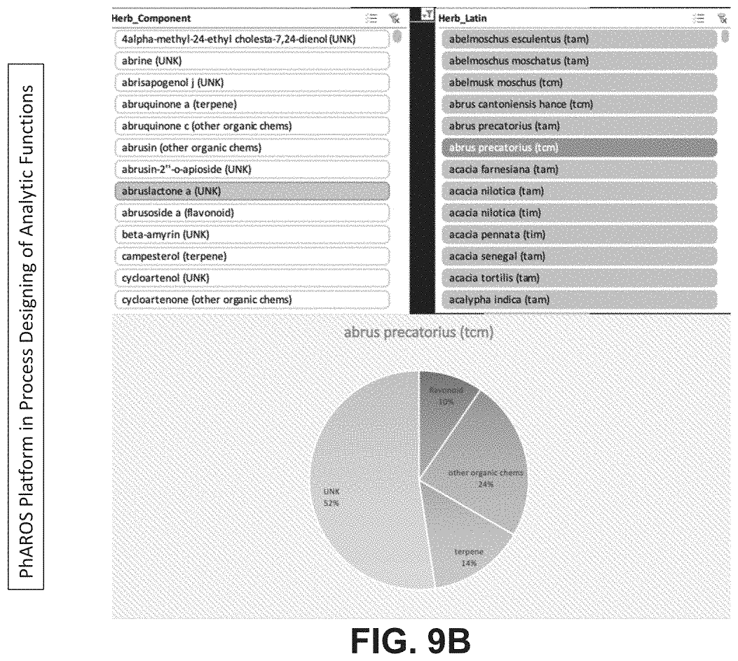

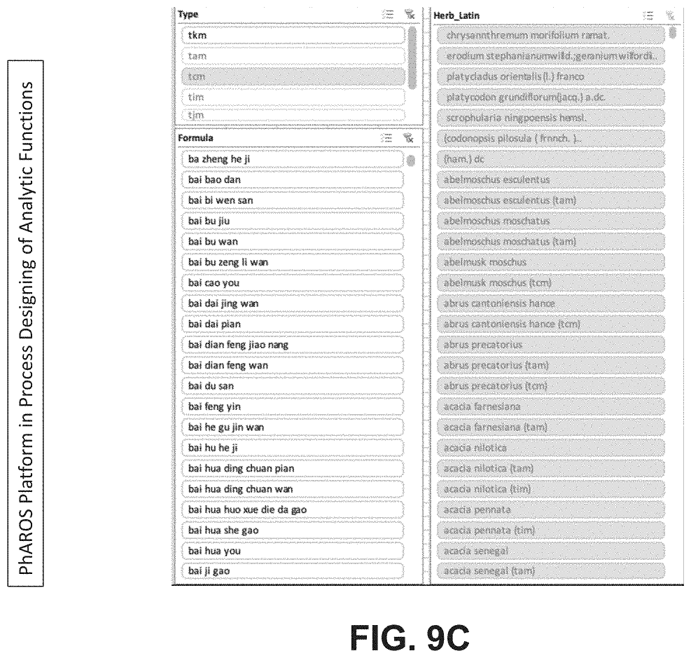

FIG. 9A-9C shows for illustrative purposes only, some in-process examples of the utility of the PhAROS platform for Drug Discovery through ease of in-process design of novel queries. FIG. 9A-9C shows the PhAROS_USER interaction dashboard with user selected features graphically displayed. FIG. 9A provides an in-process view of using the PhAROS platform to select geographical regions, type of phytochemical compounds, TRP Assoc., components, etc. for use in novel drug discovery activities. FIG. 9B. shows in process views of convergent compounds from Multiple TMS within a specific plant, Abrus precatorius. The user selected convergent compounds are shown for the example with Abrus precatorius showing a percentage pie chart of types of the selected components with this plant. This allows the user to change selections as part of their evaluation of the convergent components of one embodiment. FIG. 9 shows in-process views of interrogations of multiple TMS based on the specific Traditional Medicine formulas in the PhAROS_PHARM database in one embodiment.

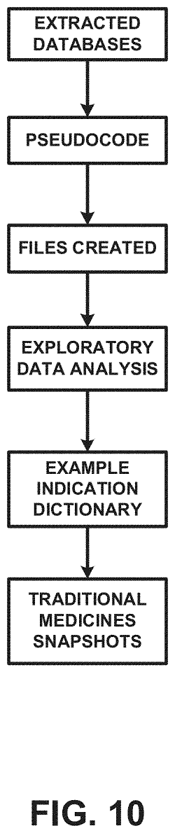

FIG. 10 shows for illustrative purposes only an example of extracted databases processing of one embodiment. FIG. 10 shows extracted databases processing. The data from the extracted databases of traditional medicines are assigned a series of pseudocode identifiers. The pseudocode identifiers are used to label the files created. Initial exploratory data analysis is performed and the exploratory data analysis is added to an example indication dictionary. The data assembled is used to create traditional medicines snapshots to provide users a brief synopsis of each traditional medicine of one embodiment.

In some embodiments, the PhAROS system enables organized input, processing and output matrices for specific types of stakeholder, allowing them to interface with, and interrogate the PhAROS system, enabling processing of data, retrieval of data, additional metadata, information, statistical analysis, and visualizations, that allows the user/stakeholder degrees of confidence in possible therapeutic potential of identified plant, organisms, compounds, mixtures, and mixture components, allowing rapid decision priorities to be made. Production of data for a given stakeholder can be achieved through either i) Administrative access to the system on behalf of the stakeholder, ii) Direct but limited access to the system as a user by the stakeholder, or iii) Direct unlimited access to the system as a user/administrator.

In some embodiments, the stakeholder has a starting point or asset with which they wish to initialize data analysis across the PhAROS system. Depending on the input type/data and quality, and the output required by the stakeholder, different components of the PhAROS system can be utilized in combination, and/or individually to produce the desired results needed by the stakeholder.

In some embodiments, the user/stakeholder has a Plant or organism name input. In such an embodiment, the PhAROS system can deliver, relevant data about this plant or organism, including but not limited to, the Chemical component list/metabolome (curated and machine readable); corresponding targets, regulated pathways, known actions, binding/docking properties; associated toxicity data, side effect, adverse event data; corresponding indications (including by convergence analysis, see below); any associated spontaneous regressions; geographical distribution and associated bio, environmental, climate data; associated microbiomes; modified Bradford-Hill decision support analysis for development.

In some embodiments, the user/stakeholder has an indication or disease input. In such an embodiment, the PhAROS system can deliver, relevant data about this indication or disease input, including but not limited to, transcultural alternative formulation datasets; predicted minimal essential component lists for indications with associated targets, actions, binding/docking properties, toxicity data, side effects, adverse event data; a plant list and/or metabolome list for component sourcing; weighed analysis for component prioritization and ranking; modified Bradford-Hill decision support analysis for development.

In some embodiments, the user/stakeholder has a metabolome input. In such an embodiment, the PhAROS system can deliver, relevant data about this metabolome input, including but not limited to, corresponding targets, regulated pathways, known actions, binding/docking properties; associated toxicity data, side effects profiles, adverse event data; indications; alternative plant and/or metabolome list.

In some embodiments, the user/stakeholder has a formulation or mixture component list input. In such an embodiment, the PhAROS system can deliver, relevant data about this formulation or mixture component list input, including but not limited to, a chemical component list; corresponding targets, regulated pathways, known actions, binding/docking properties; associated toxicity data, side effect data; plant list and/or metabolome list for component sourcing; epistemological analysis of component rationales; indications; weighting analysis for component prioritization; alternative formulations from different cultural contexts; predicted minimal essential component list for indications.

In some embodiments, the user/stakeholder has a chemical compound input. In such an embodiment, the PhAROS system can deliver, relevant data about this chemical compound input, including but not limited to, corresponding targets, regulated pathways, known actions, binding/docking properties; associated toxicity data, side effect, adverse event data; formulations; corresponding indications (including by convergence analysis, see below); epistemological analysis of rationales for inclusion in formulations; any associated spontaneous regressions; representation in metabolomes and/or plant/fungi lists for alternative sourcing; modified Bradford-Hill decision support analysis for development.

In some embodiments, the user/stakeholder has a target input. In such an embodiment, the PhAROS system can deliver, relevant data about this target input, including but not limited to, a compound list of known ligands of the target; target list for chemically similar compounds; their associated regulated pathways; list of formulations containing compounds predicted to interact with target, mapped to indications; source plants/fungi and/or metabolomes for compounds predicted to interact with target, binding/docking properties; associated toxicity data, side effect, adverse event data; formulations; corresponding indications, including by convergence analysis.

In some embodiments, the user/stakeholder has identified the need for a formulation. The PhAROS system can deliver a relevant formulation based on one or more inputs designated by the user/stakeholder. This PhAROS-informed formulation can include but not limited to, the following formation types: (A) minimal essential formulations derived from discriminating essential from non-essential components of traditional medicine formulations; (B) Transcultural de novo formulations assembled based on efficacy predictions from one or more traditional medicine approaches to a particular indication; (C) de novo formulations rationally designed based on PhAROS outputs across multiple traditional medicines; (D) A, B or C as a combination therapy with one or more additional components derived from Western pharmacopeias or drug discovery; or (E) bystander compounds or combinations identified through PhAROS analytics that have potential non-medical uses or applications.

In some embodiments, the user/stakeholder has identified the need for the PhAROS system to identify a formulation for real world uses that can include, but not limited to one or more of the following six: (1) human use pharmaceutical agents, (2) human nutraceuticals/supplements, (3) veterinary use pharmaceutical agents, (4) veterinary use nutraceuticals/supplements, (5) non-veterinary agricultural use, (6) Food additives, industrial and other uses.

In some embodiments, the user/stakeholder has identified the need for the PhAROS system to identify a formulation for use as a human pharmaceutical agent that can include, but not limited to, acute or chronic symptomatic disease management, disease and disorder treatment, or disease prevention.

In some embodiments, the user/stakeholder has identified the need for the PhAROS system to identify a formulation for use as a human nutraceuticals/supplements that can include, but not limited to, acute or chronic symptomatic disease management, disease and disorder treatment, disease prevention, human performance enhancement, or as an alternative to “non-natural” substances that would otherwise limit the user/stakeholder in being able to label their product as “natural”, “from nature”, “nature designed”, “all natural”, “no chemical additives” or similar statement.

In some embodiments the user/stakeholder has identified the need for the PhAROS system to identify a formulation for use as a veterinary pharmaceutical that can include, but not limited to, acute or chronic symptomatic disease management, disease and disorder treatment, disease prevention, or as an alternative to prohibited substances including most synthetic fertilizers and pesticides that would otherwise limit the farmer/grower in being able to label their product as organic. (i.e., at least 95% of animal feed must be grown to organic standards. No use of artificial fertilizers or pesticides on feed crops or grass is permitted.)

In some embodiments, the user/stakeholder has identified the need for the PhAROS system to identify a formulation for use as a veterinary nutraceuticals/supplement that can include, but not limited to, acute or chronic symptomatic disease management, disease and disorder treatment, disease prevention, yield improvement, performance enhancement, or as an alternative to prohibited substances including most synthetic fertilizers and pesticides that would otherwise limit the farmer/grower in being able to label their product as organic. (i.e., at least 95% of animal feed must be grown to organic standards. No use of artificial fertilizers or pesticides on feed crops or grass is permitted.)

In some embodiments, the user/stakeholder has identified the need for the PhAROS system to identify a formulation for use as an agricultural product that can include, but not limited to, plant derived insecticides, plant derived prophylactic insecticides, herbicides, fungicides, anti-parasitics, or as an alternative to prohibited substances including most synthetic fertilizers and pesticides that would otherwise limit the farmer/grower in being able to label their product as organic (i.e., at least 95% of animal feed must be grown to organic standards. No use of artificial fertilizers or pesticides on feed crops or grass is permitted).

In some embodiments, the user/stakeholder has identified the need for the PhAROS system to identify a formulation for use as a food additive, or industrial and other use, including, but not limited to, Shellac, Waxes, Natural Gums, Resins, Coatings, Adhesives, Dyes, Fragrances, Preservatives, Biodegradable polymers, Repellents, Natural fibers or as an alternative to “non-natural” substances that would otherwise limit the user/stakeholder in being able to label their product as “natural”, “from nature”, “nature designed”, “all natural”, “no chemical additives”, or similar statement.

Efficacy-based research approaches have been proposed as more appropriate for traditional Chinese medicine (TCM) rather than attempting to fit the TCM into a Western mechanism-based research framework. Tang et al. (writing in the BMJ in 2006) hypothesized that the current Western model of research, of trying out unknown treatments in animals, is not suitable for studying treatments that have long been used in humans. In some embodiments the PhAROS system is able to answer this hypothesis syncretically, allowing a diversity of inputs and pathways to outputs that can start from efficacy-based a priori assumptions or mechanistic inquiry, rather than the laborious testing of unknown compounds in animals to yield only correlative evidence that a compound made be efficacious in human therapy or treatment.

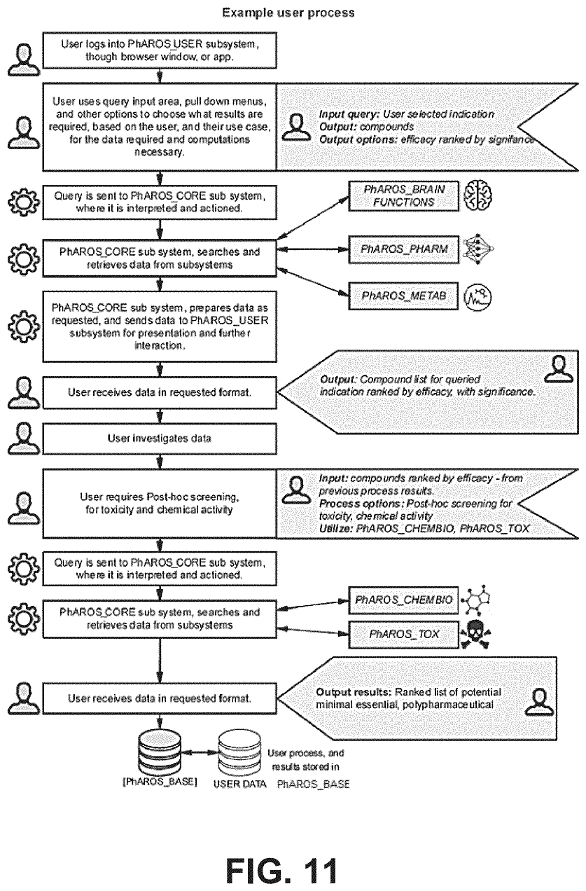

FIG. 11 shows for illustrative purposes only an example of an example of a user process with a PhAROS_METAB Subsystem of one embodiment. FIG. 11 shows an example user process with PhAROS_METAB. A user logs into PhAROS_USER subsystem, though browser window, or app. User uses query input area, pull down menus, and other options to choose what results are Input query: User selected indication required, based on the user, and their use case. Output: compounds for the data required and computations necessary. Input query: User selected indication with Output: compounds and Output options: efficacy ranked by significance. Query is sent to PhAROS_CORE sub system, where it is interpreted and actioned. PhAROS_CORE sub system searches and retrieves data from subsystems including PhAROS_BRAIN Functions, PhAROS_PHARM, and PhAROS_METAB. The PhAROS_CORE subsystem prepares data as requested, and sends data to PhAROS_USER subsystem for presentation and further interaction. User receives data in requested format. Output: compound list for queried indication ranked by efficacy, with significance.

The user investigates data. User requires Post-hoc screening, for toxicity and chemical activity Input: compounds ranked by efficacy—from previous results. Process options: Post-hoc screening for toxicity, chemical activity and utilize: PhAROS_CHEMBIO and PhAROS_TOX. A query is sent to PhAROS_CORE sub system, where it is interpreted and actioned. The PhAROS_CORE sub system searches and retrieves data from subsystems including PhAROS_CHEMBIO and PhAROS_TOX. The user receives data in requested format. Output results: Ranked list of potential minimal essential, polypharmaceutical. The user process and results are stored in PhAROS_BASE and USER DATA of one embodiment.

In some embodiments the PhAROS system can, using sub components of the system, perform in silico convergence analysis to identify minimal essential formulations of phytochemicals for specific indications. PhAROS uses algorithms within its PhAROS_BRAIN FUNCTIONS to perform a proprietary method called in silico convergence analysis (ISCA). In some embodiments the PhAROS system component PhAROS_METAB is utilized, in combination with PhAROS_USER, PhAROS_CORE. This is a pre-processed repository of integrated data of, including but not limited to, the meta-pharmacopeia with de novo metabolomic data for plants, and/organisms that are not currently in medicinal use, supplemental metabolomic data secured for known medicinal plants and/or associated organisms. Within this embodiment, PhAROS_METAB is interrogated with an indication through PhAROS_USER, and PhAROS_CORE, and a computational space is assembled where all compounds and their associated plants and formulations for that indication reside.

This dataset is then processed to identify compounds that have been arrived at as a consensus between one or more cultures, as within this convergent set are components with a significant likelihood of contributing to efficacy. Post-hoc screening using PhAROS_CHEMBIO, and PhAROS_TOX components then differentiates between bioactive or otherwise medically important (e.g., excipient) components, and excludes those that do not contribute to medicinal effects (e.g., plant structural molecules), thus the system can reduce complexity by minimizing duplication. The resulting ranked list of potential minimal essential, polypharmaceutical, mixtures can then be advanced through other PhAROS system components, and/or traditional discovery pipelines, but in a significantly de-risked fashion through the PhAROS_BRAIN FUNCTION ICSA methodology for component prioritization, and therapeutic potential indexing.

The PhAROS system has the ability to generate, de novo, transcultural ‘meta-medicines’ that hybridize evidence of efficacy across cultures, geography and time, to rationally design new poly-pharmaceuticals that are not obvious and do not pre-exist in the meta-pharmacopeia. In some embodiments the PhAROS system can undertake ‘divergence’ analysis. A significant method in de-risking components that are found in a limited subset of cultures, time periods or geographies, but have a significant likelihood of being efficacious.

In some embodiments, these plants, mixture components, and/or compounds are identified as candidates to supplement formulations from other settings or as components of novel proprietary formulations. This novel method illustrates a significant advantage over current methods, encompassing and leveraging the critical method of PhAROS' transcultural nature. That is that without analysis by the PhAROS system efficacious components that would have been limited to a particular non-Western pharmacopeia for reasons of geography, botany or environment, are now identifiable and available to supplement formulations from other locales and/or they can be contributory components to de novo proprietary and optimized formulations and mixtures.

In some embodiments, the PhAROS system can produce new formulations from convergence or divergence analyses, that are added to sub-component systems of the PhAROS system, and will join the extant formulations within the PhAROS meta-pharmacopeia to be part of a significantly large number of AI training and testing sets for AI and machine learning algorithms that are designed for prediction within the PhAROS_BRAIN subsystem.

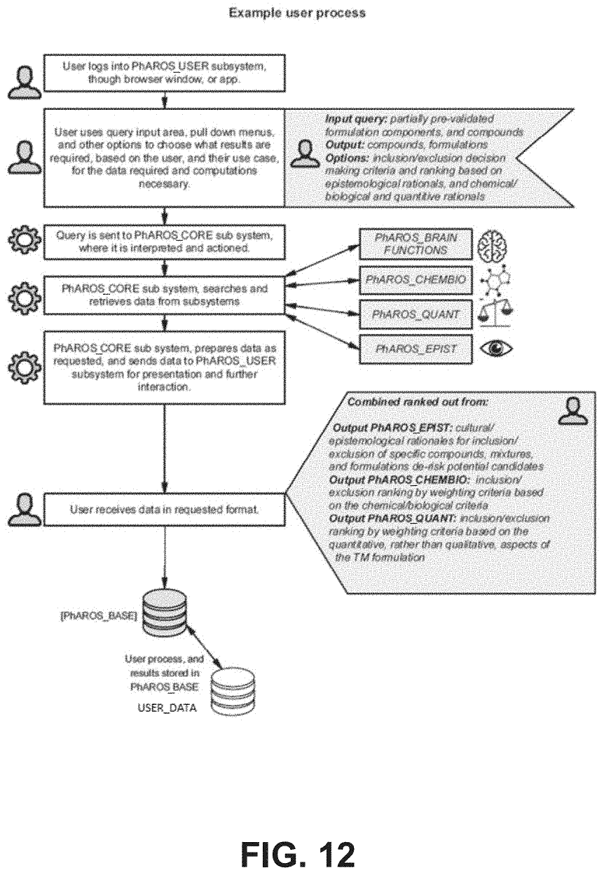

FIG. 12 shows for illustrative purposes only an example of an example of a user process with a PhAROS_EPIST Subsystem of one embodiment. FIG. 12 shows an example user process with PhAROS_EPIST. A user logs into PhAROS_USER subsystem, though browser window, or app. User uses query input area, pull down menus, and other options to choose what results are required, based on the user, and their use case for the data required and computations necessary.

Input query: partially pre-validated formulation components, and compounds. Output: compounds, formulations. Options: inclusion/exclusion decision making criteria and ranking based on epistemological rationales and chemical/biological and quantitative rationales. Query is sent to PhAROS_CORE sub system, where it is interpreted and actioned. PhAROS CORE subsystem, searches and retrieves data from subsystems including PhAROS_BRAIN, PhAROS_CHEMBIO, PhAROS_QUANT, and PhAROS_EPIST.

PhAROS_CORE sub system prepares data as requested, and sends data to PhAROS_USER subsystem for presentation and further interaction. User receives data in requested format. Combined ranked out from: Output PhAROS_EPIST: cultural/epistemological rationales for inclusion/exclusion of specific compounds, mixtures, and formulations de-risk potential candidates. Output PhAROS_CHEMBIO: inclusion/exclusion ranking by weighting criteria based on the chemical/biological criteria. Output PhAROS_QUANT: inclusion/exclusion ranking by weighting criteria based on the quantitative, rather than qualitative, aspects of the TM formulation. User process and results stored in PhAROS_BASE and USER DATA of one embodiment.

In some embodiments, the PhAROS system can, using sub components of the system, deconvole modes and mechanisms of action, generate inclusion priorities and underlying epistemology to identify minimal essential formulations of phytochemicals for specific indications. In some embodiments, the PhAROS system can contribute additional information to the transcultural pre-validation of formulations through convergence analysis, utilizing the PhAROS subsystems PhAROS_CHEMBIO, PhAROS_QUANT and PhAROS_EPIST, in combination with PhAROS_USER, PhAROS_CORE, and PhAROS_BRAIN FUNCTIONS.

In isolation and in combination these systems further de-risk potential candidates for further advancement through standard discovery pipelines. PhAROS sub-systems and methods include, but are not limited to, the PhAROS_CHEMBIO subsystem, is a pre-processed repository of chemical and biological data, including but not limited to chemical structure, physicochemical properties, known and/or algorithmically calculated or predicted PD/PK properties, putative biological effects, data informing of receptor binding, docking, regulation of signaling pathways, metabolism, drug-target relationships, mechanism of action, CYP interactions, as well as published studies and clinical trials. Using these system potential targets can be assessed and modes and mechanisms of action for candidates that are being evaluated for inclusion in, or exclusion from, minimal essential formulations can be identified.

Additional use of PhAROS_QUANT provides a second dimension to the inclusion/exclusion decision making by incorporating weighting criteria based on the quantitative, rather than qualitative, aspects of the TM formulation. PhAROS_QUANT is a pre-processed repository of integrated data of, including but not limited to, the meta-pharmacopeia with component weighting data based on either proportional components using standardized measurements and normalizations, for formulations and/or de novo quantitative analysis of formulated components.

Finally, implementing PhAROS_EPIST in this pipeline identifies cultural/epistemological rationales for inclusion/exclusion decisions which can be used to further discriminate necessary from likely unimportant components. PhAROS_EPIST is a pre-processed repository of integrated data and a data processing/assessing tool, including but not limited to, parsed of formulation components data, plant, compound, a proprietary PhAROS correlation tool, that links composition to underlying epistemology for inclusion of a component of one embodiment.

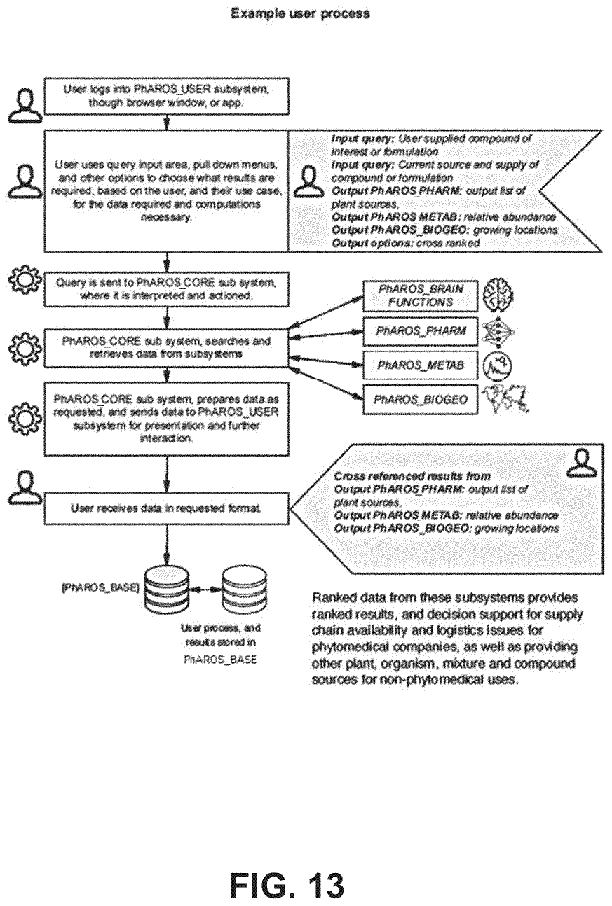

FIG. 13 shows for illustrative purposes only an example of an example of a user process with a PhAROS_BIOGEN Subsystem of one embodiment. FIG. 13 shows an example a user process with PhAROS_BIOGEN. A user logs into PhAROS_USER subsystem, though browser window, or app. User uses query input area, pull down menus, and other options to choose what results are Input query: User selected indication required, based on the user, and their use case. Output: compounds for the data required and computations necessary. Input query: user supplied component of interest or formulation. Input query: current source and supply of compound or formulation. Output PhAROS_PHARM: output list of plant sources. Output PhAROS_METAB: relative abundance. Output: PhAROS_BIOGEO: growing locations and Output options: cross ranked.

Query is sent to PhAROS_CORE sub system, where it is interpreted and actioned. PhAROS_CORE subsystem, searches and retrieves data from subsystems including PhAROS_BRAIN Functions, PhAROS_PHARM, PhAROS_METAB, and PhAROS_BIOGEN. PhAROS_CORE subsystem prepares data as requested, and sends data to PhAROS_USER subsystem for presentation and further interaction. User receives data in requested format.

Cross referenced results from output PhAROS_PHARM: output list of plant sources, Output PhAROS_METAB: relative abundance and Output PhAROS_BIOGEN: growing locations. The user process and results are stored in PhAROS_BASE and USER DATA. Ranked data from these subsystems provides ranked results and decision support for supply chain availability and logistics issues for phytomedical companies, as well as providing other plant, organism, and mixture and compound sources for non-phytomedical uses of one embodiment.

In some embodiments, the PhAROS system can, using subcomponents of the system, provide a method to diversify the supply chain of a user/stakeholder for phytomedicine plants, organisms, components and/or compounds. In citations where phytomedicines are limited by supply chain issues, there are multiple methods to alleviate supply of these components, including total synthesis, bioreactor approaches and alternate sourcing.

In some embodiments the PhAROS system and PhAROS sub-components can be used to inform on alternate sources of these components through interrogation of the PhAROS_PHARM sub-system, in combination with PhAROS_USER, PhAROS_CORE, and PhAROS_BRAIN, with a compound of interest or formulation and the generation of an output list of plant sources. In some embodiments this data can be used to integrate the PhAROS_METAB sub-system, and metabolomic data can be assessed (where available) or commissioned to evaluate for relative abundance of the compound of interest. Alternative sources of compounds of interest can then be evaluated for commercial viability. Supply chains may also be impinged, and therefore subject to availability by specific geographical, climatological, seasonal or environmental limitations if the most recognized sources of a particular phytomedical compound are associated with specific locations and seasons.

In some embodiments, the PhAROS_BIOGEO sub-system can be utilized as a method for analysis of growing conditions, in combination with a GIS framework, in order to identify new viable growing locales for plant sources of specific compounds, and alleviate supply chain availability issues. The resulting data from PhAROS, and these subsystems, will provide decision support for supply chain availability and logistics issues for phytomedical companies, as well as providing other plant, organism, mixture and compound sources for non-phytomedical uses.

The PhAROS processing pathway is utilized to provide a method to rationalize phytomedicine design and cultivation pipelines for global health issues. In some embodiments the PhAROS system can, using subcomponents of the system, provide a method to rationalize phytomedicine design and cultivation pipelines for global health issues. Phytomedicines remain as major components of medical optionality for billions of individuals in rural, developing or impoverished locations worldwide. There exists continued advocacy for equitable distribution of Western medicines, and additionally there is not only an economic exigency but an ethical responsibility to optimize formulation and improve availability and access of low cost phytomedicine alternatives to comparatively expensive Western medicines, for global health populations and rationally leverage their potential benefits.

In some embodiments, the PhAROS system can, using subcomponents of the system, provide a method to aid in democratization of optimized phytomedicines, that can also serve populations by decreasing the influence of fraudulent practitioners and eliminating the perceived need for medically-irrelevant exploitative, and sometimes abhorrent, formulation components. PhAROS systems can inform global health solutions using methods in specific sub-systems, by (1) identifying minimal essential formulations for efficacy and safety through combining data results from PhAROS_METAB, and PhAROS_CHEMBIO, and subsequently utilizing the PhAROS_BIOGEO subsystem to identify plant, mixture, component and/or compound sources, for desired formulations and matching them to growing locations, environments and seasons, to generate cultivation plans for practitioners and community members.

In one embodiment, the PhAROS processing pathway is utilized to provide a method to rationalize phytomedicine design and cultivation pipelines for global health issues. The PhAROS processing pathway is utilized to provide a method to generate compositional benchmarking for quality control, assurance and fraud detection.) In some embodiments the PhAROS system can, using subcomponents of the system, provide a method to generate compositional benchmarking for quality control, assurance and fraud detection. Currently the primary method by which medicinal approaches move from non-Western to Western settings is via their translation into nutraceuticals. Unfortunately, this nutraceuticals market is plagued by abstraction and oversimplification of formulations that nevertheless claim fidelity to the original formulation-indication relationship found in the non-Western system.

In some embodiments, the PhAROS subsystems provide the tools and methods necessary to inform the rational design of high-quality formulations for nutraceuticals that legitimately contain the minimal essential ingredient set that PhAROS identifies with the highest efficacy. This improves products produced by PhAROS stakeholders/users within the nutraceuticals industry, and significantly reduces the negative health impacts and reduced unnecessary expenditures. In addition, the PhAROS subsystems provide the tools and methods to provide a set of compositional benchmarks related to claimed indications, for consumer/industry validation of products. These benchmarks support quality and integrity of nutraceuticals and provide a validation, quality assurance index/mark/certification linked to the PhAROS system.

In some embodiments, the PhAROS processing pathway is utilized to provide a method to generate compositional benchmarking for quality control, assurance and fraud detection. In some embodiments, the PhAROS system can, using subcomponents of the system, provide a method to generate a target-oriented rational design. This is true in cases where novel information about emerging diseases (e.g., Zoonosis) can be timely and important. In some embodiments, the PhAROS system can provide a method to generate novel disease-target relationships to be used for target-oriented rational design.

An example of the potential impact of this kind of approach is illustrated by recent studies in which an enzyme key to the functioning of non-COVID 19 (but related) coronaviruses (SARS-CoV and MERS-CoV) which was identified as structurally conserved with SARS-CoV2. 3D homology modeling of the enzyme was utilized and screened against a medicinal plant library containing 32,297 individual potential anti-viral phytochemicals/traditional Chinese medicinal compounds; this resulted in 9 potential hits for further exploration. In some embodiments, the PhAROS systems would replicate these types of analyses at a much larger scale and with the additional aspect and method of utilizing an extremely large transcultural and transhistorical meta-pharmacopeia dataset as a starting point.

In some embodiments, the PhAROS processing pathway is utilized to provide a method to generate target-oriented rational design. In some embodiments the PhAROS system can, using subcomponents of the system, provide a method to test the hypothesis that across the vast geographical, cultural and historical datasets encompassed by the meta-pharmacopeia, rare, non-obvious, curative combinations of phytomedicines will have emerged at intervals. These may be manifested in historical and religious records and in modern literature/anecdotal reports including those on ‘spontaneous’ regressions/remissions where individuals and patients documented concurrent or prior use of alternative medicines associated with phytomedicine use.

In some embodiments, the PhAROS_CURE subsystem utilizes a set ethnographical, text mining and statistical analyses to evaluate connections between phytomedicines and regressions or curative events. In some embodiments, the data produced from the PhAROS_CURE subsystem can cross correlate with data from the PhAROS_METAB subsystem and PhAROS_CHEMBIO subsystem, which produces a method to then identify commonalities and potential candidates for further investigation.

In some embodiments, the PhAROS processing pathway is utilized to provide a method to test the hypothesis that across the vast geographical, cultural and historical datasets encompassed by the meta-pharmacopeia, rare, non-obvious, curative combinations of phytomedicines will have emerged at intervals.

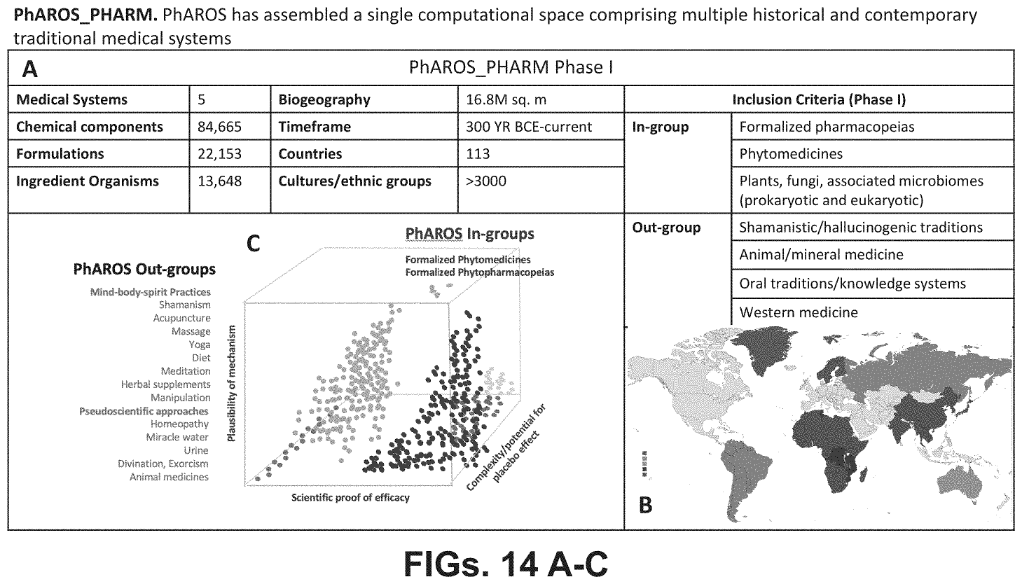

FIGS. 14-21 show for illustrative purposes only an overview of PhAROS, rationales and the conceptual basis for Transcultural formulations, Convergence analysis, Minimal essential formulations, and Clinical indication dictionaries.

FIG. 14 shows for illustrative purposes only an example of Metrics of the PhAROS computational space of one embodiment. Here, PhAROS is assembled in a single computational space comprising multiple historical and contemporary traditional medical systems. FIG. 14A summarizes the content and features of the PhAROS_PHARM proprietary data set. FIG. 14B show inclusion Criteria for Phase I development of PhAROS, including a schematic map summarize the included and excluded features of TMS in the PhAROS_PHARM proprietary data set. FIG. 14A shows a schematic representation in-group and out-group TMS features used to decide inclusion in PhAROS of one embodiment.

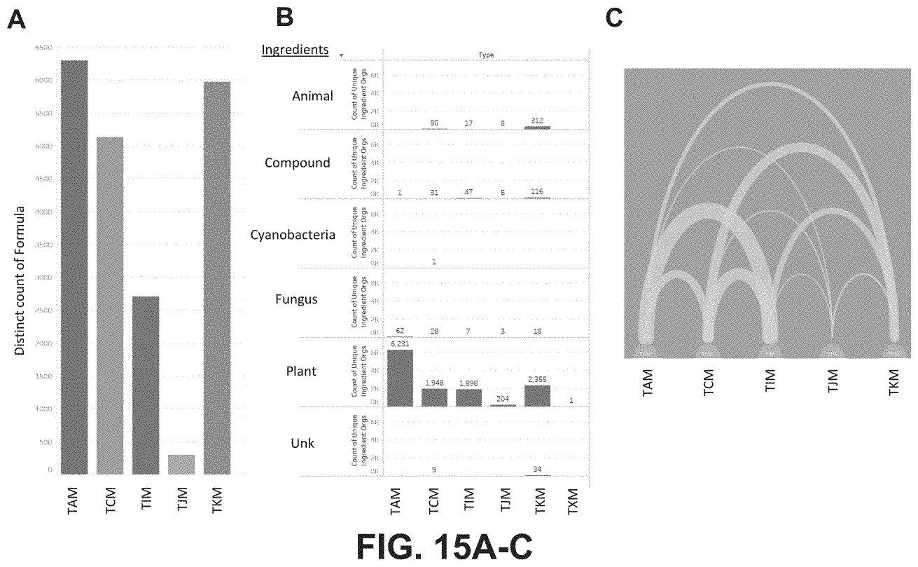

FIG. 15 shows for illustrative purposes only characterization of PhAROS computational space of one embodiment. FIG. 15A shows characterization of PhAROS computational space, including formula count by TMS. FIG. 15B shows characterization of PhAROS computational space, including ingredient organism type by TMS. FIG. 15C shows characterization of PhAROS computational space using a chord diagram representation of shared ingredient plants by occurrence in indicated TMS of one embodiment.

FIG. 16 shows for illustrative purposes only an example of a Schematic architecture of one embodiment. Overall, PhAROS includes analyzing data from a plurality of traditional medicine systems (TMS), where the analysis used transcultural dictionaries to allow searches within distinct TMS data sets embodying different epistemologies and terminologies, and where the analysis uses data returned by a query to identify alternative polypharmaceutical and/or optimized polypharmaceutical compositions. As shown in FIG. 16, the schematic architecture for PhAROS_PHARM is layered with multiple data layers for multidimensional interrogation using multiple axes of query. For example, additional data layers used in PhAROS include, without limitation, Additional data layers: PhAROS_CHEMBIO, PhAROS_TOX, PhAROS_METAB, PhAROS_BIOGEO, PhAROS_CLINICAL, PhAROS_POPGEN, and PhAROS_EPIST, among others. Schematic architecture of the PhAROS in silico drug discovery platform of one embodiment.

FIG. 17 shows for illustrative purposes only an example of a concept underlying Transcultural Formulations of one embodiment. This schematic explains the underlying hypothesis and drivers for the development of transcultural formulations. The hypothesis was that PhAROS could be used to improve on existing TMS formulations by aggregating knowledge across cultures, biogeographies and time. FIG. 17 shows an example for the anti-malarial Artemisinin. This set of maps shows overlap and disconnect in the geographies of medical need (global incidence of malaria), supply (the biogeographical distribution of the source plant Artemisa annua) and the limited number of TMS that utilize Artemisia an anti-fever and anti-malarial medications. The TMS reflect local flora and local disease burdens (FIG. 17). PhAROS is applicable here because PhAROS can abrogate these boundaries and integrate knowledge from biologically, geographically, culturally, or temporally separated contexts to build novel medicines. PhAROS outputs include: TAM: sexual incapacity, sexual asthenia, frigidity, aphrodisiac, erectile dysfunction, impotency; TIM: malaria and fever; and TCM: Zhou Hou Bei Ji anti-malarial (first identified 11thC, Nobel Prize in 1971).

FIG. 18 shows for illustrative purposes only an example of a PHAROS_CONVERGE of one embodiment. FIG. 18 shows Figure D. PHAROS_CONVERGE. The concept underlying in silico convergence analysis. This schematic representation illustrates the concept of de-risking translation of phytomedical therapies from TMS to Western pipelines through identifying commonalities in approaches from biogeographically and culturally separated locales of one embodiment. Here, convergence: commonalities are de-risked/pre-validated for entry into drug development pipeline (See FIG. 18). In addition, divergence region-specific solutions that can be included in de novo designed formulations that overcome biogeocultural boundaries are included in the analysis (see FIG. 18).

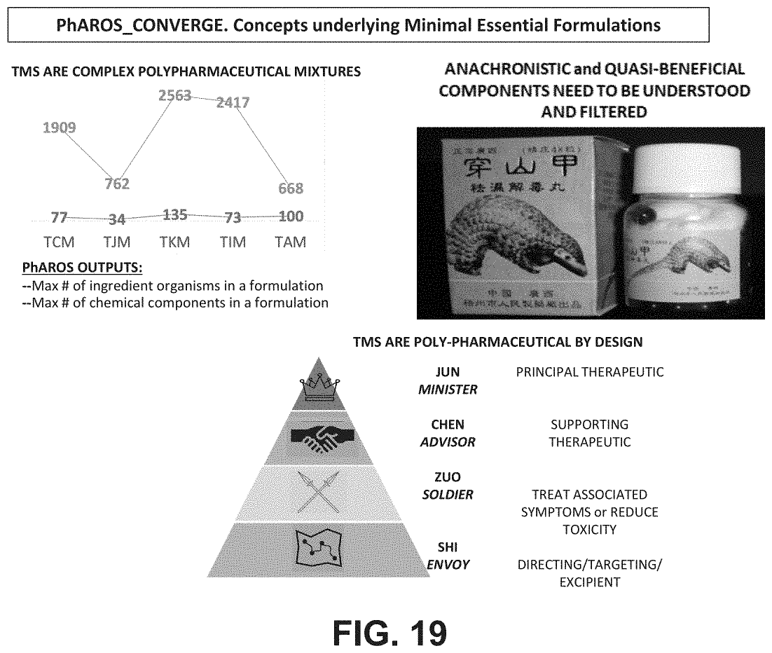

FIG. 19 shows for illustrative purposes only an example of a Minimal Essential Formulations of one embodiment. PhAROS_CONVERGE. The concept underlying Minimal Essential Formulations. This schematic representation illustrates the concept of reducing complexity of TMS polypharmaceutical preparations to identify minimal essential efficacious components that are candidates for translation from TMS to Western discovery pipelines of one embodiment. TMS are complex polypharmaceutical mixtures. Sometimes they contain anachronistic and quasi-beneficial ingredients that we sort out of the database. The Minimal Essential Formulations are guided by the principals of Jun, Chen, Zuo, and Shi (Minister, Advisor, Soldier, and Envoy), which translates to therapeutic mixtures that in practice contain a principal and a supporting therapeutic, as well as ingredients to treat associated side effects/symptoms or reduce toxicity and finally, ingredients that help with delivery of the drug mixture.

As shown in FIG. 19, the aim was to test if complexity of TMS polypharmaceutical preparations can be reduced to identify minimal essential efficacious components that are candidates for translation from TMS to Western discovery pipelines.

FIG. 20 shows for illustrative purposes only an example of PhAROS_PHARM machine learning of one embodiment. This correlation analysis performed by machine learning on the PhAROS computational space reflects high co-occurrence of major chemical types in phytomedicine, reflecting the need for simplification.

FIG. 21 shows for illustrative purposes only an example of indication dictionaries of one embodiment. Here, the aim was to use indication definitions embedded in TMS reflect modern and historical terminology, Western and non-Western epistemologies to identify of novel convergent formulation components. The approach was to generate indication dictionaries for database filtering and as features for subsequent AI/ML that reflect the knowledge systems underlying diagnosis.

In particular, FIG. 21 shows a schematic explaining that the dictionaries used to interrogate PhAROS reflect modern and historical terminology, Western and non-Western epistemologies embedded in TMS. The dictionaries are used for database filtering and as features for subsequent AI/ML. Without the clinical indication dictionaries, it would be impossible to interrogate across the cultural boundaries in many instances because different cultures use unique terms to describe clinical symptoms and disorders. Some search terms like PAIN translate fairly easily across cultural boundaries, but terms like MIGRAINE are much more varied in their clinical descriptions across cultures.

### Additional Considerations

Traditional Chinese Medicine (TCM). Mental and physical practices as well as phytomedicine, animal and mineral remedies, the Doctrines of Chinese medicine are rooted in cosmological concepts such as yin-yang and five phases known as water, wood, fire, earth and metal. TCM describes health as the harmonious interaction of these entities and the outside world, and disease as a disharmony. TCM diagnoses trace symptoms to patterns of the underlying disharmony, by measuring the physiological indicators. TCM was developed over ˜3500 years with standardization efforts from 1950s onwards in the People's Republic of China.

Kampo medicine. Kampo is a component of medical practice in contemporary Japan that has its origins in Chinese medical practices first developed in the Han Dynasty (206 BC-AD 220). The medicines and associated practices were first introduced to Japan via Korea in the seventh to ninth centuries AD, with a subsequent influx of Chinese medical practices beginning in 1498. Though Kampo shares many elements with Traditional Chinese Medicine, it also developed into a uniquely Japanese practice between the two periods of Chinese introduction and subsequent to Japan cutting off contact with outsiders in 1630 CE. During the Meiji Restoration, Kampo fell out of favor due to being perceived as not modern, and the Japanese government adopted German medical practice as the country's standard. After the end of the second world war, Kampo underwent a renaissance in popularity. In 1976, it was included in the Japan National Insurance Program, and today it is taught in all Japanese medical schools alongside Western biomedicine.

Ayurveda. Ayurveda (Mukherjee et al., 2017) is an Indian medical system, based around epistemology of three energies (doshas): Vata is the energy of movement; pitta is the energy of digestion or metabolism and kapha is the energy of lubrication and structure. The cause of disease in Ayurveda is viewed as a lack of proper cellular function due to an excess or deficiency of vata, pitta or kapha. Disease can also be caused by the presence of toxins. Balance in constitution is ideal and the natural order; imbalance is disorder. Health is order; disease is disorder. Ayurvedic therapeutic approaches include phytomedicine, meditative practices, physical manipulation, diet, environment.

Traditional European medicine. Medicine in pre-Enlightenment Europe was a combination of elements derived from Greek and Roman medical writings, acquired through translations from Greek and Arabic sources, along with a mix of relatively poorly documented indigenous practices. The more systematic of these practices were largely based on humourism, which is the belief that disease is caused by imbalance among the four “humours” (blood, phlegm, yellow bile, and black bile). Humoural medicine sought to treat disease symptoms by inducing symptoms (often with extreme methodologies such as purging and bloodletting) seen as opposite to those of diseases rather than treating the underlying causes. Disease was viewed as caused by an excess of one humour and thus would be treated by inducing its opposite, however damaging.

Unani is an Arab-Persian medical system also practiced widely in India. It is focused on prevention of disease and is similar to early European medicine in its idea of imbalances between fundamental humours. It focused on three therapeutic paths: Izalae Sabab (elimination of cause), Tadeele Akhlat (normalization of humours) and Tadeele Aza (normalization of tissues/organs).

Islamic medicine greatly informed the development of Western medicine through the dissemination of its essential texts, especially via the Ottoman Empire, and promotes holistic approaches to health as well as a ground-breaking emphasis on public hygiene and the authentication of phytomedicines.

Allopathic Western medicine. Strongly influenced by Greek philosophy and Arab/Islamic medicine prior to 1500, Allopathic Western medicine developed an increasingly evidence-based framework from the Renaissance through enlightenment and the industrial age. Allopathic Western medicine is science-based, modern medicine, that uses medications or surgery to treat or suppress symptoms or the ill effects of disease. Allopathic Western medicine utilized an evidence-based regulatory framework that demands a continuum of proofs of mechanism and efficacy prior to delivery.

A full discussion of timelines, geographies, and the complexities of comparing medical systems cross-culturally is beyond the scope of this disclosure, but Leonti and Verpoorte (2017) (Leonti and Verpoorte, 2017) includes an excellent recent review of geographic and temporal influence of different medical traditions on each other. See also Etkin, Baker, and Busch (2008) (Etkin et al., 2008) and Etkin (2006) (Etkin, 2006) for discussion of cultural factors influencing therapeutic practice, and Leslie (1998) (Leslie, 1998) on comparative study of Asian medical systems.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Embodiments are in particular disclosed in the attached claims directed to a method and a computer program product, wherein any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter which can be claimed comprises not only the combinations of features as set out in the disclosed embodiments but also any other combination of features from different embodiments. Various features mentioned in the different embodiments can be combined with explicit mentioning of such combination or arrangement in an example embodiment. Furthermore, any of the embodiments and features described or depicted herein can be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

### 6. EXAMPLES

### Example 1. Proof-of-Concept Demonstration of in Silico Convergence Analysis: Pain

In this example, PhAROS was used to identify novel convergent formulation components for pain. In particular, PhAROS was used to discover polypharmaceutical medicines for treating pain by analyzing, in a single computational space, data from a plurality of traditional medicine systems (TMS), where the analysis used transcultural dictionaries to allow searches within distinct TMS data sets embodying different epistemologies and terminologies, and where the analysis uses data returned to a query (i.e., “pain”) to identify new polypharmaceutical and/or optimized polypharmaceutical compositions.

Data analysis included a subset of the Inputs as described in FIG. 8. We analyzed, in a single computational space, data from a plurality of traditional medicine systems (TMS), including normalized formalized pharmacopeias from one or more geographic regions associated with TMS (i.e., PhAROS_PHARM) and meta-pharmacopeia, associated temporally, geographical, botanical, climatological, environmental, genomic, metagenomic, and metabolomic data on originating plants, components or other organisms (i.e., PhAROS_BIOGEO).

As part of the analysis, transcultural dictionaries that collate Western and non-Western epistemological understanding of pain and pain-like symptoms were used here. The transcultural dictionaries with additional data developed by a machine learning algorithm generated a therapeutic indication dictionary where pain was the indication.

Also as part of the analysis, a searchable repository (PhAROS_CONVERGE) included data and pre-processed data that allowed identification of commonalities in therapeutic approaches from biogeographically and culturally traditional medical systems (TMS). The data and pre-processed data of the PhAROS_CONVERGE included: (1) therapeutic indication dictionaries related to traditional medical systems that reflect modern and historical terminology, and/or Western and non-Western epistemologies; (2) medical formulation compositions related to traditional medical systems; (3) compound data sets for a given therapeutic indication; and (4) a proprietary digital composition index (n-dimensional vector and/or fingerprint).

Additionally, the data and pre-processed data of the PhAROS_CONVERGE was further configured to allow (1) identification of efficacious medical components across traditional medicine systems and (2) ranking optimization of de novo compound formulations and compound mixtures by utilizing transcultural components for subsequent preclinical and clinical testing for a given therapeutic indication.

The processed data returned by the query included: a list of compounds associated with pain, a list of prescription formulae associated with pain, a list of organisms associated with pain, a list of chemicals associated with pain, or a combination thereof.

Moreover, each TMS identified by the in silico convergent analysis described above was linked to one or more of: a number of compounds within the list of compounds associated with pain, a number of prescription formulae within the list of prescription formulae associated with pain, a number of organisms within the list of organisms associated with pain, and a number of chemicals within the list of chemicals associated with pain. Data outputted from this example is described below.

FIG. 22A shows the workflow of initial steps of in silico convergence analysis for Pain using the PhAROS methods, when the initiating step was assembly of an indications dictionary.

FIG. 22B shows the workflow of initial steps in in silico convergence analysis for Pain using PhAROS Platform, when the initiating step was identification of formulae using literature mining.

FIG. 22C shows a summary of the output from the PhAROS method when pain was the query, including the number of formulations, indications, ingredient organisms and chemical components found in PhAROS across the indicated TMS for pain.

FIG. 22D shows PhAROS outputs resulting from the in silico convergence analysis for pain for the PhAROS_PHARM database. This schematic shows that 121 compounds were indicated for pain in 4 or more traditional medicine systems (TMS).

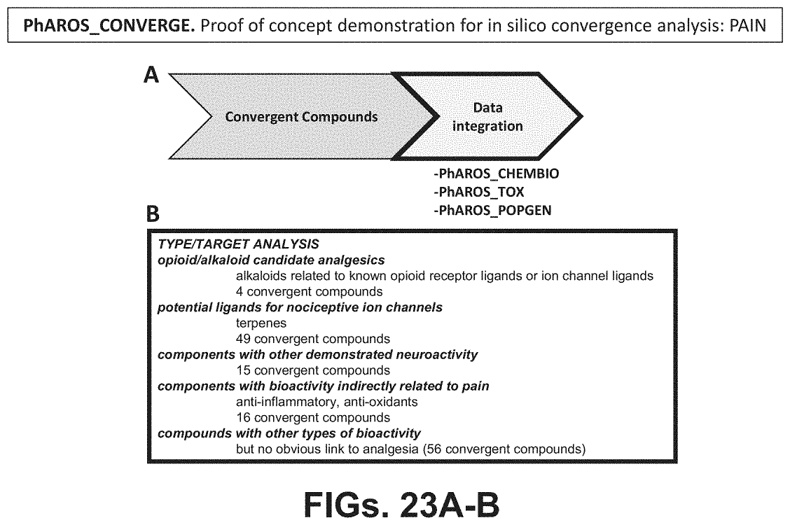

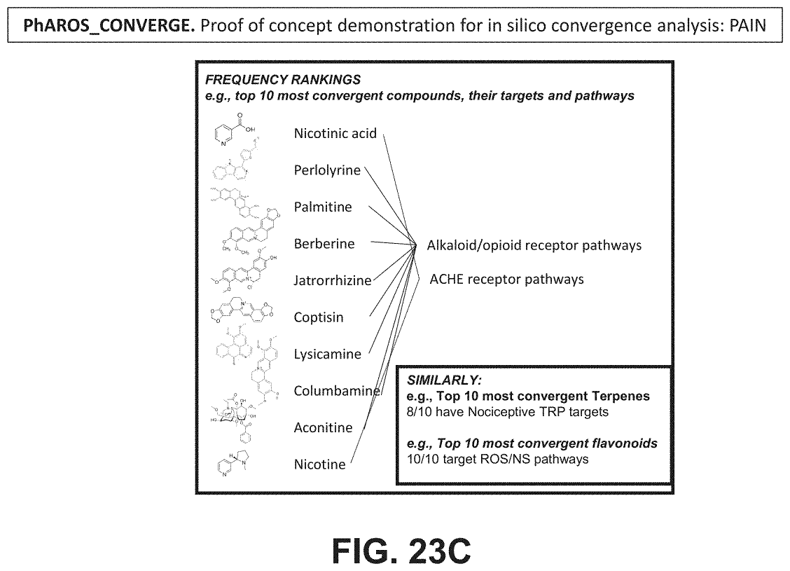

FIG. 23A shows a schematic of steps in in silico convergence analysis for Pain.

FIG. 23B shows PhAROS outputs resulting from an in silico convergence analysis for pain. This table shows the number and type of candidate analgesics identified by PhAROS in in silico convergence analysis (ISCA) for pain.

FIG. 23C shows PhAROS outputs of in silico convergence analysis for pain. FIG. 23C is an example of a ranking by PhAROS of the most convergent compounds (i.e., those compounds most frequently present across the queried TMS) in a class (alkaloids and opioids, with other classes summarized in the inset), representing the compounds with broadest agreement between TMS for inclusion in pain formulations.

FIGS. 24A-B shows a PhAROS output resulting from an in silico convergence analysis (ISCA) for pain, including a chord diagram (Circos plot) generated by PhAROS_MODVIZ to represent overlap and lineages between TMS. FIG. 24C (right panel) shows a ranking by PhAROS of the most convergent compounds in a class separated by level of agreement between TMS (convergence across 5 regions, convergence across 4 regions). PhAROS can then use this information for reducing complexity and de-risking components for further evaluation.

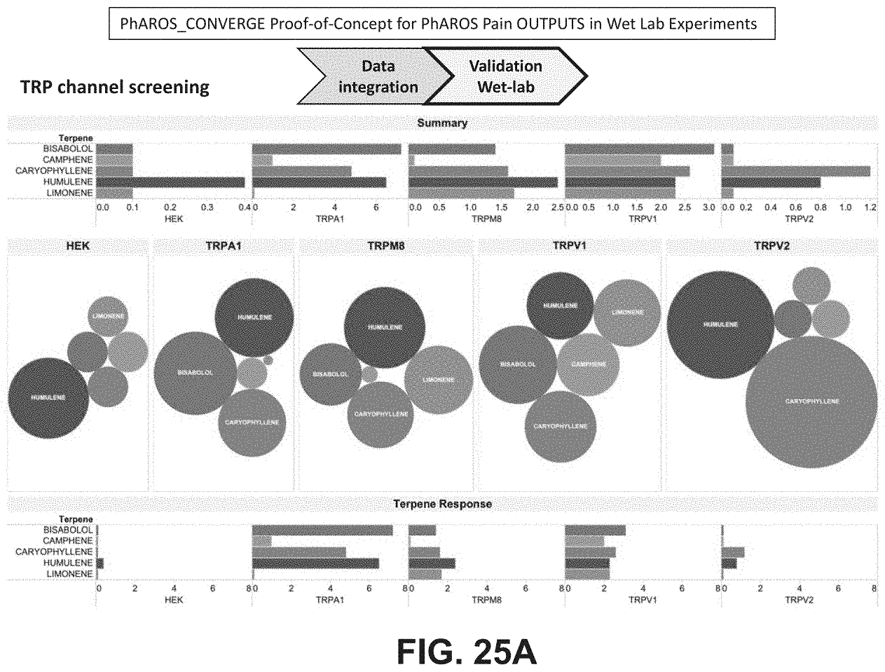

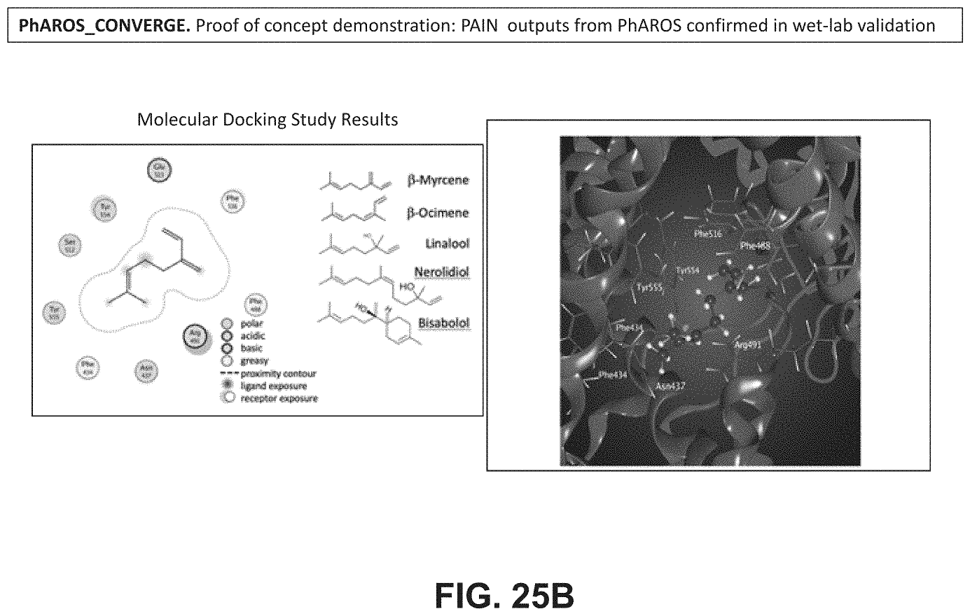

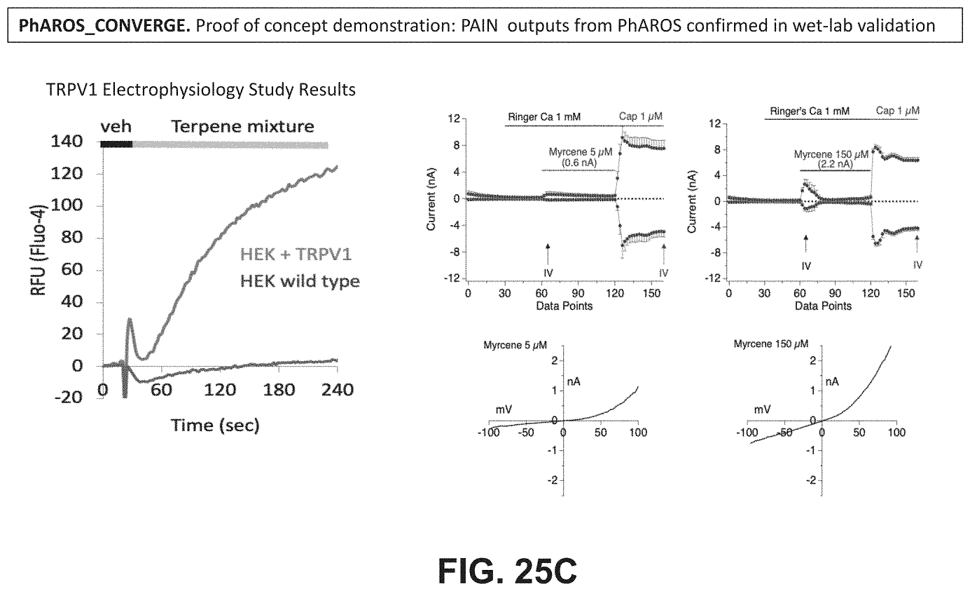

FIG. 25A shows wet-lab validation of results of in silico convergence analysis for pain. Terpenes found in the ISCA include effective ligands and potential agonists-desensitizers for nociceptive TRP channels. HEK cells inducibly expressing the indicated ion channels (i.e., TRPA1, TRPM8, TRPV1, and TRPV2) were loaded with Fluo-4 acetoxymethyl ester in a modified Ringer's solution containing 1 mM CaCl₂). Cells were stimulated with vehicle or the indicated terpene at a concentration of 1 μM, or matched vehicle, and time-resolved fluorescence measurements were collected in a Molecular Devices Flexstation 3. The peak attained increases in relative fluorescence units (RFU) were calculated, vehicle subtracted and plotted. Comparison plots in FIG. 25A show the relative intensity of the intracellular free calcium mobilization initiated by each terpene with the diameter of each circle representing the peak intensity (middle panel), and as peak intensity summarized in histograms (lower panel).

FIG. 25B shows molecular docking/modeling validation of results of in silico convergence analysis for pain. FIG. 25B left panel shows two-dimensional representation of molecular docking of Myrcene at the nociceptive ion channel TRPV1, including ligand interactions of Myrcene at binding site 4 of TRPV1. FIG. 25B left panel also shows similarities in chemical moieties between specific terpenes found in plant sources. FIG. 25B right panel shows a three-dimensional representation of Myrcene docked at binding site 4 of TRPV1.

FIG. 25C provides data on the functional effects of terpenes at the nociceptive ion channel TRPV1. FIG. 25C, left panel, shows Fluo-4 Ca2+ response in wild type HEK or HEK over-expressing TRPV1 treated with vehicle or with 10 μM mixture of terpenes derived from phytomedical plants identified using PhAROS. Using whole cell patch clamp electrophysiology, myrcene was shown to activate TRPV1 conductance (FIG. 25C, right panel).

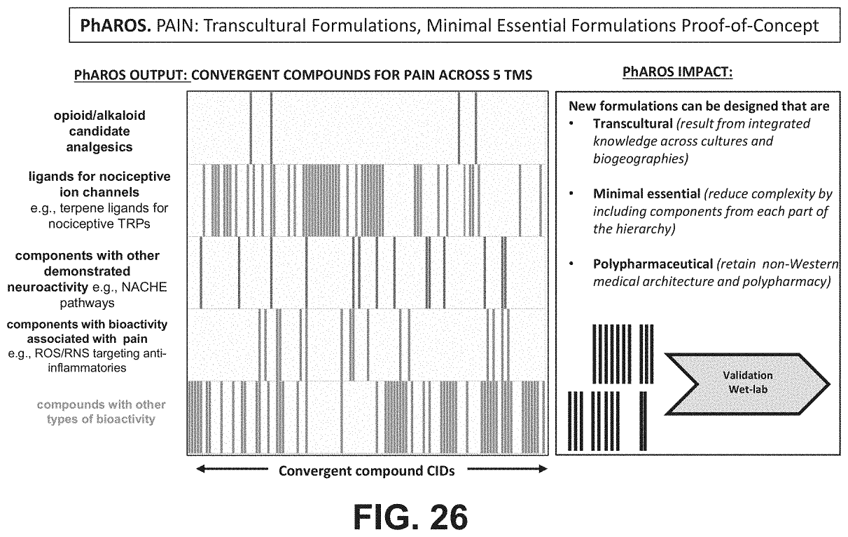

In Silico convergence analysis (ISCA) examines an indication (e.g., pain) across TM systems from multiple cultures and seeks to identify compound-level commonalities in the formulations that different cultures have arrived at through empirical/historical experimentation. FIG. 26 summarizes ISCA for two Kampo and two TCM formulations indicated for pain. Formulation component lists (˜800-2000 components) were generated using databases such as BATMAN-TCM and KAMPO-DB and triaged for non-bioactive components (leading to lists of ˜200-400 compounds). A convergent set of compounds was identified that were represented in 2 (one Kampo, one TCM) or all 4 proposed analgesic formulations. In one of the pairwise comparisons, 121 compounds were shared between the 2 (one Kampo and one TMC) formulations. These were then re-categorized using literature analysis into opioid/alkaloid candidate analgesics (alkaloids related to known opioid receptor ligands, 4 convergent compounds), potential ligands for nociceptive ion channels (terpenes, 49 convergent compounds), components with other demonstrated neuroactivity (15 convergent compounds), components with bioactivity indirectly related to pain (anti-inflammatory, anti-oxidants, 16 convergent compounds) and compounds with other types of bioactivity but no obvious link to analgesia (56 convergent compounds).

FIG. 27 shows a schematic of a process for designing opioid alternative pain medications based on PhAROS outputs.

FIG. 28A shows an example PhAROS OUTPUT for all molecular targets (data integration with GO, KEGG, others) associated with chemical components of TMS formulations indicated for pain.

### Example 2. Methods and Compositions for Novel Pain Therapies Targeted to Specific Pain Subtypes Identified Using the PhAROS in Silico Drug Discovery Platform

In this example, a PhAROS method was used to identify new polypharmaceutical compositions targeted to specific pain subtypes.

In particular, PhAROS was used to identify new polypharmaceutical compositions for treating specific pain subtypes by analyzing, in a single computational space, data from a plurality of traditional medicine systems (TMS), where the analysis used transcultural dictionaries to allow searches within distinct TMS data sets embodying different epistemologies and terminologies, and where the analysis used data returned by a query (i.e., pain type) to identify new polypharmaceutical and/or optimized polypharmaceutical compositions for specific pain subtypes.

Data analysis included a subset of the Inputs as described in FIG. 8. We analyzed, in a single computational space, data from a plurality of traditional medicine systems (TMS), including normalized formalized pharmacopeias from one or more geographic regions associated with TMS (i.e., PhAROS_PHARM).

The processed data included a list of pain types across multiple TMS. For each pain type, the processed data included a list of TMS referenced from the plurality of TMS, associated with the pain type. Additionally, for each pain type, the processed data included the identity of a plurality of TMS linked to one or more selected from: the pain type, one or more compounds associated with the pain type, and one or more organisms associated with the pain type.

PhAROS_PHARM text mining collapsed greater than 1000 pain indications across 5 TMS to 37 major categories (FIG. 29). The list of pain types included: abdominal, cardiac/chest, mouth, muscle, back, inflammation, joint, eye, chronic pain/inflammation, labor/postpartum, skin, throat, limb, bone, breast, ear, pelvic, intestinal, anal, pain sensitivity, rib, neuropathic, bladder, kidney, lung, menstruation, facial, liver, arthritis, fallopian tube, urethra, and vaginal.

The processed data revealed that PhAROS can use data from a plurality of traditional medicine systems to differentiate between pain types. FIG. 29 shows regional convergence and associated number of formulations for the 37 major pain subtypes identified using the PhAROS method. Table 3 shows the plants most broadly associated with each type of pain (ranking by Regional Convergence 3+) as identified by the PhAROS method.

TABLE 3
Plants most broadly associated with each pain type.

| Generalized Pain | | | |
|---|---|---|---|
| Indication | Ingredient Organism | Regional Convergence | Formula Count |
| abdominal | allium sativum | 4 | 3 |
| abdominal | panax ginseng | 3 | 186 |
| abdominal | cyperus rotundus | 3 | 149 |
| abdominal | zingiber officinale | 3 | 120 |
| abdominal | hordeum vulgare | 3 | 96 |
| abdominal | prunus persica | 3 | 78 |
| abdominal | morus alba | 3 | 42 |
| abdominal | curcuma longa | 3 | 17 |
| abdominal | foeniculum vulgare | 3 | 12 |
| abdominal | melia azedarach | 3 | 9 |
| abdominal | cannabis sativa | 3 | 8 |
| abdominal | acorus calamus | 3 | 7 |
| abdominal | piper nigrum | 3 | 2 |
| back | cinnamomum cassia | 4 | 266 |
| back | zingiber officinale | 4 | 105 |
| back | citrus reticulata | 3 | 383 |
| back | cyperus rotundus | 3 | 194 |
| cardiac/chest | cinnamomum cassia | 3 | 202 |
| cardiac/chest | cyperus rotundus | 3 | 137 |
| cardiac/chest | zingiber officinale | 3 | 99 |
| general | scutellaria baicalensis | 3 | 210 |
| inflammation | | | |
| general pain | cinnamomum cassia | 3 | 290 |
| general pain | zingiber officinale | 3 | 243 |
| general pain | hordeum vulgare | 3 | 112 |
| general pain | terminalia chebula | 3 | 27 |
| general pain | santalum album | 3 | 27 |
| general pain | piper longum | 3 | 15 |
| general pain | trigonella foenum- | 3 | 9 |
| | graecum | | |
| general pain | liquidambar orientalis | 3 | 9 |
| head | cinnamomum cassia | 4 | 359 |
| head | zingiber officinale | 4 | 242 |
| head | prunus persica | 4 | 98 |
| head | sesamum indicum | 4 | 15 |
| head | cyperus rotundus | 3 | 288 |
| head | alpinia officinarum | 3 | 51 |
| head | mentha piperita | 3 | 32 |
| head | datura metel | 3 | 15 |
| head | luffa cylindrica | 3 | 6 |
| head | eucalyptus globulus | 3 | 4 |
| head | oxalis corniculata | 3 | 3 |
| mouth | cinnamomum cassia | 3 | 219 |
| mouth | zingiber officinale | 3 | 104 |
| mouth | eclipta prostrata | 3 | 29 |
| mouth | piper longum | 3 | 15 |
| mouth | datura metel | 3 | 9 |
| mouth | smilax china | 3 | 4 |
| mouth | psidium guajava | 3 | 2 |
| muscle | cyperus rotundus | 3 | 199 |
| other | zingiber officinale | 3 | 129 |
| inflammation | | | |
| other | trigonella foenum- | 3 | 18 |
| inflammation | graecum | | |
| other | cannabis sativa | 3 | 14 |
| inflammation | | | |
| other | imperata cylindrica | 3 | 13 |
| inflammation | | | |
| other | ricinus communis | 3 | 12 |
| inflammation | | | |
| other | datura metel | 3 | 11 |
| inflammation | | | |
| other | centella asiatica | 3 | 9 |
| inflammation | | | |
| other | citrus medica | 3 | 6 |
| inflammation | | | |
| other pain | sesamum indicum | 4 | 13 |
| other pain | cinnamomum cassia | 3 | 250 |
| other pain | panax ginseng | 3 | 226 |
| other pain | cyperus rotundus | 3 | 200 |
| other pain | zingiber officinale | 3 | 153 |
| other pain | hordeum vulgare | 3 | 84 |
| other pain | morus alba | 3 | 51 |
| other pain | eclipta prostrata | 3 | 33 |
| other pain | sinapis alba | 3 | 20 |
| other pain | melia azedarach | 3 | 5 |
| other pain | foeniculum vulgare | 3 | 4 |
| other pain | plumbago zeylanica | 3 | 2 |

Table 4 shows compounds most broadly associated with each type of pain (ranking by Formula Count, 300+) as identified by the PhAROS method. Additional analysis was performed to identify (i) broad and narrow spectrum analgesics from the outputted data from the PhAROS method and (ii) information for reducing complexity and de-risking components for further evaluation.

TABLE 4
Compounds most broadly associated with each type of pain.

| Generalized Pain | | | |
|---|---|---|---|
| Indication | Ingredient Organism | Regional Convergence | Formula Count |
| abdominal | glycyrrhiza uralensis | 2 | 494 |
| abdominal | angelica sinensis | 2 | 392 |
| abdominal | poria cocos | 2 | 341 |
| abdominal | paeonia lactiflora | 2 | 340 |
| anal | glycyrrhiza uralensis | 1 | 477 |
| anal | angelica sinensis | 1 | 475 |
| anal | poria cocos | 1 | 388 |
| anal | paeonia lactiflora | 1 | 363 |
| anal | citrus reticulata | 1 | 334 |
| anal | ligusticum chuanxiong | 1 | 302 |
| back | glycyrrhiza uralensis | 2 | 535 |
| back | angelica sinensis | 2 | 454 |
| back | poria cocos | 2 | 422 |
| back | citrus reticulata | 3 | 383 |
| back | paeonia lactiflora | 2 | 374 |
| bone | glycyrrhiza uralensis | 2 | 703 |
| bone | angelica sinensis | 1 | 631 |
| bone | poria cocos | 2 | 509 |
| bone | paeonia lactiflora | 2 | 496 |
| bone | citrus reticulata | 1 | 464 |
| bone | ligusticum chuanxiong | 2 | 389 |
| bone | scutellaria baicalensis | 2 | 330 |
| bone | blumea balsamifera | 1 | 327 |
| bone | cocked root | 1 | 324 |
| bone | cinnamomum cassia | 2 | 301 |
| cardiac/chest | glycyrrhiza uralensis | 2 | 481 |
| cardiac/chest | angelica sinensis | 1 | 414 |
| cardiac/chest | paeonia lactiflora | 2 | 336 |
| cardiac/chest | poria cocos | 2 | 334 |
| chronic | glycyrrhiza uralensis | 1 | 481 |
| pain/inflammation | | | |
| chronic | angelica sinensis | 1 | 423 |
| pain/inflammation | | | |
| chronic | paeonia lactiflora | 1 | 344 |
| pain/inflammation | | | |
| chronic | poria cocos | 1 | 335 |
| pain/inflammation | | | |
| chronic | citrus reticulata | 1 | 313 |
| pain/inflammation | | | |
| eye | glycyrrhiza uralensis | 1 | 850 |
| eye | angelica sinensis | 1 | 777 |
| eye | poria cocos | 1 | 642 |
| eye | paeonia lactiflora | 1 | 620 |
| eye | citrus reticulata | 1 | 578 |
| eye | ligusticum chuanxiong | 1 | 465 |
| eye | cocked root | 1 | 422 |
| eye | scutellaria baicalensis | 1 | 388 |
| eye | panax ginseng | 1 | 378 |
| eye | blumea balsamifera | 1 | 378 |
| eye | cinnamomum cassia | 1 | 375 |
| eye | codonopsis pilosula | 1 | 375 |
| eye | aucklandia lappa | 1 | 348 |
| eye | platycodon grundiflorum | 1 | 333 |
| eye | dioscorea opposita | 1 | 324 |
| eye | rheum palmatum | 1 | 319 |
| eye | rehmannia glutinosa | 1 | 317 |
| eye | huang chi | 1 | 305 |
| facial | glycyrrhiza uralensis | 1 | 705 |
| facial | angelica sinensis | 1 | 677 |
| facial | poria cocos | 1 | 573 |
| facial | paeonia lactiflora | 1 | 538 |
| facial | citrus reticulata | 1 | 478 |
| facial | ligusticum chuanxiong | 1 | 429 |
| facial | cocked root | 1 | 373 |
| facial | panax ginseng | 1 | 366 |
| facial | scutellaria baicalensis | 1 | 362 |
| facial | rehmannia glutinosa | 1 | 351 |
| facial | dioscorea opposita | 1 | 324 |
| facial | angelica dahurica | 1 | 323 |
| facial | cinnamomum cassia | 1 | 322 |
| facial | blumea balsamifera | 1 | 302 |
| general | glycyrrhiza uralensis | 2 | 481 |
| inflammation | | | |
| general | angelica sinensis | 2 | 382 |
| inflammation | | | |
| general | poria cocos | 2 | 328 |
| inflammation | | | |
| general | paeonia lactiflora | 2 | 324 |
| inflammation | | | |
| general pain | glycyrrhiza uralensis | 2 | 704 |
| general pain | poria cocos | 2 | 448 |
| general pain | paeonia lactiflora | 2 | 425 |
| general pain | angelica sinensis | 2 | 392 |
| head | glycyrrhiza uralensis | 2 | 924 |
| head | angelica sinensis | 2 | 657 |
| head | poria cocos | 2 | 616 |
| head | paeonia lactiflora | 2 | 589 |
| head | citrus reticulata | 2 | 550 |
| head | ligusticum chuanxiong | 2 | 471 |
| head | scutellaria baicalensis | 2 | 401 |
| head | blumea balsamifera | 2 | 379 |
| head | cinnamomum cassia | 4 | 359 |
| head | saposhnikovia divaricata | 2 | 347 |
| head | aucklandia lappa | 2 | 332 |
| head | cocked root | 1 | 329 |
| head | panax ginseng | 2 | 324 |
| head | rehmannia glutinosa | 2 | 321 |
| head | rheum palmatum | 2 | 315 |
| intestinal | angelica sinensis | 1 | 435 |
| intestinal | glycyrrhiza uralensis | 1 | 429 |
| intestinal | aucklandia lappa | 1 | 398 |
| intestinal | poria cocos | 1 | 390 |
| intestinal | citrus reticulata | 1 | 352 |
| intestinal | paeonia lactiflora | 1 | 348 |
| intestinal | dioscorea opposita | 1 | 324 |
| liver | glycyrrhiza uralensis | 1 | 509 |
| liver | angelica sinensis | 1 | 486 |
| liver | paeonia lactiflora | 1 | 383 |
| liver | citrus reticulata | 1 | 374 |
| liver | poria cocos | 1 | 371 |
| liver | ligusticum chuanxiong | 1 | 312 |
| lung | glycyrrhiza uralensis | 1 | 614 |
| lung | angelica sinensis | 1 | 510 |
| lung | poria cocos | 1 | 412 |
| lung | citrus reticulata | 1 | 411 |
| lung | paeonia lactiflora | 1 | 394 |
| mouth | glycyrrhiza uralensis | 2 | 485 |
| mouth | angelica sinensis | 2 | 477 |
| mouth | poria cocos | 2 | 389 |
| mouth | paeonia lactiflora | 2 | 365 |
| mouth | citrus reticulata | 1 | 334 |
| mouth | ligusticum chuanxiong | 2 | 307 |
| muscle | glycyrrhiza uralensis | 1 | 526 |
| muscle | citrus reticulata | 1 | 473 |
| muscle | angelica sinensis | 1 | 452 |
| muscle | ligusticum chuanxiong | 1 | 393 |
| muscle | blumea balsamifera | 1 | 391 |
| muscle | poria cocos | 1 | 389 |
| muscle | paeonia lactiflora | 1 | 366 |
| neuropathic | glycyrrhiza uralensis | 1 | 795 |
| neuropathic | angelica sinensis | 1 | 768 |
| neuropathic | paeonia lactiflora | 1 | 595 |
| neuropathic | poria cocos | 1 | 590 |
| neuropathic | citrus reticulata | 1 | 553 |
| neuropathic | ligusticum chuanxiong | 1 | 514 |
| neuropathic | cocked root | 1 | 404 |
| neuropathic | panax ginseng | 1 | 371 |
| neuropathic | cinnamomum cassia | 1 | 367 |
| neuropathic | blumea balsamifera | 1 | 364 |
| neuropathic | scutellaria baicalensis | 1 | 348 |
| neuropathic | aucklandia lappa | 1 | 326 |
| neuropathic | rheum palmatum | 1 | 321 |
| neuropathic | codonopsis pilosula | 1 | 314 |
| neuropathic | platycodon grundiflorum | 1 | 307 |
| neuropathic | rehmannia glutinosa | 1 | 305 |
| other | angelica sinensis | 1 | 856 |
| inflammation | | | |
| other | glycyrrhiza uralensis | 1 | 612 |
| inflammation | | | |
| other | paeonia lactiflora | 1 | 566 |
| inflammation | | | |
| other | ligusticum chuanxiong | 1 | 514 |
| inflammation | | | |
| other | poria cocos | 1 | 494 |
| inflammation | | | |
| other | citrus reticulata | 1 | 412 |
| inflammation | | | |
| other | cocked root | 1 | 375 |
| inflammation | | | |
| other | blumea balsamifera | 1 | 337 |
| inflammation | | | |
| other | cinnamomum cassia | 1 | 325 |
| inflammation | | | |
| other | codonopsis pilosula | 1 | 312 |
| inflammation | | | |
| other pain | glycyrrhiza uralensis | 2 | 576 |
| other pain | angelica sinensis | 2 | 469 |
| other pain | poria cocos | 2 | 423 |
| other pain | paeonia lactiflora | 2 | 405 |
| other pain | citrus reticulata | 2 | 344 |
| other pain | ligusticum chuanxiong | 2 | 305 |
| pain insensitivity | glycyrrhiza uralensis | 2 | 509 |
| pain insensitivity | angelica sinensis | 2 | 475 |
| pain insensitivity | poria cocos | 2 | 406 |
| pain insensitivity | paeonia lactiflora | 2 | 386 |
| pain insensitivity | citrus reticulata | 2 | 335 |
| pain insensitivity | ligusticum chuanxiong | 2 | 308 |
| pelvic | glycyrrhiza uralensis | 1 | 513 |
| pelvic | angelica sinensis | 1 | 474 |
| pelvic | poria cocos | 1 | 432 |
| pelvic | paeonia lactiflora | 1 | 391 |
| pelvic | rehmannia glutinosa | 1 | 343 |
| pelvic | panax ginseng | 1 | 336 |
| pelvic | citrus reticulata | 1 | 335 |
| pelvic | dioscorea opposita | 1 | 320 |
| pelvic | angelica dahurica | 1 | 315 |
| skin | glycyrrhiza uralensis | 1 | 531 |
| skin | angelica sinensis | 1 | 477 |
| skin | poria cocos | 1 | 475 |
| skin | paeonia lactiflora | 1 | 388 |
| skin | citrus reticulata | 1 | 371 |
| skin | rehmannia glutinosa | 1 | 334 |
| skin | panax ginseng | 1 | 325 |
| skin | dioscorea opposita | 1 | 310 |
| skin | angelica dahurica | 1 | 302 |

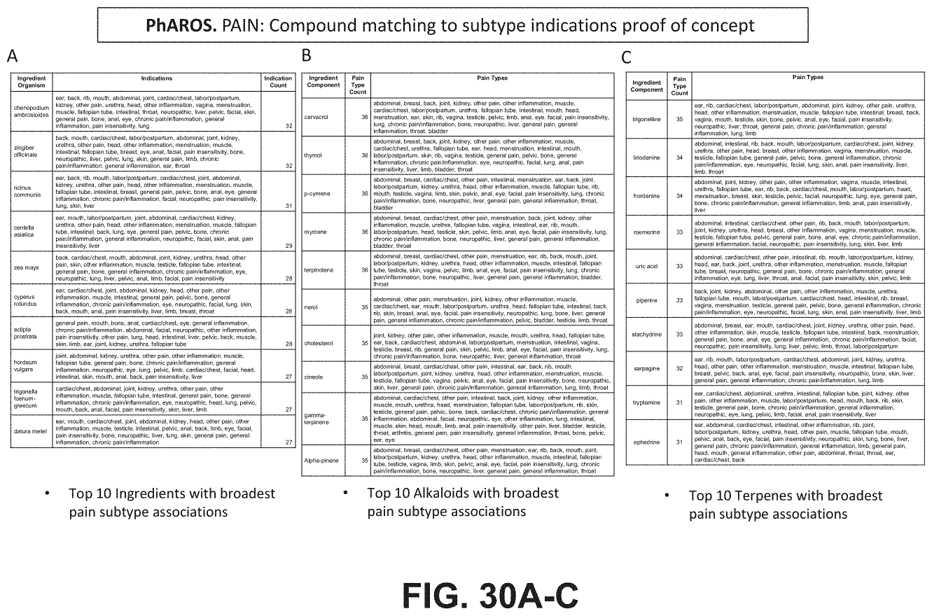

To identify putative broad spectrum analgesic candidates, the 37 categories identified above were ranked to identify putative broad spectrum analgesic candidates. FIGS. 30A-C, Tables 5-7, respectively, show top 10 ingredient organisms, alkaloids, and terpenes, respectively, associated with the broadest pain subtype associations.

TABLE 5
Top 10 Ingredients with broadest pain subtype associations

| Ingredient | | Indication |
|---|---|---|
| Organism | Indications | Count |
| chenopodium | ear, back, rib, mouth, abdominal, joint, cardiac/chest, | 32 |
| ambrosioides | labor/postpartum, kidney, other pain, urethra, head, other | |
| | inflammation, vagina, menstruation, muscle, fallopian tube, | |
| | intestinal, throat, neuropathic, liver, pelvic, facial, skin, general pain, | |
| | bone, anal, eye, chronic pain/inflammation, general inflammation, | |
| | pain insensitivity, lung | |
| zingiber | back, mouth, cardiac/chest, labor/postpartum, abdominal, joint, | 32 |
| officinale | kidney, urethra, other pain, head, other inflammation, menstruation, | |
| | muscle, intestinal, fallopian tube, breast, eye, anal, facial, pain | |
| | insensitivity, bone, neuropathic, liver, pelvic, lung, skin, general | |
| | pain, limb, chronic pain/inflammation, general inflammation, ear, | |
| | throat | |
| ricinus | ear, back, rib, mouth, labor/postpartum, cardiac/chest, joint, | 31 |
| communis | abdominal, kidney, urethra, other pain, head, other inflammation, | |
| | menstruation, muscle, fallopian tube, intestinal, breast, general pain, | |
| | pelvic, bone, anal, eye, general inflammation, chronic | |
| | pain/inflammation, facial, neuropathic, pain insensitivity, lung, skin, | |
| | liver | |
| centella | ear, mouth, labor/postpartum, joint, abdominal, cardiac/chest, | 29 |
| asiatica | kidney, urethra, other pain, head, other inflammation, menstruation, | |
| | muscle, fallopian tube, intestinal, back, lung, eye, general pain, | |
| | pelvic, bone, chronic pain/inflammation, general inflammation, | |
| | neuropathic, facial, skin, anal, pain insensitivity, liver | |
| zea mays | back, cardiac/chest, mouth, abdominal, joint, kidney, urethra, head, | 28 |
| | other pain, skin, other inflammation, muscle, testicle, fallopian tube, | |
| | intestinal, general pain, bone, general inflammation, chronic | |
| | pain/inflammation, eye, neuropathic, lung, liver, pelvic, anal, limb, | |
| | facial, pain insensitivity | |
| cyperus | ear, cardiac/chest, joint, abdominal, kidney, head, other pain, other | 28 |
| rotundus | inflammation, muscle, intestinal, general pain, pelvic, bone, general | |
| | inflammation, chronic pain/inflammation, eye, neuropathic, facial, | |
| | lung, skin, back, mouth, anal, pain insensitivity, liver, limb, breast, | |
| | throat | |
| eclipta | general pain, mouth, bone, anal, cardiac/chest, eye, general | 28 |
| prostrata | inflammation, chronic pain/inflammation, abdominal, facial, | |
| | neuropathic, other inflammation, pain insensitivity, other pain, lung, | |
| | head, intestinal, liver, pelvic, back, muscle, skin, limb, ear, joint, | |
| | kidney, urethra, fallopian tube | |
| hordeum | joint, abdominal, kidney, urethra, other pain, other inflammation, | 27 |
| vulgare | muscle, fallopian tube, general pain, bone, chronic | |
| | pain/inflammation, general inflammation, neuropathic, eye, lung, | |
| | pelvic, limb, cardiac/chest, facial, head, intestinal, skin, mouth, anal, | |
| | back, pain insensitivity, liver | |
| trigonella | cardiac/chest, abdominal, joint, kidney, urethra, other pain, other | 27 |
| foenum- | inflammation, muscle, fallopian tube, intestinal, general pain, bone, | |
| graecum | general inflammation, chronic pain/inflammation, eye, neuropathic, | |
| | head, lung, pelvic, mouth, back, anal, facial, pain insensitivity, skin, | |
| | liver, limb | |
| datura metel | ear, mouth, cardiac/chest, joint, abdominal, kidney, head, other pain, | 27 |
| | other inflammation, muscle, testicle, intestinal, pelvic, anal, back, | |
| | limb, eye, facial, pain insensitivity, bone, neuropathic, liver, lung, | |
| | skin, general pain, general inflammation, chronic pain/inflammation | |

TABLE 6
Top 10 Alkaloids with broadest pain subtype associations

| Ingredient | Pain Type | |
|---|---|---|
| Component | Count | Pain Types |
| carvacrol | 36 | abdominal, breast, back, joint, kidney, other pain, other inflammation, |
| | | muscle, cardiac/chest, labor/postpartum, urethra, fallopian tube, |
| | | intestinal, mouth, head, menstruation, ear, skin, rib, vagina, testicle, |
| | | pelvic, limb, anal, eye, facial, pain insensitivity, lung, chronic |
| | | pain/inflammation, bone, neuropathic, liver, general pain, general |
| | | inflammation, throat, bladder |
| thymol | 36 | abdominal, breast, back, joint, kidney, other pain, other inflammation, |
| | | muscle, cardiac/chest, urethra, fallopian tube, ear, head, menstruation, |
| | | intestinal, mouth, labor/postpartum, skin, rib, vagina, testicle, general |
| | | pain, pelvic, bone, general inflammation, chronic pain/inflammation, |
| | | eye, neuropathic, facial, lung, anal, pain insensitivity, liver, limb, |
| | | bladder, throat |
| p-cymene | 36 | abdominal, breast, cardiac/chest, other pain, intestinal, menstruation, |
| | | ear, back, joint, labor/postpartum, kidney, urethra, head, other |
| | | inflammation, muscle, fallopian tube, rib, mouth, testicle, vagina, limb, |
| | | skin, pelvic, anal, eye, facial, pain insensitivity, lung, chronic |
| | | pain/inflammation, bone, neuropathic, liver, general pain, general |
| | | inflammation, throat, bladder |
| myrcene | 36 | abdominal, breast, cardiac/chest, other pain, menstruation, back, joint, |
| | | kidney, other inflammation, muscle, urethra, fallopian tube, vagina, |
| | | intestinal, ear, rib, mouth, labor/postpartum, head, testicle, skin, pelvic, |
| | | limb, anal, eye, facial, pain insensitivity, lung, chronic |
| | | pain/inflammation, bone, neuropathic, liver, general pain, general |
| | | inflammation, bladder, throat |
| terpinolene | 36 | abdominal, breast, cardiac/chest, other pain, menstruation, ear, rib, |
| | | back, mouth, joint, labor/postpartum, kidney, urethra, head, other |
| | | inflammation, muscle, intestinal, fallopian tube, testicle, skin, vagina, |
| | | pelvic, limb, anal, eye, facial, pain insensitivity, lung, chronic |
| | | pain/inflammation, bone, neuropathic, liver, general pain, general |
| | | inflammation, bladder, throat |
| nerol | 35 | abdominal, other pain, menstruation, joint, kidney, other inflammation, |
| | | muscle, cardiac/chest, ear, mouth, labor/postpartum, urethra, head, |
| | | fallopian tube, intestinal, back, rib, skin, breast, anal, eye, facial, pain |
| | | insensitivity, neuropathic, lung, bone, liver, general pain, general |
| | | inflammation, chronic pain/inflammation, pelvic, bladder, testicle, limb, |
| | | throat |
| cholesterol | 35 | joint, kidney, other pain, other inflammation, muscle, mouth, urethra, |
| | | head, fallopian tube, ear, back, cardiac/chest, abdominal, |
| | | labor/postpartum, menstruation, intestinal, vagina, testicle, breast, rib, |
| | | general pain, skin, pelvic, limb, anal, eye, facial, pain insensitivity, |
| | | lung, chronic pain/inflammation, bone, neuropathic, liver, general |
| | | inflammation, throat |
| cineole | 35 | abdominal, breast, cardiac/chest, other pain, intestinal, ear, back, rib, |
| | | mouth, labor/postpartum, joint, kidney, urethra, head, other |
| | | inflammation, menstruation, muscle, testicle, fallopian tube, vagina, |
| | | pelvic, anal, eye, facial, pain insensitivity, bone, neuropathic, skin, |
| | | liver, general pain, chronic pain/inflammation, general inflammation, |
| | | lung, limb, throat |
| gamma- | 35 | abdominal, cardiac/chest, other pain, intestinal, back, joint, kidney, |
| terpinene | | other inflammation, muscle, mouth, urethra, head, menstruation, |
| | | fallopian tube, labor/postpartum, rib, skin, testicle, general pain, pelvic, |
| | | bone, back, cardiac/chest, chronic pain/inflammation, general |
| | | inflammation, abdominal, facial, neuropathic, eye, other inflammation, |
| | | lung, intestinal, muscle, skin, head, mouth, limb, anal, pain |
| | | insensitivity, other pain, liver, bladder, testicle, throat, arthritis, general |
| | | pain, pain insensitivity, general inflammation, throat, bone, pelvic, ear, |
| | | eye |
| Alpha-pinene | 35 | abdominal, breast, cardiac/chest, other pain, menstruation, ear, rib, |
| | | back, mouth, joint, labor/postpartum, kidney, urethra, head, other |
| | | inflammation, muscle, intestinal, fallopian tube, testicle, vagina, limb, |
| | | skin, pelvic, anal, eye, facial, pain insensitivity, lung, chronic |
| | | pain/inflammation, bone, neuropathic, liver, general pain, general |
| | | inflammation, throat |

TABLE 7
Top 10 Terpenes with broadest pain subtype associations

| Ingredient | Pain Type | |
|---|---|---|
| Component | Count | Pain Types |
| trigonelline | 35 | ear, rib, cardiac/chest, labor/postpartum, abdominal, joint, kidney, other |
| | | pain, urethra, head, other inflammation, menstruation, muscle, fallopian |
| | | tube, intestinal, breast, back, vagina, mouth, testicle, skin, bone, pelvic, |
| | | anal, eye, facial, pain insensitivity, neuropathic, liver, throat, general |
| | | pain, chronic pain/inflammation, general inflammation, lung, limb |
| liriodenine | 34 | abdominal, intestinal, rib, back, mouth, labor/postpartum, cardiac/chest, |
| | | joint, kidney, urethra, other pain, head, breast, other inflammation, |
| | | vagina, menstruation, muscle, testicle, fallopian tube, general pain, |
| | | pelvic, bone, general inflammation, chronic pain/inflammation, eye, |
| | | neuropathic, facial, lung, skin, anal, pain insensitivity, liver, limb, throat |
| hordenine | 34 | abdominal, joint, kidney, other pain, other inflammation, vagina, |
| | | muscle, intestinal, urethra, fallopian tube, ear, rib, back, cardiac/chest, |
| | | mouth, labor/postpartum, head, menstruation, breast, skin, testicle, |
| | | pelvic, facial, neuropathic, lung, eye, general pain, bone, chronic |
| | | pain/inflammation, general inflammation, limb, anal, pain insensitivity, |
| | | liver |
| roemerine | 33 | abdominal, intestinal, cardiac/chest, other pain, rib, back, mouth, |
| | | labor/postpartum, joint, kidney, urethra, head, breast, other |
| | | inflammation, vagina, menstruation, muscle, testicle, fallopian tube, |
| | | pelvic, general pain, bone, anal, eye, chronic pain/inflammation, |
| | | general inflammation, facial, neuropathic, pain insensitivity, lung, skin, |
| | | liver, limb |
| uric acid | 33 | abdominal, cardiac/chest, other pain, intestinal, rib, mouth, |
| | | labor/postpartum, kidney, head, ear, back, joint, urethra, other |
| | | inflammation, menstruation, muscle, fallopian tube, breast, neuropathic, |
| | | general pain, bone, chronic pain/inflammation, general inflammation, |
| | | eye, lung, liver, throat, anal, facial, pain insensitivity, skin, pelvic, limb |
| piperine | 33 | back, joint, kidney, abdominal, other pain, other inflammation, muscle, |
| | | urethra, fallopian tube, mouth, labor/postpartum, cardiac/chest, head, |
| | | intestinal, rib, breast, vagina, menstruation, testicle, general pain, |
| | | pelvic, bone, general inflammation, chronic pain/inflammation, eye, |
| | | neuropathic, facial, lung, skin, anal, pain insensitivity, liver, limb |
| stachydrine | 33 | abdominal, breast, ear, mouth, cardiac/chest, joint, kidney, urethra, |
| | | other pain, head, other inflammation, skin, muscle, testicle, fallopian |
| | | tube, intestinal, back, menstruation, general pain, bone, anal, eye, |
| | | general inflammation, chronic pain/inflammation, facial, neuropathic, |
| | | pain insensitivity, lung, liver, pelvic, limb, throat, labor/postpartum |
| sarpagine | 32 | ear, rib, mouth, labor/postpartum, cardiac/chest, abdominal, joint, |
| | | kidney, urethra, head, other pain, other inflammation, menstruation, |
| | | muscle, intestinal, fallopian tube, breast, pelvic, back, anal, eye, facial, |
| | | pain insensitivity, neuropathic, bone, skin, liver, general pain, general |
| | | inflammation, chronic pain/inflammation, lung, limb |
| tryptamine | 31 | ear, cardiac/chest, abdominal, urethra, intestinal, fallopian tube, joint, |
| | | kidney, other pain, other inflammation, muscle, labor/postpartum, head, |
| | | mouth, back, rib, skin, testicle, general pain, bone, chronic |
| | | pain/inflammation, general inflammation, neuropathic, eye, lung, |
| | | pelvic, limb, facial, anal, pain insensitivity, liver |
| ephedrine | 31 | ear, abdominal, cardiac/chest, intestinal, other inflammation, rib, joint, |
| | | labor/postpartum, kidney, urethra, head, other pain, muscle, fallopian |
| | | tube, mouth, pelvic, anal, back, eye, facial, pain insensitivity, |
| | | neuropathic, skin, lung, bone, liver, general pain, chronic |
| | | pain/inflammation, general inflammation, limb, general pain, head, |
| | | mouth, general inflammation, other pain, abdominal, throat, throat, ear, |
| | | cardiac/chest, back |

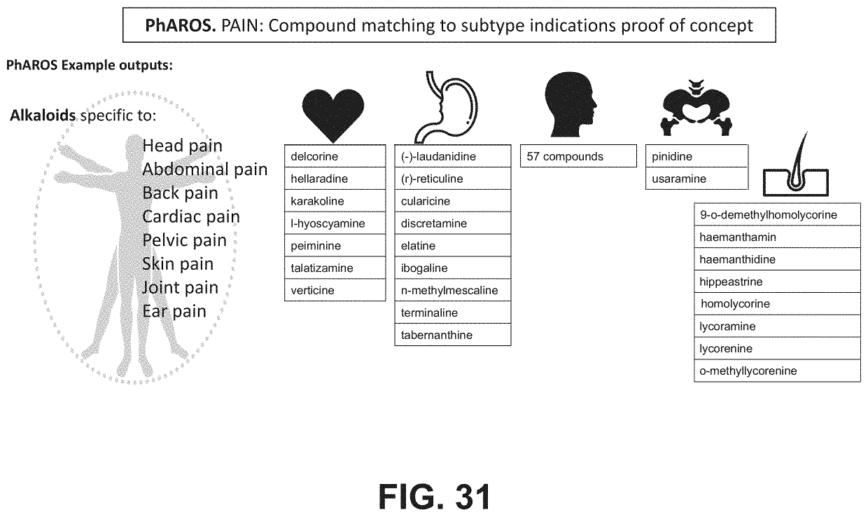

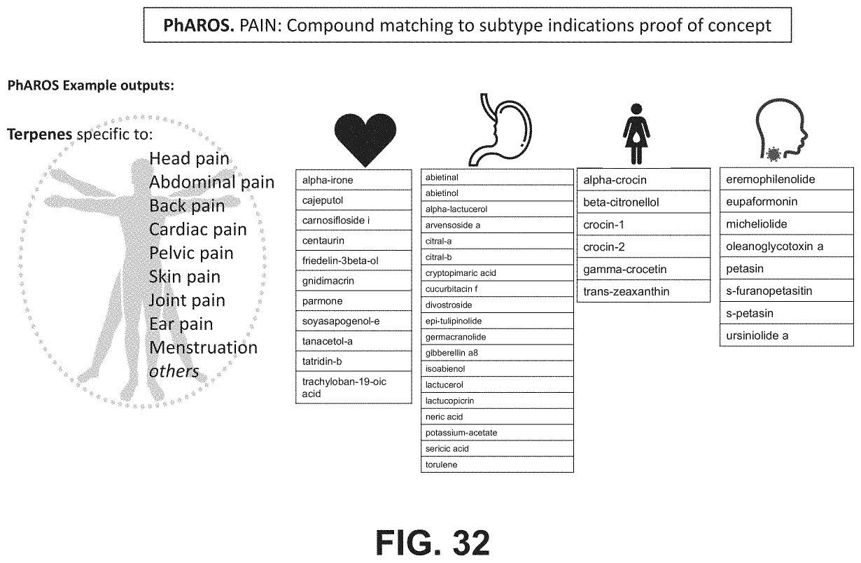

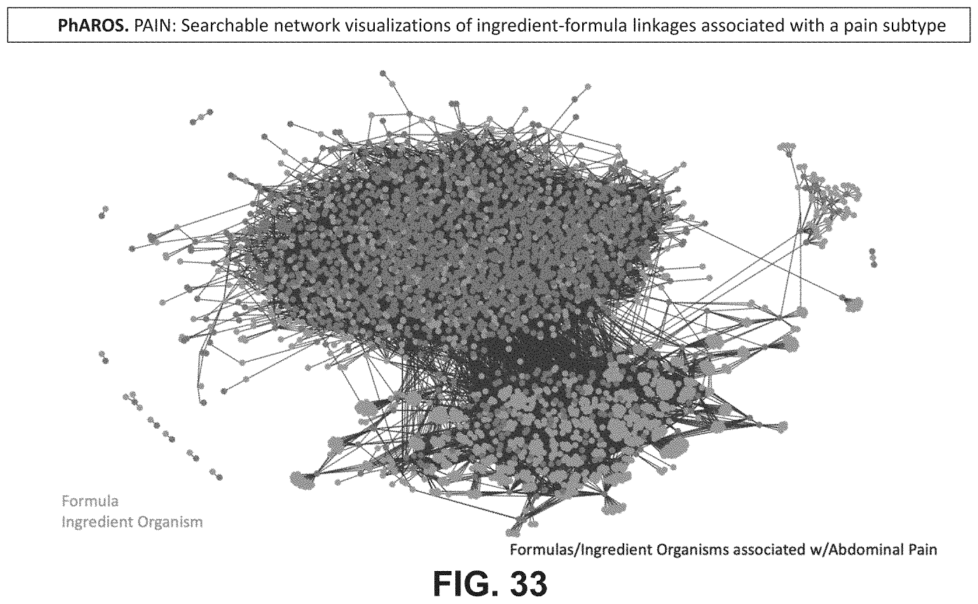

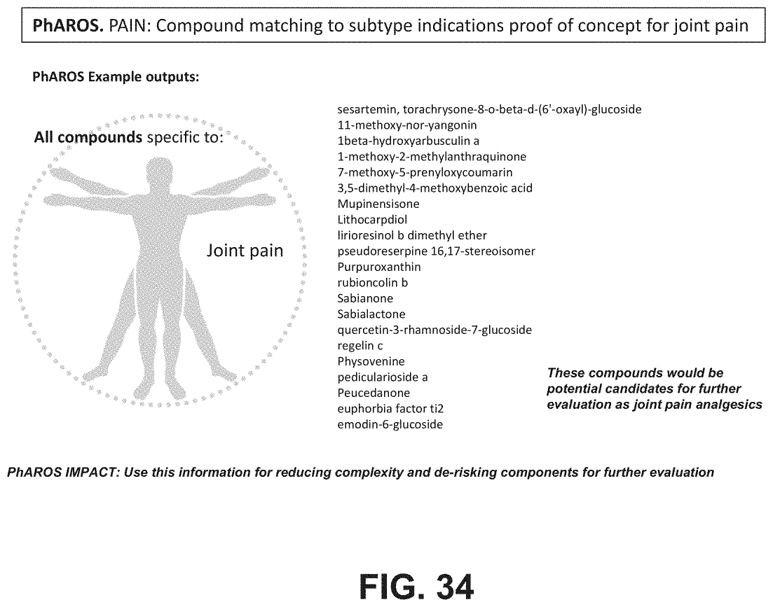

To identify putative narrow spectrum analgesic candidates, the 37 categories identified above were ranked to identify putative narrow spectrum analgesic candidates (based on narrowest pain spectrum). FIG. 31 shows the top-ranking alkaloid components associated with the indicated pain subtypes. FIG. 32 shows the top-ranking terpene chemical components associated with the indicated pain subtypes. FIG. 33 shows the searchable network of ingredient-formula linkages associated with a pain subtype. FIG. 34 shows the top-ranking chemical components associated with the joint pain subtype.

Overall, this example shows that PhAROS can use data from a plurality of traditional medicine systems to differentiate between pain types and match chemical components and ingredient organisms to specific pain types, thereby identifying new polypharmaceuticals—complex mixtures—for treating specific pain subtypes.

### Example 3. Piper Species Study

In this example, PhAROS was used to identify alternatives to Piper species for anxiety, pain, relaxation, and epilepsy. In particular, PhAROS was used to identify alternatives to P. methysticum for anxiety, pain, relaxation and epilepsy based on the restricted biogeography of P. methysticum.

The rationale for this study is provided below. (i) Piper species possess therapeutic and preventive potential against several chronic disorders. Piper species are represented in major TMS systems. (ii) Kavalactones are restricted to Piper methysticum. (iii) Piper species other than the kavalactone containing P. methysticum are indicted for pain, sedation, anxiety, depression, mood. Among the functional properties of Piper plants/extracts/active components, the antiproliferative, anti-inflammatory, and neuropharmacological activities of the extracts and extract-derived bioactive constituents are thought to be key effects for the protection against chronic conditions, based on preclinical in vitro and in vivo studies. The use of Piper species is informed by traditional and contemporary Cultural Medical Systems (CMS). Over 100 Piper species are in use in CMS in China, Korea, Japan, India, Africa and Oceania. P. methysticum has gained particular attention for anxiety and major depressive disorder based on its use in the Pacific as kava/sakai, a ritual soporific and relaxant drink. The proposed active ingredients of kava are kavalactones, but there is a paradox because many Piper spp. appear indicated for anxiety, in traditional medicine but the KL (pyrones) are thought to be restricted to P. methysticum.

Briefly, the approach used in this example included identifying medically important Piper spp. that could be used to interrogate PhAROS_PHARM and generate outputs associated with each Piper species to (1) a TMS, (2) one or more indications within the different TMS, and (3) sets of chemical components linked to each species within the databases comprising PhAROS_PHARM.

Here, PhAROS was used to discover polypharmaceutical medicines for treating pain, sedation, anxiety, depression, epilepsy, mood, and sleep by analyzing, in a single computational space, data from a plurality of traditional medicine systems (TMS), where the analysis used transcultural dictionaries to allow searches within distinct TMS data sets embodying different epistemologies and terminologies, and where the analysis uses data returned by a query (i.e., piper species) to identify alternative polypharmaceutical and/or optimized polypharmaceutical compositions to those found in Piper spp.

Data analysis included a subset of the Inputs as described in FIG. 8. We analyzed analyzed, in a single computational space, data from a plurality of traditional medicine systems (TMS), included normalized formalized pharmacopeias from one or more geographic regions associated with TMS (i.e., PhAROS_PHARM) and a pre-processed repository of integrated data, including but not limited to the meta-pharmacopeia, associated temporally, geographical, botanical, climatological, environmental, genomic, metagenomic, and metabolomic data on originating plants, components or other organisms (i.e., PhAROS_BIOGEO).

As part of the analysis, transcultural dictionaries that collate Western and non-Western epistemological understanding of piper species associated with the pain, sedation, anxiety, depression, epilepsy, mood, and sleep therapeutic indications were used here.

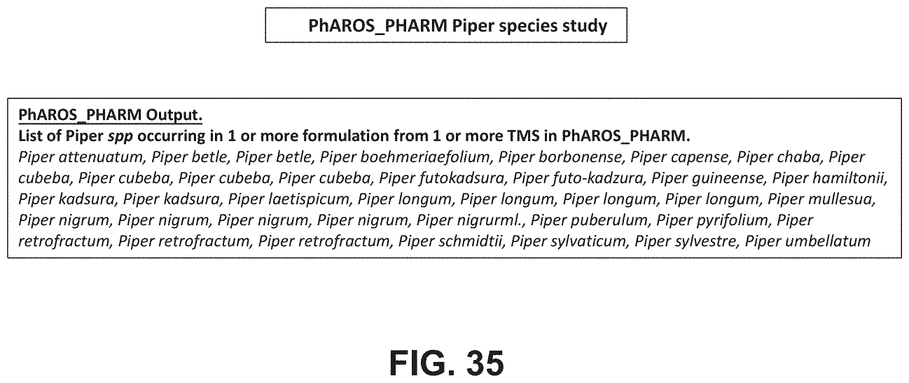

Outputting the processed data returned by the query revealed: a list of piper species associated with one or more therapeutic indications. For example, FIG. 35 provides a list of Piper species including Piper attenuatum, Piper betle, Piper betle, Piper boehmeriaefolium, Piper borbonense, Piper capense, Piper chaba, Piper cubeba, Piper cubeba, Piper cubeba, Piper cubeba, Piper futokadsura, Piper futo-kadzura, Piper guineense, Piper hamiltonii, Piper kadsura, Piper kadsura, Piper laetispicum, Piper longum, Piper longum, Piper longum, Piper longum, Piper mullesua, Piper nigrum, Piper nigrum, Piper nigrum, Piper nigrum, Piper nigrurml., Piper puberulum, Piper pyrifolium, Piper retrofractum, Piper retrofractum, Piper retrofractum, Piper schmidtii, Piper sylvaticum, Piper sylvestre, and Piper umbellatum.

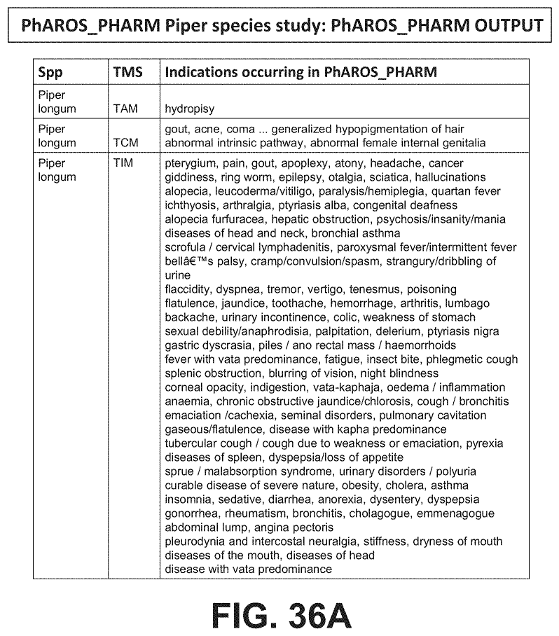

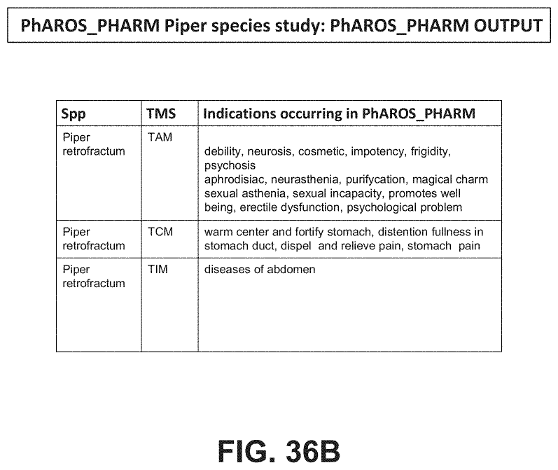

Each Piper species within the list of Piper species (FIG. 35) was associated with one or more TMS, therapeutic indications within the one or more TMS (see, e.g., FIGS. 36A-B), and sets of chemical components linked to each Piper species and associated with the therapeutic indication.

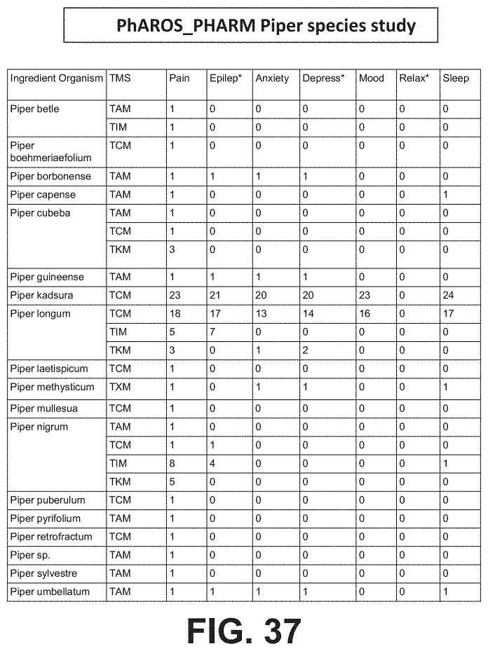

As noted above, the aim here was to identify alternatives to P. methysticum for treating anxiety, pain, relaxation and epilepsy based on the restricted biogeography pf P. methysticum. Representation of Piper spp in formulations derived from the various TMS in PhAROS_PHARM and associated with indications mined using a custom dictionary that included pain, epilepsy, anxiety, depression, mood and sleep. FIG. 37 shows representative Piper spp in formulations derived from the various TMS in PhAROS_PHARM and associated with indications mined using a custom dictionary that included pain, epilepsy, anxiety, depression, mood and sleep.

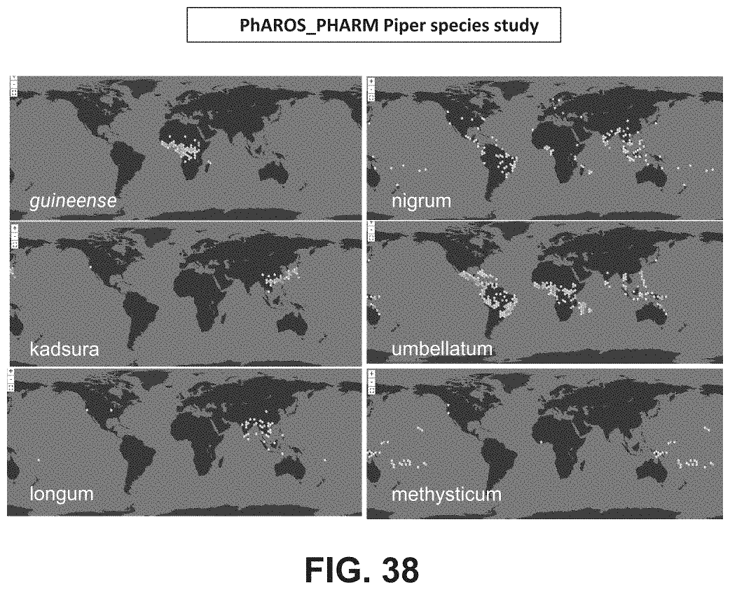

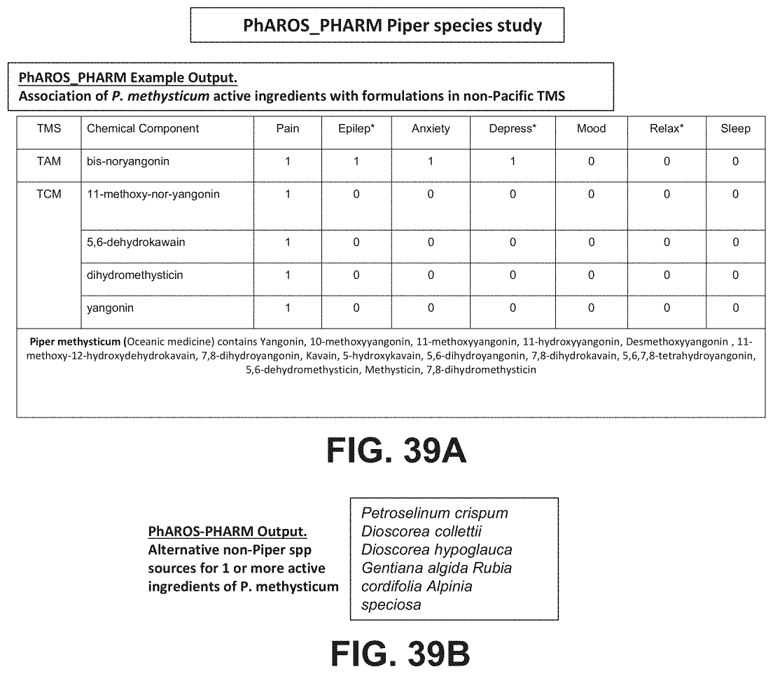

Next, PhAROS was used to inquire if TMS formulae for pain, epilepsy, anxiety, depression, mood, relaxation, and sleep contained the Kavalactones that are associated with the efficacy of the highly biogeographically-restricted and culturally-sensitive P. methysticum. In particular, the aim was to identify alternatives to P. methysticum for anxiety, pain, relaxation and epilepsy based on the restricted biogeography pf P. methysticum, as previously noted. FIG. 38 shows comparative biogeography of Piper spp that are indicated for the disorders of interest. FIG. 39A shows association of P. methysticum active ingredients with formulations in the non-Pacific TMS TAM (traditional African medicine) and TCM (traditional Chinese medicine), and FIG. 39B shows non-Piper species sources for 1 or more active ingredients of P. methysticum, selected at least in part on biogeography. FIG. 40 shows the complete compound set for all Piper ingredient organisms associated with anxiety in PhAROS_PHARM.

Overall, this example showed that PhAROS could be used to identify alternatives to P. methysticum for anxiety, pain, relaxation and epilepsy based on the restricted biogeography of P. methysticum.

### PhAROS_PHARRM Anxiety Machine Learning Study

Next, an unbiased machine learning method was used to identify alternatives to P. methysticum for anxiety, pain, relaxation, and epilepsy based on the restricted biogeography of P. methysticum. The machine learning approach was designed to treat every piece of data and metadata in the PhAROS_PHARM computational space as a feature and ask, of these features, which best predict an association with the anxiety/mood/depression dictionary. A feature's ability to predict the anxiety/mood/depression indications was normalized to all other indications.

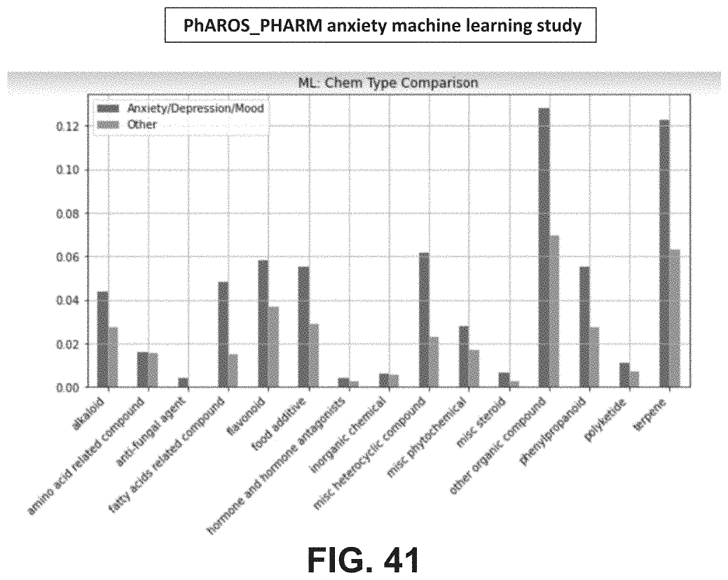

A PhAROS_PHARM machine learning output, including chemical component type classes, was assessed for the ability to predict an anxiety/mood/depression indication over all other indications. Specific chemical type features most predictive of anxiety/mood/depression utility of a formulation were: alkaloid, terpene, fatty acid-related compounds, flavonoid, and phenyl propanoid (See FIG. 41). Coincidence with use as food additives, miscellaneous heterocyclic classification, and other organic compound classification (including above classes) was observed.

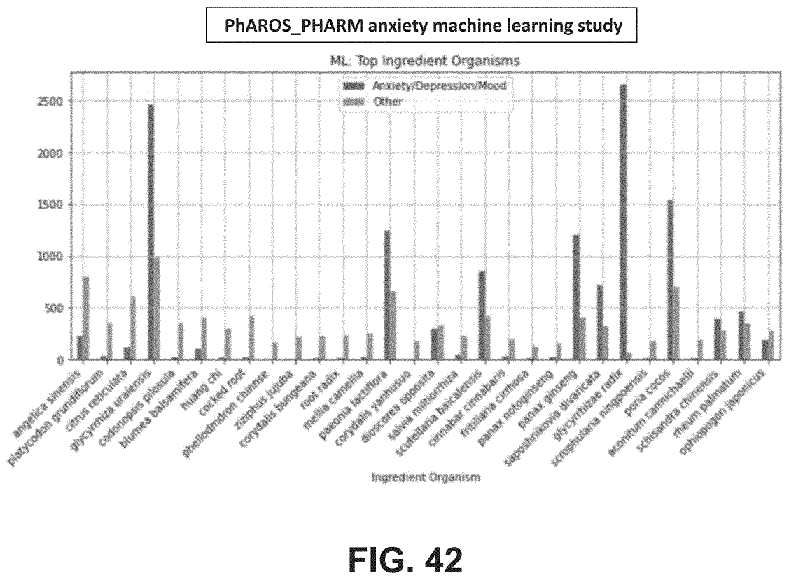

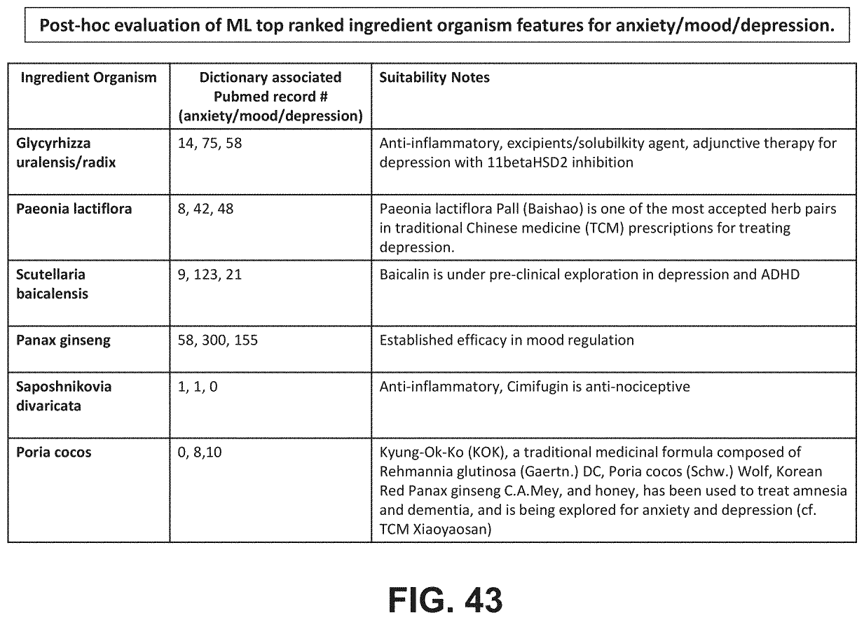

PhAROS_PHARM machine learning outputs, including ingredient organisms, were assessed for their ability to predict an anxiety/mood/depression indication over all other indications. Specific ingredient organisms most predictive of anxiety/mood/depression utility of a formulation were: Glycyrhizza uralensis radix, Paeonia lactiflora, Scutellaria baicalensis, Panax ginseng, Saposhnikovia divaicata, and Poria cocos (see FIG. 42). Post-hoc evaluation of top ranked ingredient organisms features for anxiety/mood/depression is shown in FIG. 43.

Overall, the machine learning approach identified the top ranked chemical components and specific ingredient organisms that could serve as a basis for identifying new polypharmaceuticals.

### Example 4. PhAROS_PHARM Divergence Analysis of Cancer & Pain in Database to Find Novel Cytotoxic Agents

In this example, PhAROS convergence analysis (PhAROS_CONVERGE) and PhAROS divergence analysis (PhAROS_DIVERGE) were used to identify potential cytotoxic agents that might become a part of a novel cancer therapy and, separately, within complex TMS formulations for cancer and to identify compound sets with potential for cancer pain over other pain subtypes.

In this example, the hypothesis was that TMS formulations for cancer will display significant convergence with pain since pain is likely to be a major symptom in historical and contemporary presentations by cancer patients to TM practitioners. Conversely, in the divergent compound group between cancer and pain there are likely to be cytotoxic (growth inhibitory) chemical components that may be explored for untapped therapeutic utility. Therefore, this study had two aims: (1) use in silico convergence and divergence analysis in PhAROS to identify potential cytotoxic agents within complex TMS formulations for Cancer and (2) identify compound sets with potential for cancer pain over other pain subtypes.

Here, PhAROS was used to discover polypharmaceutical medicines for treating cancer by analyzing, in a single computational space, data from a plurality of traditional medicine systems (TMS), where the analysis used transcultural dictionaries to allow searches within distinct TMS data sets embodying different epistemologies and terminologies, and where the analysis uses data returned by a query to identify new polypharmaceutical and/or optimized polypharmaceutical compositions for use in treating cancer pain over other pain subtypes. The query(s) included three clinical indications (i) cancer, (ii) cancer pain, and (iii) cancer and cancer pain.

In this example, data analysis included a subset of the Inputs as described in FIG. 8. For example, we analyzed, in a single computational space, data from a plurality of traditional medicine systems (TMS), included normalized formalized pharmacopeias from one or more geographic regions associated with TMS (i.e., PhAROS_PHARM).

As part of the analysis, transcultural dictionaries that collate Western and non-Western epistemological understanding of cancer, cancer-like patient presentations, cytotoxic agents within TMS formulations for cancer, and cancer pain were part of the TMS used here. The transcultural dictionaries included a list of compounds associated with cancer pain, and a list of compounds known for treating pain. In addition, the transcultural dictionaries were further populated with additional data developed by a machine learning algorithm that generated a therapeutic indication dictionary for: cancer, cancer pain, and cancer and cancer pain.

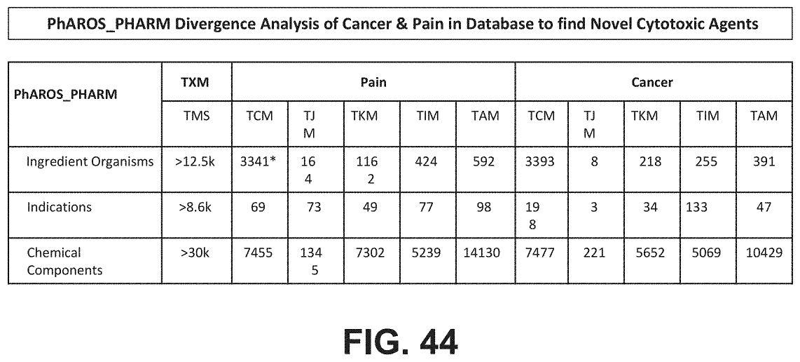

Outputting the processed data returned by the query (i.e., clinical indications including cancer, cancer pain, and cancer and cancer pain) produced a list of compounds associated with the user selected clinical indications, a list of prescription formulae for a given TMS, and a list of organisms associated with the user selected clinical indication (FIG. 44).

ML predictions showed that >80% of the chemical components of cancer medications in PhAROS are also found in pain medication.

The outputted, processed data included cytotoxic agents within the list of compounds that are indicated for pain and cancer across one or more TMS. This created a CANCERPAIN master list of compounds for subsequent comparison with ALLPAIN.

Divergence analysis of the compound list included identifying a list of compounds associated with a first user-selected clinical indication (i.e., cancer), where the list of compounds that is associated with the first user-selected clinical indication (i.e., cancer) does not overlap with a list of compounds that is associated with a second user-selected indication (i.e., pain).

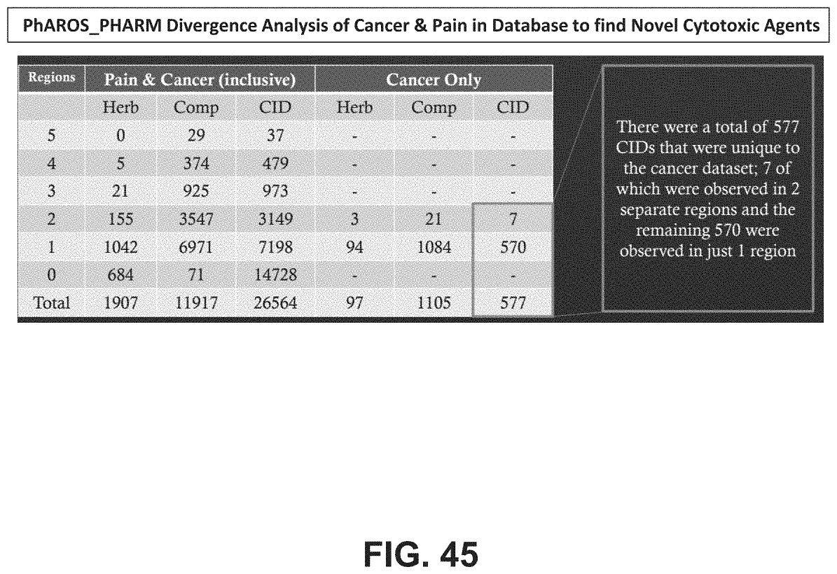

The divergence analysis identified a divergent chemical component subset between cancer and pain indications, which can now be mined for cytotoxic components using PhAROS_CHEMBIO and PhAROS_TOX (FIG. 45).

Results for the ML predictions included: cancer and pain medicine component overlap most of the time; a CANCERPAIN master list of compounds that is available for subsequent comparison with ALLPAIN; ML predictions show that >80% of the chemical components of cancer medications in PhAROS are also found in pain medication; a divergent chemical component subset has been identified between cancer and pain indications, which can now be mined for cytotoxic components using PhAROS_CHEMBIIO and PhAROS_TOX; and ML can assess ingredient organisms most likely to contain chemical components that diverge between cancer and pain (i.e., most likely cytotoxic or non-analgesic ingredients).

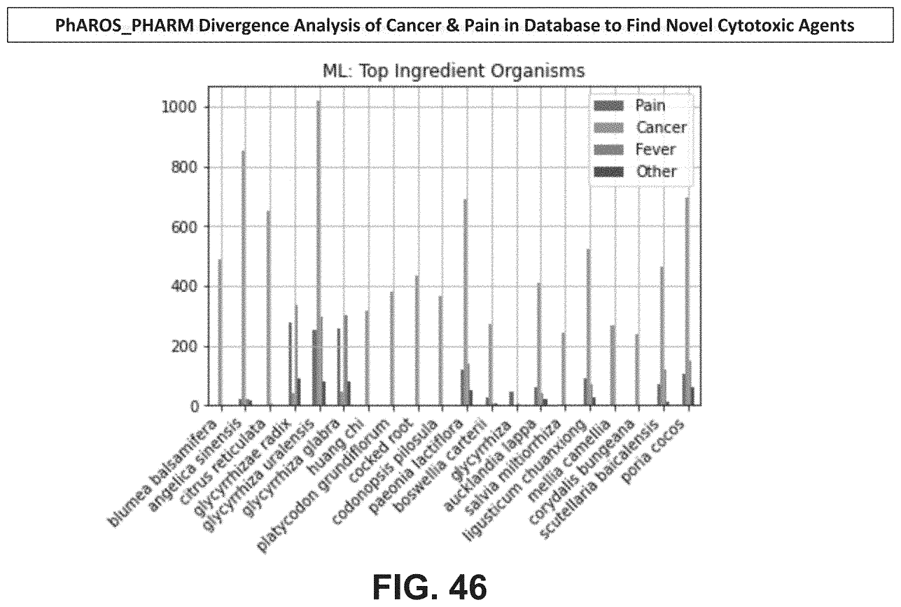

Further assessment using machine learning of ingredient organisms most likely to contain chemical components that diverge between cancer and pain (i.e., most likely cytotoxic or non-analgesic ingredients) is shown in FIG. 46. This analysis suggests that PhAROS can now be used to assess the most likely compounds within the top performing ingredient organisms as demonstrated in prior examples.

Overall, this example showed that PhAROS-based divergence analysis can be used to identify potential cytotoxic agents within complex TMS formulations for cancer and identify compound sets with particular potential for cancer pain over other pain subtypes.

### Example 5. World Health Initiatives & Alternative Supply Chain Proof-of Concept

In this example, PhAROS was used to identify alternative sources for medically important phytochemicals that have distinct biogeographies.

Polypharmaceuticals (phytomedicines) are limited by supply chain issues. There is a constellation of approaches to this challenge including total synthesis, bioreactor approaches and alternate sourcing. The PhAROS methods as described herein can be used to inform the latter, through interrogation of PhAROS_PHARM with a compound of interest or formulation and the generation of an output list of plant sources. Within PhAROS, data can come from metabolomic data (PhAROS_METAB) (where available) or commissioned to evaluate for relative abundance of the compound of interest. In addition, as supply chains have geographical, climatological and environmental limitations, the most recognized sources of a particular phytomedical compound are associated with specific locales. Therefore, using PhAROS_BIOGEO enables analysis of growing conditions overlaid on a geographic information system (GIS) framework to identify viable growing locales for plant sources of specific compounds. Overall, PhAROS outputs based on the analysis described herein can provide decision support for supply chain and logistics issues for phytomedical companies.

In order to widely adopt phytomedical components into mainstream medicine the issue of supply chain availability needs to be addressed because: (1) the best understood plant sources may be endangered or geographically-restricted, (2) alternative sources may be easier to extract leading to production efficiencies, (3) many complex phytotherapeutics are not amenable to total synthesis so supply chain expansion would be needed for their eventual widespread usage.

In this example, a list of phytomedically important compounds for indications ranging from cancer to pain was assembled using PubMed searches. This test set (user input query) was used to interrogate PHAROS_PhARM to identify plant sources, known indications and TMS systems in which the compound was used, and for what indications. Data integration via Global Biodiversity Information Facility (GBIF) was used to assess biogeography.

In particular, the PhAROS method was used to identify (discover) alternative sources of phytochemicals by analyzing in a single computational space, data from a plurality of traditional medicine systems (TMS), where the analysis used transcultural dictionaries to allow searches within distinct TMS data sets embodying different epistemologies and terminologies, and where the analysis uses data returned by a query to identify new polypharmaceutical and/or optimized polypharmaceutical compositions, including alternative sources for phytochemicals included in the polypharmaceutical compositions. A list of phytomedically important compounds for indications ranging from cancer to pain was assembled using PubMed searches. This test set was used to interrogate PhAROS_PHARM to identify plant sources, known indications and TMS systems in which the compound was used, and for what indication.

The query was to identify alternative sources for the set of compounds or formulations. The compounds/formulations were identified using PubMed searches for compounds treating indications ranging from cancer to pain.

Data analysis included a subset of the Inputs as described in FIG. 8. For example, we analyzed, in a single computational space, data from a plurality of traditional medicine systems (TMS), including normalized formalized pharmacopeias from one or more geographic regions associated with TMS (i.e., PhAROS_PHARM); meta-pharmacopeia with de novo metabolomic data for plants, and organisms that are not currently in medicinal use, supplemental metabolomic data secured for known medicinal plants and/or associated organisms (i.e., PhAROS_METAB); and meta-pharmacopeia, associated temporally, geographical, botanical, climatological, environmental, genomic, metagenomic, and metabolomic data on originating plants, components or other organisms (i.e., PhAROS_BIOGEO).

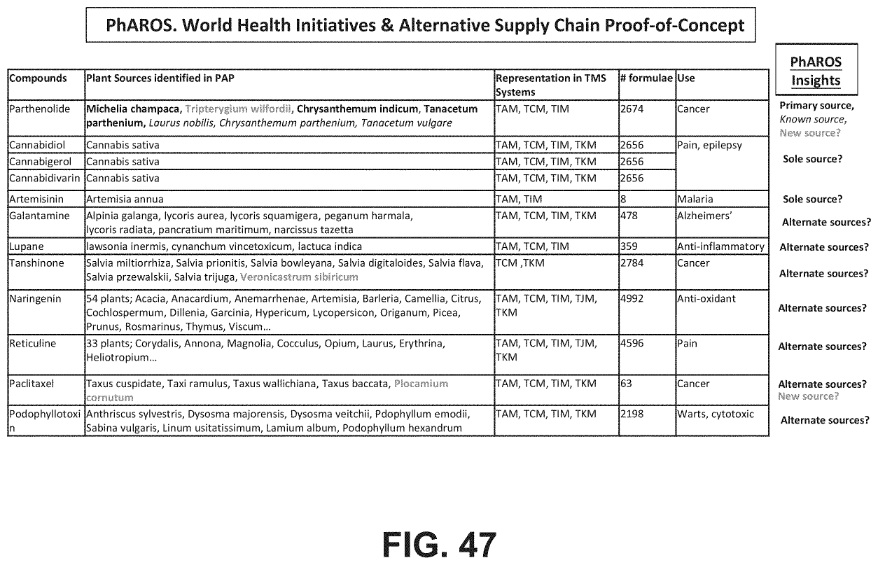

The output returned by the first user input query (i.e., the list of one or more phytochemical compounds or formulations) produced a list of plant sources, known clinical indications associated with the phytomedical compounds or formulations, the TMS in which each compound was referenced, and a relative abundance of the one or more compounds or formulations available (See FIG. 47). FIGS. 48A-B show processed data for compounds: parthenolide, paclitaxel, and tanshinone, including growing locations of the plant sources (FIG. 48B).

### Analysis of Potential Supply Chain Species for Parthenolide

As an example, we further explored the potential supply chain species for Parthenolide (PTL), a sequiterpene lactone from Tanacetum parthenium (Feverfew). It has been used across traditional and indigenous Western systems for analgesia and anti-inflammatory properties. The historical pharmacological knowledge underlying this application has been validated in the last two decades by controlled, mechanistic scientific studies that show efficacy in migraine (through the targeting of TRP Transient Receptor Potential ion channels) and inflammation (e.g. in rheumatoid arthritis, inflammation associated with cystic fibrosis and the murine EAE MS model). Feverfew itself is well-tolerated with minor adverse events (flatulence, bloating, heartburn, diarrhea). There are isolated reports of Feverfew acting as a contact allergen and exerting anti-platelet effects which require monitoring or cessation of Feverfew extract exposure. These side effects create a potential clinical need to identify alternative sources of PTL, while supply chain, logistic and local production issues would also motivate the identification of sources outside Feverfew.

PTL, considered to be the main active ingredient in Feverfew, is a sesquiterpene with 15 carbon atoms, 3 isoprene units and an alpha methylene-gamma lactone moiety (a cyclic ester). PTL appears to have direct cytotoxic effects and its anti-inflammatory effects may also decrease tumor success due to the close linkages between oncogenic proliferation and inflammation. PTL interrupts cell cycle progression and induces apoptosis and there is evidence that PTL decreases tumor size in vivo. Guzman et al. have shown effectiveness of PTL in AML, where effectiveness appears to relate to the constitutive activation of NFκB in AML cells compared to normal myeloid cells. PTL is likely to impact transformed cells in multiple ways, including the fact that through acting as a Michael acceptor it can participate in adduct formation which in turn can target enzymes such as DNA polymerase. However, the primary target protein for the cytotoxic effects of sesquiterpene lactones including PTL is NFκB, which is central to cell cycle progression and cell growth and is an anti-oncogene. Importantly, the co-targeting of proliferation and inflammation through NFκB gives PTL the potential for a ‘one-two punch’ for cancer—hitting both uncontrolled proliferation and the facilitating inflammatory milieu in which tumors tend to be more successful. Moreover, studies show that PTL can critically inhibit Cancer Stem Cells (CSC) in the context of non-small cell lung carcinoma, melanoma, multiple myeloma and nasopharyngeal carcinoma, again working via NFκB inhibition. This multi-faceted potential of PTLs creates their potential to be truly blockbusting, game changing drugs in difficult-to-treat cancers.

The biogeographical analyses in FIGS. 48A-B show that the additional species identified as parthenolide sources in PhAROS alter dramatically the geographical range of the PTL supply chain when compared to the archetypal source Feverfew.

As shown in FIGS. 49A-B, comparison of processed data from PhAROS with data from literature sources revealed PhAROS can be used to identify alternative organisms as sources of phytomedically-important compounds; new or relatively understudied organism sources of phytomedically-important compounds; and sources of phytomedically-important compounds linked to specific growing locations to inform supply chain design.

### Example 6. Migraine: Transcultural Formulations, Minimal Essential Formulations

In this example, PhAROS was used to design new polypharmaceutical approaches for treating migraine.

There is an unmet need for migraine treatments for at least several reasons. First, triptans are not effective against all migraines. Second, opioids and barbiturates have high addiction potential. Third, ergotamine has nausea, vomiting and cardiovascular side effects, and is contraindicated for use in combination with a range of common drugs (antibiotics, anti-retrovirals, antidepressants). As such, the aim of this study was to identify transcultural and minimal essential components to design new polypharmaceutical approaches to migraine. The approach used was to apply migraine dictionary to PhAROS_PHARM and to perform data integration with PhAROS_MOLBIO, etc.

Briefly, here, the PhAROS method was used to discover polypharmaceutical medicines for treating migraine by analyzing, in a single computational space, data from a plurality of traditional medicine systems (TMS) (e.g., including, without limitation, normalized formalized pharmacopeias from one or more geographic regions associated with TMS (i.e., PhAROS_PHARM) and medical compound data sets comprising chemical and biological data of medical compounds (i.e., PhAROS_CHEMBIO)), where the analysis used transcultural dictionaries to allow searches within distinct TMS data sets embodying different epistemologies and terminologies, and where the analysis uses data returned by a query to identify new polypharmaceutical and/or optimized polypharmaceutical compositions.

As the clinical indication is migraine, the query (i.e., the first user input query) is to identify new polypharmaceutical and/or optimized polypharmaceutical compositions for migraine.

Data analysis included a subset of the Inputs as described in FIG. 8. We analyzed, in a single computational space, data from a plurality of traditional medicine systems (TMS), including normalized formalized pharmacopeias from one or more geographic regions associated with TMS (i.e., PhAROS_PHARM) and medical compound data sets comprising chemical and biological data of medical compounds (i.e., PhAROS_CHEMBIO).

As part of the analysis, transcultural dictionaries that collate Western and non-Western epistemological understanding of migraine and migraine-like patient presentations were used here. The transcultural dictionaries with additional data developed by a machine learning algorithm generated a therapeutic indication dictionary where migraine was the indication. FIG. 50A shows an example therapeutic indication dictionary for migraine.

Outputting the processed data returned by the first user input query (i.e., migraine as the clinical indication) produced a list of compounds associated with the user selected clinical indication (i.e., migraine), and a list of prescription formulae for any given TMS associated with the user selected clinical indication. FIG. 50B shows a summary of the processed data grouped by region, formulations that contain a migraine indication dictionary hit, and the total formulas. Here, 26 alkaloid or terpene compounds solely indicated for migraine with maximal convergence=2 TMS. The regions are represented by TAM (traditional African medicine), TCM (traditional Chinese medicine), TIM (traditional Indian medicine), TIM (traditional Japanese medicine), and TKM (traditional Korean medicine).

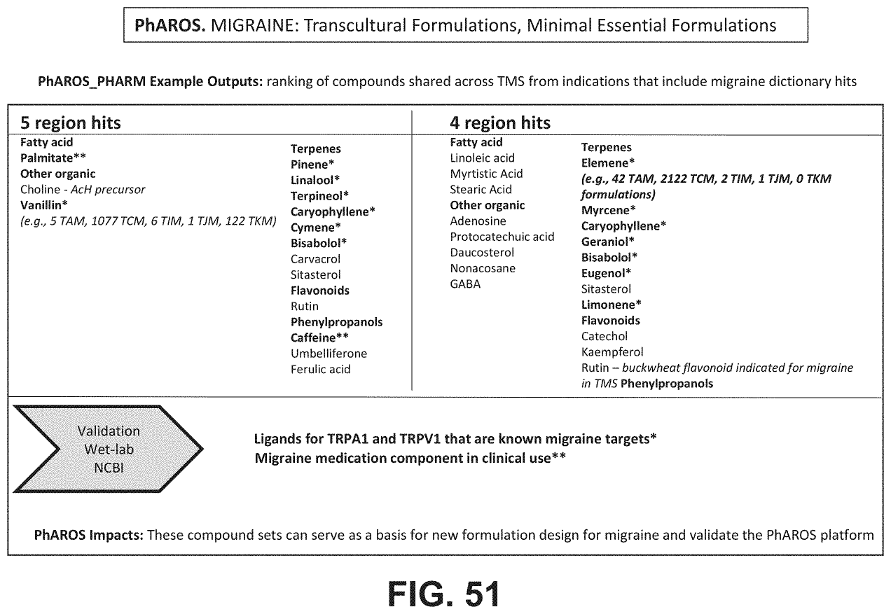

The processed data also included a list of molecular targets for the list of compounds that are clinically indicated for migraine across one or more TMS. FIG. 50C shows the molecular targets for all compounds identified in this Example (see, e.g., FIG. 50B for a summary and FIG. 51 for a subset of the compounds clinically indicated for migraine).

FIG. 51 shows a subset of the list of compounds associated with the user selected migraine indication where the compounds are ranked by efficacy and grouped by the number of geographic regions from which the compound can be found in a TMS data set. Left panel of FIG. 51 shows compounds ranked by efficacy and identified in 5 geographic regions, meaning the TMS data sets from which these compounds were identified originated from at least 5 geographic regions. Right panel of FIG. 51 shows compounds ranked by efficacy and identified in TMS data sets from four geographic regions. These compounds can serve as a basis for new formulation design for migraine and used to validate the PhAROS platform.

### Neurotropic Fungi-Derived Components of a Novel Polypharmaceutical Formulation to Further Evaluate for Migraine

In this example, the hypothesis was that the PhAROS method could identify alternatives to ergotamine. In particular, the aim was to identify neurotropic fungi indicated for migraines in TMS using PhAROS to output data.

First, using text mining, 209 neurotropic fungi were identified, including: Claviceps, Cordyceps, Gerronema, Mycena, Amanita, Pluteus, Copelandia, Panacolina, Panaeolus, Agrocybe, Conocybe, Hypholom, Psilocybe, Gymnopilus, Inocybe, Boletus, Hemiella, Russula, Lycoperdon, Vascellum. The 209 neutrotropic fungi were assessed against TCM, TKM, TIM, TAM, and TJM using PhAROS.

Only two neurotropic fungi (Claviceps purpurea (TCM) and Amanita muscaria (TIM)) appeared in any TMS associated with migraine.

Indications for Claviceps purpurea (TCM) and Amanita muscaria (TIM) include migraine pain, migraine pain and post-partum bleeding, and anti-poison.

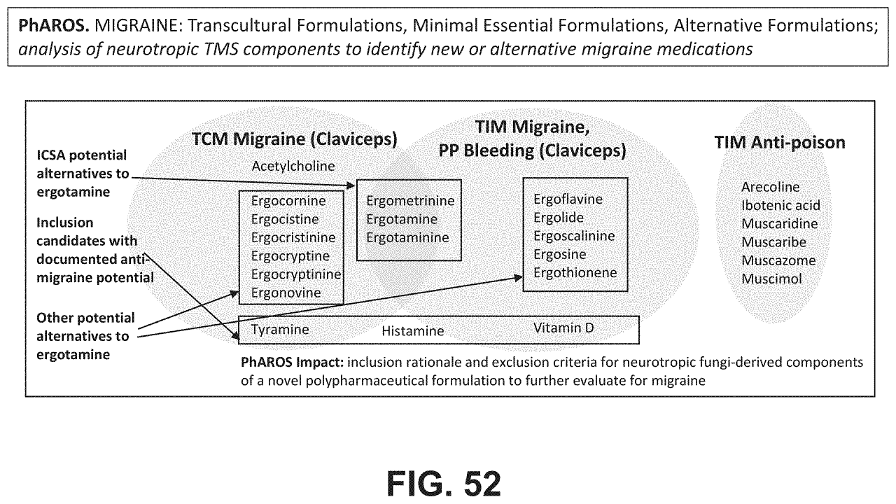

As shown in FIG. 52, analysis of the compounds from each of these indications revealed three compounds common across TCM/TIM migraine: ergometrinine, ergotamine and ergotaminine, two of which could serve as potential alternatives to ergotamine. Additionally, the analysis shown in FIG. 52 revealed identification of other candidates with documented anti-migraine potential and other potential alternative to ergotamine.

This example showed that PhAROS can identify new polypharmaceuticals for treating migraine that will be validated using traditional wet lab processes.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A phytomedicine analytics for research optimization at scale (PhAROS) method for discovering and/or optimizing polypharmaceutical medicines, the PhAROS method comprising:
analyzing, in a single computational space, data from a plurality of traditional medicine systems (TMS),
wherein the analysis uses transcultural dictionaries to allow searches within distinct TMS data sets embodying different epistemologies and terminologies,
wherein the analysis uses data returned by a query to identify new polypharmaceutical and/or optimized polypharmaceutical compositions.

2. The method of claim 1, wherein the data from the plurality of TMS comprise at least one of: medical formulations; organisms; medical compound data sets; therapeutic indications; processed and normalized formalized pharmacopeias from one or more geographic regions associated with TMS; therapeutic indication dictionaries related to traditional medical systems that reflect modern and historical terminology; Western and non-Western epistemologies; temporal and geographical data indicating historical and contemporary geographical, cultural and epistemology origins; raw and optionally pre-processed data from a plurality of traditional medicine data sets, plant data sets, and literature-based text documents (corpus).

3. (canceled)

4. The method of claim 2, wherein the one or more processed and normalized formalized pharmacopeias comprises at least one of processed data, translated normalized data, individual published datasets, or case reports in the scientific literature that document relationships between medicinal plants and disease indications.

5. The method of claim 2, wherein the one or more processed and normalized formalized pharmacopeias comprises at least one of processed data, curated ethical partnerships, indigenous phytomedical formulations, and cultural (African, Oceanic) phytomedical formulations.

6. The method of claim 1, wherein the one or more processed and normalized formalized pharmacopeias comprises processed contemporary and historical herbologies that document relationships between medicinal plants and disease indications, wherein the herbologies are optionally selected from Hildegard of Bingen, Causae et Curae, and Physica.

7. The method of claim 2, wherein the one or more processed and normalized formalized pharmacopeias comprises processed translations from original languages, wherein the process uses methods selected from one or more of:
machine literal translation, natural language processing, multilingual concept extraction or conventional translation, Optical character recognition (OCR) of historical materials, and artificial intelligence (AI)-driven intent translation.

8. The method of claim 7, wherein the medical compound data sets comprise chemical and biological data of medical compounds, wherein the chemical and biological data of medical compounds comprise one or more of: chemical structure, physicochemical properties, known and/or algorithmically calculated or predicted PD/PK properties, putative biological effects, data with respect to receptor binding, docking, regulation of signaling pathways, metabolism, drug-target relationships, mechanism of action, CYP interactions, or published studies and clinical trials of the medical compounds.

9. (canceled)

10. The method of claim 1, wherein the raw and optionally pre-processed data normalized from a plurality of traditional medicine data sets comprises one or more of:
meta-pharmacopeia associated temporally, geographical, botanical, climatological, environmental, genomic, metagenomic, and metabolomic data on originating plants, components or other organisms;
meta-pharmacopeias with de novo metabolomic data for plants and organisms that are not currently in medicinal use, supplemental metabolomic data secured for known medicinal plants and/or associated organisms; and
toxicological and side-effect profile data of medical compound data sets, de novo experimentally-derived data of medical compound data sets, and/or in silico predicted toxicological and side-effect data of medical compound data sets.

11. The method of claim 1, wherein analyzing comprises, first, receiving a user query from a user.

12-16. (canceled)

17. The method of claim 11, wherein processing the searched data comprises performing an in silico convergence analysis to search drug-target-indication relationships associated with the user query input.

18-22. (canceled)

23. The method of claim 11, wherein processing the searched data comprises performing an in silico divergence analysis comprising identifying alternative compounds derived from one or more organisms, and therapeutic approaches from biogeographically and culturally separated locales across the plurality of TMS.

24-32. (canceled)

33. The method of claim 1, wherein the optimized polypharmaceutical composition comprises a reduced number of compounds within the optimized polypharmaceutical composition as compared to an existing transcultural medicinal formulation, wherein the optimized polypharmaceutical composition comprises a minimal number of essential compounds to achieve a therapeutic outcome.

34. The method of claim 33, wherein said further analysis comprises, after outputting one or more selected from:
developing training data sets for one or more machine learning models to optimize the transcultural dictionaries;
populating the transcultural dictionaries with additional data developed by a machine learning algorithm; and
creating, updating, annotating, processing, downloading, analyzing, or manipulating the data from the plurality of TMS.

35-39. (canceled)

40. The method of claim 1, wherein at least one transcultural dictionary of the transcultural dictionaries comprises a search dictionary that collates Western and non-Western epistemological understanding of migraine and migraine-like patient presentations.

41-58. (canceled)

59. The method of claim 11, wherein the user query input comprises one or more phytomedical compounds or formulations, and optionally a current source (plant or animal) and supply of the compound or formulation.

60-66. (canceled)

67. The method of claim 34, wherein populating the transcultural dictionaries with additional data developed by the machine learning algorithm comprises generating a therapeutic indication dictionary.

68. (canceled)

69. The method of claim 40, wherein the first user input query comprises a user selected clinical indication, wherein the user selected clinical indication is pain.

70-96. (canceled)

97. The method of claim 1, wherein at least one transcultural dictionary of the transcultural dictionaries comprises a search dictionary that collates Western and non-Western epistemological understanding of piper species associated with a therapeutic indication.

98-120. (canceled)

121. The method of claim 34, wherein populating the transcultural dictionaries with additional data developed by the machine learning algorithm comprises generating a therapeutic indication dictionary.

122-131. (canceled)

132. A phytomedicine analytics for research optimization at scale (PhAROS) system for analyzing a plurality of traditional medical systems in a single computational space, the PhAROS system comprising:
a computer server configured to communicate with one or more user clients (PhAROS_USER), comprising:
(a) a database (PhAROS_BASE) comprising a memory configured to store a collection of data, the collection of data comprising:
raw and optionally pre-processed data from a plurality of traditional medicine data sets; and
optionally one or more of:
plant data sets;
literature-based text documents (corpus); and
machine learning data sets;
(b) a computer core processor (PhAROS_CORE), wherein the PhAROS_CORE is configured to receive and process the collection of data from the PhAROS_BASE to generate processed data;
(c) one or more searchable repositories having data and optionally pre-processed data, wherein each searchable repository comprises a memory configured to store data entries,
wherein the PhAROS_CORE is configured to send the processed data to and receive data from each of the searchable repositories,
wherein each of the searchable repositories is configured to receive processed data from the PhAROS_CORE and send data and optionally pre-processed data to the PhAROS_CORE;
(d) a computer-readable storage medium storing executable instructions that, when executed by a hardware processor, cause the PhAROS_CORE to communicate with the PhAROS_BASE and one or more of the searchable repositories to analyze data from a plurality of the traditional medicine data sets to produce an output responsive to a user query input into the PhAROS system.

133-152. (canceled)
